(19) United States
(12) Patent Application Publication
(10) Pub. No.: US 2011/0091971 A1
(43) Pub. Date: Apr. 21, 2011

(54) Differentiation of Pluripotent Stem Cells

(76) Inventors: Janet Davis, Skillman, NJ (US); Jiajian Liu, Skillman, NJ (US); Gopalan Raghunathan, San Diego, CA (US); Michael Joseph Hunter, San Diego, CA (US); Jose Pardinas, San Diego, CA (US); Judith Ann Connor, San Diego, CA (US); Ronald Vernon Swanson, San Diego, CA (US); Ellen Chi, San Diego, CA (US)

(21) Appl. No.: 12/493,741

(22) Filed: Jun. 29, 2009

Related U.S. Application Data
(60) Provisional application No. 61/076,889, filed on Jun. 30, 2008.

(51) Int. Cl. C12N 5/0735; C12N 5/071

(52) U.S. Cl. 435/377

(57) ABSTRACT

The present invention is directed to methods to differentiate pluripotent stem cells. In particular, the present invention is directed to methods and compositions to differentiate pluripotent stem cells into cells expressing markers characteristic of the definitive endoderm lineage. The present invention also provides methods to generate and purify agents capable of differentiating pluripotent stem cells into cells expressing markers characteristic of the definitive endoderm lineage.

The present invention claims priority to application Ser. No. 61/076,889, filed Jun. 30, 2008.

### FIELD OF THE INVENTION

The present invention is directed to methods to differentiate pluripotent stem cells. In particular, the present invention is directed to methods and compositions to differentiate pluripotent stem cells into cells expressing markers characteristic of the definitive endoderm lineage. The present invention also provides methods to generate and purify agents capable of differentiating pluripotent stem cells into cells expressing markers characteristic of the definitive endoderm lineage.

### BACKGROUND

Advances in cell-replacement therapy for Type I diabetes mellitus and a shortage of transplantable islets of Langerhans have focused interest on developing sources of insulin-producing cells, or β cells, appropriate for engraftment. One approach is the generation of functional β cells from pluripotent stem cells, such as, for example, embryonic stem cells.

In vertebrate embryonic development, a pluripotent cell gives rise to a group of cells comprising three germ layers (ectoderm, mesoderm, and endoderm) in a process known as gastrulation. Tissues such as, for example, thyroid, thymus, pancreas, gut, and liver, will develop from the endoderm, via an intermediate stage. The intermediate stage in this process is the formation of definitive endoderm. Definitive endoderm cells express a number of markers, such as, for example, HNF-3beta, GATA4, MIXL1, CXCR4 and SOX17.

Formation of the pancreas arises from the differentiation of definitive endoderm into pancreatic endoderm. Cells of the pancreatic endoderm express the pancreatic-duodenal homeobox gene, PDX1. In the absence of PDX1, the pancreas fails to develop beyond the formation of ventral and dorsal buds. Thus, PDX1 expression marks a critical step in pancreatic organogenesis. The mature pancreas contains, among other cell types, exocrine tissue and endocrine tissue. Exocrine and endocrine tissues arise from the differentiation of pancreatic endoderm.

Cells bearing the features of islet cells have reportedly been derived from embryonic cells of the mouse. For example, Lumelsky et al. (Science 292:1389, 2001) report differentiation of mouse embryonic stem cells to insulin-secreting structures similar to pancreatic islets. Soria et al. (Diabetes 49:157, 2000) report that insulin-secreting cells derived from mouse embryonic stem cells normalize glycemia in streptozotocin-induced diabetic mice.

In one example, Hori et al. (PNAS 99: 16105, 2002) discloses that treatment of mouse embryonic stem cells with inhibitors of phosphoinositide 3-kinase (LY294002) produced cells that resembled β cells.

In another example, Blyszczuk et al. (PNAS 100:998, 2003) reports the generation of insulin-producing cells from mouse embryonic stem cells constitutively expressing Pax4.

Micallef et al. reports that retinoic acid can regulate the commitment of embryonic stem cells to form PDX1 positive pancreatic endoderm. Retinoic acid is most effective at inducing PDX1 expression when added to cultures at day 4 of embryonic stem cell differentiation during a period corresponding to the end of gastrulation in the embryo (Diabetes 54:301, 2005).

Miyazaki et al. reports a mouse embryonic stem cell line over-expressing Pdx1. Their results show that exogenous Pdx1 expression clearly enhanced the expression of insulin, somatostatin, glucokinase, neurogenin3, p48, Pax6, and HNF6 genes in the resulting differentiated cells (Diabetes 53: 1030, 2004).

Skoudy et al. reports that activin A (a member of the TGF-β superfamily) upregulates the expression of exocrine pancreatic genes (p48 and amylase) and endocrine genes (Pdx1, insulin, and glucagon) in mouse embryonic stem cells.

The maximal effect was observed using 1 nM activin A. They also observed that the expression level of insulin and Pdx1 mRNA was not affected by retinoic acid; however, 3 nM FGF7 treatment resulted in an increased level of the transcript for Pdx1 (Biochem. J. 379: 749, 2004).

Shiraki et al. studied the effects of growth factors that specifically enhance differentiation of embryonic stem cells into PDX1 positive cells. They observed that TGFβ2 reproducibly yielded a higher proportion of PDX1 positive cells (Genes Cells. 2005 June; 10(6): 503-16).

Gordon et al. demonstrated the induction of brachyury [positive]/HNF-3beta [positive] endoderm cells from mouse embryonic stem cells in the absence of serum and in the presence of activin along with an inhibitor of Wnt signaling (US 2006/0003446A 1).

Gordon et al. (PNAS, Vol 103, page 16806, 2006) states: “Wnt and TGF beta/nodal/activin signaling simultaneously were required for the generation of the anterior primitive streak.”

However, the mouse model of embryonic stem cell development may not exactly mimic the developmental program in higher mammals, such as, for example, humans.

Thomson et al. isolated embryonic stem cells from human blastocysts (Science 282:114, 1998). Concurrently, Gearhart and coworkers derived human embryonic germ (hEG) cell lines from fetal gonadal tissue (Shamblott et al., Proc. Natl. Acad. Sci. USA 95:13726, 1998). Unlike mouse embryonic stem cells, which can be prevented from differentiating simply by culturing with Leukemia Inhibitory Factor (LIF), human embryonic stem cells must be maintained under very special conditions (U.S. Pat. No. 6,200,806; WO 99/20741; WO 01/51616).

D'Amour et al. describes the production of enriched cultures of human embryonic stem cell-derived definitive endoderm in the presence of a high concentration of activin and low serum (D'Amour K A et al. 2005). Transplanting these cells under the kidney capsule of mice resulted in differentiation into more mature cells with characteristics of some endodermal organs. Human embryonic stem cell-derived definitive endoderm cells can be further differentiated into PDX1 positive cells after addition of FGF-10 (US 2005/0266554A 1).

D'Amour et al. (Nature Biotechnology—24, 1392-1401 (2006)) states: “We have developed a differentiation process that converts human embryonic stem (hES) cells to endocrine cells capable of synthesizing the pancreatic hormones insulin, glucagon, somatostatin, pancreatic polypeptide and ghrelin. This process mimics in vivo pancreatic organogenesis by directing cells through stages resembling definitive endoderm, gut-tube endoderm, pancreatic endoderm and endocrine precursor en route to cells that express endocrine hormones.”

In another example, Fisk et al. reports a system for producing pancreatic islet cells from human embryonic stem cells (US2006/0040387A1). In this case, the differentiation pathway was divided into three stages. Human embryonic stem cells were first differentiated to endoderm using a combination of n-butyrate and activin A. The cells were then cultured with TGFβ antagonists such as Noggin in combination with EGF or betacellulin to generate PDX1 positive cells. The terminal differentiation was induced by nicotinamide.

In one example, Benvenistry et al. states: “We conclude that over-expression of PDX1 enhanced expression of pancreatic enriched genes, induction of insulin expression may require additional signals that are only present in vivo” (Benvenistry et al., Stem Cells 2006; 24:1923-1930).

Activin A is a TGF-β family member that exhibits a wide range of biological activities including regulation of cellular proliferation and differentiation, and promotion of neuronal survival. Activin A is a homo-dimer, consisting of two activin βA subunits, encoded by the inhibin A gene. Other activins are known consisting of homo- or hetero-dimers of βA βC, βD, and βE subunits. For example, activin B consists of a homo-dimer of two βB subunits. The peptides comprising the βA subunit and the βB subunit are 63% identical and the positions of eight cysteines are conserved in both peptide sequences.

Activin A exerts its effect on cells by binding to a receptor. The receptor consists of a heteromeric receptor complex consisting of two types of receptor, type 1 (ActR-I) and type II (ActR-II), each containing an intracellular serine/threonine kinase domain. These receptors are structurally similar with small cysteine-rich extracellular regions and intracellular regions consisting of kinase domains. ActR-I, but not ActR-II, has a region rich in glycine and serine residues (GS domain) in the juxtamembrane domain. Activin A binds first with ActR-II, which is present in the cell membrane as an oligomeric form with a constitutively active kinase. ActR-1, which also exists as an oligomeric form, cannot bind activin A in the absence of ActR-II. ActR-I is recruited into a complex with ActR-II after activin A binding. ActR-II then phosphorylates ActR-I in the GS domain and activates its corresponding kinase.

Isolation and purification of activin A is often complex and can often result in poor yields. For example, Pangas, S. A. and Woodruff, T. K states: “Inhibin and activin are protein hormones with diverse physiological roles including the regulation of pituitary FSH secretion. Like other members of the transforming growth factor-β gene family, they undergo processing from larger precursor molecules as well as assembly into functional dimers. Isolation of inhibin and activin from natural sources can only produce limited quantities of bioactive protein.” (J. Endocrinol. 172 (2002) 199-210).

In another example, Arai, K. Y. et al states: “Activins are multifunctional growth factors belonging to the transforming growth factor-β superfamily. Isolation of activins from natural sources requires many steps and only produces limited quantities. Even though recombinant preparations have been used in recent studies, purification of recombinant activins still requires multiple steps.” (Protein Expression and Purification 49 (2006) 78-82).

There have been considerable efforts to develop a more potent or cheaper alternative to activin A. For example, U.S. Pat. No. 5,215,893 discloses methods for making proteins in recombinant cell culture which contain the β or β chains of inhibin. In particular, it relates to methods for obtaining and using DNA which encodes inhibin, and for making inhibin variants that depart from the amino acid sequence of natural animal or human inhibins and the naturally-occurring alleles thereof.

In another example, U.S. Pat. No. 5,716,810 discloses methods for making proteins in recombinant cell culture which contain the α or β chains of inhibin. In particular, it relates to methods for obtaining and using DNA which encodes inhibin, and for making inhibin variants that depart from the amino acid sequence of natural animal or human inhibins and the naturally-occurring alleles thereof.

In another example, U.S. Pat. No. 5,525,488 discloses methods for making proteins in recombinant cell culture which contain the α or β chains of inhibin. In particular, it relates to methods for obtaining and using DNA which encodes inhibin, and for making inhibin variants that depart from the amino acid sequence of natural animal or human inhibins and the naturally-occurring alleles thereof.

In another example, U.S. Pat. No. 5,665,568 discloses methods for making proteins in recombinant cell culture which contain the α or β chains of inhibin. In particular, it relates to methods for obtaining and using DNA which encodes inhibin, and for making inhibin variants that depart from the amino acid sequence of natural animal or human inhibins and the naturally-occurring alleles thereof.

In another example, U.S. Pat. No. 4,737,578 discloses proteins with inhibin activity having a weight of about 32,000 daltons. The molecule is composed of two chains having molecular weights of about 18,000 and about 14,000 daltons, respectively, which are bound together by disulfide bonding. The 18K chain is obtained from the human inhibin gene and has the formula: H-Ser-Thr-Pro-Leu-Met-Ser-Trp-Pro-Trp-Ser-Pro-Ser-Ala-Leu-Arg-Leu-Leu-Gln-Arg-Pro-Pro-Glu-Glu-Pro-Ala-Ala-His-Ala-Asn-Cys-His-Arg-Val-Ala-Leu-Asn-Ile-Ser-Phe-Gln-Glu-Leu-Gly-Trp-Glu-Arg-Trp-Ile-Val-Tyr-Pro-Pro-Ser-Phe-R.sub.6 5-Phe-His-Tyr-Cys-His-Gly-Gly-Cys-Gly-Leu-His-Ile-Pro-Pro-Asn-Leu-Ser-Leu-Pro-Val-Pro-Gly-Ala-Pro-Pro-Thr-Pro-Ala-Gln-Pro-Tyr-Ser-Leu-Leu-Pro-Gly-Ala-Gln-Pro-Cys-Cys-Ala-Ala-Leu-Pro-Gly-Thr-Met-Arg-Pro-Leu-His-Val-Arg-Thr-Thr-Ser-Asp-Gly-Gly-Tyr-Ser-Phe-Lys-Tyr-Glu-Thr-Val-Pro-Asn-Leu-Leu-Thr-Gln-His-Cys-Ala-Cys-Ile-OH, wherein R.sub.65 is Ile or Arg. The 18K chain is connected by disulfide bonding to the 14K chain.

Therefore, there still remains a significant need for cheaper, more potent alternatives for activin A to facilitate the differentiation of pluripotent stem cells.

### SUMMARY

The present invention provides compounds capable of differentiating pluripotent stem cells into cells expressing markers characteristic of the definitive endoderm lineage. In one embodiment, the compounds capable of differentiating pluripotent stem cells into cells expressing markers characteristic of the definitive endoderm lineage are peptides comprising the amino acid sequence of activin A containing at least one point mutation.

In one embodiment, the present invention provides a method to differentiate pluripotent stem cells into cells expressing markers characteristic of the definitive endoderm lineage, comprising treating the pluripotent stem cells with a medium containing a peptide comprising the amino acid sequence of activin A containing at least one point mutation, for a period of time sufficient for the pluripotent stem cells to differentiate into cells expressing markers characteristic of the definitive endoderm lineage.

### BRIEF DESCRIPTION OF THE DRAWINGS

FIG. 1 shows the phylogenetic tree of peptides ACTN 2 to ACTN 48.

FIG. 2 shows the phylogenetic tree of peptides ACTN 49 to ACTN 94.

FIG. 3 shows the nucleic acid sequence of the pro-region of wildtype activin A that was cloned into pcDNA3.1(−).

FIG. 4 shows the nucleic acid sequence of the mature region of ACTN 1, cloned into pcDNA3.1(−).

FIG. 5 shows the nucleic acid sequence of the full-length gene for ACTN 1, containing the pro-region and the mature region, cloned into pcDNA3.1(−).

FIG. 6 shows the ability of ACTN 1 (♦ ACTN 1 WT) and a control activin A (▪ and ▴ OriGene WT) to differentiate human embryonic stem cells into cells expressing markers characteristic of the definitive endoderm lineage. ACTN 1 (♦ ACTN 1 WT) and a wildtype control activin A (▪ OriGene WT), as cloned into their respective mammalian expression vectors, were transfected into HEK293-E cells, and supernatants obtained neat (not conc) or concentrated (conc) were added to human embryonic stem cells at the assay dilutions shown. Differentiation was determined by measuring SOX17 intensity expression relative to untreated control cells.

FIG. 7 shows the expression constructs used to obtain the peptides of the present invention. Panel A shows the nucleic acid sequence of the full-length gene for ACTN 1, containing the pro-region and the mature region, cloned into pUNDER. A pictorial representation of the expression vector is shown in Panel B.

FIG. 8 shows the ability of ACTN 1 and a wildtype activin A control cloned into their respective mammalian expression vectors to differentiate human embryonic stem cells into cells expressing markers characteristic of the definitive endoderm lineage. Panel A shows the effect of supernatants on assay cell number. ACTN 1 cloned into pUNDER and a wildtype activin A control (OriGene) cloned into pCMV6-XL4 were transfected into HEK293-F cells (white bars) and CHO-S cells (black bars), and supernatants collected neat, or concentrated 10-fold were tested at the dilutions shown in the definitive endoderm bioassay. Data shown represent changes relative to untreated cells. Panel B shows the effect of supernatants on SOX17 expression. ACTN 1 cloned into pUNDER and a wildtype activin A control (OriGene) cloned into pCMV6-XL4 were transfected into HEK293-F cells (white bars) and CHO-S cells (black bars), and supernatants collected neat, or concentrated 10-fold were tested at the dilutions shown in the definitive endoderm bioassay. Data shown represent changes relative to untreated cells.

FIG. 9 shows the expression of the peptides of the present invention in supernatants of HEK293-F cells transfected with pUNDER vectors containing the genes encoding the full length peptides indicated (ACTN 2, ACTN 4, ACTN 5, ACTN 6, ACTN 7, and ACTN 8). Supernatants were obtained, and analyzed by Western blot; the membrane was probed with an anti-activin A antibody.

FIG. 10 shows the expression of the peptides of the present invention in supernatants of HEK293-F cells transfected with pUNDER vectors containing the genes encoding the full length peptides indicated (ACTN 9, ACTN 10, ACTN 11, ACTN 12, ACTN 14, ACTN 16, ACTN 17, ACTN 18, ACTN 19, ACTN 20, ACTN 21, ACTN 22 and ACTN 23). Supernatants were obtained, and analyzed by Western blot; the membrane was probed with an anti-activin A antibody.

FIG. 11 shows the expression of peptides of the present invention that were further modified to contain histidine substitutions. HEK293-F cells were transfected with pUNDER vectors containing the genes encoding ACTD 17, ACTD 18, ACTD 19, ACTD 20, ACTD 21, and ACTD 22. Supernatants were obtained, and analyzed by Western blot; the membrane was probed with an anti-activin A antibody (Mab 3381—left hand side), or an anti-precursor antibody (Mab 1203—right hand side).

FIG. 12 shows a representative IMAC purification profile for ACTD 20. After loading, the column was washed and protein eluted with a linear gradient of imidazole (0-500 mM) over 20 column volumes.

FIG. 13 shows the Western blot elution profiles for Imidazole fractions for ACTD 17, ACTD 18, ACTD 19, ACTD 20, ACTD 21, and ACTD 22.

FIG. 14 shows a representative Western blot for follistatin variant expression from the supernatants of HEK293-F cells transfected with vectors containing the follistatin genes ACTA 1, ACTA 2 and ACTA 3. The membrane was probed with the antibodies indicated.

FIG. 15 shows a representative IMAC purification profile for ACTA 3 (Panel A). After loading, the column was washed and protein eluted with a step gradient of Imidazole (10 mM, 50 mM, 150 mM, 250 mM and 500 mM). Panel B shows a silver stain gel of the elution profile for the IMAC purification.

FIG. 16 shows a Western blot (Panels A and B) of a representative purification of peptide variant ACTN 1 using an ACTA 3 affinity column. The membranes were probed with the antibodies indicated. Panel C shows a silver stain gel a representative purification of peptide variant ACTN 1 using an ACTA 3 affinity column.

FIG. 17 shows the differentiation of human embryonic stem cells into cells expressing markers characteristic of the definitive endoderm lineage. Differentiation was determined by measuring cell number (Panel A) and SOX17 intensity (Panel B) using an IN Cell Analyzer 1000 (GE Healthcare). Human embryonic stem cells were treated for four days with medium containing 20 ng/ml Wnt3a plus activin A at the concentrations indicated (black bars) or medium lacking Wnt3a but with activin A at the concentrations indicated (white bars).

FIG. 18 shows the ability of ACTN 1 (white bars) and a control activin A (hatched bars and solid bars) to differentiate human embryonic stem cells into cells expressing markers characteristic of the definitive endoderm lineage. Supernatants from HEK293-E cells transfected with ACTN 1 (white bars) and a control activin A (hatched bars), cloned into pcDNA3.1(−) were obtained and concentrated, then added to human embryonic stem cells at the dilutions shown. Differentiation was determined by measuring SOX17 intensity.

FIG. 19 shows the differentiation of human embryonic stem cells into cells expressing markers characteristic of the definitive endoderm lineage using activin A. Panel A shows a standard curve for human embryonic stem cell differentiation using commercial recombinant human activin A and measuring SOX17 intensity. Cells were treated with activin A at the concentrations indicated for four days. Data shown are mean expression levels of SOX17, as detected using an IN Cell Analyzer 1000 (GE Healthcare). Panel B shows the ability of ACTN 1 to differentiate human embryonic stem cells into cells expressing markers characteristic of the definitive endoderm lineage. Supernatants from HEK293-F cells (white bars) and CHO-S cells (black bars) transfected with ACTN 1 cloned into pUNDER (pUNDER), and a wildtype activin A control (OriGene) cloned into pCMV6-XL4 were added to human embryonic stem cells at the concentrations indicated, and SOX 17 expression levels were determined four days later.

FIG. 20 shows the standard curve of recombinant human activin A as supplied by the manufacturer of an activin A ELISA (Panel A). Panel B compares the standard curves of two commercial recombinant human activin A standards in an activin A ELISA, where open squares (□) indicate the activin A standard supplied by the manufacturer (R&D Systems) and closed triangles (▴) indicate activin A purchased from Peprotech.

FIG. 21 shows results using flow cytometric analysis for CXCR4 expression after various treatments during the first step of differentiation. Histograms with percentages of CXCR4 positive cells are shown for treatment with activin A, or no activin A or two variant histidine peptides (ACTD3 and ACTD8), tested as unpurified supernatant stocks or IMAC purified material.

FIG. 22 panels A through I, show relative percent intensity for SOX17 expression versus a dose titration of given peptide concentrations, where peptide concentrations were previously calculated from ELISA results. In each panel, representative curves compare wildtype activin A peptide (ACTN1) to a variant peptide. Relative fit for each of the curves is shown by representative R2 values.

FIG. 23 shows results at the conclusion of the first step of differentiation using flow cytometric, PCR, and high content measure for multiple markers representative of definitive endoderm. Panel A shows FACS analysis for CXCR4 expression using a commercial source of activin A or wild type ACTN1 peptide during differentiation treatment. Panel B shows CXCR4 expression for two variant peptides (ACTN4 and ACTN48) compared to the wild type ACTN1 peptide. Panels C through F show high content analysis for cell number and SOX17 expression at the end of the first step of differentiation after treatment with wildtype activin A or individual variant peptides. Panels G and H show RT-PCR results for SOX17 and FOXA2 gene expression at the conclusion of the first step of differentiation after treatment with wildtype ACTN1 or variant peptides ACTN4 or ACTN48. The inset box shows CT values for each of the gene markers.

FIG. 24 shows results at the conclusion of the third step of differentiation after treatment with wildtype ACTN1 or variant peptides ACTN4 or ACTN48 during the first step of differentiation. Results depict high content analysis for cell number (panels A and B), PDX1 protein expression (panels C and D), CDX2 protein expression (panels E and F), or RT-PCR results for PDX1 or CDX2 (panels G and H). The inset box shows CT values for each of the gene markers.

FIG. 25 shows RT-PCR results at the conclusion of step four of differentiation after treatment with wildtype ACTN1 or. variant peptides ACTN4 or ACTN48 during the first step of differentiation. The inset box shows CT values for each of the gene markers.

### DETAILED DESCRIPTION

For clarity of disclosure, and not by way of limitation, the detailed description of the invention is divided into the following subsections that describe or illustrate certain features, embodiments or applications of the present invention.

### DEFINITIONS

Stem cells are undifferentiated cells defined by their ability at the single cell level to both self-renew and differentiate to produce progeny cells, including self-renewing progenitors, non-renewing progenitors, and terminally differentiated cells. Stem cells are also characterized by their ability to differentiate in vitro into functional cells of various cell lineages from multiple germ layers (endoderm, mesoderm and ectoderm), as well as to give rise to tissues of multiple germ layers following transplantation and to contribute substantially to most, if not all, tissues following injection into blastocysts.

Stem cells are classified by their developmental potential as: (1) totipotent, meaning able to give rise to all embryonic and extraembryonic cell types; (2) pluripotent, meaning able to give rise to all embryonic cell types; (3) multipotent, meaning able to give rise to a subset of cell lineages but all within a particular tissue, organ, or physiological system (for example, hematopoietic stem cells (HSC) can produce progeny that include HSC (self-renewal), blood cell restricted oligopotent progenitors, and all cell types and elements (e.g., platelets) that are normal components of the blood); (4) oligopotent, meaning able to give rise to a more restricted subset of cell lineages than multipotent stem cells; and (5) unipotent, meaning able to give rise to a single cell lineage (e.g., spermatogenic stem cells).

Differentiation is the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

“β-cell lineage” refers to cells with positive gene expression for the transcription factor

PDX1 and at least one of the following transcription factors: NGN3, NKX2.2, NKX6.1, NEUROD, ISL1, HNF-3 beta, MAFA, PAX4, or PAX6. Cells expressing markers characteristic of the β cell lineage include β cells.

“Cells expressing markers characteristic of the definitive endoderm lineage”, or “Stage 1 cells”, or “Stage I”, as used herein, refers to cells expressing at least one of the following markers: SOX17, GATA4, HNF-3 beta, GSC, CERT, Nodal, FGF8, Brachyury, Mix-like homeobox protein, FGF4 CD48, eomesodermin (EOMES), DKK4, FGF17, GATA6, CXCR4, C-Kit, CD99, or OTX2. Cells expressing markers characteristic of the definitive endoderm lineage include primitive streak precursor cells, primitive streak cells, mesendoderm cells and definitive endoderm cells.

“Cells expressing markers characteristic of the pancreatic endoderm lineage”, as used herein, refers to cells expressing at least one of the following markers: PDX1, HNF-1 beta, PTF-1 alpha, HNF6, or HB9. Cells expressing markers characteristic of the pancreatic endoderm lineage include pancreatic endoderm cells, primitive gut tube cells, and posterior foregut cells.

“Cells expressing markers characteristic of the pancreatic endocrine lineage”, or “Stage 5 cells”, or “Stage 5”, as used herein, refers to cells expressing at least one of the following markers: NGN3, NEUROD, ISL1, PDX1, NKX6.1, PAX4, or PTF-1 alpha. Cells expressing markers characteristic of the pancreatic endocrine lineage include pancreatic endocrine cells, pancreatic hormone expressing cells, and pancreatic hormone secreting cells, and cells of the β-cell lineage.

“Definitive endoderm”, as used herein, refers to cells which bear the characteristics of cells arising from the epiblast during gastrulation and which form the gastrointestinal tract and its derivatives. Definitive endoderm cells express the following markers: HNF-3 beta, GATA4, SOX17, Cerberus, OTX2, goosecoid, C-Kit, CD99, and MIXL1.

“Extraembryonic endoderm”, as used herein, refers to a population of cells expressing at least one of the following markers: SOX7, AFP, or SPARC.

“Markers”, as used herein, are nucleic acid or polypeptide molecules that are differentially expressed in a cell of interest. In this context, differential expression means an increased level for a positive marker and a decreased level for a negative marker. The detectable level of the marker nucleic acid or polypeptide is sufficiently higher or lower in the cells of interest compared to other cells, such that the cell of interest can be identified and distinguished from other cells using any of a variety of methods known in the art.

“Mesendoderm cell”, as used herein, refers to a cell expressing at least one of the following markers: CD48, eomesodermin (EOMES), SOX17, DKK4, HNF-3 beta, GSC, FGF17, or GATA6.

“Pancreatic endocrine cell”, or “pancreatic hormone expressing cell”, as used herein, refers to a cell capable of expressing at least one of the following hormones: insulin, glucagon, somatostatin, or pancreatic polypeptide.

“Pancreatic endoderm cell”, or “Stage 4 cells”, or “Stage 4”, as used herein, refers to a cell capable of expressing at least one of the following markers: NGN3, NEUROD, ISL1, PDX1, PAX4, or NKX2.2.

“Pancreatic hormone producing cell”, as used herein, refers to a cell capable of producing at least one of the following hormones: insulin, glucagon, somatostatin, or pancreatic polypeptide.

“Pancreatic hormone secreting cell”, as used herein, refers to a cell capable of secreting at least one of the following hormones: insulin, glucagon, somatostatin, or pancreatic polypeptide.

“Posterior foregut cell” or “Stage 3 cells”, or “Stage 3”, as used herein, refers to a cell capable of secreting at least one of the following markers: PDX1, HNF1, PTF1 alpha, HB9, or PROX1.

“Pre-primitive streak cell”, as used herein, refers to a cell expressing at least one of the following markers: Nodal, or FGF8.

“Primitive gut tube cell” or “Stage 2 cells”, or “Stage2”, as used herein, refers to a cell capable of secreting at least one of the following markers: HNF1, or HNF4 alpha.

“Primitive streak cell”, as used herein, refers to a cell expressing at least one of the following markers: Brachyury, Mix-like homeobox protein, or FGF4.

### The Peptides of the Present Invention

The present invention provides peptides capable of differentiating pluripotent stem cells into cells expressing markers characteristic of the definitive endoderm lineage. In one embodiment, the peptides of the present invention are peptides comprising the amino acid sequence of activin A containing at least one point mutation. The at least one point mutation may be within the region of activin A that facilitates binding to the receptor. Alternatively, the at least one point mutation may be within the region of activin A that is within the homo-dimer interface.

The peptides of the present invention may contain one point mutation. Alternatively, the peptides of the present invention may contain multiple point mutations. In one embodiment, the at least one point mutation is determined by analyzing the crystallographic structure of activin A, wherein specific amino acid residues are chosen for mutation. The at least one point mutation may be in the form of an insertion of at least one amino acid residue. Alternatively, the at least one point mutation may be in the form of a deletion of at least one amino acid residue. Alternatively, the at least one point mutation may be in the form of a substitution of at least one amino acid residue.

The substitution of the at least one amino acid may be in the form of a substitution of at least one random amino acid at the specific location. Alternatively, the substitution of the at least one amino acid may be in the form of a substitution of at least one specific amino acid at the specific location. In one embodiment, the at least one specific amino acid used to substitute is chosen using a computational prediction that the at least one specific amino acid would have on the resulting homo-dimer formation.

In one embodiment, at least one point mutation was introduced into the amino acid sequence of activin A at least one amino acid residue selected from the group consisting of: 10I, 16F, 39Y, 41E, 43E, 74F, 75A, 76N, 77L, 78K, 79S, and 82V.

In one embodiment, at least one point mutation was introduced into the amino acid sequence of activin A at least one amino acid residue selected from the group consisting of: 16F, 18V, 19S, 20F, 37A, 38N, 39Y, 41E, 74F, 82V, 107N, 109I, 110V, and 116S.

The amino acid sequences of the peptides of the present invention may be found in Table 1.

In one embodiment, the amino acid sequences of the peptides of the present invention are back-translated into a nucleic acid sequence. The nucleic acid sequence may be synthesized and inserted into an expression vector to allow expression in mammalian cells. The nucleic acid sequence may be inserted into the expression vector pcDNA3.1(−). Alternatively, the nucleic acid sequence may be inserted into a variant of the pcDNA3.1(−) vector, wherein the vector has been altered to enhance the expression of the inserted nucleic acid sequence in mammalian cells. In one embodiment, the variant of the pcDNA3.1(−) vector is known as pUNDER.

The nucleic acid sequences of the peptides of the present invention may be found in Table 2.

The expression vector, containing a nucleic acid sequence of a peptide of the present invention may be transiently transfected into a mammalian cell. Alternatively, the expression vector, containing a nucleic acid sequence of a peptide of the present invention may be stably transfected into a mammalian cell. Any transfection method is suitable for the present invention. Such transfection method may be, for example, CaCl₂-mediated transfection, or LIPOFECTAMINE™-mediated transfection. See Example 2, for an example of a suitable transfection method.

The mammalian cell may be cultured in suspension, or, alternatively, as a monolayer. An example of a mammalian cell that may be employed for the present invention may be found in Example 2, and an alternative mammalian cell that may be employed for the present invention may be found in Example 3.

In an alternate embodiment, the peptides of the present invention may be expressed in an insect cell expression system, such as, for example, the system described in Kron, R et al (Journal of Virological Methods 72 (1998) 9-14).

### Purification of the Peptides of the Present Invention

The peptides of the present invention may be isolated from the mammalian cells wherein they are expressed. In one embodiment, the mammalian cells are fractionated, and the supernatants containing the peptides of the present invention are removed. The peptides may be purified from the supernatants. Alternatively, the supernatants may be used directly. In the case where the supernatants are used directly, the supernatant is applied directly to human pluripotent stem cells. In one embodiment, the supernatant is concentrated prior to application to human pluripotent stem cells.

In the case where the peptides of the present invention are purified from the supernatant, the peptides may be purified using any suitable protein purification technique, such as, for example, size exclusion chromatography. In one embodiment, the peptides of the present invention are purified by affinity chromatography.

In one embodiment, the peptides of the present invention are purified by affinity chromatography by a method comprising the steps of:
a. Transfecting cells with a vector encoding a peptide of the present invention,
b. Allowing the expression of the peptide in the cells,
c. Fractionating the cells and collecting the supernatant containing the peptide,
d. Passing the supernatant through an affinity purification column, that is packed with a solid matrix containing a ligand that is capable of specifically binding the peptide, and
e. Eluting the bound peptide off the solid matrix, therein obtaining a purified preparation of the peptide.

In one embodiment, the ligand that is capable of specifically binding the peptides of the present invention is follistatin.

In one embodiment, the peptides of the present invention are further modified to contain at least one region that is capable of specifically binding to the ligand on the solid substrate in the affinity purification column. In one embodiment, the peptides of the present invention are further modified to contain at least one metal binding site within their amino acid sequence. The further modification may consist of deleting amino acid resides to form the region that is capable of specifically binding to the ligand on the solid substrate in the affinity purification column. Alternatively, the further modification may consist of inserting amino acid resides to form the region that is capable of specifically binding to the ligand on the solid substrate in the affinity purification column. Alternatively, the further modification may consist of substituting amino acid resides to form the region that is capable of specifically binding to the ligand on the solid substrate in the affinity purification column. In one embodiment, the at least one metal binding site consists of two histidine residues. In one embodiment, the histidine residues are substituted into the amino acid sequence of the peptide comprising the amino acid sequence of activin A containing at least one point mutation. Table 3 lists peptides of the present invention that have been further modified to contain metal binding sites. In these embodiments, the ligand that is capable of specifically binding the peptide is nickel.

In an alternate embodiment, the peptides of the present invention are purified according to the methods described in Pangas, S. A. and Woodruff (J. Endocrinol. 172 (2002) 199-210).

In an alternate embodiment, the peptides of the present invention are purified according to the methods described in Arai, K. Y. et al (Protein Expression and Purification 49 (2006) 78-82).

### Isolation, Expansion and Culture of Pluripotent Stem Cells

### Characterization of Pluripotent Stem Cells

The pluripotency of pluripotent stem cells can be confirmed, for example, by injecting cells into severe combined immunodeficient (SCID) mice, fixing the teratomas that form using 4% paraformaldehyde, and then examining them histologically for evidence of cell types from the three germ layers. Alternatively, pluripotency may be determined by the creation of embryoid bodies and assessing the embryoid bodies for the presence of markers associated with the three germinal layers.

Propagated pluripotent stem cell lines may be karyotyped using a standard G-banding technique and compared to published karyotypes of the corresponding primate species. It is desirable to obtain cells that have a “normal karyotype,” which means that the cells are euploid, wherein all human chromosomes are present and not noticeably altered.

### Sources of Pluripotent Stem Cells

The types of pluripotent stem cells that may be used include established lines of pluripotent cells derived from tissue formed after gestation, including pre-embryonic tissue (such as, for example, a blastocyst), embryonic tissue, or fetal tissue taken any time during gestation, typically but not necessarily before approximately 10 to 12 weeks gestation. Non-limiting examples are established lines of human embryonic stem cells or human embryonic germ cells, such as, for example, the human embryonic stem cell lines H1, H7, and H9 (WiCell). Also contemplated is use of the compositions of this disclosure during the initial establishment or stabilization of such cells, in which case the source cells would be primary pluripotent cells taken directly from the source tissues. Also suitable are cells taken from a pluripotent stem cell population already cultured in the absence of feeder cells. Also suitable are mutant human embryonic stem cell lines, such as, for example, BG01v (BresaGen, Athens, Ga.).

In one embodiment, human embryonic stem cells are prepared as described by Thomson et al. (U.S. Pat. No. 5,843,780; Science 282:1145, 1998; Curr. Top. Dev. Biol. 38:133 ff., 1998; Proc. Natl. Acad. Sci. U.S.A. 92:7844, 1995).

In one embodiment, pluripotent stem cells are prepared as, described by Takahashi et al. (Cell 131: 1-12, 2007).

### Culture of Pluripotent Stem Cells

In one embodiment, pluripotent stem cells are typically cultured on a layer of feeder cells that support the pluripotent stem cells in various ways. Alternatively, pluripotent stem cells are cultured in a culture system that is essentially free of feeder cells but nonetheless supports proliferation of pluripotent stem cells without undergoing substantial differentiation. The growth of pluripotent stem cells in feeder-free culture without differentiation is supported using a medium conditioned by culturing previously, with another cell type. Alternatively, the growth of pluripotent stem cells in feeder-free culture without differentiation is supported using a chemically defined medium.

The pluripotent stem cells may be plated onto a suitable culture substrate. In one embodiment, the suitable culture substrate is an extracellular matrix component, such as, for example, those derived from basement membrane or that may form part of adhesion molecule receptor-ligand couplings. In one embodiment, the suitable culture substrate is MATRIGEL® (Becton Dickenson). MATRIGEL® is a soluble preparation from Engelbreth-Holm-Swarm tumor cells that gels at room temperature to form a reconstituted basement membrane.

Other extracellular matrix components and component mixtures are suitable as an alternative. Depending on the cell type being proliferated, this may include laminin, fibronectin, proteoglycan, entactin, heparan sulfate, and the like, alone or in various combinations.

The pluripotent stem cells may be plated onto the substrate in a suitable distribution and in the presence of a medium that promotes cell survival, propagation, and retention of the desirable characteristics. All these characteristics benefit from careful attention to the seeding distribution and can readily be determined by one of skill in the art.

Suitable culture media may be made from the following components, such as, for example, Dulbecco's modified Eagle's medium (DMEM), Gibco #11965-092; Knockout Dulbecco's modified Eagle's medium (KO DMEM), Gibco #10829-018; Ham's F12/50% DMEM basal medium; 200 mM L-glutamine, Gibco #15039-027; non-essential amino acid solution, Gibco 11140-050; β-mercaptoethanol, Sigma #M7522; human recombinant basic fibroblast growth factor (bFGF), Gibco #13256-029.

### Formation of Pancreatic Hormone Producing Cells from Pluripotent Stem Cells

In one embodiment, the present invention provides a method for producing pancreatic hormone producing cells from pluripotent stem cells, comprising the steps of:
a. Culturing pluripotent stem cells,
b. Differentiating the pluripotent stem cells into cells expressing markers characteristic of the definitive endoderm lineage,
c. Differentiating the cells expressing markers characteristic of the definitive endoderm lineage into cells expressing markers characteristic of the pancreatic endoderm lineage, and
d. Differentiating the cells expressing markers characteristic of the pancreatic endoderm lineage into cells expressing markers characteristic of the pancreatic endocrine lineage.

In one aspect of the present invention, the pancreatic endocrine cell is a pancreatic hormone producing cell. In an alternate aspect, the pancreatic endocrine cell is a cell expressing markers characteristic of the β-cell lineage. A cell expressing markers characteristic of the β-cell lineage expresses PDX1 and at least one of the following transcription factors: NGN3, NKX2.2, NKX6.1, NEUROD, ISL1, HNF-3 beta, MAFA, PAX4, or PAX6. In one aspect of the present invention, a cell expressing markers characteristic of the β-cell lineage is a β-cell.

Pluripotent stem cells suitable for use in the present invention include, for example, the human embryonic stem cell line H9 (NIH code: WA09), the human embryonic stem cell line H1 (NIH code: WA01), the human embryonic stem cell line H7 (NIH code: WA07), and the human embryonic stem cell line SA002 (Cellartis, Sweden). Also suitable for use in the present invention are cells that express at least one of the following markers characteristic of pluripotent cells: ABCG2, cripto, CD9, FOXD3, Connexin43, Connexin45, OCT4, SOX2, Nanog, hTERT, UTF-1, ZFP42, SSEA-3, SSEA-4, Tra1-60, or Tra1-81.

Markers characteristic of the definitive endoderm lineage are selected from the group consisting of SOX17, GATA4, HNF-3beta, GSC, CER1, Nodal, FGF8, Brachyury, Mix-like homeobox protein, FGF4 CD48, eomesodermin (EOMES), DKK4, FGF17, GATA6, CXCR4, C-Kit, CD99, and OTX2. Suitable for use in the present invention is a cell that expresses at least one of the markers characteristic of the definitive endoderm lineage. In one aspect of the present invention, a cell expressing markers characteristic of the definitive endoderm lineage is a primitive streak precursor cell. In an alternate aspect, a cell expressing markers characteristic of the definitive endoderm lineage is a mesendoderm cell. In an alternate aspect, a cell expressing markers characteristic of the definitive endoderm lineage is a definitive endoderm cell.

Markers characteristic of the pancreatic endoderm lineage are selected from the group consisting of PDX1, HNF-1beta, PTF1 alpha, HNF6, HB9 and PROX1. Suitable for use in the present invention is a cell that expresses at least one of the markers characteristic of the pancreatic endoderm lineage. In one aspect of the present invention, a cell expressing markers characteristic of the pancreatic endoderm lineage is a pancreatic endoderm cell.

Markers characteristic of the pancreatic endocrine lineage are selected from the group consisting of NGN3, NEUROD, ISL1, PDX1, NKX6.1, PAX4, and PTF-1 alpha. In one embodiment, a pancreatic endocrine cell is capable of expressing at least one of the following hormones: insulin, glucagon, somatostatin, and pancreatic polypeptide. Suitable for use in the present invention is a cell that expresses at least one of the markers characteristic of the pancreatic endocrine lineage. In one aspect of the present invention, a cell expressing markers characteristic of the pancreatic endocrine lineage is a pancreatic endocrine cell. The pancreatic endocrine cell may be a pancreatic hormone expressing cell. Alternatively, the pancreatic endocrine cell may be a pancreatic hormone secreting cell.

### Formation of Cells Expressing Markers Characteristic of the Definitive Endoderm Lineage

In one aspect of the present invention, pluripotent stem cells may be differentiated into cells expressing markers characteristic of the definitive endoderm lineage by treating the pluripotent stem cells with medium containing a peptide of the present invention, for an amount of time sufficient to enable the pluripotent stem cells to differentiate into cells expressing markers characteristic of the definitive endoderm lineage.

The pluripotent stem cells may be treated with medium containing a peptide of the present invention for about one day to about seven days. Alternatively, the pluripotent stem cells may be treated with medium containing a peptide of the present invention for about one day to about six days. Alternatively, the pluripotent stem cells may be treated with medium containing a peptide of the present invention for about one day to about five days. Alternatively, the pluripotent stem cells may be treated with medium containing a peptide of the present invention for about one day to about four days. Alternatively, the pluripotent stem cells may be treated with medium containing a peptide of the present invention for about one day to about three days. Alternatively, the pluripotent stem cells may be treated with medium containing a peptide of the present invention for about one day to about two days. In one embodiment, the pluripotent stem cells may be treated with medium containing a peptide of the present invention for about four days.

The pluripotent stem cells may be cultured on a feeder cell layer. Alternatively, the pluripotent stem cells may be cultured on an extracellular matrix.

In one aspect of the present invention, the pluripotent stem cells are cultured and differentiated on a tissue culture substrate coated with an extracellular matrix. The extracellular matrix may be a solubilized basement membrane preparation extracted from mouse sarcoma cells (as sold by BD Biosciences under the trade name MATRIGEL™). Alternatively, the extracellular matrix may be growth factor-reduced MATRIGEL™. Alternatively, the extracellular matrix may be fibronectin. In an alternate embodiment, the pluripotent stem cells are cultured and differentiated on tissue culture substrate coated with human serum.

The extracellular matrix may be diluted prior to coating the tissue culture substrate. Examples of suitable methods for diluting the extracellular matrix and for coating the tissue culture substrate may be found in Kleinman, H. K., et al., Biochemistry 25:312 (1986), or Hadley, M. A., et al., J. Cell. Biol. 101:1511 (1985).

In one embodiment, the extracellular matrix is MATRIGEL™. In one embodiment, the tissue culture substrate is coated with MATRIGEL™ at a 1:10 dilution. In an alternate embodiment, the tissue culture substrate is coated with MATRIGEL™ at a 1:15 dilution. In an alternate embodiment, the tissue culture substrate is coated with MATRIGEL™ at a 1:30 dilution. In an alternate embodiment, the tissue culture substrate is coated with MATRIGEL™ at a 1:60 dilution.

In one embodiment, the extracellular matrix is growth factor-reduced MATRIGEL™. In one embodiment, the tissue culture substrate is coated with growth factor-reduced MATRIGEL™ at a 1:10 dilution. In an alternate embodiment, the tissue culture substrate is coated with growth factor-reduced MATRIGEL™ at a 1:15 dilution. In an alternate embodiment, the tissue culture substrate is coated with growth factor-reduced MATRIGEL™ at a 1:30 dilution. In an alternate embodiment, the tissue culture substrate is coated with growth factor-reduced MATRIGEL™ at a 1:60 dilution.

The pluripotent stem cells may be treated with medium containing a peptide of the present invention that has been purified from the supernatant of the cell that expressed the peptide. Alternatively, the pluripotent stem cells may be treated with medium containing a peptide of the present invention that has been not purified from the supernatant of the cell that expressed the peptide.

In the case where the pluripotent stem cells are treated with medium containing a peptide of the present invention that has been not purified from the supernatant of the cell that expressed the peptide, the supernatant may be used at a final concentration of about 1:10 dilution to about 1:100. In one embodiment, supernatant may be used at a final concentration of about 1:10 dilution to about 1:50. In one embodiment, supernatant may be used at a final concentration of about 1:10 dilution to about 1:40. In one embodiment, supernatant may be used at a final concentration of about 1:20 dilution to about 1:50.

In one embodiment, pluripotent stem cells are treated with medium containing the following peptide:

GLECDGKVNICCKKQFFVSFKDIGWNDWIIAPSGYHANYCEGECPSHIAG
TSGSSLSFHSTVINHYRMRGHSPFANLKSCCVPTKLRPMSMLYYDDGQNI
IKKDIQNMIVEECGCS.

In one embodiment, pluripotent stem cells are treated with medium containing the following peptide:

GLECDGKVNLCCKKQNFVSFKDIGWNDWIIAPSGYHANECTGKCPSHIAG
TSGSSLSFHSTVINHYRMRGHSPFSDLGSCCIPTKLRPMSMLYYDDGQNI
IKKDIQNMIVEECGCS.

In one embodiment, pluripotent stem cells are treated with medium containing the following peptide:

GLECDGKVNLCCKKQDFVSFKDIGWNDWIIAPSGYHANRCSGKCPSHIAG
TSGSSLSFHSTVINHYRMRGHSPFSNMGSCCIPTKLRPMSMLYYDDGQNI
IKKDIQNMIVEECGCS.

In one embodiment, pluripotent stem cells are treated with medium containing the following peptide:

GLECDGKVNLCCKKQLFVSFKDIGWNDWIIAPSGYHANHCSGLCPSHIAG
TSGSSLSFHSTVINHYRMRGHSPFSDMGACCVPTKLRPMSMLYYDDGQNI
IKKDIQNMIVEECGCS.

In one embodiment, pluripotent stem cells are treated with medium containing the following peptide:

GLECDGKVNYCCKKQNFVSFKDIGWNDWIIAPSGYHANECSGKCPSHIAG
TSGSSLSFHSTVINHYRMRGHSPFSNLGACCIPTKLRPMSMLYYDDGQNI
IKKDIQNMIVEECGCS.

In one embodiment, pluripotent stem cells are treated with medium containing the following peptide:

GLECDGKVNLCCKKQWFVSFKDIGWNDWIIAPSGYHANRCSGKCPSHIAG
TSGSSLSFHSTVINHYRMRGHSPFADMGACCIPTKLRPMSMLYYDDGQNI
IKKDIQNMIVEECGCS.

In one embodiment, pluripotent stem cells are treated with medium containing the following peptide:

GLECDGKVNYCCKKQHFVSFKDIGWNDWIIAPSGYHANSCSGLCPSHI
AGTSGSSLSFHSTVINHYRMRGHSPFSQMGSCCIPTKLRPMSMLYYDD
GQNIIKKDIQNMIVEECGCS.

In one embodiment, pluripotent stem cells are treated with medium containing the following peptide:

GLECDGKVNYCCKKQDFVSFKDIGWNDWIIAPSGYHANKCSGKCPSHI
AGTSGSSLSFHSTVINHYRMRGHSPFSDMGSCCVPTKLRPMSMLYYDD
GQNIIKKDIQNMIVEECGCS.

In one embodiment, pluripotent stem cells are treated with medium containing the following peptide:

GLECDGKVNYCCKKQDFVSFKDIGWNDWIIAPSGYHANKCTGKCPSHI
AGTSGSSLSFHSTVINHYRMRGHSPFADLGACCIPTKLRPMSMLYYDD
GQNIIKKDIQNMIVEECGCS.

In one embodiment, pluripotent stem cells are treated with medium containing the following peptide:

GLECDGKVNYCCKKQDFVSFKDIGWNDWIIAPSGYHANRCSGLCPSHI
AGTSGSSLSFHSTVINHYRMRGHSPFAQMGSCCIPTKLRPMSMLYYDD
GQNIIKKDIQNMIVEECGCS.

In one embodiment, pluripotent stem cells are treated with medium containing the following peptide:

GLECDGKVNLCCKKQLFVSFKDIGWNDWIIAPSGYHANHCSGLCPSHI
AGTSGSSLSFHSTVINHYRMRGHSPFAQMGACCIPTKLRPMSMLYYDD
GQNIIKKDIQNMIVEECGCS.

In one embodiment, pluripotent stem cells are treated with medium containing the following peptide:

GLECDGKVNYCCKKQNFVSFKDIGWNDWIIAPSGYHANECSGLCPSHI
AGTSGSSLSFHSTVINHYRMRGHSPFSQMGSCCIPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANKCSGKCPSHI
AGTSGSSLSFHSTVINHYRMRGHSPFSDMGSCCIPTKLRPMSMLYYDD
GQNIIKKDIQNMIVEECGCS.

In one embodiment, pluripotent stem cells are treated with medium containing the following peptide:

GLECDGKVNLCCKKQHFVSFKDIGWNDWIIAPSGYHANRCDGLCPSHI
AGTSGSSLSFHSTVINHYRMRGHSPFAQMGSCCVPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANRCSGLCPSHI
AGTSGSSLSFHSTVINHYRMRGHSPFSNMGSCCIPTKLRPMSMLYYDD
GQNIIKKDIQNMIVEECGCS.

In one embodiment, pluripotent stem cells are treated with medium containing the following peptide:

GLECDGKVNLCCKKQLFVSFKDIGWNDWIIAPSGYHANHCSGLCPSHI
AGTSGSSLSFHSTVINHYRMRGHSPFSQMGACCVPTKLRPMSMLYYDD
GQNIIKKDIQNMIVEECGCS.

In one embodiment, pluripotent stem cells are treated with medium containing the following peptide:

GLECDGKVNYCCKKQDFVSFKDIGWNDWIIAPSGYHANKCGGKCPSHI
AGTSGSSLSFHSTVINHYRMRGHSPFSDMGACCVPTKLRPMSMLYYDD
GQNIIKKDIQNMIVEECGCS.

In one embodiment, pluripotent stem cells are treated with medium containing the following peptide:

GLECDGKVNYCCKKQNFVSFKDIGWNDWIIAPSGYHANKCSGKCPSHI
AGTSGSSLSFHSTVINHYRMRGHSPFSDMGACCIPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANKCGGKCPSHI
AGTSGSSLSFHSTVINHYRMRGHSPFANMGSCCIPTKLRPMSMLYYDD
GQNIIKKDIQNMIVEECGCS.

In one embodiment, pluripotent stem cells are treated with medium containing the following peptide:

GLECDGKVNLCCKKQNFVSFKDIGWNDWIIAPSGYHANKCGGLCPSHI
AGTSGSSLSFHSTVINHYRMRGHSPFAQMGACCIPTKLRPMSMLYYDD
GQNIIKKDIQNMIVEECGCS.

In one embodiment, pluripotent stem cells are treated with medium containing the following peptide:

GLECDGKVNYCCKKQWFVSFKDIGWNDWIIAPSGYHANKCSGKCPSHI
AGTSGSSLSFHSTVINHYRMRGHSPFANMGSCCIPTKLRPMSMLYYDD
QNIIKKDIQNMIVEECGCS.

In one embodiment, pluripotent stem cells are treated with medium containing the following peptide:

GLECDGKVNLCCKKQDFVSFKDIGWNDWIIAPSGYHANRCDGLCPSHI
AGTSGSSLSFHSTVINHYRMRGHSPFALMGACCIPTKLRPMSMLYYDD
GQNIIKKDIQNMIVEECGCS.

In one embodiment, pluripotent stem cells are treated with medium containing the following peptide:

GLECDGKVNYCCKKQDFVSFKDIGWNDWIIAPSGYHANKCDGKCPSHI
AGTSGSSLSFHSTVINHYRMRGHSPFSDMGSCCIPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANRCSGRCPSHI
AGTSGSSLSFHSTVINHYRMRGHSPFSDMGSCCVPTKLRPMSMLYYDD
GQNIIKKDIQNMIVEECGCS.

In one embodiment, pluripotent stem cells are treated with medium containing the following peptide:

GLECDGKVNLCCKKQNFVSFKDIGWNDWIIAPSGYHANECSGLCPSHI
AGTSGSSLSFHSTVINHYRMRGHSPFSQMGSCCIPTKLRPMSMLYYDD
GQNIIKKDIQNMIVEECGCS.

In one embodiment, pluripotent stem cells are treated with medium containing the following peptide:

GLECDGKVNLCCKKQHFVSFKDIGWNDWIIAPSGYHANRCDGKCPSHI
AGTSGSSLSFHSTVINHYRMRGHSPFANMGACCIPTKLRPMSMLYYDD
GQNIIKKDIQNMIVEECGCS.

In one embodiment, pluripotent stem cells are treated with medium containing the following peptide:

GLECDGKVNLCCKKQLFVSFKDIGWNDWIIAPSGYHANHCDGKCPSHI
AGTSGSSLSFHSTVINHYRMRGHSPFANRGACCIPTKLRPMSMLYYDD
GQNIIKKDIQNMIVEECGCS.

In one embodiment, pluripotent stem cells are treated with medium containing the following peptide:

GLECDGKVNYCCKKQLFVSFKDIGWNDWIIAPSGYHANHCSGKCPSHI
AGTSGSSLSFHSTVINHYRMRGHSPFANMGSCCIPTKLRPMSMLYYDD
GQNIIKKDIQNMIVEECGCS.

In one embodiment, pluripotent stem cells are treated with medium containing the following peptide:

GLECDGKVNLCCKKQLFVSFKDIGWNDWIIAPSGYHANHCSGKCPSHI
AGTSGSSLSFHSTVINHYRMRGHSPFSDMGSCCIPTKLRPMSMLYYDD
GQNIIKKDIQNMIVEECGCS.

In one embodiment, pluripotent stem cells are treated with medium containing the following peptide:

GLECDGKVNYCCKKQNFVSFKDIGWNDWIIAPSGYHANKCSGLCPSHI
AGTSGSSLSFHSTVINHYRMRGHSPFSKMGACCVPTKLRPMSMLYYDD
GQNIIKKDIQNMIVEECGCS.

In one embodiment, pluripotent stem cells are treated with medium containing the following peptide:

GLECDGKVNTCCKKQLFVSFKDIGWNDWIIAPSGYHANHCSGKCPSHI
AGTSGSSLSFHSTVINHYRMRGHSPFSDMGSCCIPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANRCGGKCPSHI
AGTSGSSLSFHSTVINHYRMRGHSPFSNMGSCCIPTKLRPMSMLYYDD
GQNIIKKDIQNMIVEECGCS.

In one embodiment, pluripotent stem cells are treated with medium containing the following peptide:

GLECDGKVNLCCKKQNFVSFKDIGWNDWIIAPSGYHANECMGKCPSHI
AGTSGSSLSFHSTVINHYRMRGHSPFSDMGACCIPTKLRPMSMLYYDD
GQNIIKKDIQNMIVEECGCS.

In one embodiment, pluripotent stem cells are treated with medium containing the following peptide:

GLECDGKVNYCCKKQLFVSFKDIGWNDWIIAPSGYHANHCTGKCPSHI
AGTSGSSLSFHSTVINHYRMRGHSPFSDLGSCCVPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANRCSGKCPSHI
AGTSGSSLSFHSTVINHYRMRGHSPFSDMGSCCVPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANRCSGKCPSHI
AGTSGSSLSFHSTVINHYRMRGHSPFANMGACCVPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANRCSGKCPSHI
AGTSGSSLSFHSTVINHYRMRGHSPFSDMGACCIPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANKCGGKCPSHI
AGTSGSSLSFHSTVINHYRMRGHSPFSQLGACCVPTKLRPMSMLYYDD
GQNIIKKDIQNMIVEECGCS.

In one embodiment, pluripotent stem cells are treated with medium containing the following peptide:

GLECDGKVNLCCKKQNFVSFKDIGWNDWIIAPSGYHANECSGLCPSHI
AGTSGSSLSFHSTVINHYRMRGHSPFSDMGSCCVPTKLRPMSMLYYDD
GQNIIKKDIQNMIVEECGCS.

In one embodiment, pluripotent stem cells are treated with medium containing the following peptide:

GLECDGKVNLCCKKQLFVSFKDIGWNDWIIAPSGYHANHCAGLCPSHI
AGTSGSSLSFHSTVINHYRMRGHSPFSNMGSCCVPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANSCSGLCPSHI
AGTSGSSLSFHSTVINHYRMRGHSPFSDRGACCIPTKLRPMSMLYYDD
GQNIIKKDIQNMIVEECGCS.

In one embodiment, pluripotent stem cells are treated with medium containing the following peptide:

GLECDGKVNYCCKKQDFVSFKDIGWNDWIIAPSGYHANKCSGKCPSHI
AGTSGSSLSFHSTVINHYRMRGHSPFSDMGSCCIPTKLRPMSMLYYDD
GQNIIKKDIQNMIVEECGCS.

In one embodiment, pluripotent stem cells are treated with medium containing the following peptide:

GLECDGKVNYCCKKQDFVSFKDIGWNDWIIAPSGYHANRCDGKCPSHI
AGTSGSSLSFHSTVINHYRMRGHSPFSDMGACCVPTKLRPMSMLYYDD
GQNIIKKDIQNMIVEECGCS.

In one embodiment, pluripotent stem cells are treated with medium containing the following peptide:

GLECDGKVNLCCKKQNFVSFKDIGWNDWIIAPSGYHANECSGKCPSHI
AGTSGSSLSFHSTVINHYRMRGHSPFSDMGACCIPTKLRPMSMLYYDD
GQNIIKKDIQNMIVEECGCS.

In one embodiment, pluripotent stem cells are treated with medium containing the following peptide:

GLECDGKVNTCCKKQNFVSFKDIGWNDWIIAPSGYHANECSGKCPSHI
AGTSGSSLSFHSTVINHYRMRGHSPFANMGACCIPTKLRPMSMLYYDD
GQNIIKKDIQNMIVEECGCS.

In one embodiment, pluripotent stem cells are treated with medium containing the following peptide:

GLECDGKVNLCCKKQNFVSFKDIGWNDWIIAPSGYHANECGGLCPSHI
AGTSGSSLSFHSTVINHYRMRGHSPHANRGACCIPTKLRPMSMLYYDD
GQNIIKKDIQNMIVEECGCS.

In one embodiment, pluripotent stem cells are treated with medium containing the following peptide:

GLECDGKVNYCCKKQNFVSFKDIGWNDWIIAPSGYHANECSGKCPSHI
AGTSGSSLSFHSTVINHYRMRGHSPFSDMGSCCIPTKLRPMSMLYYDD
GQNIIKKDIQNMIVEECGCS.

In one embodiment, pluripotent stem cells are treated with medium containing the following peptide:

GLECDGKVNLCCKKQNFVSFKDIGWNDWIIAPSGYHANECSGLCPSHI
AGTSGSSLSFHSTVINHYRMRGHSPFANMGACCIPTKLRPMSMLYYDD
GQNIIKKDIQNMIVEECGCS.

In one embodiment, pluripotent stem cells are treated with medium containing the following peptide:

GLECDGKVNLCCKKQDFVSFKDIGWNDWIIAPSGYHANRCDGLCPSHI
AGTSGSSLSFHSTVINHYRMRGHSPFSDMGSCCVPTKLRPMSMLYYDD
GQNIIKKDIQNMIVEECGCS.

In one embodiment, pluripotent stem cells are treated with medium containing the following peptide:

GLECDGKVNICCKKQLFGRTKDIGWNDWIIAPSGYHGGGCSGECPSHI
AGTSGSSLSFHSTVINHYRMRGHSPVANLKSCCSPTKLRPMSMLYYDD
GQNIIKKDIQNMKVEECGCT.

In one embodiment, pluripotent stem cells are treated with medium containing the following peptide:

GLECDGKVNICCKKQSFGQTKDIGWNDWIIAPSGYHGGGCSGECPSHI
AGTSGSSLSFHSTVINHYRMRGHSPFANLKSCCSPTKLRPMSMLYYDD
GQNIIKKDIQRMVVEECGCT.

In one embodiment, pluripotent stem cells are treated with medium containing the following peptide:

GLECDGKVNICCKKQLFGKTKDIGWNDWIIAPSGYHGGSCTGECPSHI
AGTSGSSLSFHSTVINHYRMRGHSPNANLKSCCSPTKLRPMSMLYYDD
GQNIIKKDIQGMKVEECGCT.

In one embodiment, pluripotent stem cells are treated with medium containing the following peptide:

GLECDGKVNICCKKQEFGQAKDIGWNDWIIAPSGYHGGGCSGECPSHI
AGTSGSSLSFHSTVINHYRMRGHSPWANLKSCCSPTKLRPMSMLYYDD
GQNIIKKDIQGMKVEECGCT.

In one embodiment, pluripotent stem cells are treated with medium containing the following peptide:

GLECDGKVNICCKKQSFAQTKDIGWNDWIIAPSGYHGGGCTGECPSHI
AGTSGSSLSFHSTVINHYRMRGHSPFANLKSCCSPTKLRPMSMLYYDD
GQNIIKKDIQNMVVEECGCT.

In one embodiment, pluripotent stem cells are treated with medium containing the following peptide:

GLECDGKVNICCKKQSFGQTKDIGWNDWIIAPSGYHGGGCTGECPSHI
AGTSGSSLSFHSTVINHYRMRGHSPWANLKSCCSPTKLRPMSMLYYDD
GQNIIKKDIQGMVVEECGCT.

In one embodiment, pluripotent stem cells are treated with medium containing the following peptide:

GLECDGKVNICCKKQSFGQAKDIGWNDWIIAPSGYHGGSCTGECPSHI
AGTSGSSLSFHSTVINHYRMRGHSPFANLKSCCAPTKLRPMSMLYYDD
GQNIIKKDIQNMVVEECGCV.

In one embodiment, pluripotent stem cells are treated with medium containing the following peptide:

GLECDGKVNICCKKQSFGQAKDIGWNDWIIAPSGYHGGGCSGECPSHI
AGTSGSSLSFHSTVINHYRMRGHSPWANLKSCCSPTKLRPMSMLYYDD
GQNIIKKDIQNMKVEECGCT.

In one embodiment, pluripotent stem cells are treated with medium containing the following peptide:

GLECDGKVNICCKKQSFGQTKDIGWNDWIIAPSGYHGGGCTGECPSHI
AGTSGSSLSFHSTVINHYRMRGHSPWANLKSCCSPTKLRPMSMLYYDD
GQNIIKKDIQNMKVEECGCT.

In one embodiment, pluripotent stem cells are treated with medium containing the following peptide:

GLECDGKVNICCKKQLFGQTKDIGWNDWIIAPSGYHGGGCTGECPSHI
AGTSGSSLSFHSTVINHYRMRGHSPNANLKSCCAPTKLRPMSMLYYDD
GQNIIKKDIQGMKVEECGCV.

In one embodiment, pluripotent stem cells are treated with medium containing the following peptide:

GLECDGKVNICCKKQSFGQTKDIGWNDWIIAPSGYHGGGCSGECPSHI
AGTSGSSLSFHSTVINHYRMRGHSPWANLKSCCAPTKLRPMSMLYYDD
GQNIIKKDIQNMVVEECGCV.

In one embodiment, pluripotent stem cells are treated with medium containing the following peptide:

GLECDGKVNICCKKQLFGQTKDIGWNDWIIAPSGYHGGSCTGECPSHI
AGTSGSSLSFHSTVINHYRMRGHSPNANLKSCCSPTKLRPMSMLYYDD
GQNIIKKDIQNMKVEECGCT.

In one embodiment, pluripotent stem cells are treated with medium containing the following peptide:

GLECDGKVNICCKKQSFGQAKDIGWNDWIIAPSGYHGGGCTGECPSHI
AGTSGSSLSFHSTVINHYRMRGHSPWANLKSCCSPTKLRPMSMLYYDD
GQNIIKKDIQNMVVEECGCT.

In one embodiment, pluripotent stem cells are treated with medium containing the following peptide:

GLECDGKVNICCKKQSFSQAKDIGWNDWIIAPSGYHGGGCTGECPSHI
AGTSGSSLSFHSTVINHYRMRGHSPFANLKSCCSPTKLRPMSMLYYDD
GQNIIKKDIQGMVVEECGCT.

In one embodiment, pluripotent stem cells are treated with medium containing the following peptide:

GLECDGKVNICCKKQSFGQTKDIGWNDWIIAPSGYHGGGCSGECPSHI
AGTSGSSLSFHSTVINHYRMRGHSPWANLKSCCSPTKLRPMSMLYYDD
GQNIIKKDIQGMVVEECGCT.

In one embodiment, pluripotent stem cells are treated with medium containing the following peptide:

GLECDGKVNICCKKQSFGQTKDIGWNDWIIAPSGYHGGSCSGECPSHI
AGTSGSSLSFHSTVINHYRMRGHSPFANLKSCCSPTKLRPMSMLYYDD
GQNIIKKDIQGMKVEECGCT.

In one embodiment, pluripotent stem cells are treated with medium containing the following peptide:

GLECDGKVNICCKKQMFGQAKDIGWNDWIIAPSGYHGGGCTGECPSHI
AGTSGSSLSFHSTVINHYRMRGHSPWANLKSCCSPTKLRPMSMLYYDD
GQNIIKKDIQNMVVEECGCT.

In one embodiment, pluripotent stem cells are treated with medium containing the following peptide:

GLECDGKVNICCKKQSFGQTKDIGWNDWIIAPSGYHGGGCSGECPSHI
AGTSGSSLSFHSTVINHYRMRGHSPWANLKSCCSPTKLRPMSMLYYDD
GQNIIKKDIQNMKVEECGCT.

In one embodiment, pluripotent stem cells are treated with medium containing the following peptide:

GLECDGKVNICCKKQSFGKAKDIGWNDWIIAPSGYHGGGCTGECPSHI
AGTSGSSLSFHSTVINHYRMRGHSPFANLKSCCSPTKLRPMSMLYYDD
GQNIIKKDIQGMKVEECGCT.

In one embodiment, pluripotent stem cells are treated with medium containing the following peptide:

GLECDGKVNICCKKQSFGKTKDIGWNDWIIAPSGYHGGGCTGECPSHI
AGTSGSSLSFHSTVINHYRMRGHSPFANLKSCCSPTKLRPMSMLYYDD
GQNIIKKDIQQMVVEECGCT.

In one embodiment, pluripotent stem cells are treated with medium containing the following peptide:

GLECDGKVNICCKKQLFGQAKDIGWNDWIIAPSGYHGGGCSGECPSHI
AGTSGSSLSFHSTVINHYRMRGHSPVANLKSCCSPTKLRPMSMLYYDD
GQNIIKKDIQGMVVEECGCT.

In one embodiment, pluripotent stem cells are treated with medium containing the following peptide:

GLECDGKVNICCKKQSFGQAKDIGWNDWIIAPSGYHGGGCTGECPSHI
AGTSGSSLSFHSTVINHYRMRGHSPFANLKSCCSPTKLRPMSMLYYDD
GQNIIKKDIQNMKVEECGCT.

In one embodiment, pluripotent stem cells are treated with medium containing the following peptide:

GLECDGKVNICCKKQLFGQAKDIGWNDWI1APSGYHGGSCTGECPSHI
AGTSGSSLSFHSTVINHYRMRGHSPNANLKSCCSPTKLRPMSMLYYDD
GQNIIKKDIQNMVVEECGCT.

In one embodiment, pluripotent stem cells are treated with medium containing the following peptide:

GLECDGKVNICCKKQSFGQAKDIGWNDWIIAPSGYHGGGCSGECPSHI
AGTSGSSLSFHSTVINHYRMRGHSPFANLKSCCSPTKLRPMSMLYYDD
GQNIIKKDIQGMVVEECGCT.

In one embodiment, pluripotent stem cells are treated with medium containing the following peptide:

GLECDGKVNICCKKQSFGRAKDIGWNDWIIAPSGYHGGGCTGECPSHI
AGTSGSSLSFHSTVINHYRMRGHSPFANLKSCCSPTKLRPMSMLYYDD
GQNIIKKDIQGMVVEECGCT.

In one embodiment, pluripotent stem cells are treated with medium containing the following peptide:

GLECDGKVNICCKKQSFGQAKDIGWNDWIIAPSGYHGGGCSGECPSHI
AGTSGSSLSFHSTVINHYRMRGHSPWANLKSCCSPTKLRPMSMLYYDD
GQNIIKKDIQRMKVEECGCT.

In one embodiment, pluripotent stem cells are treated with medium containing the following peptide:

GLECDGKVNICCKKQLFGQTKDIGWNDWIIAPSGYHGGGCSGECPSHI
AGTSGSSLSFHSTVINHYRMRGHSPNANLKSCCSPTKLRPMSMLYYDD
GQNIIKKDIQGMKVEECGCT.

In one embodiment, pluripotent stem cells are treated with medium containing the following peptide:

GLECDGKVNICCKKQMFGKAKDIGWNDWIIAPSGYHGGGCTGECPSHI
AGTSGSSLSFHSTVINHYRMRGHSPWANLKSCCSPTKLRPMSMLYYDD
GQNIIKKDIQNMVVEECGCT.

In one embodiment, pluripotent stem cells are treated with medium containing the following peptide:

GLECDGKVNICCKKQLFGQAKDIGWNDWIIAPSGYHGGGCSGECPSHI
AGTSGSSLSFHSTVINHYRMRGHSPVANLKSCCSPTKLRPMSMLYYDD
GQNIIKKDIQNMVVEECGCT.

In one embodiment, pluripotent stem cells are treated with medium containing the following peptide:

GLECDGKVNICCKKQSFGQAKDIGWNDWIIAPSGYHGGGCTGECPSHI
AGTSGSSLSFHSTVINHYRMRGHSPWANLKSCCSPTKLRPMSMLYYDD
GQNIIKKDIQRMVVEECGCT.

In one embodiment, pluripotent stem cells are treated with medium containing the following peptide:

GLECDGKVNICCKKQSFGQTKDIGWNDWIIAPSGYHGGGCSGECPSHI
AGTSGSSLSFHSTVINHYRMRGHSPWANLKSCCAPTKLRPMSMLYYDD
GQNIIKKDIQNMKVEECGCV.

In one embodiment, pluripotent stem cells are treated with medium containing the following peptide:

GLECDGKVNICCKKQLFGKTKDIGWNDWIIAPSGYHGGGCTGECPSHI
AGTSGSSLSFHSTVINHYRMRGHSPVANLKSCCSPTKLRPMSMLYYDD
GQNIIKKDIQRMVVEECGCT.

In one embodiment, pluripotent stem cells are treated with medium containing the following peptide:

GLECDGKVNICCKKQSFGQTKDIGWNDWIIAPSGYHGGGCTGECPSHI
AGTSGSSLSFHSTVINHYRMRGHSPWANLKSCCSPTKLRPMSMLYYDD
GQNIIKKDIQGMKVEECGCT.

In one embodiment, pluripotent stem cells are treated with medium containing the following peptide:

GLECDGKVNICCKKQSFGRAKDIGWNDWIIAPSGYHGGGCTGECPSHI
AGTSGSSLSFHSTVINHYRMRGHSPFANLKSCCSPTKLRPMSMLYYDD
GQNIIKKDIQGMKVEECGCT.

In one embodiment, pluripotent stem cells are treated with medium containing the following peptide:

GLECDGKVNICCKKQSFGQAKDIGWNDWIIAPSGYHGGGCSGECPSHI
AGTSGSSLSFHSTVINHYRMRGHSPFANLKSCCSPTKLRPMSMLYYDD
GQNIIKKDIQNMVVEECGCT.

In one embodiment, pluripotent stem cells are treated with medium containing the following peptide:

GLECDGKVNICCKKQLFGQAKDIGWNDWIIAPSGYHGGGCTGECPSHI
AGTSGSSLSFHSTVINHYRMRGHSPVANLKSCCSPTKLRPMSMLYYDD
GQNIIKKDIQNMKVEECGCT.

In one embodiment, pluripotent stem cells are treated with medium containing the following peptide:

GLECDGKVNICCKKQSFGKTKDIGWNDWIIAPSGYHGGGCSGECPSHI
AGTSGSSLSFHSTVINHYRMRGHSPWANLKSCCSPTKLRPMSMLYYDD
GQNIIKKDIQNMVVEECGCT.

In one embodiment, pluripotent stem cells are treated with medium containing the following peptide:

GLECDGKVNICCKKQSFGQAKDIGWNDWIIAPSGYHGGSCSGECPSHI
AGTSGSSLSFHSTVINHYRMRGHSPFANLKSCCSPTKLRPMSMLYYDD
GQNIIKKDIQNMVVEECGCT.

In one embodiment, pluripotent stem cells are treated with medium containing the following peptide:

GLECDGKVNICCKKQSFGQTKDIGWNDWIIAPSGYHGGGCSGECPSHI
AGTSGSSLSFHSTVINHYRMRGHSPWANLKSCCSPTKLRPMSMLYYDD
GQNIIKKDIQNMVVEECGCT.

In one embodiment, pluripotent stem cells are treated with medium containing the following peptide:

GLECDGKVNICCKKQLFGQTKDIGWNDWIIAPSGYHGGGCSGECPSHI
AGTSGSSLSFHSTVINHYRMRGHSPNANLKSCCSPTKLRPMSMLYYDD
GQNIIKKDIQNMKVEECGCT.

In one embodiment, pluripotent stem cells are treated with medium containing the following peptide:

GLECDGKVNICCKKQSFGQTKDIGWNDWIIAPSGYHGGSCSGECPSHI
AGTSGSSLSFHSTVINHYRMRGHSPFANLKSCCSPTKLRPMSMLYYDD
GQNIIKKDIQNMKVEECGCT.

In one embodiment, pluripotent stem cells are treated with medium containing the following peptide:

GLECDGKVNICCKKQSFGRTKDIGWNDWIIAPSGYHGGGCSGECPSHI
AGTSGSSLSFHSTVINHYRMRGHSPWANLKSCCSPTKLRPMSMLYYDD
GQNIIKKDIQNMVVEECGCT.

In one embodiment, pluripotent stem cells are treated with medium containing the following peptide:

GLECDGKVNICCKKQSFGQAKDIGWNDWIIAPSGYHGGGCSGECPSHI
AGTSGSSLSFHSTVINHYRMRGHSPFANLKSCCSPTKLRPMSMLYYDD
GQNIIKKDIQGMKVEECGCT.

In one embodiment, pluripotent stem cells are treated with medium containing the following peptide:

GLECDGKVNICCKKQSFGQTKDIGWNDWIIAPSGYHGGGCTGECPSHI
AGTSGSSLSFHSTVINHYRMRGHSPFANLKSCCSPTKLRPMSMLYYDD
GQNIIKKDIQNMKVEECGCT.

In one embodiment, pluripotent stem cells are treated with medium containing the following peptide:

GLECDGKVNICCKKQSFGQAKDIGWNDWIIAPSGYHGGGCTGECPSHI
AGTSGSSLSFHSTVINHYRMRGHSPWANLKSCCSPTKLRPMSMLYYDD
GQNIIKKDIQRMVAEECGCT.

### Detection of Cells Expressing Markers Characteristic of the Definitive Endoderm Lineage

Formation of cells expressing markers characteristic of the definitive endoderm lineage may be determined by testing for the presence of the markers before and after following a particular protocol. Pluripotent stem cells typically do not express such markers. Thus, differentiation of pluripotent cells is detected when cells begin to express them.

The efficiency of differentiation may be determined by exposing a treated cell population to an agent (such as an antibody) that specifically recognizes a protein marker expressed by cells expressing markers characteristic of the definitive endoderm lineage.

Methods for assessing expression of protein and nucleic acid markers in cultured or isolated cells are standard in the art. These include quantitative reverse transcriptase polymerase chain reaction (RT-PCR), Northern blots, in situ hybridization (see, e.g., Current Protocols in Molecular Biology (Ausubel et al., eds. 2001 supplement)), and immunoassays such as immunohistochemical analysis of sectioned material, Western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998)).

For example, characteristics of pluripotent stem cells are well known to those skilled in the art, and additional characteristics of pluripotent stem cells continue to be identified. Pluripotent stem cell markers include, for example, the expression of one or more of the following: ABCG2, cripto, FOXD3, Connexin43, Connexin45, OCT4, SOX2, Nanog, hTERT, UTF1, ZFP42, SSEA-3, SSEA-4, Tra1-60, or Tra1-81.

After treating pluripotent stem cells with the methods of the present invention, the differentiated cells may be purified by exposing a treated cell population to an agent (such as an antibody) that specifically recognizes a protein marker, such as CXCR4, expressed by cells expressing markers characteristic of the definitive endoderm lineage.

### Formation of Cells Expressing Markers Characteristic of the Pancreatic Endoderm Lineage

Cells expressing markers characteristic of the definitive endoderm lineage may be differentiated into cells expressing markers characteristic of the pancreatic endoderm lineage by any method in the art or by any method proposed in this invention.

For example, cells expressing markers characteristic of the definitive endoderm lineage may be differentiated into cells expressing markers characteristic of the pancreatic endoderm lineage according to the methods disclosed in D'Amour et al, Nature Biotechnology 24, 1392-1401 (2006).

For example, cells expressing markers characteristic of the definitive endoderm lineage are further differentiated into cells expressing markers characteristic of the pancreatic endoderm lineage, by treating the cells expressing markers characteristic of the definitive endoderm lineage with a fibroblast growth factor and the hedgehog signaling pathway inhibitor KAAD-cyclopamine, then removing the medium containing the fibroblast growth factor and KAAD-cyclopamine and subsequently culturing the cells in medium containing retinoic acid, a fibroblast growth factor and KAAD-cyclopamine. An example of this method is disclosed in Nature Biotechnology 24, 1392-1401 (2006).

In one aspect of the present invention, cells expressing markers characteristic of the definitive endoderm lineage are further differentiated into cells expressing markers characteristic of the pancreatic endoderm lineage, by treating the cells expressing markers characteristic of the definitive endoderm lineage with retinoic acid and at least one fibroblast growth factor for a period of time, according to the methods disclosed in U.S. patent application Ser. No. 11/736,908, assigned to LifeScan, Inc.

In one aspect of the present invention, cells expressing markers characteristic of the definitive endoderm lineage are further differentiated into cells expressing markers characteristic of the pancreatic endoderm lineage, by treating the cells expressing markers characteristic of the definitive endoderm lineage with retinoic acid and at least one fibroblast growth factor for a period of time, according to the methods disclosed in U.S. patent application Ser. No. 11/779,311, assigned to LifeScan, Inc.

In one aspect of the present invention, cells expressing markers characteristic of the definitive endoderm lineage are further differentiated into cells expressing markers characteristic of the pancreatic endoderm lineage, by treating the cells expressing markers characteristic of the definitive endoderm lineage according to the methods disclosed in U.S. patent application Ser. No. 60/990,529.

Cells expressing markers characteristic of the definitive endoderm lineage may be treated with at least one other additional factor that may enhance the formation of cells expressing markers characteristic of the pancreatic endoderm lineage. Alternatively, the at least one other additional factor may enhance the proliferation of the cells expressing markers characteristic of the pancreatic endoderm lineage formed by the methods of the present invention. Further, the at least one other additional factor may enhance the ability of the cells expressing markers characteristic of the pancreatic endoderm lineage formed by the methods of the present invention to form other cell types, or improve the efficiency of any other additional differentiation steps.

The at least one additional factor may be, for example, nicotinamide, members of TGF-β family, including TGF-β1, 2, and 3, serum albumin, members of the fibroblast growth factor family, platelet-derived growth factor-AA, and -BB, platelet rich plasma, insulin growth factor (IGF-I, II), growth differentiation factor (such as, for example, GDF-5, -6, -8, -10, -11), glucagon like peptide-I and II (GLP-I and II), GLP-1 and GLP-2 MIMETOBODY™, Exendin-4, retinoic acid, parathyroid hormone, insulin, progesterone, aprotinin, hydrocortisone, ethanolamine, beta mercaptoethanol, epidermal growth factor (EGF), gastrin I and II, copper chelators such as, for example, triethylene pentamine, forskolin, Na-Butyrate, activin, betacellulin, ITS, noggin, neurite growth factor, nodal, valproic acid, trichostatin A, sodium butyrate, hepatocyte growth factor (HGF), sphingosine-1, VEGF, MG132 (EMD, CA), N2 and B27 supplements (Gibco, CA), steroid alkaloid such as, for example, cyclopamine (EMD, CA), keratinocyte growth factor (KGF), Dickkopf protein family, bovine pituitary extract, islet neogenesis-associated protein (INGAP), Indian hedgehog, sonic hedgehog, proteasome inhibitors, notch pathway inhibitors, sonic hedgehog inhibitors, or combinations thereof.

The at least one other additional factor may be supplied by conditioned media obtained from pancreatic cells lines such as, for example, PANC-1 (ATCC No: CRL-1469), CAPAN-1 (ATCC No: HTB-79), BxPC-3 (ATCC No: CRL-1687), HPAF-11 (ATCC No: CRL-1997), hepatic cell lines such as, for example, HepG2 (ATCC No: HTB-8065), and intestinal cell lines such as, for example, FHs 74 (ATCC No: CCL-241).

### Detection of Cells Expressing Markers Characteristic of the Definitive Endoderm Lineage

Markers characteristic of the pancreatic endoderm lineage are well known to those skilled in the art, and additional markers characteristic of the pancreatic endoderm lineage continue to be identified. These markers can be used to confirm that the cells treated in accordance with the present invention have differentiated to acquire the properties characteristic of the pancreatic endoderm lineage. Pancreatic endoderm lineage specific markers include the expression of one or more transcription factors such as, for example, Hlxb9, PTF-1a, PDX-1, HNF-1beta.

The efficiency of differentiation may be determined by exposing a treated cell population to an agent (such as an antibody) that specifically recognizes a protein marker expressed by cells expressing markers characteristic of the pancreatic endoderm lineage.

Methods for assessing expression of protein and nucleic acid markers in cultured or isolated cells are standard in the art. These include quantitative reverse transcriptase polymerase chain reaction (RT-PCR), Northern blots, in situ hybridization (see, e.g., Current Protocols in Molecular Biology (Ausubel et al., eds. 2001 supplement)), and immunoassays such as immunohistochemical analysis of sectioned material, Western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998)).

### Formation of Cells Expressing Markers Characteristic of the Pancreatic Endocrine Lineage

Cells expressing markers characteristic of the pancreatic endoderm lineage may be differentiated into cells expressing markers characteristic of the pancreatic endocrine lineage by any method in the art or by any method disclosed in this invention.

For example, cells expressing markers characteristic of the pancreatic endoderm lineage may be differentiated into cells expressing markers characteristic of the pancreatic endocrine lineage according to the methods disclosed in D'Amour et al, Nature Biotechnology 24, 1392-1401 (2006).

For example, cells expressing markers characteristic of the pancreatic endoderm lineage are further differentiated into cells expressing markers characteristic of the pancreatic endocrine lineage, by culturing the cells expressing markers characteristic of the pancreatic endoderm lineage in medium containing DAPT and exendin 4, then removing the medium containing DAPT and exendin 4 and subsequently culturing the cells in medium containing exendin 1, IGF-1 and HGF. An example of this method is disclosed in Nature Biotechnology 24, 1392-1401 (2006).

For example, cells expressing markers characteristic of the pancreatic endoderm lineage are further differentiated into cells expressing markers characteristic of the pancreatic endocrine lineage, by culturing the cells expressing markers characteristic of the pancreatic endoderm lineage in medium containing exendin 4, then removing the medium containing exendin 4 and subsequently culturing the cells in medium containing exendin 1, IGF-1 and HGF. An example of this method is disclosed in D'Amour et al, Nature Biotechnology, 2006.

For example, cells expressing markers characteristic of the pancreatic endoderm lineage are further differentiated into cells expressing markers characteristic of the pancreatic endocrine lineage, by culturing the cells expressing markers characteristic of the pancreatic endoderm lineage in medium containing DAPT and exendin 4. An example of this method is disclosed in D'Amour et al, Nature Biotechnology, 2006.

For example, cells expressing markers characteristic of the pancreatic endoderm lineage are further differentiated into cells expressing markers characteristic of the pancreatic endocrine lineage, by culturing the cells expressing markers characteristic of the pancreatic endoderm lineage in medium containing exendin 4. An example of this method is disclosed in D'Amour et al, Nature Biotechnology, 2006.

In one aspect of the present invention, cells expressing markers characteristic of the pancreatic endoderm lineage are further differentiated into cells expressing markers characteristic of the pancreatic endocrine lineage, by treating the cells expressing markers characteristic of the pancreatic endoderm lineage with a factor that inhibits the Notch signaling pathway, according to the methods disclosed in U.S. patent application Ser. No. 11/736,908, assigned to LifeScan, Inc.

In one aspect of the present invention, cells expressing markers characteristic of the pancreatic endoderm lineage are further differentiated into cells expressing markers characteristic of the pancreatic endocrine lineage, by treating the cells expressing markers characteristic of the pancreatic endoderm lineage with a factor that inhibits the Notch signaling pathway, according to the methods disclosed in U.S. patent application Ser. No. 11/779,311, assigned to LifeScan, Inc.

In one aspect of the present invention, cells expressing markers characteristic of the pancreatic endoderm lineage are further differentiated into cells expressing markers characteristic of the pancreatic endocrine lineage, by treating the cells expressing markers characteristic of the pancreatic endoderm lineage with a factor that inhibits the Notch signaling pathway, according to the methods disclosed in U.S. patent application Ser. No. 60/953,178, assigned to LifeScan, Inc.

In one aspect of the present invention, cells expressing markers characteristic of the pancreatic endoderm lineage are further differentiated into cells expressing markers characteristic of the pancreatic endocrine lineage, by treating the cells expressing markers characteristic of the pancreatic endoderm lineage according to the methods disclosed in U.S. patent application Ser. No. 60/990,529.

In one aspect of the present invention, the present invention provides a method for increasing the expression of markers associated with the pancreatic endocrine lineage comprising treating cells expressing markers characteristic of the pancreatic endocrine lineage with medium comprising a sufficient amount of a TGF-β receptor agonist to cause an increase in expression of markers associated with the pancreatic endocrine lineage.

The TGF-β receptor agonist may be any agent capable of binging to, and activating the TGF-β receptor. In one embodiment, the TGF-β receptor agonist is selected from the group consisting of activin A, activin B, and activin C.

In an alternate embodiment, the TGF-β receptor agonist may be a peptide variant of activin A. Examples of such peptide variants are disclosed in U.S. patent application Ser. No. 61/076,889, assigned to Centocor R&D, Inc.

Cells expressing markers characteristic of the pancreatic endoderm lineage may be treated with at least one other additional factor that may enhance the formation of cells expressing markers characteristic of the pancreatic endocrine lineage. Alternatively, the at least one other additional factor may enhance the proliferation of the cells expressing markers characteristic of the pancreatic endocrine lineage formed by the methods of the present invention. Further, the at least one other additional factor may enhance the ability of the cells expressing markers characteristic of the pancreatic endocrine lineage formed by the methods of the present invention to form other cell types or improve the efficiency of any other additional differentiation steps.

The at least one additional factor may be, for example, nicotinamide, members of TGF-β family, including TGF-β1, 2, and 3, serum albumin, members of the fibroblast growth factor family, platelet-derived growth factor-AA, and -BB, platelet rich plasma, insulin growth factor (IGF-I, II), growth differentiation factor (such as, for example, GDF-5, -6, -8, -10, -11), glucagon like peptide-I and II (GLP-I and II), GLP-1 and GLP-2 MIMETOBODY™, Exendin-4, retinoic acid, parathyroid hormone, insulin, progesterone, aprotinin, hydrocortisone, ethanolamine, beta mercaptoethanol, epidermal growth factor (EGF), gastrin I and II, copper chelators such as, for example, triethylene pentamine, forskolin, Na-Butyrate, activin, betacellulin, ITS, noggin, neurite growth factor, nodal, valproic acid, trichostatin A, sodium butyrate, hepatocyte growth factor (HGF), sphingosine-1, VEGF, MG132 (EMD, CA), N2 and B27 supplements (Gibco, CA), steroid alkaloid such as, for example, cyclopamine (EMD, CA), keratinocyte growth factor (KGF), Dickkopf protein family, bovine pituitary extract, islet neogenesis-associated protein (INGAP), Indian hedgehog, sonic hedgehog, proteasome inhibitors, notch pathway inhibitors, sonic hedgehog inhibitors, or combinations thereof.

The at least one other additional factor may be supplied by conditioned media obtained from pancreatic cells lines such as, for example, PANC-1 (ATCC No: CRL-1469), CAPAN-1 (ATCC No: HTB-79), BxPC-3 (ATCC No: CRL-1687), HPAF-II (ATCC No: CRL-1997), hepatic cell lines such as, for example, HepG2 (ATCC No: HTB-8065), and intestinal cell lines such as, for example, FHs 74 (ATCC No: CCL-241).

### Detection of Cells Expressing Markers Characteristic of the Pancreatic Endocrine Lineage

Markers characteristic of cells of the pancreatic endocrine lineage are well known to those skilled in the art, and additional markers characteristic of the pancreatic endocrine lineage continue to be identified. These markers can be used to confirm that the cells treated in accordance with the present invention have differentiated to acquire the properties characteristic of the pancreatic endocrine lineage. Pancreatic endocrine lineage specific markers include the expression of one or more transcription factors such as, for example, NGN3, NEUROD, or ISL1.

Markers characteristic of cells of the β cell lineage are well known to those skilled in the art, and additional markers characteristic of the β cell lineage continue to be identified. These markers can be used to confirm that the cells treated in accordance with the present invention have differentiated to acquire the properties characteristic of the β-cell lineage. β cell lineage specific characteristics include the expression of one or more transcription factors such as, for example, PDX1 (pancreatic and duodenal homeobox gene-1), NKX2.2, NKX6.1, ISL1, PAX6, PAX4, NEUROD, HNF1 beta, HNF6, HNT3 beta, or MAFA, among others. These transcription factors are well established in the art for identification of endocrine cells. See, for example, Edlund (Nature Reviews Genetics 3: 524-632 (2002)).

The efficiency of differentiation may be determined by exposing a treated cell population to an agent (such as an antibody) that specifically recognizes a protein marker expressed by cells expressing markers characteristic of the pancreatic endocrine lineage. Alternatively, the efficiency of differentiation may be determined by exposing a treated cell population to an agent (such as an antibody) that specifically recognizes a protein marker expressed by cells expressing markers characteristic of the β cell lineage.

Methods for assessing expression of protein and nucleic acid markers in cultured or isolated cells are standard in the art. These include quantitative reverse transcriptase polymerase chain reaction (RT-PCR), Northern blots, in situ hybridization (see, e.g., Current Protocols in Molecular Biology (Ausubel et al., eds. 2001 supplement)), and immunoassays such as immunohistochemical analysis of sectioned material, Western blotting, and for markers that are accessible in intact cells, flow cytometry analysis (FACS) (see, e.g., Harlow and Lane, Using Antibodies: A Laboratory Manual, New York: Cold Spring Harbor Laboratory Press (1998)).

In one aspect of the present invention, the efficiency of differentiation is determined by measuring the percentage of insulin positive cells in a given cell culture following treatment. In one embodiment, the methods of the present invention produce about 100% insulin positive cells in a given culture. In an alternate embodiment, the methods of the present invention produce about 90% insulin positive cells in a given culture. In an alternate embodiment, the methods of the present invention produce about 80% insulin positive cells in a given culture. In an alternate embodiment, the methods of the present invention produce about 70% insulin positive cells in a given culture. In an alternate embodiment, the methods of the present invention produce about 60% insulin positive cells in a given culture. In an alternate embodiment, the methods of the present invention produce about 50% insulin positive cells in a given culture. In an alternate embodiment, the methods of the present invention produce about 40% insulin positive cells in a given culture. In an alternate embodiment, the methods of the present invention produce about 30% insulin positive cells in a given culture. In an alternate embodiment, the methods of the present invention produce about 20% insulin positive cells in a given culture. In an alternate embodiment, the methods of the present invention produce about 10% insulin positive cells in a given culture. In an alternate embodiment, the methods of the present invention produce about 5% insulin positive cells in a given culture.

In one aspect of the present invention, the efficiency of differentiation is determined by measuring glucose-stimulated insulin secretion, as detected by measuring the amount of C-peptide released by the cells. In one embodiment, cells produced by the methods of the present invention produce about 1000 ng C-peptide/pg DNA. In an alternate embodiment, cells produced by the methods of the present invention produce about 900 ng C-peptide/pg DNA. In an alternate embodiment, cells produced by the methods of the present invention produce about 800 ng C-peptide/pg DNA. In an alternate embodiment, cells produced by the methods of the present invention produce about 700 ng C-peptide/pg DNA. In an alternate embodiment, cells produced by the methods of the present invention produce about 600 ng C-peptide/pg DNA. In an alternate embodiment, cells produced by the methods of the present invention produce about 500 ng C-peptide/pg DNA. In an alternate embodiment, cells produced by the methods of the present invention produce about 400 ng C-peptide/pg DNA. In an alternate embodiment, cells produced by the methods of the present invention produce about 500 ng C-peptide/pg DNA. In an alternate embodiment, cells produced by the methods of the present invention produce about 400 ng C-peptide/pg DNA. In an alternate embodiment, cells produced by the methods of the present invention produce about 300 ng C-peptide/pg DNA. In an alternate embodiment, cells produced by the methods of the present invention produce about 200 ng C-peptide/pg DNA. In an alternate embodiment, cells produced by the methods of the present invention produce about 100 ng C-peptide/pg DNA. In an alternate embodiment, cells produced by the methods of the present invention produce about 90 ng C-peptide/pg DNA. In an alternate embodiment, cells produced by the methods of the present invention produce about 80 ng C-peptide/pg DNA. In an alternate embodiment, cells produced by the methods of the present invention produce about 70 ng C-peptide/pg DNA. In an alternate embodiment, cells produced by the methods of the present invention produce about 60 ng C-peptide/pg DNA. In an alternate embodiment, cells produced by the methods of the present invention produce about 50 ng C-peptide/pg DNA. In an alternate embodiment, cells produced by the methods of the present invention produce about 40 ng C-peptide/pg DNA. In an alternate embodiment, cells produced by the methods of the present invention produce about 30 ng C-peptide/pg DNA. In an alternate embodiment, cells produced by the methods of the present invention produce about 20 ng C-peptide/pg DNA. In an alternate embodiment, cells produced by the methods of the present invention produce about 10 ng C-peptide/pg DNA.

### Therapies

In one aspect, the present invention provides a method for treating a patient suffering from, or at risk of developing, Type 1 diabetes. This method involves culturing pluripotent stem cells, differentiating the pluripotent stem cells in vitro into a β-cell lineage, and implanting the cells of a β-cell lineage into a patient.

In yet another aspect, this invention provides a method for treating a patient suffering from, or at risk of developing, Type 2 diabetes. This method involves culturing pluripotent stem cells, differentiating the cultured cells in vitro into a β-cell lineage, and implanting the cells of a β-cell lineage into the patient.

If appropriate, the patient can be further treated with pharmaceutical agents or bioactives that facilitate the survival and function of the transplanted cells. These agents may include, for example, insulin, members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -7, -8, -10, -15), vascular endothelial cell-derived growth factor (VEGF), pleiotrophin, endothelin, among others. Other pharmaceutical compounds can include, for example, nicotinamide, glucagon like peptide-I (GLP-1) and II, GLP-1 and 2 MIMETOBODY™, Exendin-4, retinoic acid, parathyroid hormone, MAPK inhibitors, such as, for example, compounds disclosed in U.S. Published Application 2004/0209901 and U.S. Published Application 2004/0132729.

The pluripotent stem cells may be differentiated into an insulin-producing cell prior to transplantation into a recipient. In a specific embodiment, the pluripotent stem cells are fully differentiated into β-cells, prior to transplantation into a recipient. Alternatively, the pluripotent stem cells may be transplanted into a recipient in an undifferentiated or partially differentiated state. Further differentiation may take place in the recipient.

Definitive endoderm cells or, alternatively, pancreatic endoderm cells, or, alternatively, β cells, may be implanted as dispersed cells or formed into clusters that may be infused into the hepatic portal vein. Alternatively, cells may be provided in biocompatible degradable polymeric supports, porous non-degradable devices or encapsulated to protect from host immune response. Cells may be implanted into an appropriate site in a recipient. The implantation sites include, for example, the liver, natural pancreas, renal subcapsular space, omentum, peritoneum, subserosal space, intestine, stomach, or a subcutaneous pocket.

To enhance further differentiation, survival or activity of the implanted cells, additional factors, such as growth factors, antioxidants or anti-inflammatory agents, can be administered before, simultaneously with, or after the administration of the cells. In certain embodiments, growth factors are utilized to differentiate the administered cells in vivo. These factors can be secreted by endogenous cells and exposed to the administered cells in situ. Implanted cells can be induced to differentiate by any combination of endogenous and exogenously administered growth factors known in the art.

The amount of cells used in implantation depends on a number of various factors including the patient's condition and response to the therapy, and can be determined by one skilled in the art.

In one aspect, this invention provides a method for treating a patient suffering from, or at risk of developing diabetes. This method involves culturing pluripotent stem cells, differentiating the cultured cells in vitro into a β-cell lineage, and incorporating the cells into a three-dimensional support. The cells can be maintained in vitro on this support prior to implantation into the patient. Alternatively, the support containing the cells can be directly implanted in the patient without additional in vitro culturing. The support can optionally be incorporated with at least one pharmaceutical agent that facilitates the survival and function of the transplanted cells.

Support materials suitable for use for purposes of the present invention include tissue templates, conduits, barriers, and reservoirs useful for tissue repair. In particular, synthetic and natural materials in the form of foams, sponges, gels, hydrogels, textiles, and nonwoven structures, which have been used in vitro and in vivo to reconstruct or regenerate biological tissue, as well as to deliver chemotactic agents for inducing tissue growth, are suitable for use in practicing the methods of the present invention. See, for example, the materials disclosed in U.S. Pat. No. 5,770,417, U.S. Pat. No. 6,022,743, U.S. Pat. No. 5,567,612, U.S. Pat. No. 5,759,830, U.S. Pat. No. 6,626,950, U.S. Pat. No. 6,534,084, U.S. Pat. No. 6,306,424, U.S. Pat. No. 6,365,149, U.S. Pat. No. 6,599,323, U.S. Pat. No. 6,656,488, U.S. Published Application 2004/0062753 A1, U.S. Pat. No. 4,557,264 and U.S. Pat. No. 6,333,029.

To forma support incorporated with a pharmaceutical agent, the pharmaceutical agent can be mixed with the polymer solution prior to forming the support. Alternatively, a pharmaceutical agent could be coated onto a fabricated support, preferably in the presence of a pharmaceutical carrier. The pharmaceutical agent may be present as a liquid, a finely divided solid, or any other appropriate physical form. Alternatively, excipients may be added to the support to alter the release rate of the pharmaceutical agent. In an alternate embodiment, the support is incorporated with at least one pharmaceutical compound that is an anti-inflammatory compound, such as, for example compounds disclosed in U.S. Pat. No. 6,509,369.

The support may be incorporated with at least one pharmaceutical compound that is an anti-apoptotic compound, such as, for example, compounds disclosed in U.S. Pat. No. 6,793,945.

The support may also be incorporated with at least one pharmaceutical compound that is an inhibitor of fibrosis, such as, for example, compounds disclosed in U.S. Pat. No. 6,331,298.

The support may also be incorporated with at least one pharmaceutical compound that is capable of enhancing angiogenesis, such as, for example, compounds disclosed in U.S. Published Application 2004/0220393 and U.S. Published Application 2004/0209901.

The support may also be incorporated with at least one pharmaceutical compound that is an immunosuppressive compound, such as, for example, compounds disclosed in U.S. Published Application 2004/0171623.

The support may also be incorporated with at least one pharmaceutical compound that is a growth factor, such as, for example, members of the TGF-β family, including TGF-β1, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -8, -10, -15), vascular endothelial cell-derived growth factor (VEGF), pleiotrophin, endothelin, among others. Other pharmaceutical compounds can include, for example, nicotinamide, hypoxia inducible factor 1-alpha, glucagon like peptide-I (GLP-1), GLP-1 and GLP-2 MIMETOBODY™, and II, Exendin-4, nodal, noggin, NGF, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, MAPK inhibitors, such as, for example, compounds disclosed in U.S. Published Application 2004/0209901 and U.S. Published Application 2004/0132729.

The incorporation of the cells of the present invention into a scaffold can be achieved by the simple depositing of cells onto the scaffold. Cells can enter into the scaffold by simple diffusion (J. Pediatr. Surg. 23 (1 Pt 2): 3-9 (1988)). Several other approaches have been developed to enhance the efficiency of cell seeding. For example, spinner flasks have been used in seeding of chondrocytes onto polyglycolic acid scaffolds (Biotechnol. Prog. 14(2): 193-202 (1998)). Another approach for seeding cells is the use of centrifugation, which yields minimum stress to the seeded cells and enhances seeding efficiency. For example, Yang et al. developed a cell seeding method (J. Biomed. Mater. Res. 55(3): 379-86 (2001)), referred to as Centrifugational Cell Immobilization (CCl).

The present invention is further illustrated, but not limited by, the following examples.

### EXAMPLES

### Example 1

### Design of the Peptides of the Present Invention

The aim of this work was to design variant peptides of activin A, based on the available structural information for ligands and respective ligand-receptor interactions of the known activin peptides and other members of the TGF-β family. Analysis of two crystal structures of activin A (1nyu and 1s4Y, located at the Protein databank: http://www.rcsb.org), identified a number of amino acid residues that may be mutated. Residues that were located at the homo-dimer interface were selected for mutation. Even though a portion of the dimer interface residues are common, the relative orientation of the monomers in the crystals differs significantly. Therefore, two separate sets of residues were chosen, one based on each crystal structure. Cysteine, glycine and proline residues were not varied because these often play distinct structural roles in proteins, such as, for example, formation of disulphide bonds, in the case of cysteine residues, or the adoption of specific backbone angles inaccessible by other residues, in the case of glycine and proline residues.

Using the crystal structure of the activin A complex with pdb code 1nyu, the following sites were targeted for mutations: 10I, 16F, 39Y, 41E, 43E, 74F, 75A, 76N, 77L, 78K, 79S, 82V. Using the crystal structure of the activin A complex with pdb code 1s4y, the following sites were targeted for mutations: 16F, 18V, 19S, 20F, 37A, 38N, 39Y, 41E, 74F, 82V, 107N, 109I, 110V, 116S.

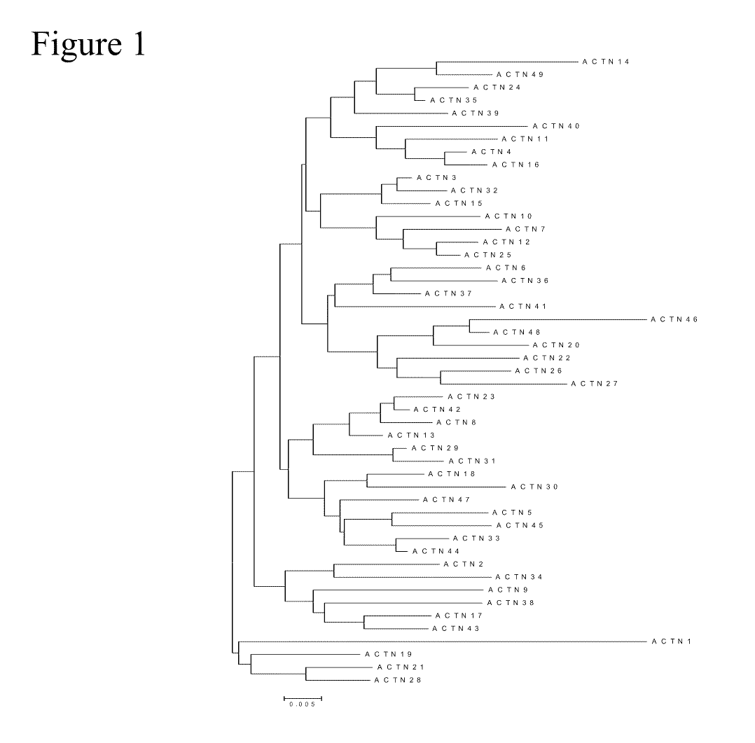

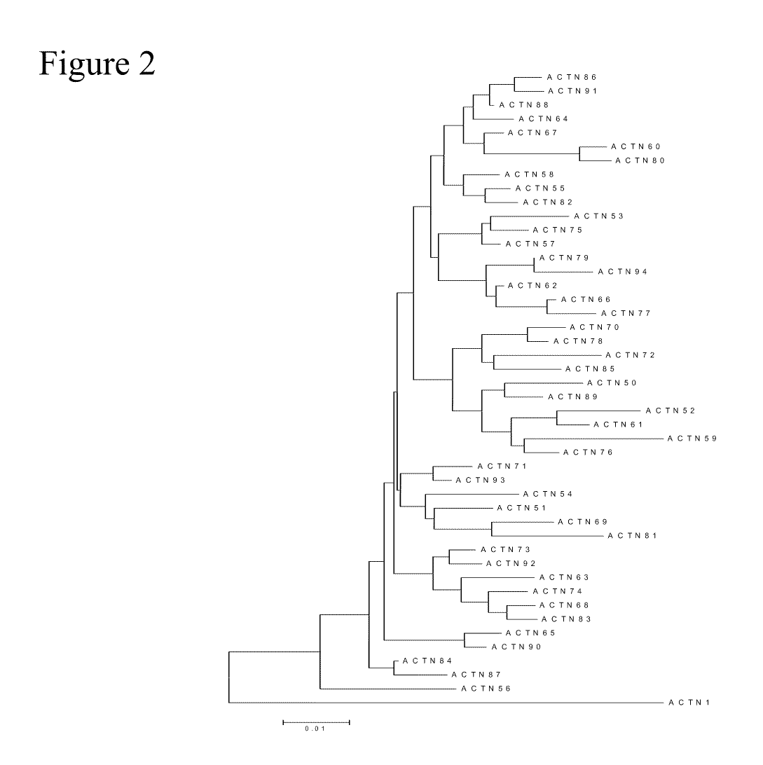

The program Rosetta (see, for example Simons, et al, Mol Biol, 268, 209-225, 1997, and Simons, K. T., et al, Proteins, 34, 82-95, 1999) was used to make combinatorial mutations of the selected residues in both monomeric chains of the activin ligand. The program chose rotamers of side chain conformations for each of the 20 amino acids and explored energetically favorable conformations using a Metropolis Monte Carlo procedure. A total of 93 designs were chosen along with the wildtype activin A peptide sequence. These were tested according to the methods of the present invention. Table 1 lists the amino acid sequences of the peptides of the present invention. ACTN1 is the wildtype activin molecule. ACTN 2 to ACTN 48 are peptide sequences of the present invention that were calculated using the crystal structure 1nyu. ACTN 49 to ACTN 94 are peptide sequences of the present invention that were calculated using the crystal structure 1s4y. No two peptide sequences were identical. Variability in the peptide sequences is shown as a phylogenetic tree in FIG. 1 for ACTN 2 to ACTN 48, and FIG. 2 for ACTN 49 to ACTN 94.

### Example 2

### Cloning and Expression of the Peptides of the Present Invention

Genes encoding the peptides listed in Table 1 were designed for cloning into an expression vector. Based on the scientific literature for the proteolytic processing of precursor forms of activin A and other members of the TGF-beta family, the expression constructs were designed to contain the full precursor form of activin A (pro region plus the mature protein). The wild type activin A precursor expression construct was created to allow the subsequent construction of all activin A variant expression constructs by cloning of the coding sequences containing only the mature protein region into the wild type activin A construct. All expression constructs, therefore, have the identical activin A pro region.

The amino acid sequences for wild type activin A and all 93 designed variants in Table I were back-translated into DNA sequence using human codon preference, using the methods disclosed in U.S. Pat. Nos. 6,670,127 and 6,521,427, assigned to Centocor R&D Inc. DNA sequences are listed in Table 2. The native amino acid and native DNA sequence, without back-translation, for the pro region of wild type activin A were used and are listed in Tables 1 and 2, respectively. Each DNA sequence, consisting of the single pro domain and the 94 mature protein domains, was then generated by parsing the sequence into smaller fragments and synthesizing these as oligonucleotides using GENEWRITER™ technology (Centocor R&D, US) then purified by RP HPLC (Dionex, Germany). The purified oligonucleotides for each DNA sequence were then independently assembled into a full-length DNA fragment using the methods disclosed in U.S. Pat. Nos. 6,670,127 and 6,521,427, assigned to Centocor R&D Inc.

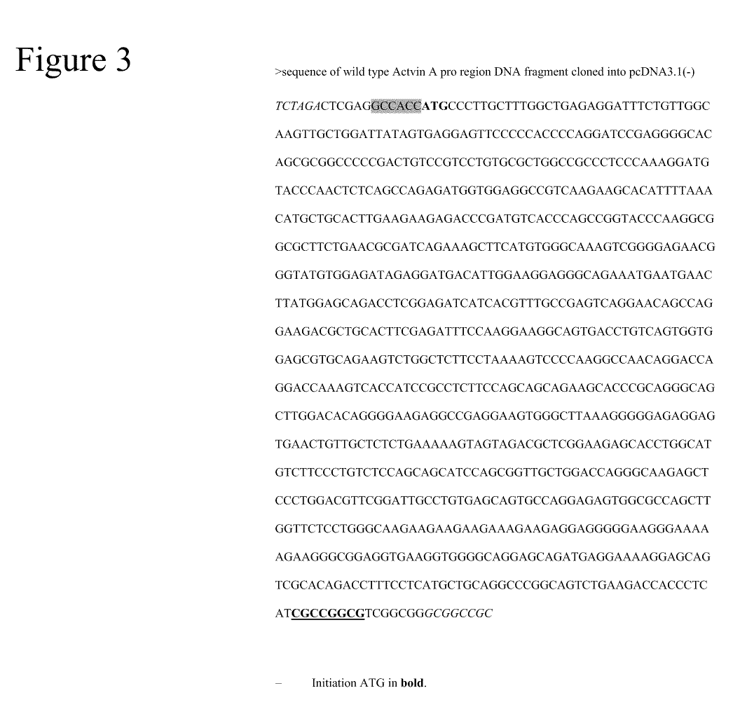

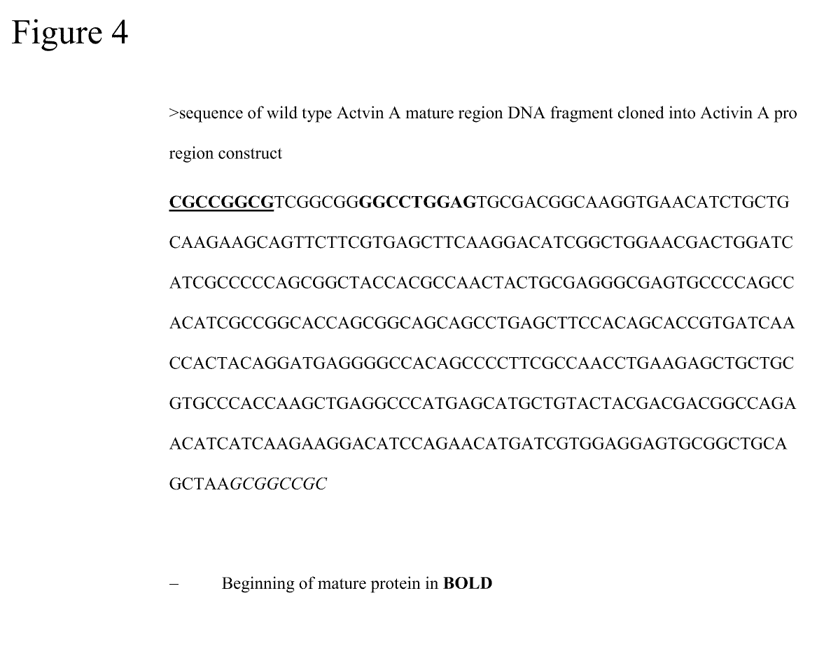

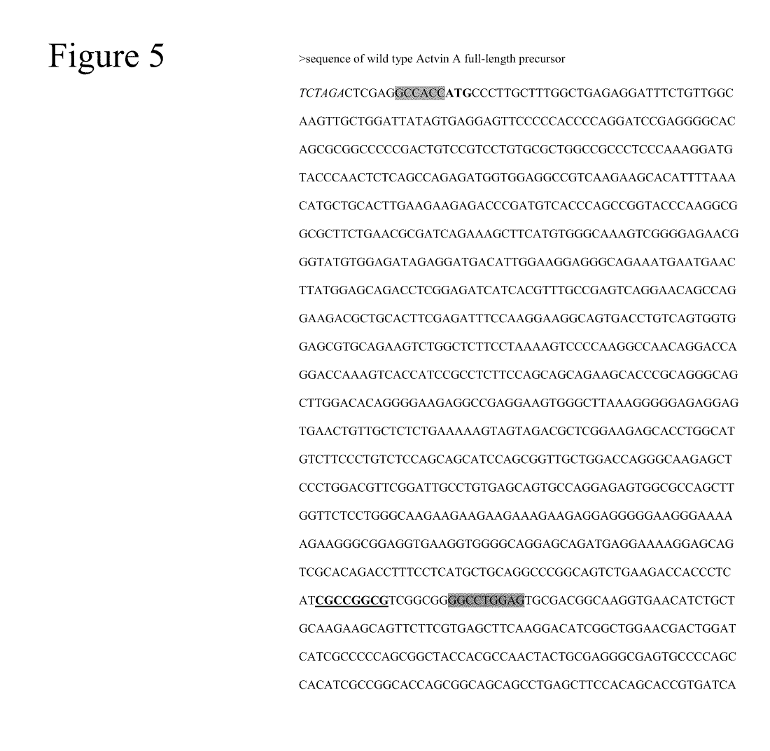

As a first step, an expression construct containing wild type activin A (ACTN 1) was prepared to evaluate the expression system before proceeding with the entire library of variants. The activin A pro region DNA fragment was cloned into pcDNA3.1(−) (Invitrogen, Cat. No. V795-20) using XbaI and NotI sites (in italics, FIG. 3). The DNA fragment encoding the activin A mature protein was then cloned into this pro region construct and fused in frame to the pro region using SgrAI (in bold underscored) and NotI sites (FIG. 4), generating a full-length precursor expression construct (FIG. 5). The DNA fragments encoding the mature protein of variants ACTN 2 to ACTN 8 were then cloned in a similar manner into the pro region construct to generate precursor expression constructs of these variants. As a positive control, a commercially available human activin A expression construct was obtained from OriGene Technologies, Inc. (Cat. No. TC118774). The accession number for the mRNA of the human activin A in this clone is NM_{—}002192.2, and the mammalian expression vector is pCMV6-XL4.

Transfection and expression of gene constructs: The expression and activity of the ACTN 1 and OriGene wild type activin A precursor constructs were compared to determine if the ACTN 1 construct would produce an active molecule.

Cell Maintenance: HEK293-E cells were grown in 293 FreeStyle medium (Invitrogen; Cat #12338). Cells were diluted when the cell concentration was between 1.5 and 2.0×10⁶ cells per ml to 2.0×10⁵ cells per ml. The cells were grown in a humidified incubator shaking at 125 RPM at 37° C. and 8% CO₂.

Transfection of Activin A Variants: Variants were transfected into HEK293-E cells in separate 125 ml shake flasks (Corning; Cat #431143) containing 20 ml of medium. The cells were diluted to 1.0×10⁶ cells per ml. Total DNA (25 μg) was diluted in 1.0 ml of Opti-Pro (Invitrogen; Cat #12309), and 25 μl of FreeStyle Max transfection reagent (Invitrogen; Cat #16447) was diluted in 1.0 ml of Opti-Pro. The diluted DNA was added to the diluted Max reagent and incubated for 10 minutes at room temperature. An aliquot of 2 ml of the DNA Max complex was added to the flask of cells and placed in an incubator for 96 hours shaking at 125 RPM at 37° C. and 8% CO₂.

The supernatant was separated from the cells by centrifugation at 5,000×g for 10 minutes and filtered through a 0.2 μm filter (Corning; Cat #431153), then concentrated 10 and 50 fold using an Amicon Ultra Concentrator 10K (Cat #UFC901096), and centrifuging for approximately 10 minutes at 3,750×g.

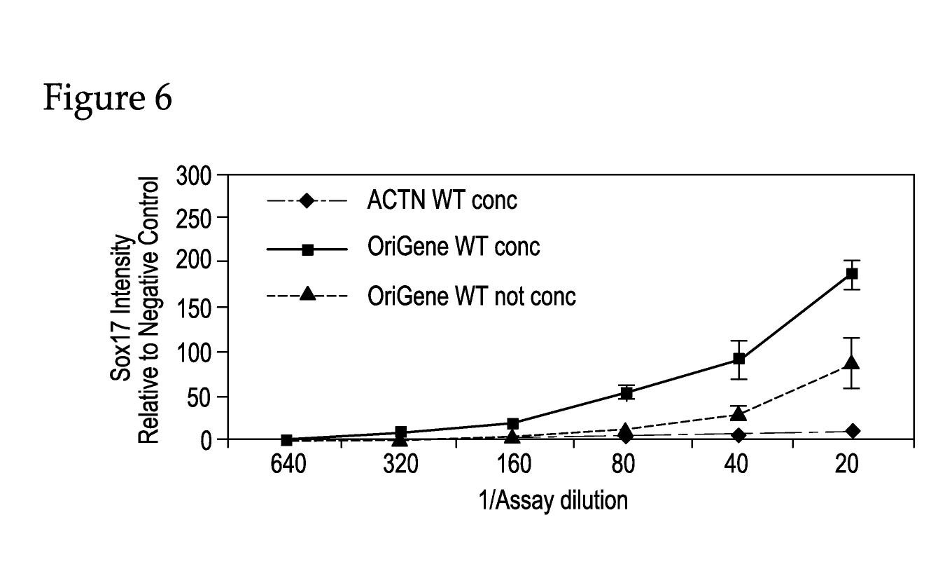

Concentrated and unconcentrated supernatants were checked for activin A activity in a cell-based assay, measuring the ability of the peptides of the present invention to differentiate human embryonic stem cells into cells expressing markers characteristic of the definitive endoderm lineage (see Example 6) with SOX17 intensity as the readout. Both the concentrated and unconcentrated supernatants from the OriGene wildtype construct had much greater activity (SOX17 intensity) than the concentrated supernatant from the ACTN 1 construct (FIG. 6). This result led to the decision to change the ACTN 1 construct from the pcDNA3.1(−) expression vector to the Centocor mammalian expression vector pUnder, which has consistently better expression characteristics, likely due to inclusion of a complete Intron A upstream of the core CMV promoter.

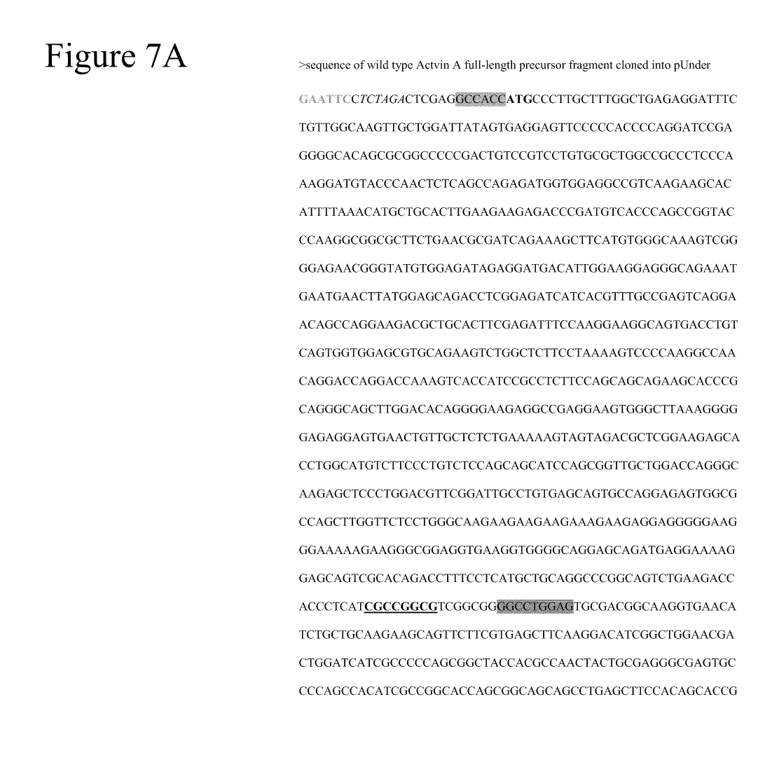

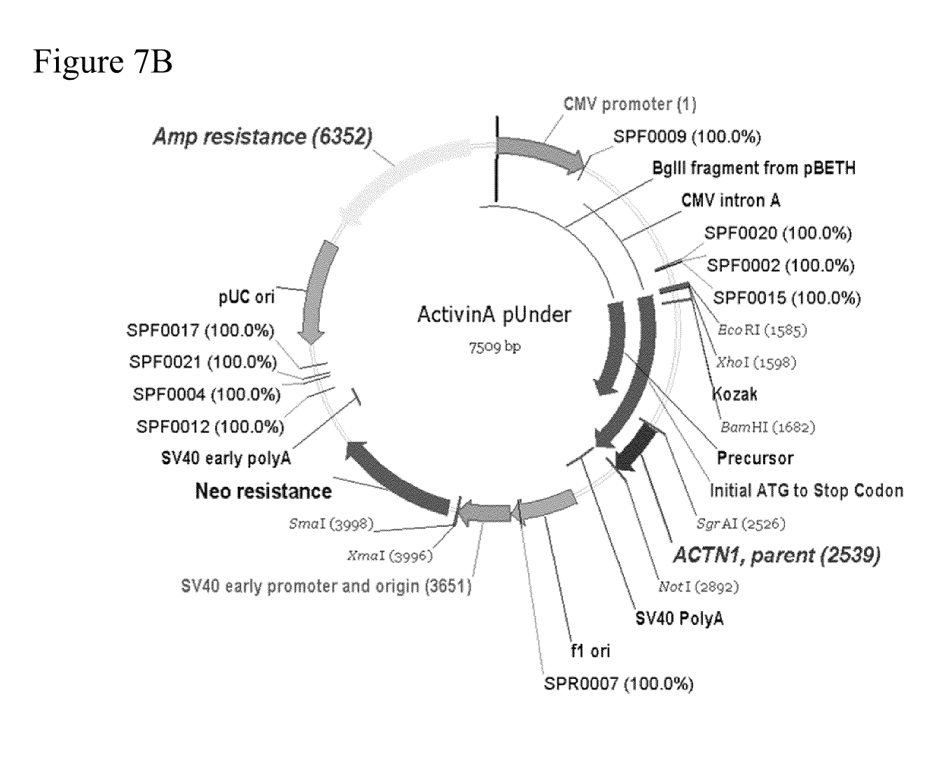

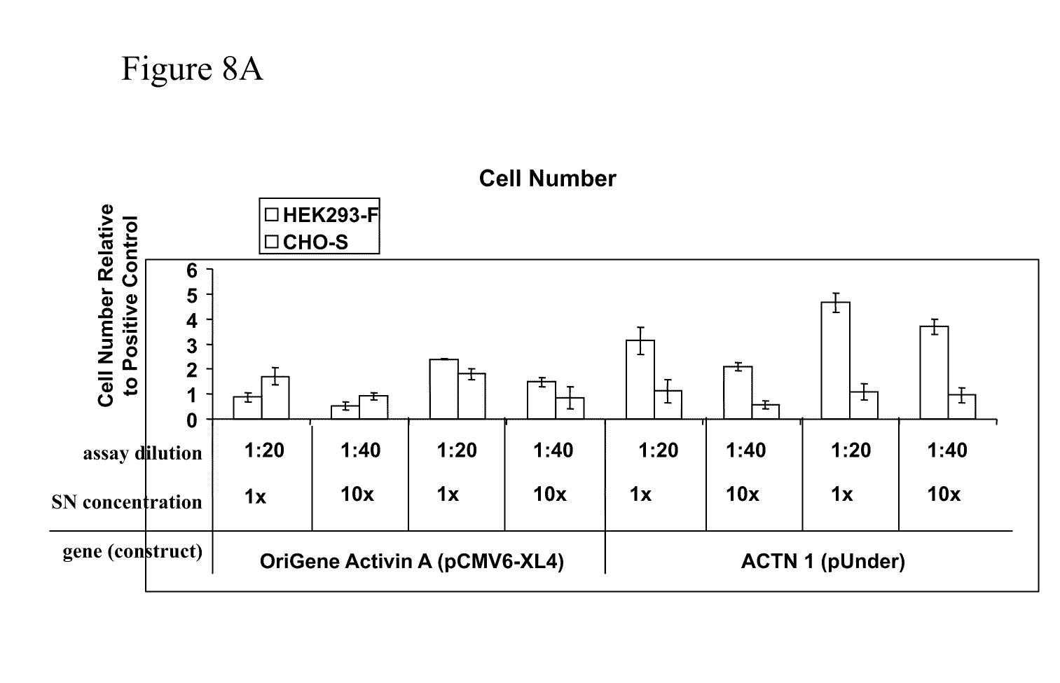

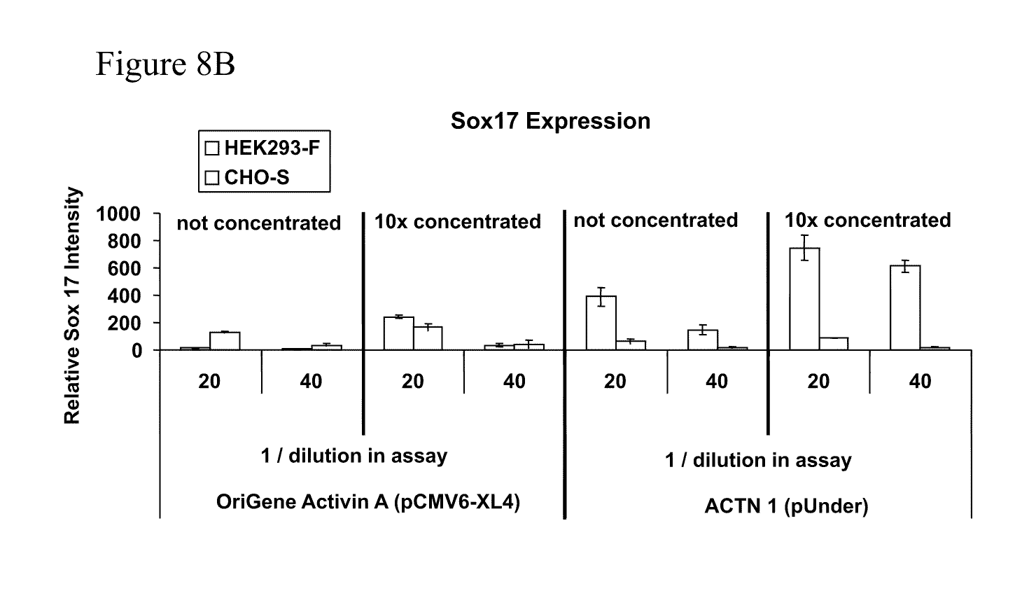

The full-length ACTN 1 precursor gene was subcloned from pcDNA3.1(−) into pUnder using EcoRI and HindIII sites (in bold grey, FIGS. 7A and 7B). Both this new ACTN 1 wild type activin A construct along with the OriGene construct were separately transfected into CHO-S or HEK293-F cells. Supernatants were prepared as above and tested for activin A activity. Supernatants from the ACTN 1 pUnder construct were found to have greater activity in the cell-based assay, as judged by cell number and SOX17 intensity increases, than supernatants from the OriGene wildtype construct (FIGS. 8A and 8B).

As the expression of ACTN 1 from the pUnder construct resulted in supernatants with higher levels of activity than from the corresponding pcDNA3.1(−) construct, full-length precursor expression constructs were then generated for the entire library of activin A variants in pUnder. The DNA fragments of the variants spanning only the mature protein region were each subcloned into pUnder using the SgrAI and NotI cloning sites (in bold underscore and italics, FIGS. 7A and 7B), replacing the ACTN 1 mature protein coding sequence, while still leaving the pro-region intact.

Transfection of Activin-A Variants: Variants were transfected using HEK293-F cells in separate 125 ml shake flasks (Corning; Cat #431143) with 20 ml of medium. The cells were diluted to 1.0×10⁶ cells per ml. Total DNA (25 μg) was diluted in 1.0 ml of Opti-Pro (Invitrogen; Cat #12309), and 25 μl of FreeStyle Max transfection reagent (Invitrogen; Cat #16447) was diluted in 1.0 ml of Opti-Pro. The diluted DNA was added to the diluted Max reagent and incubated for 10 minutes at room temperature. An aliquot of 2 ml of the DNA Max complex was added to the flask of cells and placed in an incubator for 96 hours shaking at 125 RPM, 37° C. and 8% CO₂.

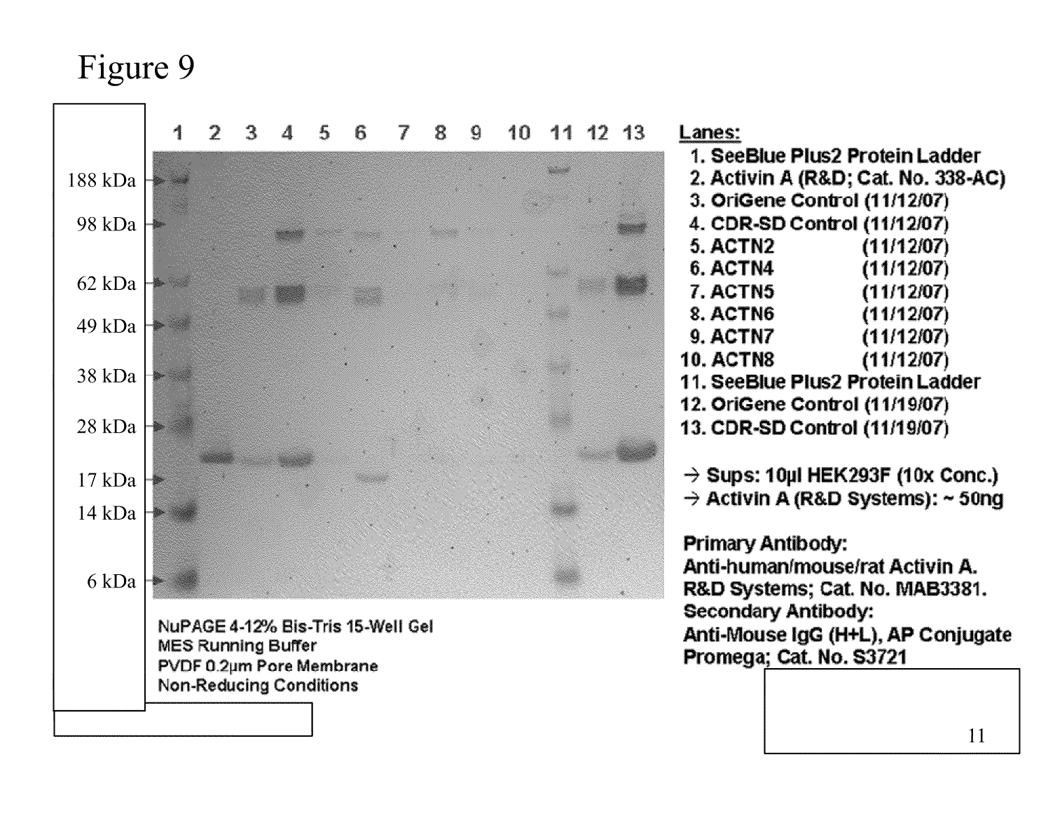

Western blot analysis was carried out on supernatants generated using the pUnder expression constructs of the first seven activin A variants (ACTN 2 to ACTN 8). The OriGene and ACTN 1 activin A wildtype controls were included. The apparent molecular mass of these two control proteins were similar and were consistent with a calculated molecular mass of 26 kD. Expression from several of the variants was observed, although expression levels were inconsistent between variants (FIG. 9). This indicated overall that the amino acid substitutions in some of the variants did not impact expression and still allowed for the correct processing of the precursor protein.

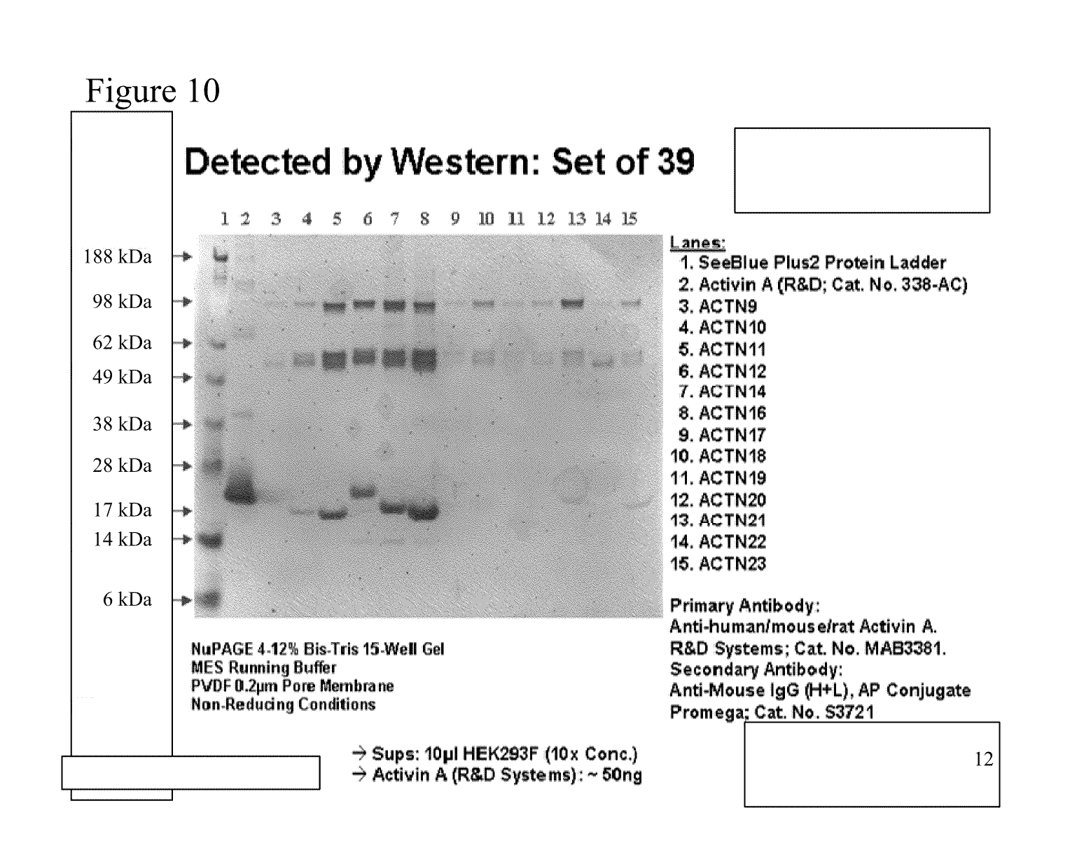

A second group of supernatants from pUnder expression constructs (39 in all) was also analyzed by Western blot. Expression from most of the variants was not detectable (data not shown). A Western blot of only those variants with detectable signal is shown in FIG. 10. The remaining variants were not analyzed for expression in this manner.

### Background to Examples 3 and 4

### Affinity Purification of the Peptides of the Present Invention

The objective in this section was to develop a means of affinity purification for the activin A variants. The first approach, termed bis-his, was to introduce metal binding sites into the amino acid sequence of the peptides of the present invention that would allow each variant to bind selectively to a metal affinity matrix. If a bis-his variant could be identified that bound with high affinity to the matrix and was applicable to all activin A variants, this bis-his site could be incorporated at the point of gene assembly. However, since these variants would bind at lower affinity than proteins with poly-histidine tags, clear separation from other endogenous proteins with similar metal binding sites was uncertain. To address this, a follistatin affinity matrix was also employed that would specifically bind all activin A variants. Although this approach involves expressing and purifying follistatin and then generating a follistatin affinity matrix, it also may facilitate the purification of other TGF-β family members. These two approaches are outlined below in Examples 3 and 4.

### Example 3

### Metal-Chelate Purification of the Peptides of the Present Invention

The first approach involves engineering the molecule to selectively bind a metal affinity chromatography matrix. Engineered proteins can be tagged with a peptide sequence that enhances the purification of the protein. Integration of a series of histidine residues into the peptide sequence is one example whereby the protein of interest can be purified using immobilized metal affinity chromatography (IMAC). IMAC is based on coordinate covalent binding of histidine residues to metals, such as, for example, cobalt, nickel, or zinc. After binding, the protein of interest may be eluted through a change of pH or by adding a competitive molecule, such as imidazole, thereby providing a degree of purification. Typically the histidine residues are introduced at either the N or C terminus. However, since activin A is expressed as a precursor peptide, wherein the N-terminus is cleaved, an N-terminus tag would be lost during intracellular processing. Furthermore, addition of a C-terminus tag was suspected to prevent correct dimerization and processing of the molecule. See, for example, Pangas, S. and Woodruff, T.; J. Endocrinology, vol 172, pgs 199-210, 2002. Therefore, internal positions within the mature activin A sequence were selected for substitution with histidine residues to create a synthetic metal binding site. This approach introduces two solvent-exposed histidine residues separated either by a single turn of an alpha-helix (His-X3-His) or at two positions apart in a beta-sheet (His-X-His). See, for example, Suh et al., Protein Engineering, vol. 4, no. 3, pgs 301-305, 1991. Table 3 shows the amino acid sequence of selected peptides in which histidine substitutions have been made.

Transfection of the peptides of the present invention containing histidine substitutions: Gene sequences, encoding the peptides listed in Table 3, were generated and inserted into the pUnder vector according to the methods described in Example 2. HEK293-F cells were transiently transfected as follows: on the day of transfection, cells were diluted to 1.0×10⁶ cells per ml in 750 ml of medium in separate 2 L shake flasks (one per vector) (Corning; Cat #431255). Total DNA (937.5 μl) was diluted in 7.5 ml of Opti-Pro (Invitrogen; Cat #12309), and 937.5 μl of FreeStyle Max transfection reagent (Invitrogen; Cat #16447) was diluted in 7.5 ml of Opti-Pro. The diluted DNA was added to the diluted Max reagent and incubated for 10 minutes at room temperature. An aliquot of 15 ml of the DNA Max complex was added to the flask of cells and placed in an incubator for 96 hours shaking at 125 RPM, 37° C. and 8% CO₂.

Purification of the peptides of the present invention containing histidine substitutions: Purifications using immobilized metal-chelate affinity chromatography (IMAC) were performed on an AKTA FPLC chromatography system using GE Healthcare's Unicorn™ software.

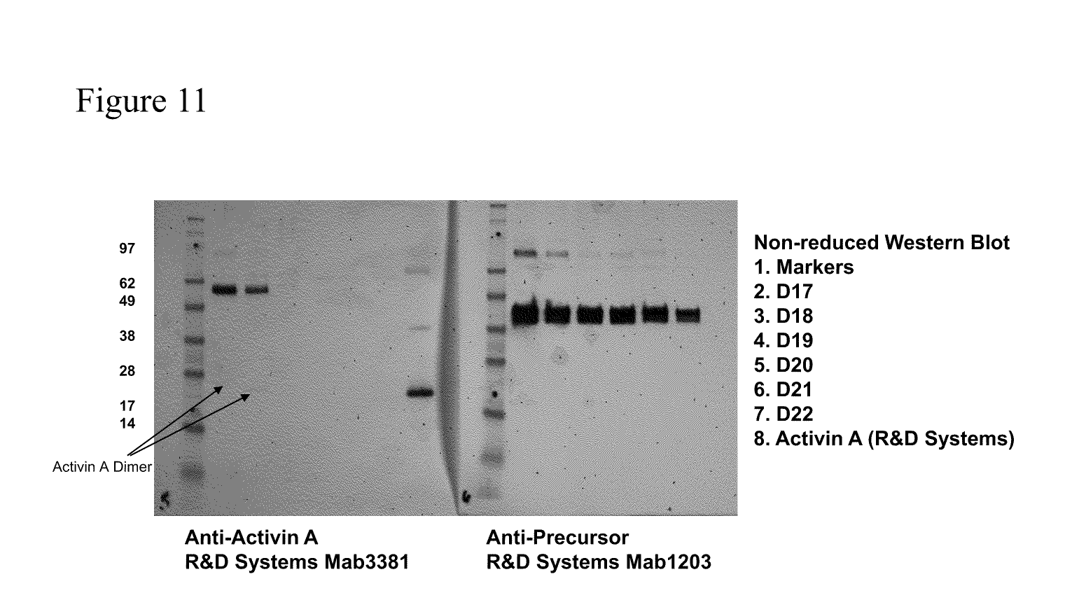

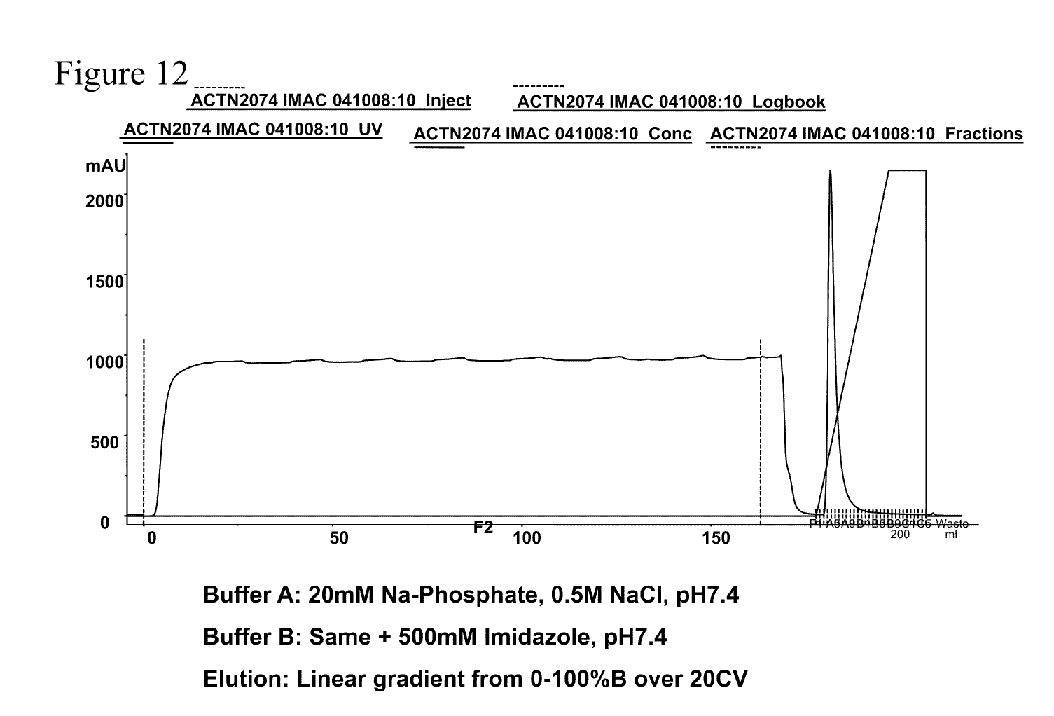

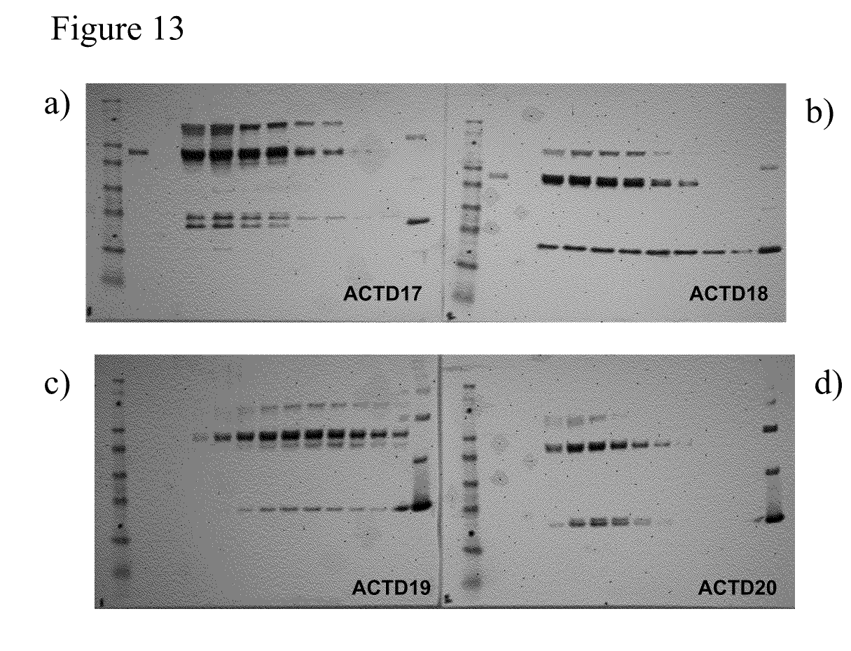

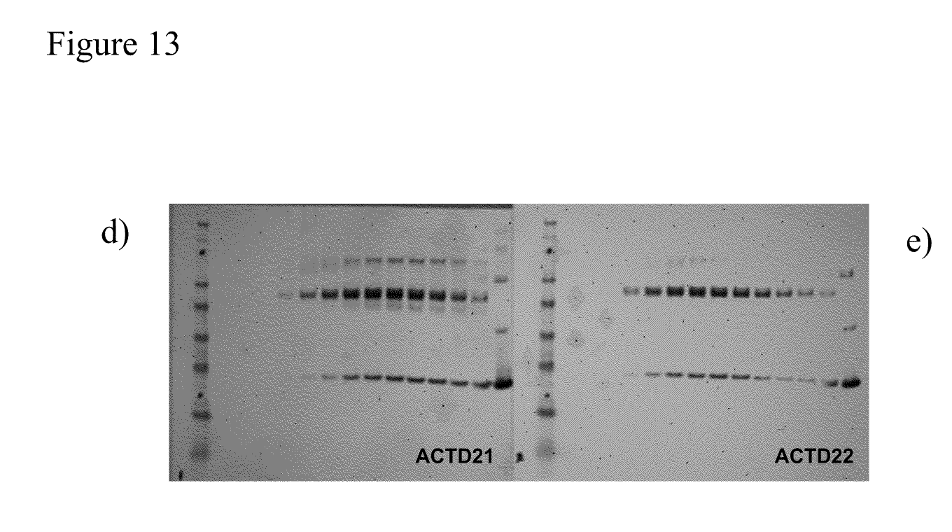

Briefly, cell supernatants from transiently transfected HEK293-F cells were harvested four days after transfection, clarified by centrifugation (30 min, 6000 rpm), and filtered (0.2 μm PES membrane, Corning). The relative amount of specific protein was determined using an activin A ELISA (R&D Systems; Cat #DY338) as per manufacturer's instructions. The samples were concentrated 4-fold using an LV Centramate (Pall) concentrator and checked by Western blot using anti-activin A antibody (R&D Systems; Cat #3381) or anti-activin A precursor antibody (R&D Systems; Cat #1203) for detection. FIG. 11 shows a representative profile of several peptide variants after 4-fold concentration of the respective supernatants. The concentrated samples were then diluted with 10×PBS to a final concentration of 1×PBS, and again 0.2 μm filtered. Diluted supernatants were loaded onto an equilibrated (20 mM Na-Phosphate, 0.5M NaCl, pH7.4) HisTrap column (GE Healthcare) at a relative concentration of approximately 10 mg protein per ml of resin. After loading, the column was washed and protein eluted with a linear gradient of imidazole (0-500 mM) over 20 column volumes. FIG. 12 shows a representative IMAC purification profile for the peptide variant ACTD20. Peak fractions were pooled and dialyzed against PBS, pH 7 overnight at 4° C. The proteins were removed from dialysis, filtered (0.2 μm), and the total protein concentration determined by absorbance at 280 nm on a NANODROP™ spectrophotometer (Thermo Fisher Scientific). Specific protein concentration was determined using an activin A ELISA, as stated previously. If necessary, the purified proteins were concentrated with a 10K molecular weight cut-off (MWCO) centrifugal concentrator (Millipore). The quality of the purified proteins was assessed by SDS-PAGE and Western blot using anti activin A antibody (R&D Systems; Cat #3381) or anti-activin A precursor (R&D Systems; Cat #1203) for detection. FIG. 13 shows the Western blot elution profiles for imidazole fractions from six representative peptide purifications. Purified proteins were stored at 4° C.

All single bis-his pair constructs examined were retarded on a metal affinity chromatography matrix as anticipated. However, since these point mutations result in a single metal binding site, binding to the matrix was non-specific, and the variants co-eluted with other endogenous proteins containing similar sites. In order to enhance specific binding and retention, an additional pair of histidine residues was added to each of the K7H/N9H single pair constructs (Table 4). Again, each double bis-his construct exhibited clear enrichment on a metal affinity matrix as well as a distinct separation from non-specifically bound proteins. A third pair of histidine residues was also added to the best separated of these constructs (ACTD 23 from ACTN 34) in an attempt to further increase the separation from non-specifically bound proteins. This molecule, however, did not exhibit specific retention above the double bis-his construct.

### Example 4

### Follistatin Purification of the Peptides of the Present Invention

A second approach towards purifying a range of activin A variants was taken to exploit the high affinity interaction between follistatin and activin A. Follistatin is a natural activin A antagonist, inhibiting both type I and type II receptor interactions. Since the variants in the present invention encompass changes in the dimer interface and not the receptor binding surfaces, follistatin was a logical choice for an affinity matrix since changes were not made to the receptor binding surfaces. Follistatin 288 and 315 (residues 1-288 and 1-315 of follistatin, respectively) bind activin A at very high affinity (approximately 300 pM) while follistatin 12 and 123 (residues 64-212 and residues 64-288 of follistatin, respectively) bind with moderate affinity (approximately 400 nM). The follistatin constructs tested included follistatin 12 (FS12), follistatin 288 (FS288) and follistatin 315 (FS315), see Table 5. Each of these constructs was designed for mammalian expression and contained a poly histidine tag for metal affinity purification.

Cloning of follistatin variants: The protein and gene sequences for three poly histidine tagged, designed follistatin gene variants, ACTA 1, ACTA2, and ACTA 3, are given in Tables 6 and 7, respectively. The genes were synthesized and assembled as described for the activin A gene variants in Example 2. The assembled genes were cloned, using EcoRI and HindIII restriction sites that precede and follow each of the gene sequences, into the Centocor pUnder mammalian expression vector (detailed in Example 2), utilizing the unique EcoRI and HindIII restriction sites of the vector.

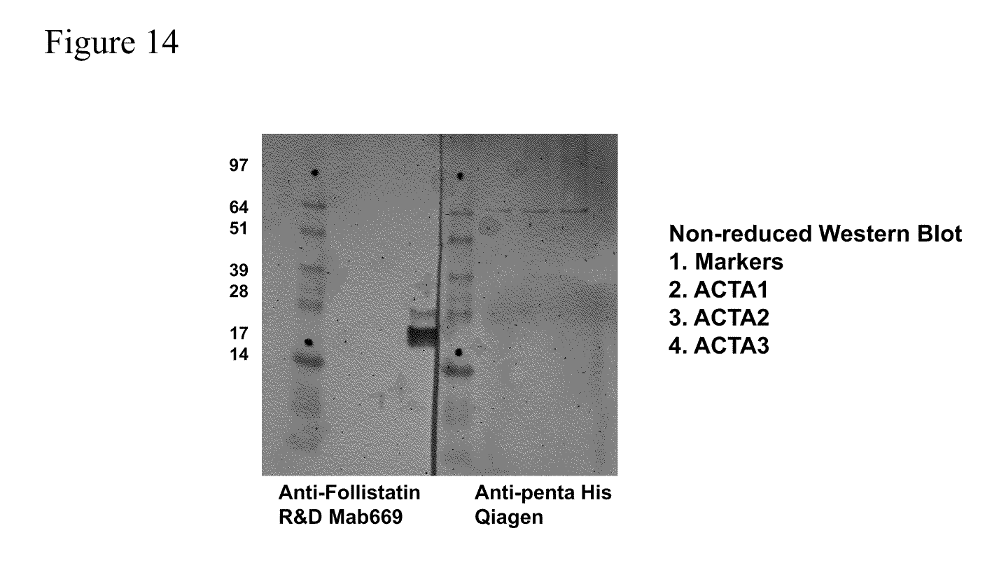

Evaluation of expression of follistatin variants: Variants (ACTA 1, ACTA2 and ACTA3) were transfected using HEK293-F cells in separate 2 L shake flasks (one per vector) (Corning; Cat #431255) with 750 ml of medium. The cells were diluted to 1.0×10⁶ cells per ml. Total DNA (937.5 μg) was diluted in 7.5 ml of Opti-Pro (Invitrogen; Cat #12309), and 937.5 μl of FreeStyle Max transfection reagent (Invitrogen; Cat #16447) was diluted in 7.5 ml of Opti-Pro. The diluted DNA was added to the diluted Max reagent and incubated for 10 minutes at room temperature. An aliquot of 15 ml of the DNA Max complex was added to the flask of cells and placed in an incubator for 96 hours shaking at 125 RPM, 37° C. and 8% CO₂. Cell supernatants were harvested four days after transfection, clarified by centrifugation (30 min, 6000 rpm), and filtered (0.2 μm PES membrane, Corning). Expression of follistatin variants was checked by Western blot using anti-Follistatin antibody (R&D Systems; Cat #669) or anti-penta-Histidine antibody (Qiagen; Cat #34660) for detection. FIG. 14 shows a representative Western blot for follistatin variant expression from the culture supernatants. One variant, ACTA 3, was selected for scale-up expression and purification.

Scale-up expression of ACTA 3: HEK293-F cells were transiently transfected in an Applikon bioreactor. The bioreactor was seeded at 4.0×10⁶ cells per ml the day prior to transfection. The bioreactor was controlled with air in the headspace; O₂ was monitored and controlled at 50% through the sparge. The pH was controlled by CO₂ and sodium bicarbonate. The cells were stirred with a marine impeller at 115 RPM. Prior to transfection the pH was maintained at 7.2 then lowered to 6.8 at the time of transfection.

At the time of transfection, cell concentration was 1.0×10⁶ cells per ml. Total DNA (1.25 mg/L) was diluted in 50 ml/L of Opti-Pro, and 1.25 ml/L of FreeStyle Max transfection reagent was diluted in 50 ml/L of Opti-Pro. The diluted DNA was added to the diluted Max reagent and incubated for 10 minutes at room temperature. An aliquot of 100 ml/L of the DNA Max complex was added to the bioreactor and grown for 96 hours.

Metal-chelate purification of ACTA3: Purifications were performed on an AKTA FPLC chromatography system using GE Healthcare's Unicorn™ software.

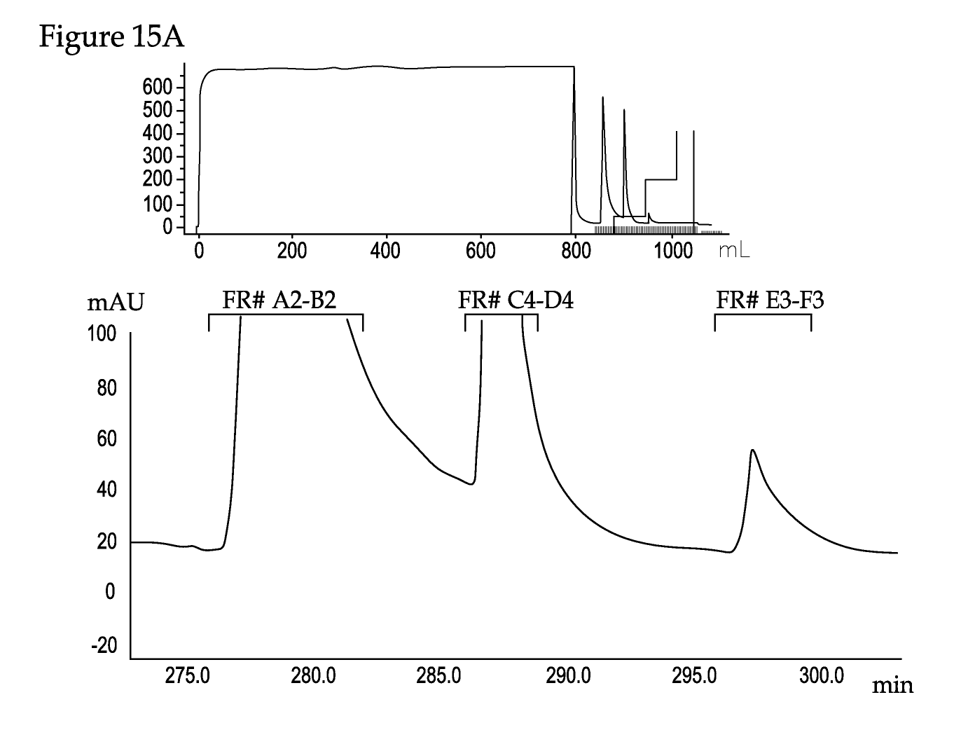

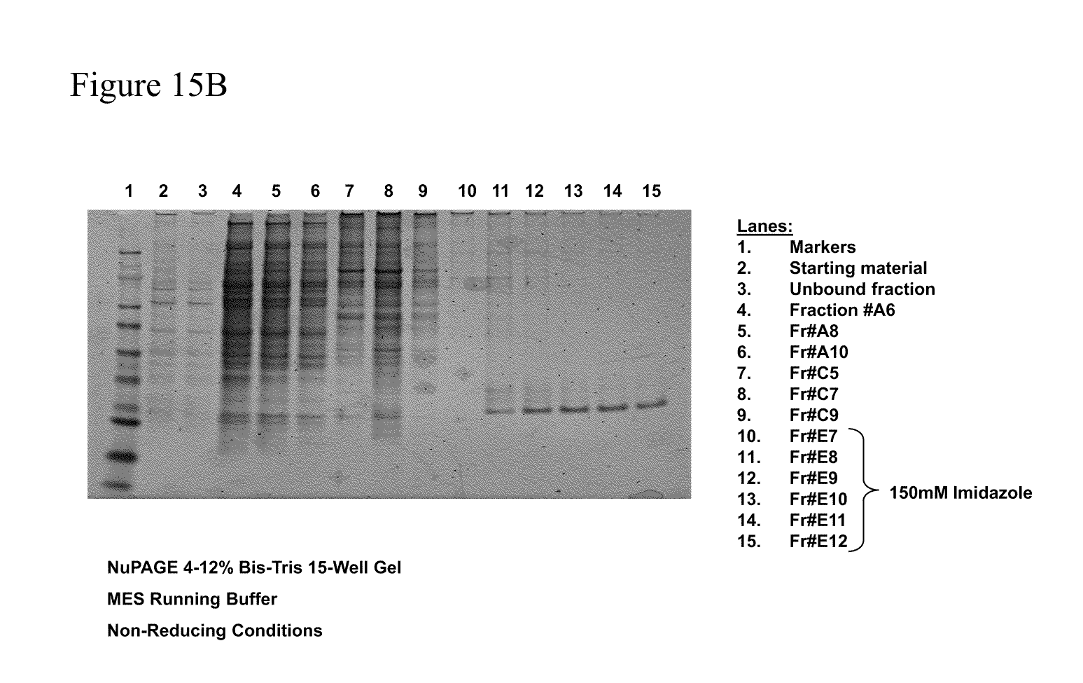

The cell supernatant was harvested four days after transfection, clarified by centrifugation (30 min, 6000 rpm), filtered (0.2 μm PES membrane, Corning), and concentrated to less than 1 L using a Centramate (Pall) concentrator. The concentrated sample was then diluted with 10×PBS to a final concentration of 1×PBS, and again 0.2 μm filtered. Diluted supernatant was loaded onto an equilibrated (20 mM Na-Phosphate, 0.5M NaCl, pH7.4) HisTrap column (GE Healthcare) at a relative concentration of approximately 10 mg protein per ml of resin. After loading, the column was washed and protein was eluted with a step gradient of Imidazole (10 mM, 50 mM, 150 mM, 250 mM and 500 mM). FIG. 15A shows a representative IMAC purification profile for the follistatin variant ACTA3. FIG. 15B shows the SDSPAGE of the elution profile for the IMAC purification in the previous figure. Peak fractions that eluted with 150 mM Imidazole were pooled and concentrated with a 10K MWCO centrifugal concentrator (Millipore). Concentrated material was loaded onto an equilibrated (PBS, pH7) 26/60 Superdex 200 column (GE Healthcare) and purified by size exclusion chromatography. Fractions containing ACTA3 were pooled and concentrated with a 10K MWCO centrifugal concentrator (Millipore). The concentration of the purified ACTA3 was determined by absorbance at 280 nm on a NANODROP™ spectrophotometer (Thermo Fisher Scientific). The quality of the purified protein was assessed by SDS-PAGE. Purified protein was stored at 4° C.

Coupling ACTA 3 to NHS-Sepharose: Coupling to NHS-Sepharose (GE Healthcare) was performed according to the manufacturer's instructions provided with the resin.

Briefly, purified follistatin was dialyzed overnight at 4° C. into the coupling buffer (0.2M NaHCO₃, 0.5M NaCl pH8.3). NHS-Sepharose was prepared according to the manufacturer's instructions and added to the dialyzed protein. The coupling reaction took place overnight at 4° C. The next day the follistatin-NHS-Sepharose resin was washed according to the manufacturer's instructions and equilibrated with PBS, pH7.

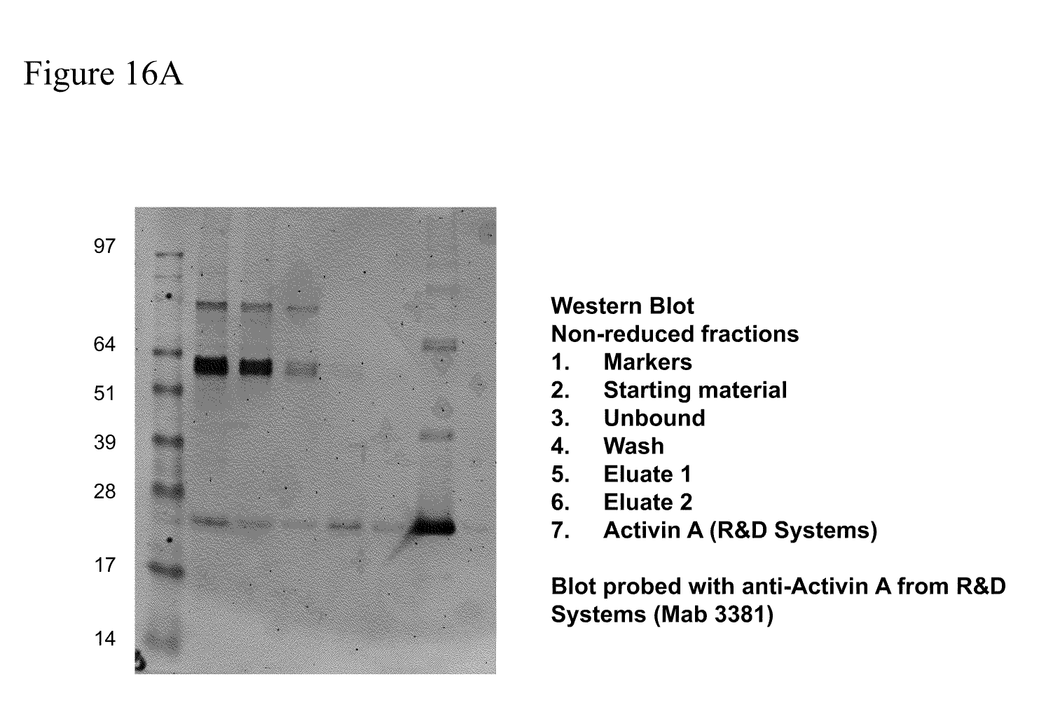

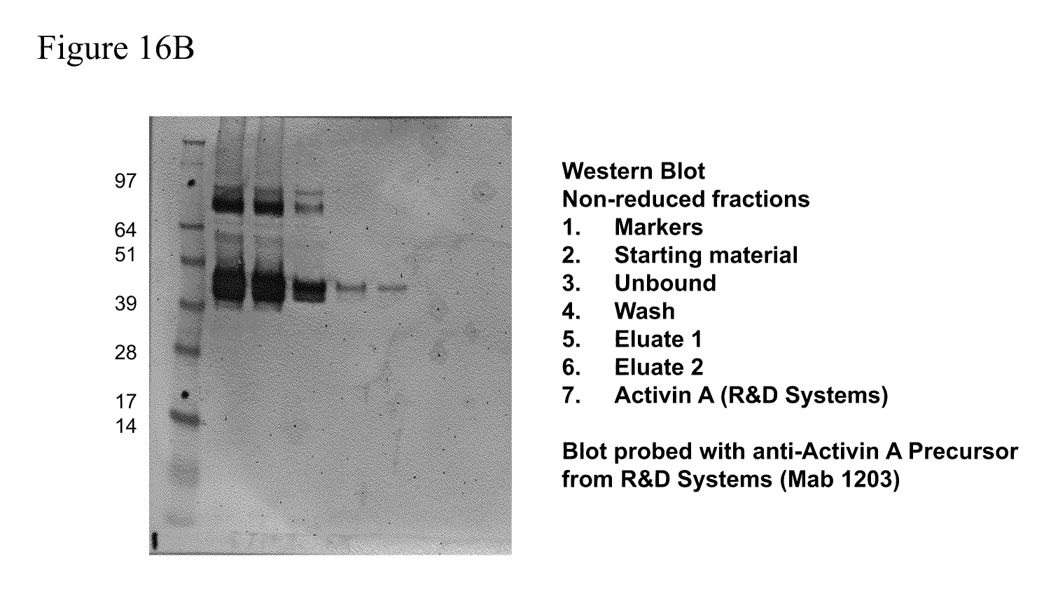

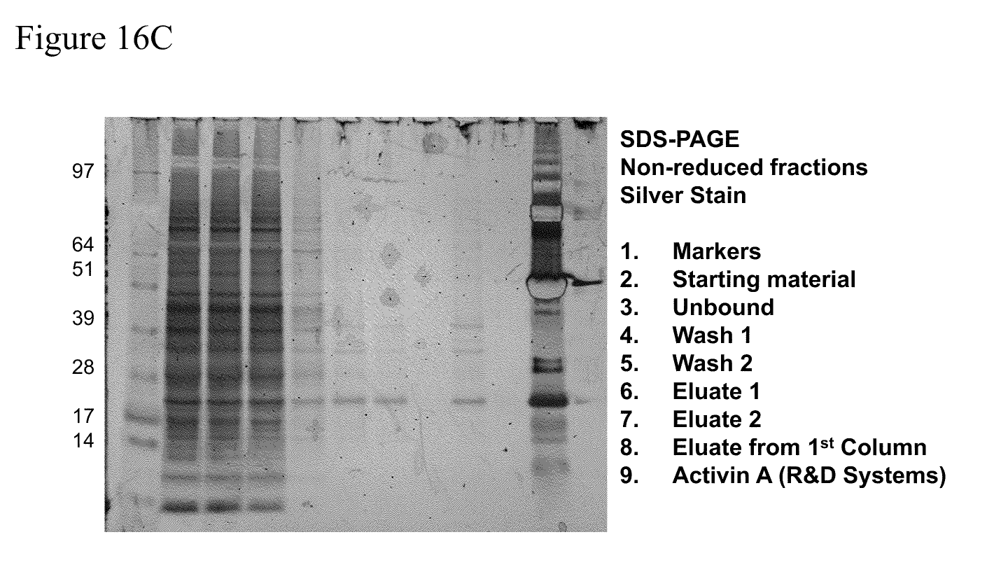

Purification of the peptides of the present invention using ACTA 3 Affinity Chromatography: Briefly, cell supernatants from transiently transfected HEK293-F cells were harvested 4 days after transfection, clarified by centrifugation (30 min, 6000 rpm), and filtered (0.2 μm PES membrane, Corning). The relative amount of specific protein was determined by activin A ELISA (R&D Systems; Cat #DY338) as per manufacturer's instructions. Samples were concentrated to less than or equal to 100 ml using an LV Centramate (Pall) concentrator. The concentrated samples were then diluted with 10×PBS to a final concentration of 1×PBS and again 0.2 μm filtered. Equilibrated ACTA 3 affinity resin was added to the diluted supernatants, and the slurry was incubated overnight at 4° C. The following day, the column was washed and protein was eluted with 10 column volumes of 0.1 M Glycine, pH 2.5. The eluted protein fractions were neutralized immediately by elution into tubes containing 1.0 M Tris, pH 9 at 10% fraction volume; i.e., if 1 ml of eluate was collected, the tubes were pre-filled with 0.1 ml Tris buffer. Peak fractions were pooled and dialyzed against PBS, pH 7 overnight at 4° C. The dialyzed proteins were removed, filtered (0.2 μm), and the protein concentration determined by absorbance at 280 nm on a NANODROP™ spectrophotometer (Thermo Fisher Scientific). If necessary, the purified proteins were concentrated with a 10K molecular weight cut-off (MWCO) centrifugal concentrator (Millipore). The quality of the purified proteins was assessed by SDSPAGE and Western blot. FIG. 16 shows a representative purification of peptide variant ACTN 1 using anti activin A antibody in FIG. 16A (R&D Systems; Cat #3381) or anti-precursor antibody in FIG. 16B (R&D Systems; Cat #1203) for detection by Western blot or silver stain in FIG. 16C. Purified proteins were stored at 4° C.

### Example 5

### Human Embryonic Stem Cell Culture

The human embryonic stem cell lines H1, H7, and H9 were obtained from WiCell Research Institute, Inc., (Madison, Wis.) and cultured according to the instructions provided by the source institute. The human embryonic stem cells were also seeded on plates coated with a 1:30 dilution of growth factor-reduced MATRIGEL™ (BD Biosciences; Cat #356231) and cultured in MEF-conditioned medium supplemented with 8 ng/ml bFGF (R&D Systems; Cat #233-FB). The cells cultured on growth factor-reduced MATRIGEL™ were routinely passaged with collagenase IV (Invitrogen/GIBCO; Cat #17104-019), Dispase (Invitrogen; Cat #17105-041) or Liberase CI enzyme (Roche; Cat #11814435001).

### Example 6

### Activin A Bioassay

Activin A is an important mediator of differentiation in a broad range of cell types. When human embryonic stem cells are treated with a combination of activin A and Wnt3a, various genes representative of definitive endoderm are up-regulated. A bioassay that measures this differentiation in human embryonic stem cells was adapted in miniaturized format to 96-well plates for screening purposes. Validation was completed using treatment with commercial sources of activin A and Wnt3a recombinant proteins and measuring protein expression of the transcription factor SOX17, which is considered a representative marker of definitive endoderm.

Live Cell Assay: Briefly, clusters of H1 or H9 human embryonic stem cells were grown on growth factor-reduced MATRIGEL™ (Invitrogen; Cat #356231)-coated tissue culture plastic. Cells were passaged using collagenase (Invitrogen; Cat # Cat #17104-019) treatment and gentle scraping, washed to remove residual enzyme, and plated in a ratio of 1:1 (surface area) on growth factor-reduced MATRIGEL™-coated 96-well plates (black, 96-well; Packard ViewPlates; Cat #6005182). Cells were allowed to attach as clusters and then recover log phase growth over a 1 to 3 day period, feeding daily with MEF conditioned medium supplemented with 8 ng/ml bFGF (R&D Systems; Cat #233-FB).

The assay was initiated by washing the wells of each plate twice in PBS and followed by adding an aliquot (100 μl) of test sample in DMEM:F12 basal medium (Invitrogen; Cat #11330-032) to each well. Test conditions were performed in triplicate, feeding on alternating days by aspirating and replacing the medium from each well with test samples over a total four day assay period. On the first and second day of assay, test samples added to the assay wells were diluted in DMEM:F12 with 0.5% FCS (HyClone; Cat #SH30070.03) and 20 ng/ml Wnt3a (R&D Systems; Cat #1324-WN). On the third and fourth day of assay, test samples added to the assay wells were diluted in DMEM:F12 with 2% FCS, without any Wnt3a. Positive control samples consisted of recombinant human activin A (Peprotech; Cat #120-14) added at a concentration of 100 ng/ml throughout assay plus Wnt3a (20 ng/ml) on days 1 and 2. Negative control samples omitted treatment with both activin A and Wnt3a.

High Content Analysis: At the conclusion of four days of culture, assay plates were washed twice with PBS, fixed with 4% paraformaldehyde at room temperature for 20 minutes, then washed three times with PBS and permeabilized with 0.5% Triton X-100 for 20 minutes at room temperature. Cells were washed again three times with PBS and blocked with 4% chicken serum (Invitrogen; Cat #16110082) in PBS for 30 minutes at room temperature. Primary antibody (goat anti-human SOX17; R&D Systems; cat #AF1924) was diluted 1:100 in 4% chicken serum and added to each well for one hour at room temperature. Alexa Fluor 488 conjugated secondary antibody (chicken anti-goat IgG; Molecular Probes; Cat #) was diluted 1:200 in PBS and added to each sample well after washing three times with PBS. To counterstain nuclei, 4 μg/ml Hoechst 33342 (Invitrogen; Cat #H3570) was added for ten minutes at room temperature. Plates were washed once with PBS and left in 100 μl/well PBS for imaging.

Imaging was performed using an IN Cell Analyzer 1000 (GE Healthcare) utilizing the 51008bs dichroic for cells stained with Hoechst 33342 and Alexa Fluor 488. Exposure times were optimized from positive control wells and from untreated negative control wells stained with secondary antibody alone. Images from 15 fields per well were acquired to compensate for any cell loss during the bioassay and subsequent staining procedures. Measurements for total cell number and total SOX17 intensity were obtained from each well using IN Cell Developer Toolbox 1.7 (GE Healthcare) software. Segmentation for the nuclei was determined based on grayscale levels (baseline range 100-300) and nuclear size. Averages and standard deviations were calculated for each replicate data set. Total SOX17 protein expression was reported as total intensity or integrated intensity, defined as total fluorescence of the cell multiplied by the area of the cell. Background was eliminated based on acceptance criteria of gray-scale ranges between 200 to 3500. Total intensity data were normalized by dividing total intensities for each well by the average total intensity for the positive control. Normalized data were calculated for averages and standard deviations for each replicate set.

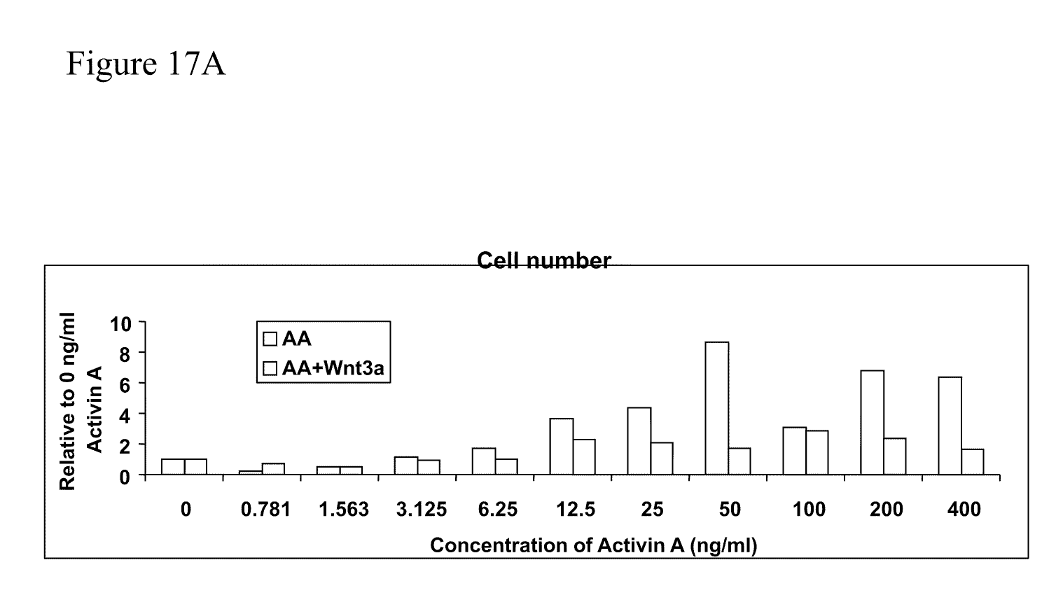

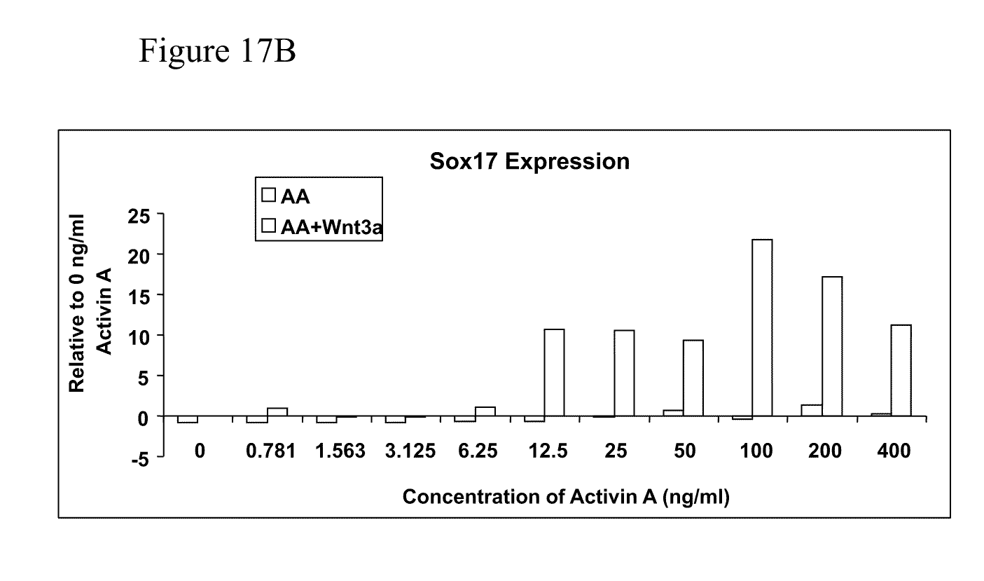

FIG. 17 shows validation of the screening assay, testing a two-fold dilution curve of a commercial source of activin A (Peprotech) and measuring both cell number (FIG. 17A) and SOX17 intensity (FIG. 17B). Optimal activin A effects for induction of SOX17 expression were generally observed in the 100-200 ng/ml range with an EC₅₀ of 30-50 ng/ml. Omitting Wnt3a from treatment on days 1 and 2 of assay failed to produce measurable SOX17 expression. Absence of activin A also failed to yield SOX17 expression.

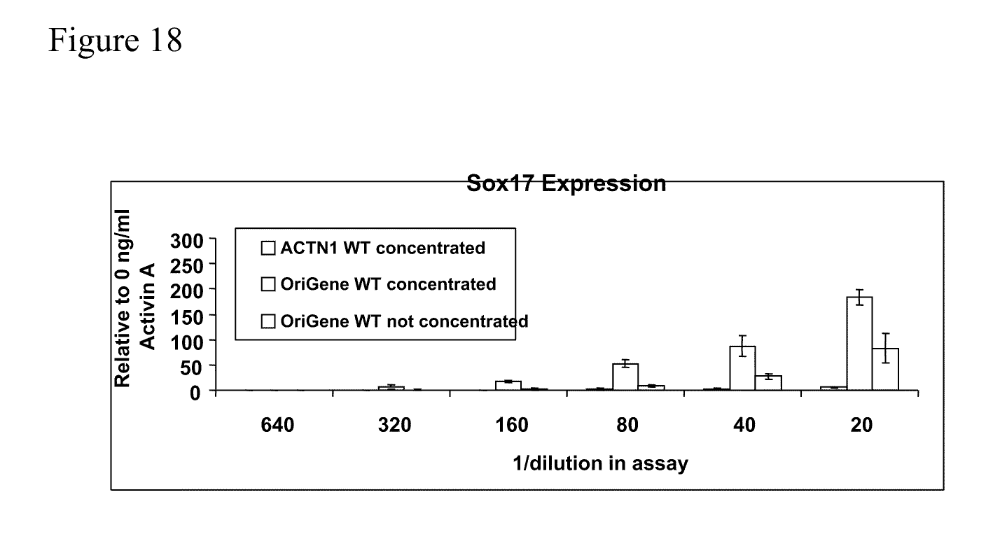

Testing wildtype activin A standards: Two wildtype activin A gene constructs were expressed and tested for functional activity: OriGene activin A in the pCMV6-XL4 mammalian expression vector (Cat #SC 118774) and ACTN1 in the pcDNA3.1(−) mammalian expression vector. Both constructs utilize a CMV promoter in their respective expression vectors, and both were expressed in HEK293-E cells. Culture supernatants were collected after 96 hours and tested for functional activity. Supernatants received as 1× (neat) or 4× concentrated stocks were diluted 1:4 in DMEM:F12 to create an intermediate stock and then further diluted two-fold in series before finally diluting 1:5 into each well containing cells and assay medium (final assay dilution range was 1:20 to 1:640). Results for human embryonic stem cell differentiation to definitive endoderm, as measured by SOX17 expression levels, are shown in FIG. 18. In this assay, the OriGene wildtype activin A expression system was superior to ACTN1 expression. Concentrating the OriGene wildtype supernatant improved functional activity approximately 1.5-fold.

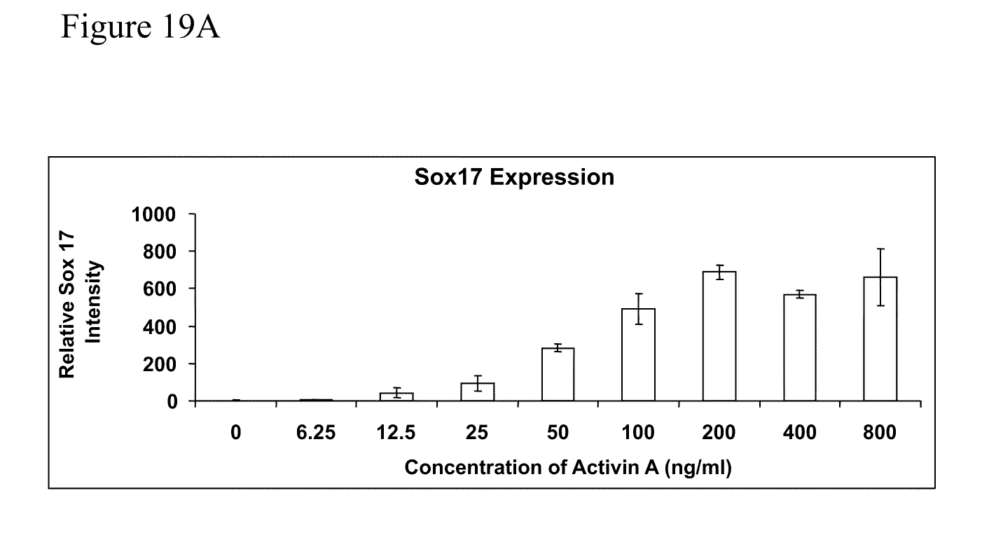

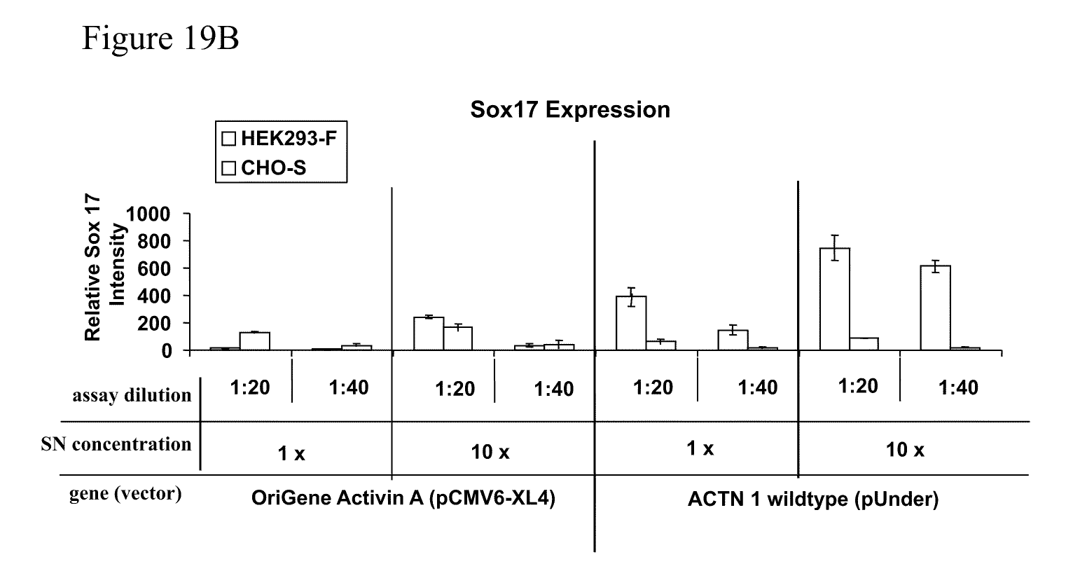

In an effort to improve the expression system, the ACTN 1 construct was subsequently moved to the pUnder mammalian expression vector. The full-length ACTN 1 precursor gene was subcloned from pcDNA3.1(−) into pUnder using EcoRI and HindIII sites, as described in Example 2. Both this new ACTN 1 wild type activin A construct along with the OriGene construct were separately transfected into CHO-S or HEK293-F cells. Supernatants harvested at 96 hours were prepared as described in Example 2 and tested for activin A activity. Supernatants received as 1× (neat) or 10× concentrated stocks were diluted 1:4 or 1:8 in DMEM:F12 to create intermediate dilutions and then further diluted 1:5 into each assay well containing cells and assay medium (final assay dilution range 1:20 or 1:40). A standard curve for human embryonic stem cell differentiation using commercial recombinant human activin A (Peprotech) in this assay is shown in FIG. 19A, measuring SOX17 expression levels as a marker of definitive endoderm differentiation. Results comparing OriGene activin A and ACTN 1 in the various expression systems used in this assay are shown in FIG. 19B. ACTN1 expression was significantly improved using the pUnder expression vector and HEK293F cells; ACTN 1 expression in CHOS cells showed weak or negligible results, even after concentrating the supernatant.

### Example 7

### The Ability of the Peptides of the Present Invention to Differentiate Human Embryonic Stem Cells into Cells Expressing Markers Characteristic of the Definitive Endoderm Lineage

Alteration of specific amino acid residues in the activin A sequence may have profound effects on the functional properties of the molecule and may thereby alter various biological outcomes. Changes may, for example, modify receptor binding affinity or dimer stability, either in a positive or negative manner. It was important to measure functional activity of expressed variants in a bioassay and determine whether patterns in the modification of specific residues correlated with enhanced or decreased function, relative to a wildtype standard.

Screening: Cell clusters, obtained from the human embryonic stem cell line H1 were plated and assayed as described above in Examples 5 and 6. Briefly, clusters of H1 human embryonic stem cells were grown on growth factor-reduced MATRIGEL™-coated tissue culture plastic. Cells were passaged using collagenase treatment and gentle scraping, washed to remove residual enzyme, and plated at a ratio of 1:1 (surface area) on growth factor-reduced MATRIGEL™-coated 96-well plates. Cells were allowed to attach as clusters and then recover log phase growth over a 1 to 3 day period, feeding daily with MEF conditioned medium supplemented with 8 ng/ml bFGF (R&D Systems; Cat #233-FB).

The assay was initiated by washing the wells of each plate twice in PBS followed by adding an aliquot (100 μl) of test sample in DMEM:F12 basal medium (Invitrogen: Cat #11330-032) to each well. Test conditions were performed in triplicate, feeding on alternating days by aspirating and replacing the medium from each well with test samples over a total four-day assay period. On the first and second day of assay, test samples added to the assay wells were diluted in DMEM:F12 with 0.5% FCS (HyClone; Cat #SH30070.03) and 20 ng/ml Wnt3a (R&D Systems; Cat #1324-WN). On the third and fourth day of assay, test samples added to the assay wells were diluted in DMEM:F12 with 2% FCS, without any Wnt3a. Positive control samples consisted of recombinant human activin A added at a concentration of 100 ng/ml throughout assay plus Wnt3a (20 ng/ml) on days 1 and 2. Negative control samples omitted treatment with both activin A and Wnt3a.

Supernatants of each expressed variant peptide were received as neat, 10×, or 50× concentrated stocks. Test supernatants were diluted 1:4 or 1:8 in DMEM:F12 to create intermediate dilutions and then further diluted 1:5 into each well containing cells and assay medium (final dilution range 1:20 or 1:40). Supernatants from the OriGene or ACTN 1 (each corresponding to activin A wildtype) expression constructs served as positive controls for these assays.

High Content Analysis: At the conclusion of four days of culture, assay plates were washed twice with PBS, fixed with 4% paraformaldehyde at room temperature for 20 minutes, then washed three times with PBS and permeabilized with 0.5% Triton X-100 for 20 minutes at room temperature. Cells were washed again three times with PBS and blocked with 4% chicken serum (Invitrogen; Cat #16110082) in PBS for 30 minutes at room temperature. Primary antibody (goat anti-human SOX17; R&D Systems; Cat #AF1924) was diluted 1:100 in 4% chicken serum and added to each well for one hour at room temperature. Alexa Fluor 488 conjugated secondary antibody (chicken anti-goat IgG; Molecular Probes; Cat #) was diluted 1:200 in PBS and added to each sample well after washing three times with PBS. To counterstain nuclei, 4 μg/ml Hoechst 33342 (Invitrogen; Cat #H3570) was added for ten minutes at room temperature. Plates were washed once with PBS and left in 100 μl/well PBS for imaging.

Imaging was performed using an IN Cell Analyzer 1000 (GE Healthcare) utilizing the 51008bs dichroic for cells stained with Hoechst 33342 and Alexa Fluor 488. Exposure times were optimized from positive control wells and from untreated negative control wells stained with secondary antibody alone. Images from 15 fields per well were acquired to compensate for any cell loss during the bioassay and subsequent staining procedures. Measurements for total cell number and total SOX17 intensity were obtained from each well using IN Cell Developer Toolbox 1.7 (GE Healthcare) software. Segmentation for the nuclei was determined based on grayscale levels (baseline range 100-300) and nuclear size. Averages and standard deviations were calculated for each replicate data set. Total SOX17 protein expression was reported as total intensity or integrated intensity, defined as total fluorescence of the cell multiplied by the area of the cell. Background was eliminated based on acceptance criteria of gray-scale ranges between 200 to 3500. Total intensity data were normalized by dividing total intensities for each well by the average total intensity for the positive control. Normalized data were calculated for averages and standard deviations for each replicate set.

Results for the differentiation of human embryonic stem cells to definitive endoderm, as measured by SOX17 expression levels, are shown in Table 8. From the screening, supernatants corresponding to a subset of variant peptides could be identified as having significant functional activity in the definitive endoderm bioassay. In some cases, the functional activity for some peptide variants showed a dose titration effect, having more activity where the supernatant was concentrated 10× or 50× relative to neat, unconcentrated samples; for example, sample supernatants for ACTN 4 showed a 2.6-fold higher potency and ACTN 16 showed a 4-fold improvement when concentrated 10× relative to their corresponding unconcentrated supernatants. Some samples failed to demonstrate any functional activity or had marginal functional activity relative to the positive control. This may reflect differences in protein expression or alternatively, may reflect a negative impact of the mutations on proper folding, dimer formation, or orientation and affinity of the activin A peptide variant with its respective receptors. The screening results, however, do identify a subset of variant peptides having significant function in the definitive endoderm bioassay. Table 9 displays this subset of hits. Without additional information regarding protein expression levels in these supernatant samples, the list may not be comprehensive and cannot be used to rank order the potencies of the variant peptides relative to each other or relative to a wildtype standard.

### Example 8

### Determination of the Protein Concentration of the Peptides of the Present Invention

It was important to be able to measure total amounts of activin A protein in the cell culture supernatants (neat or concentrated) as well as in samples subjected to various purification strategies. This was a necessary step towards being able to compare variant peptides to each other as well as the wildtype control or commercial sources of activin A. To that end, a commercial ELISA kit for human activin A was validated with both the kit standard and a different commercial source of activin A used in the bioassay described above. In a subsequent step, expressed and purified samples of activin A plus the variant peptides of this invention were tested in the ELISA assay to determine a measure of total protein in each sample.

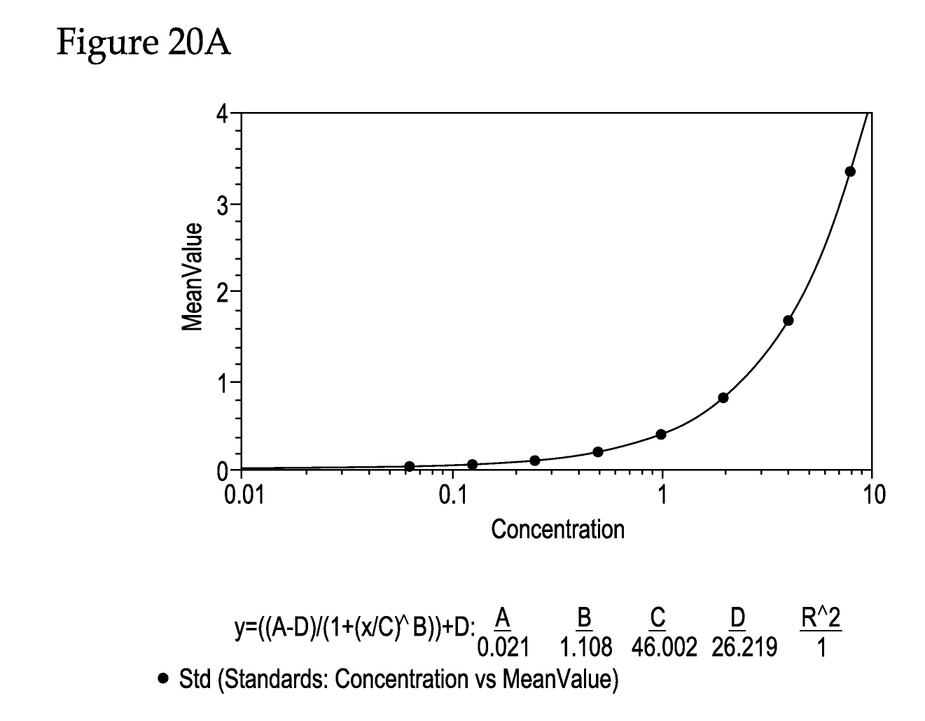

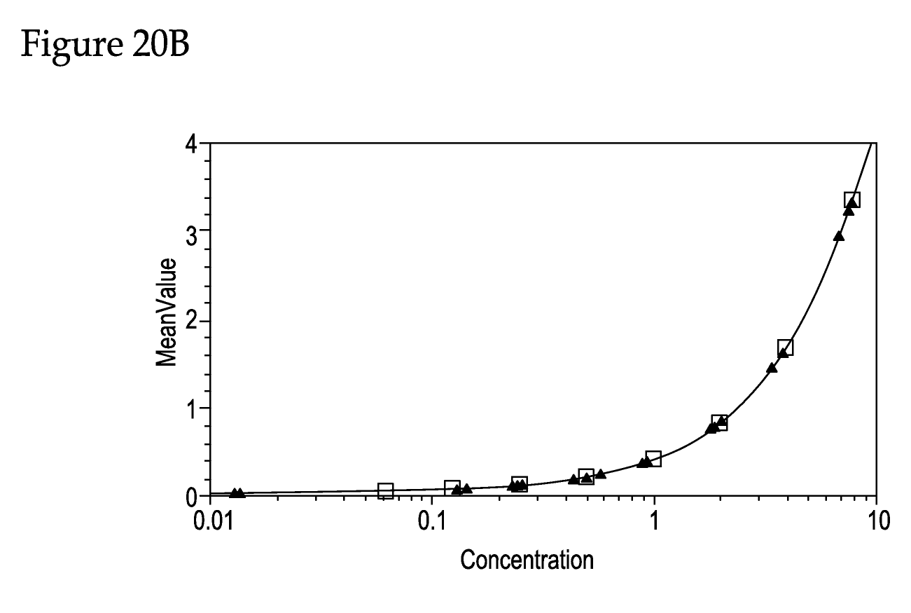

Cell culture supernatants (neat samples), concentrated supernatants, and purified material were assayed for total activin A protein using a commercial DuoSet kit for human activin A (R&D Systems, Cat #DY338) according to instructions supplied by the manufacturer, with the exception that wash steps were performed four times at each recommended step. Reagents not included in the kit and purchased from other commercial sources included BSA Fraction IV (RIA grade; Sigma; Cat #A7888), TMB solution (Sigma; Cat #T0440), PBS (Invitrogen; Cat #14190), Tween-20 (JT Baker; Cat #X251-07), sulfuric acid (JT Baker; Cat #9681-00), and urea (BioRad; Cat #161-0731). Recombinant human activin A as supplied by the manufacturer in the kit was used as a reference standard for ELISA validation. This material was diluted two-fold in series to generate a seven-point standard curve with a high standard of 8 ng/ml, as shown in FIG. 20A. Another commercial source of recombinant human activin A (Peprotech; Cat #120-14) was also tested in parallel with the kit standard and generated an identical standard curve, as shown in FIG. 20B, indicating the high degree of reproducibility of this assay. Cell culture supernatants (neat or concentrated) and purified material (from IMAC or ACTA 3 affinity purification columns) were diluted in series such that concentrations could be calculated from the linear portion of the standard curve. ELISA results from all samples are shown in Table 10.

A series of variant peptides from the primary screening was chosen for follow up evaluation. Variants were transfected as before using the corresponding pUnder vector and HEK293-F cells in shake flasks. Briefly, cells were diluted to 1.0×10⁶ cells per ml. An aliquot of total DNA was diluted in Opti-Pro (Invitrogen; Cat #12309), and an aliquot of FreeStyle Max transfection reagent (Invitrogen; Cat #16447) was diluted in Opti-Pro. The diluted DNA was added to the diluted Max reagent and incubated for 10 minutes at room temperature followed by addition of the DNA Max complex to the flask of cells and incubation for 96 hours shaking at 125 RPM, 37° C. and 8% CO₂. The supernatant was separated from the cells by centrifugation at 5,000×g for 10 minutes and filtered through a 0.2 μm filter (Corning; Cat #431153), then concentrated 10 fold using an Amicon Ultra Concentrator 10K (Cat #UFC901096), centrifuging for approximately 10 minutes at 3,750×g. Samples were stored at 4° C.

Cell culture supernatants were diluted in series such that concentrations could be calculated from the linear portion of the standard curve. ELISA results from all samples are shown in Tables 10 and 11. Table 10 shows a first attempt to dilute the samples across a large range to find an appropriate dilution for each sample within the linear portion of the standard curve. This was important in order to be able to accurately calculate the sample concentration. Table 11 shows a second experiment using the appropriate dilution series and the final calculated concentration for each respective sample.

### Example 9

### Correlation of Protein Concentration and Functional Activity for the Peptides of the Present Invention

It was important to show that variant peptides of the present invention that had been altered with histidine residues for ease of purification also had activity in the definitive endoderm differentiation assay and that this activity correlated with relative amounts of specific protein. A subset of variant peptides identified from primary screening in Example 5 above was selected for additional bis-his mutation. After expression and concentration of the corresponding culture supernatants, samples were assayed for total activin A protein and functional effects.

Transfection of the peptides of the present invention containing histidine insertions: Gene sequences, encoding the bis-his peptides ACTD 2 through ACTD 16 and their respective parent constructs (ACTN 1, ACTN 16, and ACTN 34) as listed in Table 2, were generated and inserted into the pUnder vector according to the methods described in Example 2. HEK293-F cells were transiently transfected as follows: on the day of transfection, cells were diluted to 1.0×10⁶ cells per ml in medium in a shake flask. Total DNA was diluted in Opti-Pro, and FreeStyle Max transfection reagent was diluted in Opti-Pro. The diluted DNA was added to the diluted Max reagent and incubated for 10 minutes at room temperature. An aliquot of DNA Max complex was added to the flask of cells and placed in an incubator for 96 hours shaking at 125 RPM, 37° C. and 8% CO₂.

Cell supernatants from transiently transfected HEK293-F cells were harvested four days after transfection, clarified by centrifugation (30 min, 6000 rpm), and filtered (0.2 μm PES membrane, Corning). The samples were concentrated 4-fold or 10-fold using an LV Centramate (Pall) concentrator and stored at 4° C.

ELISA protein quantification: Concentrated cell culture supernatants were assayed for total activin A protein using a commercial DuoSet kit for human activin A (R&D Systems; Cat #DY338) and according to instructions supplied by the manufacturer, with the exception that wash steps were performed four times at each recommended step. Recombinant human activin A supplied by the kit manufacturer was used as a reference standard for ELISA validation. Calculated ELISA activin A protein concentrations for each sample are shown in Table 12.

Live Cell Assay: Briefly, clusters of H1 human embryonic stem cells were grown on growth factor-reduced MATRIGEL™ (BD Biosciences; Cat #356231)-coated tissue culture plastic, according to the methods described in Example 5. Cells were passaged using collagenase treatment and gentle scraping, washed to remove residual enzyme, and plated in a ratio of 1:1 (surface area) on growth factor-reduced MATRIGEL™-coated 96-well plates. Cells were allowed to attach as clusters and then recover log phase growth over a 1 to 3 day period, feeding daily with MEF conditioned medium supplemented with 8 ng/ml bFGF (R&D Systems; Cat #233-FB).

Assay was initiated by washing the wells of each plate twice in PBS followed by adding an aliquot (100 μl) of test sample in DMEM:F12 basal medium to each well. Test conditions were performed in triplicate, feeding on alternating days by aspirating and replacing the medium from each well with test samples over a total four day assay period. On the first and second day of assay, test samples added to the assay wells were diluted in DMEM:F12 with 0.5% FCS (HyClone; Cat #SH30070.03) and 20 ng/ml Wnt3a (R&D Systems; Cat #1324-WN). On the third and fourth day of assay, test samples added to the assay wells were diluted in DMEM:F12 with 2% FCS, without any Wnt3a. Positive control samples consisted of recombinant human activin A (Peprotech; Cat #120-14) added at a concentration of 100 ng/ml throughout assay plus Wnt3a (20 ng/ml) on days 1 and 2. Negative control samples omitted treatment with both activin A and Wnt3a.

High Content Analysis: At the conclusion of four days of culture, assay plates were washed twice with PBS, fixed with 4% paraformaldehyde at room temperature for 20 minutes, then washed three times with PBS and permeabilized with 0.5% Triton X-100 for 20 minutes at room temperature. Cells were washed again three times with PBS and blocked with 4% chicken serum (Invitrogen; Cat #16110082) in PBS for 30 minutes at room temperature. Primary antibody (goat anti-human SOX17; R&D Systems; Cat #AF1924) was diluted 1:100 in 4% chicken serum and added to each well for one hour at room temperature. Alexa Fluor 488 conjugated secondary antibody (chicken anti-goat IgG; Molecular Probes; Cat #) was diluted 1:200 in PBS and added to each sample well after washing three times with PBS. To counterstain nuclei, 4 μg/ml Hoechst 33342 (Invitrogen; Cat #H3570) was added for ten minutes at room temperature. Plates were washed once with PBS and left in 100 μl/well PBS for imaging.

Imaging was performed using an IN Cell Analyzer 1000 (GE Healthcare) utilizing the 51008bs dichroic for cells stained with Hoechst 33342 and Alexa Fluor 488. Exposure times were optimized from positive control wells and from untreated negative control wells stained with secondary antibody alone. Images from 15 fields per well were acquired to compensate for any cell loss during the bioassay and subsequent staining procedures. Measurements for total cell number and total SOX17 intensity were obtained from each well using IN Cell Developer Toolbox 1.7 (GE Healthcare) software. Segmentation for the nuclei was determined based on grayscale levels (baseline range 100-300) and nuclear size. Averages and standard deviations were calculated for each replicate data set. Total SOX17 protein expression was reported as total intensity or integrated intensity, defined as total fluorescence of the cell multiplied by the area of the cell. Background was eliminated based on acceptance criteria of gray-scale ranges between 200 to 3500. Total intensity data were normalized by dividing total intensities for each well by the average total intensity for the positive control. Normalized data were calculated for averages and standard deviations for each replicate set.

Table 12 shows activity results for various activin A peptide variants, where results for both cell number and SOX17 expression after definitive endoderm formation in this assay are correlated with the estimated activin A concentration from ELISA results. Clearly in the case of the wildtype family of peptide variants (ACTN1 and bis-his variants ACTD2-6), extra histidine substituents had little or no impact on functional activity with respect to definitive endoderm formation. The same was also true for the other peptide variant families (ACTN16 and ACTN34) and their respective bis-his variants (ACTD7-11 and ACTD12-16, respectively) where adequate amounts of protein were added to the functional assay.

### Example 10

### FACS Analysis of Cells Expressing Markers Characteristic of the Definitive Endoderm Lineage that were Formed by Treating Human Embryonic Stem Cells with the Peptides of the Present Invention

It was important to show that variant peptides of the present invention could support definitive endoderm differentiation as denoted by other biomarkers. CXCR4 is a surface protein commonly associated with definitive endoderm. It was also important to show that variant peptides with additional histidine substitutions embedded for ease of purification did not impact functional properties of the activin A molecule. In this example, human embryonic stem cells were subjected to the definitive endoderm differentiation protocol using a series of bis-his prototypes of the native wildtype and two variant molecules.

Transfection of the peptides of the present invention containing histidine insertions: Gene sequences, encoding the bis-his peptides ACTD3 and ACTD8 as listed in Table 3, were generated and inserted into the pUnder vector according to the methods described in Example 2. HEK293-F cells were transiently transfected as follows: On the day of transfection, cells were diluted to 1.0×10⁶ cells per ml in medium in separate shake flasks. Total DNA was diluted in Opti-Pro, and FreeStyle Max transfection reagent was diluted in Opti-Pro. The diluted DNA was added to the diluted Max reagent and incubated for 10 minutes at room temperature. An aliquot of DNA Max complex was added to the flask of cells and placed in an incubator for 96 hours shaking at 125 RPM, 37° C. and 8% CO₂.

Purification of peptides containing histidine insertions: Purifications using immobilized metal-chelate affinity chromatography (IMAC) were performed on an AKTA FPLC chromatography system using GE Healthcare's UNICORN™ software.

Cell supernatants from transiently transfected HEK293-F cells were harvested four days after transfection, clarified by centrifugation (30 min, 6000 rpm), and filtered (0.2 μm PES membrane, Corning). The relative amount of specific protein was determined by ELISA using the methods described in Example 6. The samples were concentrated 4-fold or 10-fold using an LV Centramate (Pall) concentrator and checked by Western blot using anti-activin A antibody (R&D Systems; Cat #3381) or anti activin A precursor antibody (R&D Systems; Cat #1203) for detection. An aliquot of ACTD3 and ACTD8 concentrated samples was saved without further purification at this point for live cell assay. The concentrated samples were then diluted with 10×PBS to a final concentration of 1×PBS and again 0.2 g filtered. Diluted supernatants were loaded onto an equilibrated (20 mM Na-Phosphate, 0.5M NaCl, pH7.4) HisTrap column (GE Healthcare) at a relative concentration of approximately 10 mg protein per ml of resin. After loading, the column was washed and protein eluted with a linear gradient of imidazole (0-500 mM) over 20 column volumes. Peak fractions were pooled and dialyzed against PBS pH 7 overnight at 4° C. The dialyzed proteins were removed from dialysis, filtered (0.2 μm), and the total protein concentration determined by absorbance at 280 nm on a NANODROP™ spectrophotometer (Thermo Fisher Scientific). The quality of the purified proteins was assessed by SDS-PAGE and Western blot using an anti activin A antibody (R&D Systems; Cat #3381) or anti activin A precursor (R&D Systems; Cat #1203) for detection. If necessary, the purified proteins were concentrated with a 10K molecular weight cut-off (MWCO) centrifugal concentrator (Millipore). Samples were stored at 4° C.

ELISA Assay: Culture supernatants of ACTD3 (4-fold concentrate), ACTD8 (10-fold concentrate), and IMAC purified material of each were tested in ELISA to measure total protein concentration. Samples were assayed for total activin A protein using a commercial DuoSet kit for human activin A (R&D Systems; Cat #DY338) and according to instructions supplied by the manufacturer, with the exception that wash steps were performed four times at each recommended step. Recombinant human activin A supplied by the kit manufacturer was used as a reference standard for ELISA validation. Concentrated supernatant of ACTD3 was present in insufficient amount to measure by ELISA. Calculated protein concentrations for the remaining samples were as follows: ACTD8 (10× supernatant concentrate) 361 ng/ml; ACTD8 (IMAC purified) 1,893 ng/ml; ACTD3 (IMAC purified) 57,956 ng/ml.

Live Cell Assay: Briefly, clusters of H1 human embryonic stem cells were grown on growth factor-reduced MATRIGEL™ (BD Biosciences; Cat #356231)-coated tissue culture plastic, according to the methods described in Example 5. Cells were passaged using collagenase treatment and gentle scraping, washed to remove residual enzyme, and plated in a ratio of 1:1 (surface area) on growth factor-reduced MATRIGEL™-coated 96-well plates. Cells were allowed to attach as clusters and then recover log phase growth over a 1 to 3 day period, feeding daily with MEF conditioned medium supplemented with 8 ng/ml bFGF (R&D Systems; Cat #233-FB).

The assay was initiated by washing the wells of each plate twice in PBS followed by adding an aliquot (100 μl) of test sample in DMEM:F12 basal medium to each well. Test conditions were performed in replicate sets of nine wells, feeding on alternating days by aspirating and replacing the medium from each well with test samples over a total four day assay period. On the first and second day of assay, test samples added to the assay wells were diluted in DMEM:F12 with 0.5% FCS (HyClone; Cat #SH30070.03) and 20 ng/ml Wnt3a (R&D Systems; Cat #1324-WN). On the third and fourth day of assay, test samples added to the assay wells were diluted in DMEM:F12 with 2% FCS, without any Wnt3a. A positive control sample consisted of recombinant human activin A (Peprotech; Cat #120-14) added at a concentration of 100 ng/ml throughout assay plus Wnt3a (20 ng/ml) on days 1 and 2. A negative control sample omitted treatment with both activin A and Wnt3a. Each concentrated supernatant or IMAC purified sample was diluted 1:16 in DMEM:F12 to create intermediate dilutions and then further diluted 1:5 into each well containing cells and assay medium (final dilution 1:80). At the conclusion of four days of culture, assay wells were washed with PBS, and cells from nine replicate wells for each treatment condition were collected as a single cell suspension by brief treatment with TrypLE™ (Invitrogen; Cat #12604-013) for 3-5 minutes. Cells were washed once in PBS prior to FACS analysis.

FACS Analysis: Cells for FACS analysis were blocked in a 1:5 solution of 0.5% human gamma-globulin (Sigma; Cat #G-4386) in PBS (Invitrogen; Cat #14040-133): BD FACS staining buffer-BSA (BD; Cat #554657) for 15 minutes at 4° C. Cells were then stained with antibodies for CD9 PE (BD; Cat #555372), CD99 PE (Caltag; Cat #MHCD9904) and CXCR4 APC(R&D Systems; Cat #FAB173A) for 30 minutes at 4° C. After a series of washes in BD FACS staining buffer, the cells were stained for viability with 7-AAD (BD; Cat #559925) and run on a BD FACSArray. A mouse IgG1K Isotype control antibody for both PE and APC was used to gate percent positive cells.

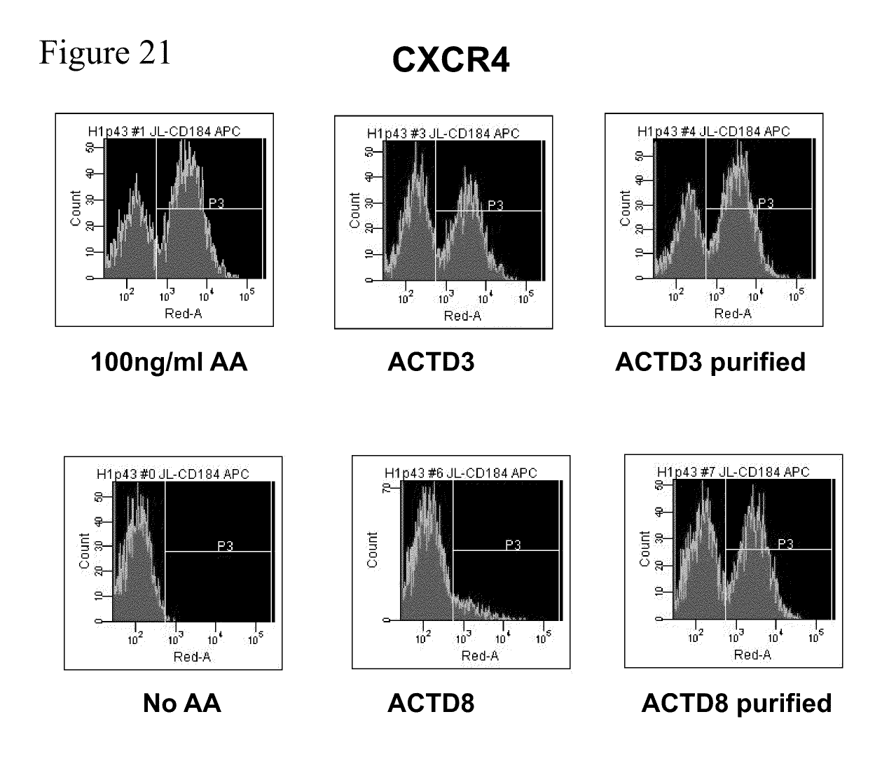

The results shown in FIG. 21 suggest that the purified material from each of the bis-his constructs have functional activity and can induce definitive endoderm formation from human embryonic stem cells.

### Example 11

### The Potency of the Peptides of the Present Invention

It was important to evaluate the relative activity and potency of each of the variant peptides compared to the wild type activin A molecule (ACTN 1). In this example, 15 variant peptides were selected and expressed, and the secreted products were each quantified by ELISA from concentrated culture supernatants. A dose titration of each peptide was then assayed for functional activity in a definitive endoderm differentiation protocol using human embryonic stem cells.

Transfection of the peptides of the present invention: Gene sequences, encoding the bis-peptides listed in Table 13, were generated and inserted into the pUnder vector according to the methods described in Example 2. HEK 293F cells were transiently transfected using Freestyle Max transfection reagent (Invitrogen; Cat #16447). The cells were diluted to 1.0×10⁶ cells per ml prior to transfection for a 20 ml transfection volume. On the day of transfection 1.25 μg per ml of transfection was diluted in 1.0 ml of OPTIPRO (Invitrogen; Cat #12309) and 1.25 ml of Max transfection reagent was diluted in 1.0 ml of OPTIPRO. The DNA and Max transfection reagent were added together to form a lipid complex and incubated for 10 minutes at room temperature. The lipid complex was then added to the cells and placed in the incubator for 4 days, shaking at 125 RPM, 37° C. and 8% CO₂. Cells were harvested four days after transfection, clarified by centrifugation (30 min, 6000 rpm), and filtered (0.2 μm PES membrane, Corning). The relative amount of specific protein was determined by ELISA using the methods described in Example 6. If necessary, the protein supernatants were concentrated 20 fold using an Amicon Ultra Concentrator 3K (Millipore; Cat #UFC900396), centrifuging for approximately 40 minutes at 3,500 RCF, and checked by Western blot using anti-activin-A antibody (R&D Systems; Cat #3381) or anti activin-A precursor antibody (R&D Systems; Cat #1203) for detection. Aliquots of ACTD3 and ACTD8 concentrated samples were saved without further purification at this point for live cell assay. 10×PBS was added to the concentrated samples to a final concentration of 1×PBS, then passed through a 0.2μ filter. If necessary, the proteins were concentrated 20 fold. Samples were stored at 4° C.

On the day of transfection, cells were diluted to 1.0×10⁶ cells per ml in medium in separate shake flasks. Total DNA was diluted in Opti-Pro, and FreeStyle Max transfection reagent was diluted in Opti-Pro. The diluted DNA was added to the diluted Max reagent and incubated for 10 minutes at room temperature. An aliquot of DNA Max complex was added to the flask of cells and placed in an incubator for 96 hours shaking at 125 RPM, 37° C. and 8% CO₂.

Cell supernatants from transiently transfected HEK293-F cells were harvested four days after transfection, clarified by centrifugation (30 min, 6000 rpm), and filtered (0.2 μm PES membrane, Corning). The relative amount of specific protein was determined by ELISA using the methods described in Example 6. The samples were concentrated 4-fold or 10-fold using an LV Centramate (Pall) concentrator and checked by Western blot using anti-activin A antibody (R&D Systems; Cat #3381) or anti activin A precursor antibody (R&D Systems; Cat #1203) for detection. An aliquot of ACTD3 and ACTD8 concentrated samples was saved without further purification at this point for live cell assay. The concentrated samples were then diluted with 10×PBS to a final concentration of 1×PBS and again 0.2μ filtered. Diluted supernatants were loaded onto an equilibrated (20 mM Na-Phosphate, 0.5M NaCl, pH7.4) HisTrap column (GE Healthcare) at a relative concentration of approximately 10 mg protein per ml of resin. After loading, the column was washed and protein eluted with a linear gradient of imidazole (0-500 mM) over 20 column volumes. Peak fractions were pooled and dialyzed against PBS pH 7 overnight at 4° C. The dialyzed proteins were removed from dialysis, filtered (0.2 μm), and the total protein concentration determined by absorbance at 280 nm on a NANODROP™ spectrophotometer (Thermo Fisher Scientific). The quality of the purified proteins was assessed by SDS-PAGE and Western blot using an anti activin A antibody (R&D Systems; Cat #3381) or anti activin A precursor (R&D Systems; Cat #1203) for detection. If necessary, the purified proteins were concentrated with a 10K molecular weight cut-off (MWCO) centrifugal concentrator (Millipore). Samples were stored at 4° C.

ELISA Assay: Culture supernatants of 15 different ACTN peptides, in addition to the wild type ACTN1 molecule, were tested in ELISA to measure total protein concentrations. Samples were assayed using a commercial DuoSet kit for human activin A (R&D Systems; Cat #DY338) according to instructions supplied by the manufacturer, with the exception that wash steps were performed four times at each recommended step. Recombinant human activin A supplied by the kit manufacturer was used as a reference standard for the ELISA validation. Concentrated supernatants of ACTN56, ACTN65, and ACTN69 were not present in sufficient amounts to measure by ELISA. Calculated protein concentrations for the remaining samples are shown in Table 13.

Live Cell Assay: Briefly, clusters of H1 human embryonic stem cells were grown on growth factor-reduced MATRIGEL™ (BD Biosciences; Cat #356231) coated tissue culture plastic, according to the methods described in Example 5. Cells were passaged using collagenase treatment and gentle scraping, washed to remove residual enzyme, and plated at a ratio of 1:1 (surface area) on growth factor-reduced MATRIGEL™ coated 96-well plates (PerkinElmer; Cat #6005182) in volumes of 0.1 ml/well. Cells were allowed to attach as clusters and then recover log phase growth over a one to three day period, feeding daily with MEF conditioned medium supplemented with 8 ng/ml bFGF (R&D Systems; Cat #233-FB). Plates were maintained at 37° C., 5% CO₂ throughout assay.

The assay was initiated by washing the wells of each plate twice in PBS followed by adding an aliquot (100 μl) of test sample to each well. Test conditions were performed in triplicate over a total four day assay period, feeding on day 1 and day 3 by aspirating and replacing the medium from each well with fresh test medium. Based on ELISA results for each of the ACTN concentrated supernatants, a two-fold dilution series, ranging from 3.1 ng/ml to 400 ng/ml, was constructed in appropriate medium for addition to assay on day 1 and day 3. On the first and second day of assay, test samples added to the assay wells were diluted in DMEM:F12 supplemented with 0.5% FCS (HyClone; Cat #SH30070.03) and 20 ng/ml Wnt3a (R&D Systems; Cat #1324-WN). On the third and fourth day of assay, test samples added to the assay wells were diluted in DMEM:F12 supplemented with 2% FCS, without any Wnt3a. A positive control sample consisted of recombinant human activin A (Peprotech; Cat #120-14) added at a concentration of 100 ng/ml throughout assay and Wnt3a (20 ng/ml) added only on days 1 and 2. A negative control sample consisted of assay medium without any growth factors.

High Content Analysis: At the conclusion of culture, assay plates were washed once with PBS (Invitrogen; Cat #14190), fixed with 4% paraformaldehyde (Alexis Biochemical; Cat #ALX-350-011) at room temperature for 20 minutes, then washed three times with PBS and permeabilized with 0.5% Triton X-100 (Sigma; Cat #T8760-2) for 20 minutes at room temperature. Cells were washed again three times with PBS and blocked with 4% chicken serum (Invitrogen; Cat #16110082) in PBS for 30 minutes at room temperature. Primary antibody (goat anti-human SOX17; R&D Systems; Cat #AF1924) was diluted 1:100 in 4% chicken serum and added to each well for two hours at room temperature. After washing three times with PBS, Alexa Fluor 488 conjugated secondary antibody (chicken anti-goat IgG; Invitrogen; Cat #A21467) diluted 1:200 in PBS was added to each well. To counterstain nuclei, 5 μg/ml Hoechst 33342 (Invitrogen; Cat #H3570) was added for fifteen minutes at room temperature. Plates were washed once with PBS and left in 100 μl/well PBS for imaging.

Imaging was performed using an IN Cell Analyzer 1000 (GE Healthcare) utilizing the 51008bs dichroic for cells stained with Hoechst 33342 and Alexa Fluor 488. Images were acquired from 25 fields per well. Measurements for total intensity were obtained from each well using IN Cell Developer Toolbox 1.7 (GE Healthcare) software. Segmentation for the nuclei was determined based on gray-scale levels (baseline range 100-300) and nuclear size. Averages and standard deviations were calculated for each replicate data set. Total protein expression was reported as total intensity or integrated intensity, defined as total fluorescence of the cell multiplied by the area of the cell. Background was eliminated based on acceptance criteria for gray-scale ranges between 200 and 4500. Total intensity data were normalized by dividing total intensities for each well by the average total intensity for the positive control.

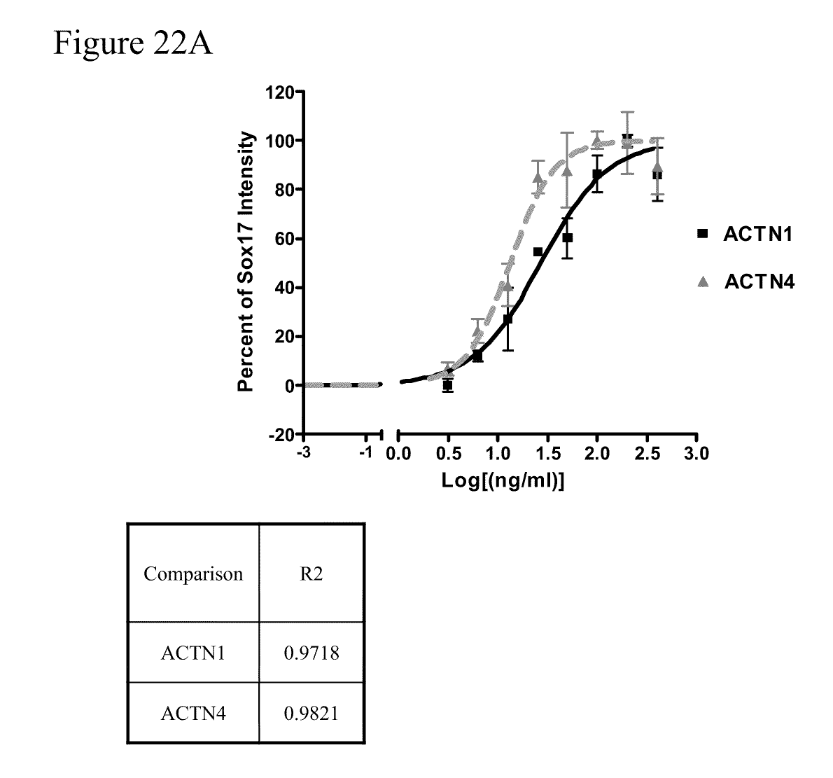

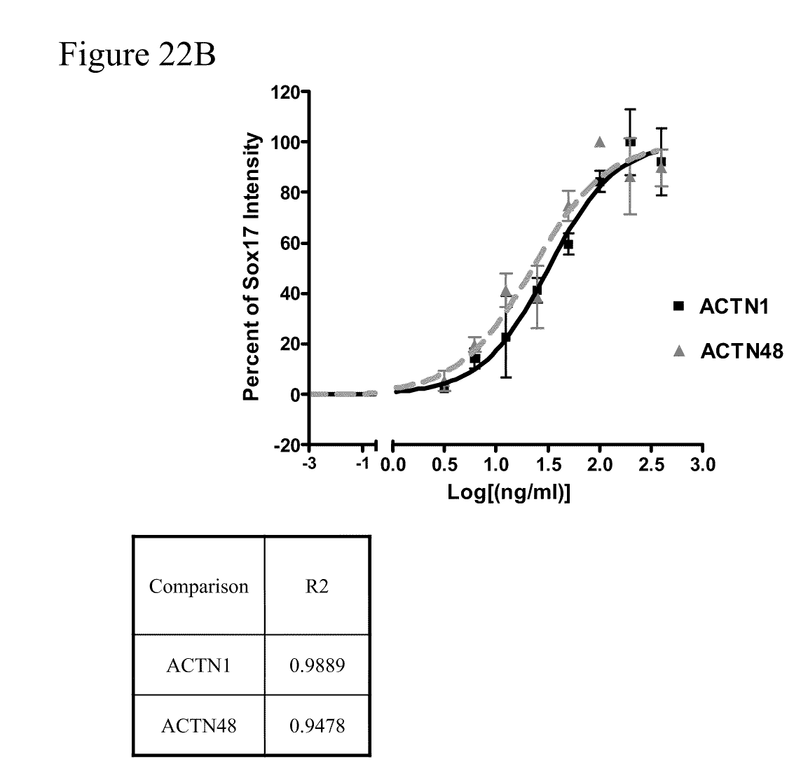

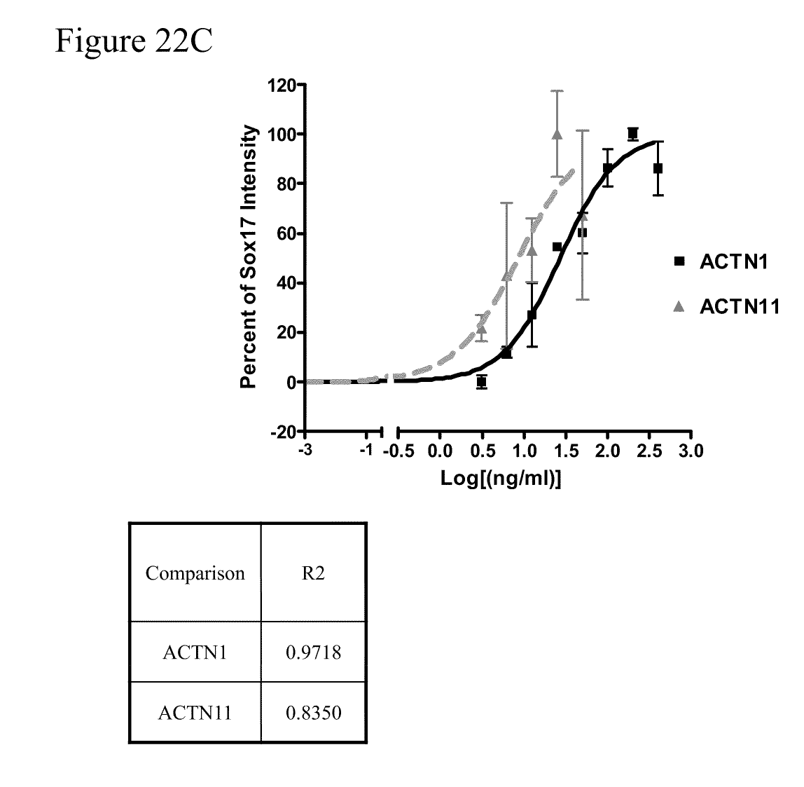

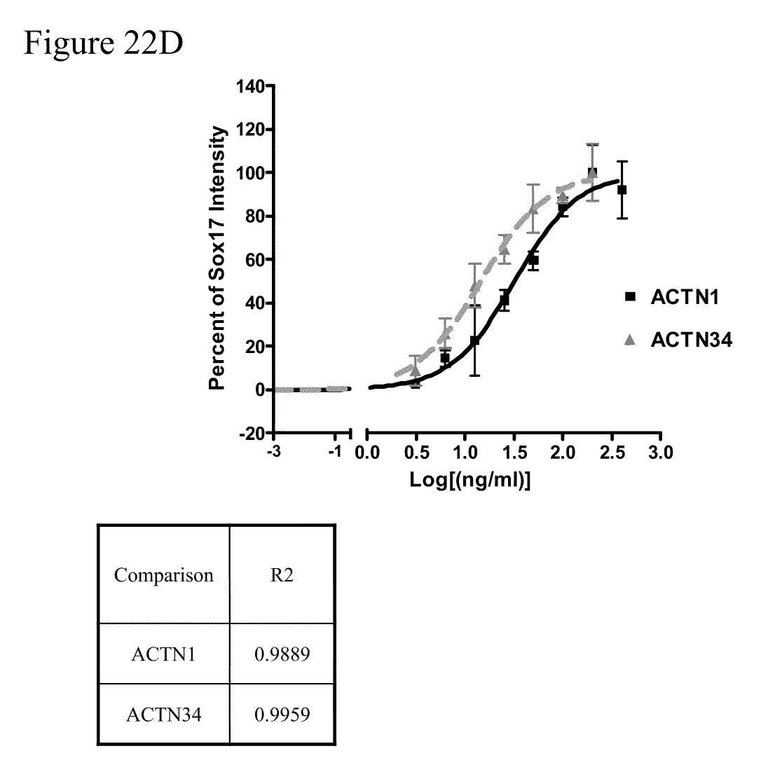

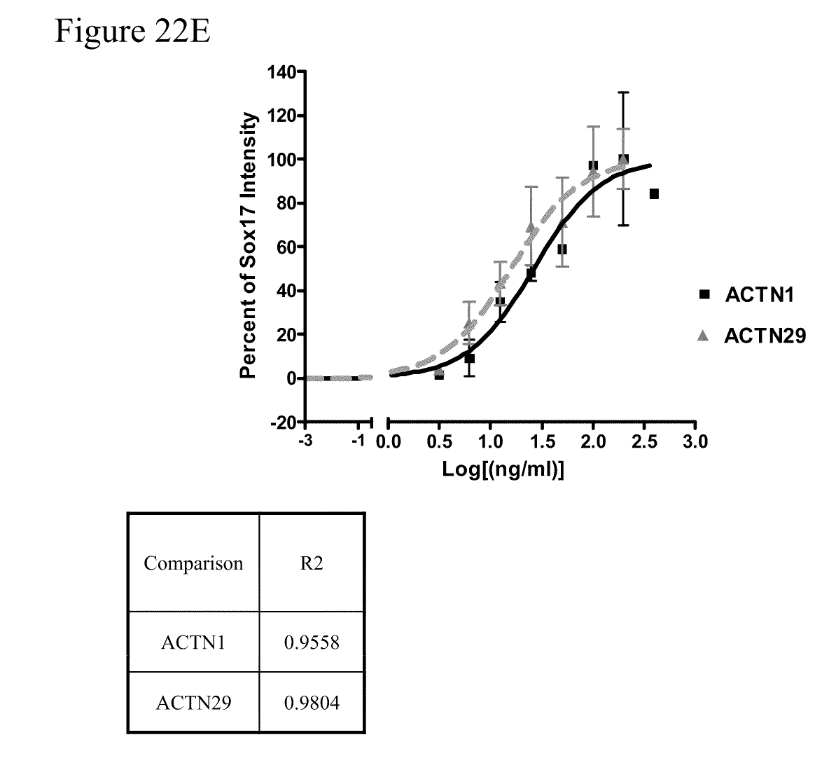

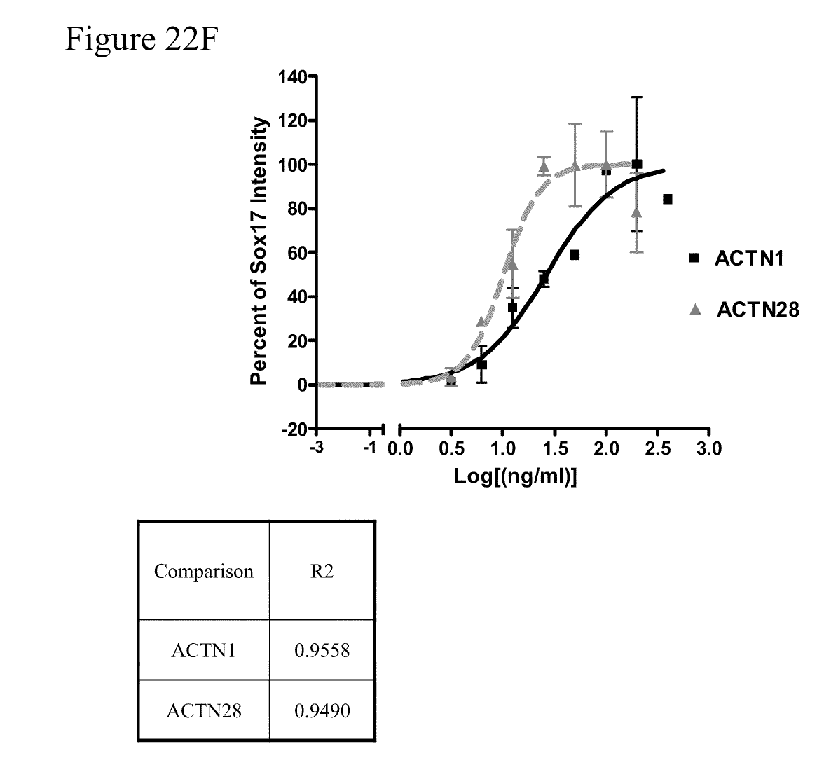

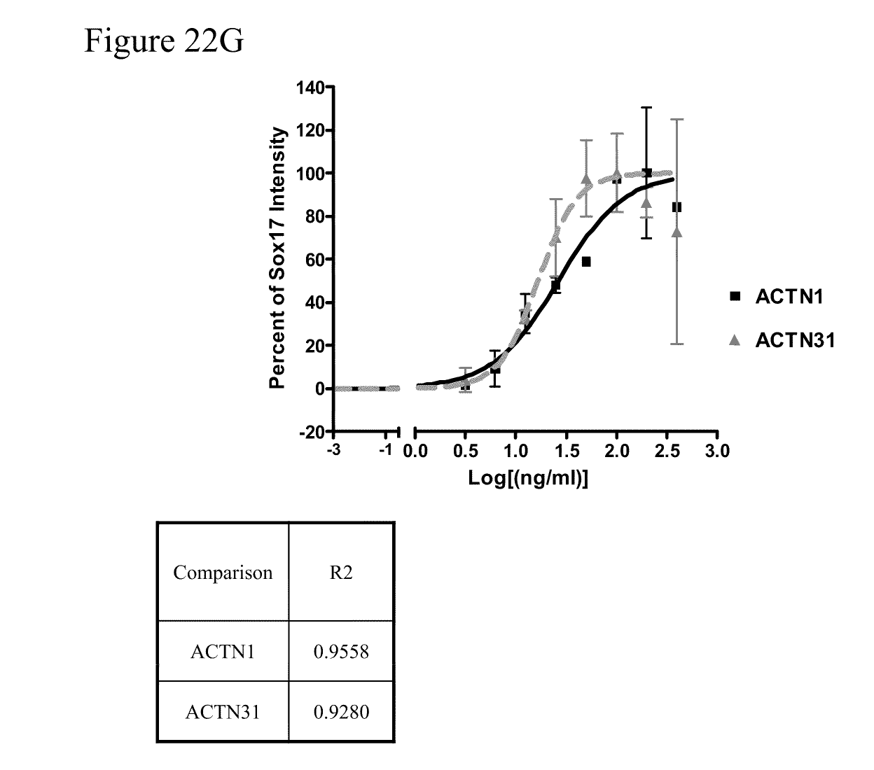

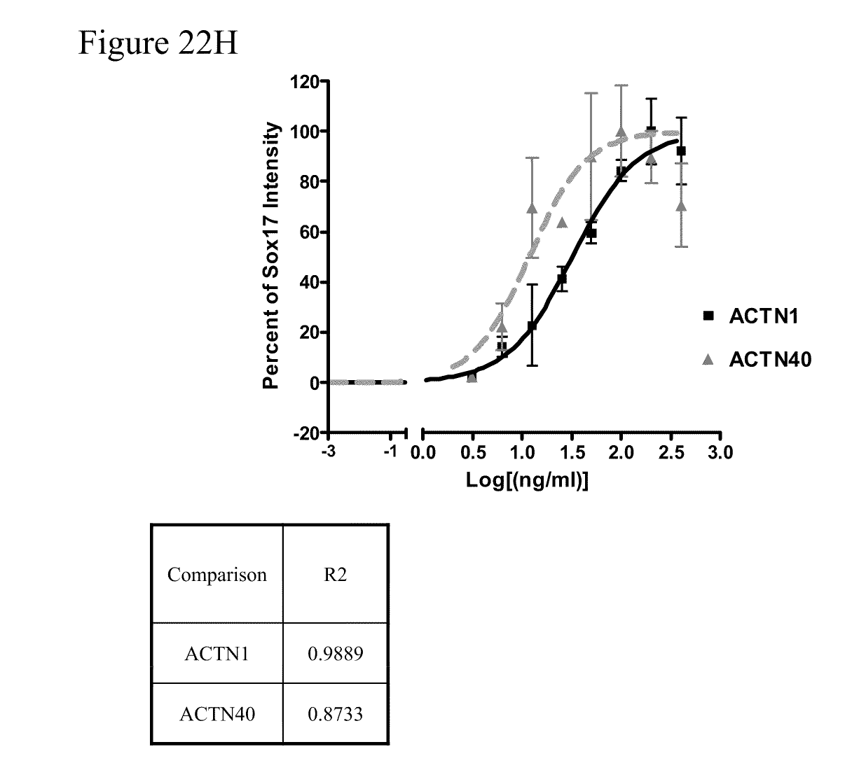

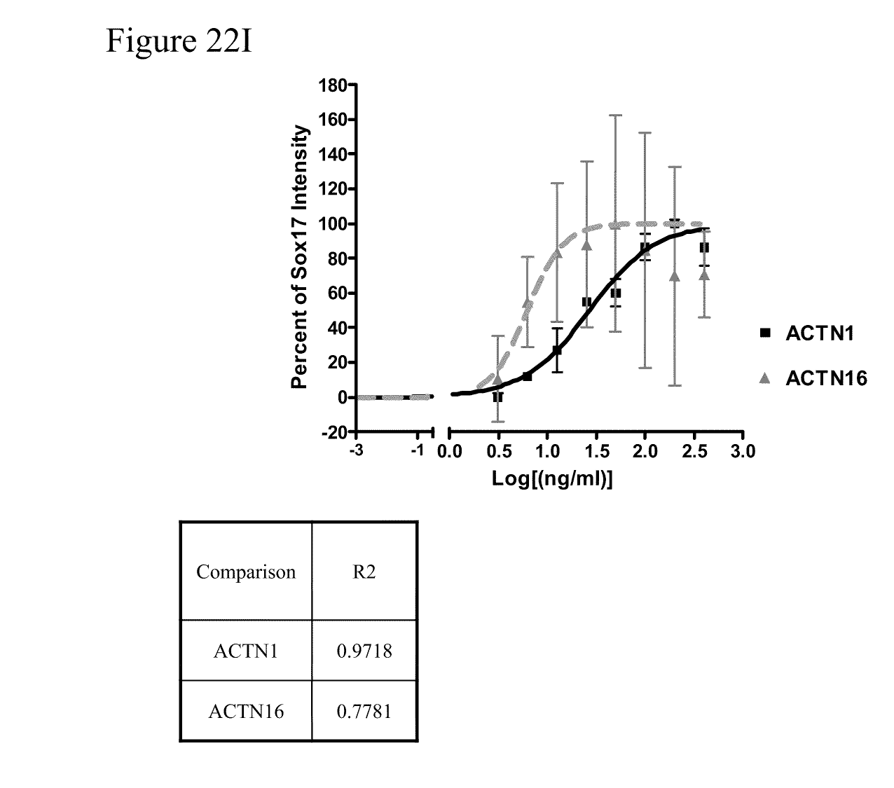

In FIG. 22, panels a through i, assay results depict percent SOX17 expression versus peptide concentration. For each of the variant ACTN peptides, a dose titration curve is shown relative to a similar curve for the wild type ACTN1 peptide. Values for curve fit (R² values) are also indicated. Dose titration results for all of the variant ACTN peptides closely match the wild type ACTN1 peptide dose titration, where the variability in curve shift is within the standard error range for each of the representative curves. These data suggest that the potency of each variant ACTN peptide is similar or equivalent to the wild type ACTN1 peptide.

### Example 12

### ACTN Variant Peptides can Mediate Downstream Pancreatic Differentiation

It was important to demonstrate that treating human embryonic stem cells with a peptide of the present invention would not prevent further differentiation toward endoderm and endocrine lineages. Two representative ACTN peptides were used to differentiate human embryonic stem cells into cells expressing markers characteristic of the definitive endoderm lineage. Thereafter, a step-wise differentiation protocol was applied to treated cells to promote differentiation toward pancreatic endoderm and endocrine lineages. A parallel control sample of cells treated with the ACTN1 wild type peptide was used for comparison throughout the step-wise differentiation process. Samples were taken at every stage of the differentiation to determine the appearance of proteins and mRNA biomarkers representative of the various stages of differentiation.

Preparation of Cells for Assay: Stock Cultures of Human Embryonic Stem Cells (H1 hESC line) were maintained in an undifferentiated, pluripotent state on growth factor-reduced MATRIGEL™-coated dishes in MEF conditioned medium supplemented with bFGF (PeproTech; Cat #100-18B) with passage on average every, four days. Passage was performed by exposing cell cultures to a solution of 1 mg/ml collagenase (Invitrogen, Cat #17104-019) for five to seven minutes at 37° C. followed by rinsing the monolayer with MEF conditioned medium and gentle scraping to recover cell clusters. Clusters were centrifuged at low speed to collect a cell pellet and remove residual collagenase. Cell clusters were split at a 1:3 or 1:4 ratio for routine maintenance culture or a 1:1 ratio for immediate assay. All human ES cell lines were maintained at passage numbers less than 50 and routinely evaluated for normal karyotypic phenotype and for absence of mycoplasma contamination.

Cell clusters were evenly resuspended in MEF conditioned medium supplemented with 8 ng/ml bFGF and seeded onto growth factor-reduced MATRIGEL™-coated 24-well, black wall culture plates (Arctic White; Cat #AWLS-303012) in volumes of 0.5 ml/well. Daily feeding was conducted by aspirating spent culture medium from each well and replacing with an equal volume of fresh medium. Plates were maintained at 37° C., 5% CO₂ throughout the duration of assay.

Assay: The assay was initiated by aspirating culture medium from each well and adding back an aliquot (0.5 ml) of test medium. Test conditions for the first step of differentiation were conducted over a three-day period, feeding daily by aspirating and replacing the medium from each well with fresh test medium. Concentrated supernatants of the ACTN peptides were evaluated for protein concentration using a DuoSet ELISA kit for human activin A (R&D Systems; Cat #DY338), as previously described in Example 11. On the first day of assay, ACTN peptides were diluted to a final concentration of 100 ng/ml in RPMI 1640 medium (Invitrogen; Cat #: 22400) with 2% Albumin Bovine Fraction V, Fatty Acid Free (FAF BSA) (MP Biomedicals, Inc; Cat #152401), 8 ng/ml bFGF, and 20 ng/ml Wnt3a (R&D Systems; Cat #1324-WN/CF) and then added to the assay wells. On the second and third day of assay, ACTN peptides were diluted into RPMI 1640 medium supplemented with 2% fatty acid free BSA and 8 ng/ml bFGF, without any Wnt3a and then added to the assay wells. A positive control sample included a commercial source of activin A (PeproTech; Cat #12 0-14) diluted in culture medium with growth factors as indicated. At the conclusion of three days culture, cells from some wells were harvested for analysis by flow cytometry to evaluate levels of CXCR4, a marker of definitive endoderm formation. Additional wells were harvested for RT-PCR analysis of other markers of differentiation. Other culture wells were subjected to high content analysis for protein expression levels of SOX17.

At the conclusion of the first step of the differentiation protocol, replicate sets of parallel wells for each treatment group were subjected to further step-wise differentiation. It is important to note that after the first three days, all wells undergoing continuing culture and differentiation received the same treatment. The protocol for this continuing differentiation is described below.

During the second step of differentiation, cultures were grown for two days in DMEM:F12 medium (Invitrogen; Cat #11330-032) supplemented with 2% Albumin Bovine Fraction V, Fatty Acid Free (FAF BSA) (MP Biomedicals, Inc; Cat #152401), 50 ng/ml FGF7 (PeproTech; Cat #100-19), and 250 nM cyclopamine (Calbiochem; Cat #239804). Medium in each well was aspirated and replaced with a fresh aliquot (0.5 ml) on both days.

Step 3 of the differentiation protocol was carried out over four days. Cells were fed daily by aspirating medium from each well and replacing with a fresh aliquot (0.5 ml) of DMEM-high glucose (Invitrogen; Cat #10569) supplemented with 1% B27 (Invitrogen; Cat #17504-044), 50 ng/ml FGF7, 100 ng/ml Noggin (R&D Systems; Cat #3344-NG), 250 nM KAAD-cyclopamine (Calbiochem; Cat #239804), and 2 μM all-trans retinoic acid (RA) (Sigma-Aldrich; Cat #R2625). At the conclusion of the third step of differentiation, cells from some wells were harvested for analysis by RT-PCR to measure markers of differentiation. Other culture wells were subjected to high content image analysis for protein expression levels of PDX1, a transcription factor correlated with pancreatic endoderm differentiation, and CDX2, a transcription factor associated with intestinal endoderm.

Step 4 of the differentiation protocol was carried out over three days. Cells were fed daily by aspirating the medium from each well and replacing with a fresh aliquot (0.5 ml) of DMEM-high glucose supplemented with 1% B27, 100 ng/ml Noggin, 100 ng/ml Netrin-4 (R&D Systems; Cat #), 1 μM DAPT, and 1 μM Alk 5 inhibitor (Axxora; Cat #ALX-270-445). At the conclusion of the fourth step of differentiation, cells from some wells were harvested for analysis by RT-PCR to measure markers of differentiation.

FACS Analysis: Cells for FACS analysis were blocked in a 1:5 solution of 0.5% human gamma-globulin (Sigma; Cat #G-4386) in PBS (Invitrogen; Cat #14040-133): BD FACS staining buffer-BSA (BD; Cat #554657) for 15 minutes at 4° C. Cells were then stained with an antibody for CXCR4 APC(R&D Systems; Cat#FAB 173A) for 30 minutes at 4° C. After a series of washes in BD FACS staining buffer, the cells were stained for viability with 7-AAD (BD; Cat #559925) and run on a BD FACSArray. A mouse IgG1K Isotype control antibody for APC was used to gate percent positive cells.

RT-PCR Analysis: RNA samples were purified by binding to a silica-gel membrane (Rneasy Mini Kit, Qiagen, CA) in the presence of an ethanol-containing, high-salt buffer followed by washing to remove contaminants. The RNA was further purified using a TURBO DNA-free kit (Ambion, INC), and high-quality RNA was then eluted in water. Yield and purity were assessed by A260 and A280 readings on a spectrophotometer. CDNA copies were made from purified RNA using an ABI (ABI, CA) high capacity cDNA archive kit.

Unless otherwise stated, all reagents were purchased from Applied Biosystems. Real-time PCR reactions were performed using the ABI PRISM® 7900 Sequence Detection System. TAQMAN® UNIVERSAL PCR MASTER MIX® (ABI, CA) was used with 20 ng of reverse transcribed RNA in a total reaction volume of 20 μl. Each cDNA sample was run in duplicate to correct for pipetting errors. Primers and FAM-labeled TAQMAN® probes were used at concentrations of 200 nM. The level of expression for each target gene was normalized using a human glyceraldehyde-3-phosphate dehydrogenase (GAPDH) endogenous control previously developed by Applied Biosystems. Primer and probe sets were as follows: GAPDH (Applied Biosystems), FOXA2 (Hs00232764_m1, Applied Biosystems), SOX17 (Hs00751752_s1, Applied Biosystems), CDX2 (Hs00230919_m1, Applied Biosystems), PDX1 (Hs00236830_m1, Applied Biosystems), NGN3 (Hs00360700_g1, Applied Biosystems), NKX6.1 (Hs00232355_m1, Applied Biosystems), and PTF1 alpha (Hs00603586_g1, Applied Biosystems). After an initial incubation at 50° C. for 2 min followed by 95° C. for 10 min, samples were cycled 40 times in two stages—a denaturation step at 95° C. for 15 sec followed by an annealing/extension step at 60° C. for 1 min. Data analysis was carried out using GENEAMP®7000 Sequence Detection System software. For each primer/probe set, a Ct value was determined as the cycle number at which the fluorescence intensity reached a specific value in the middle of the exponential region of amplification. Relative gene expression levels were calculated using the comparative Ct method. Briefly, for each cDNA sample, the endogenous control Ct value was subtracted from the gene of interest Ct to give the delta Ct value (ΔCt). The normalized amount of target was calculated as 2-ΔCt, assuming amplification to be 100% efficiency. Final data were expressed relative to a calibrator sample.

High Content Analysis: At the conclusion of three days of culture, assay plates were washed once with PBS (Invitrogen; Cat #14190), fixed with 4% paraformaldehyde (Alexis Biochemical; Cat #ALX-350-011) at room temperature for 20 minutes, then washed three times with PBS and permeabilized with 0.5% Triton X-100 (Sigma; Cat #T8760-2) for 20 minutes at room temperature. Cells were washed again three times with PBS and blocked with 4% chicken serum (Invitrogen; Cat #16110082) in PBS for 30 minutes at room temperature. Primary antibody (goat anti-human SOX17; R&D Systems; Cat #AF1924) was diluted 1:100 in 4% chicken serum and added to each well for two hours at room temperature. After washing three times with PBS, Alexa Fluor 488 conjugated secondary antibody (chicken anti-goat IgG; Invitrogen; Cat #A21467) diluted 1:200 in PBS was added to each well. To counterstain nuclei, 5 μg/ml Hoechst 33342 (Invitrogen; Cat #H3570) was added for fifteen minutes at room temperature. Plates were washed once with PBS and left in 100 μl/well PBS for imaging. Other primary antibodies used for analysis included 1:100 dilution mouse anti-human CDX2 (Invitrogen; Cat #397800), 1:100 dilution goat anti-human PDX1 (Santa Cruz Biotechnology; Cat #SC-14664), 1:200 dilution rabbit anti-human insulin (Cell Signaling; Cat #C27C9), and 1:1500 dilution mouse anti-human glucagon (Sigma-Aldrich; Cat #G2654). Secondary antibodies used for analysis included 1:400 dilution Alexa Fluor 647 chicken anti-mouse IgG (Invitrogen; Cat #A-21463), 1:200 dilution Alexa Fluor 488 donkey anti-goat IgG (Invitrogen; Cat #A11055), 1:1000 dilution Alexa Fluor 647 chicken anti-rabbit IgG (Invitrogen; Cat #A21443), and 1:1000 dilution Alexa Fluor 488 chicken anti-mouse IgG (Invitrogen; Cat #A21200).

Imaging was performed using an IN Cell Analyzer 1000 (GE Healthcare) utilizing the 51008bs dichroic for cells stained with Hoechst 33342 and Alexa Fluor 488. Images were acquired from 25 fields per well. Measurements for total intensity were obtained from each well using IN Cell Developer Toolbox 1.7 (GE Healthcare) software. Segmentation for the nuclei was determined based on gray-scale levels (baseline range 100-300) and nuclear size. Averages and standard deviations were calculated for each replicate data set. Total protein expression was reported as total intensity or integrated intensity, defined as total fluorescence of the cell multiplied by the area of the cell. Background was eliminated based on acceptance criteria of gray-scale ranges between 200 and 4500. Total intensity data were normalized by dividing total intensities for each well by the average total intensity for the positive control.

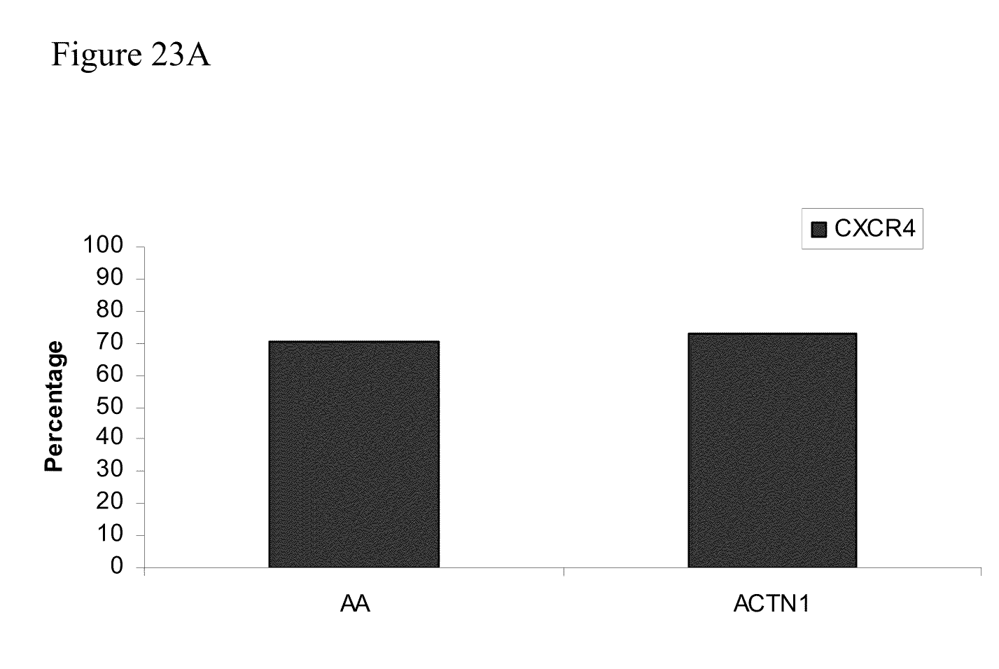

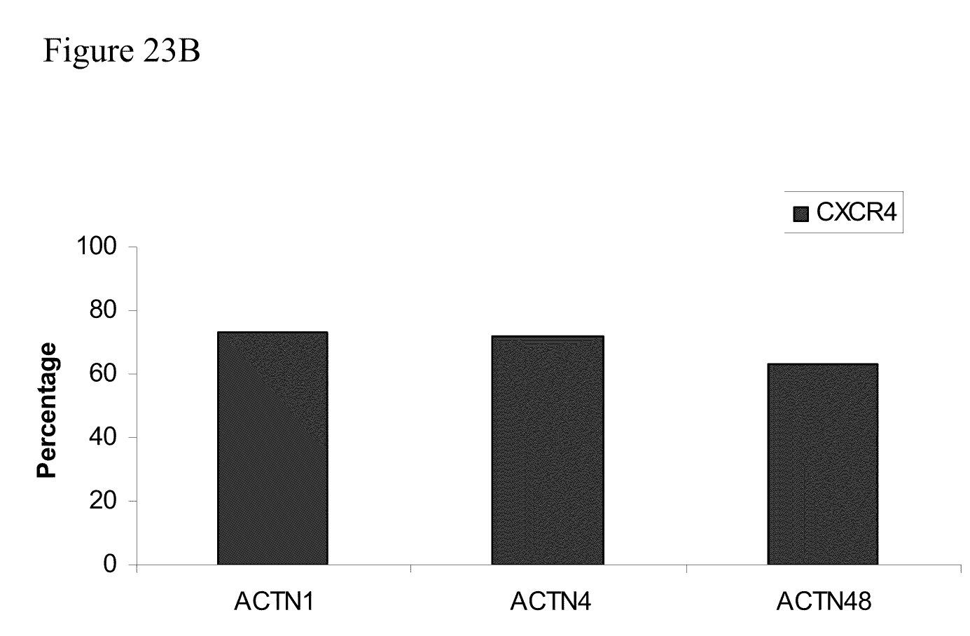

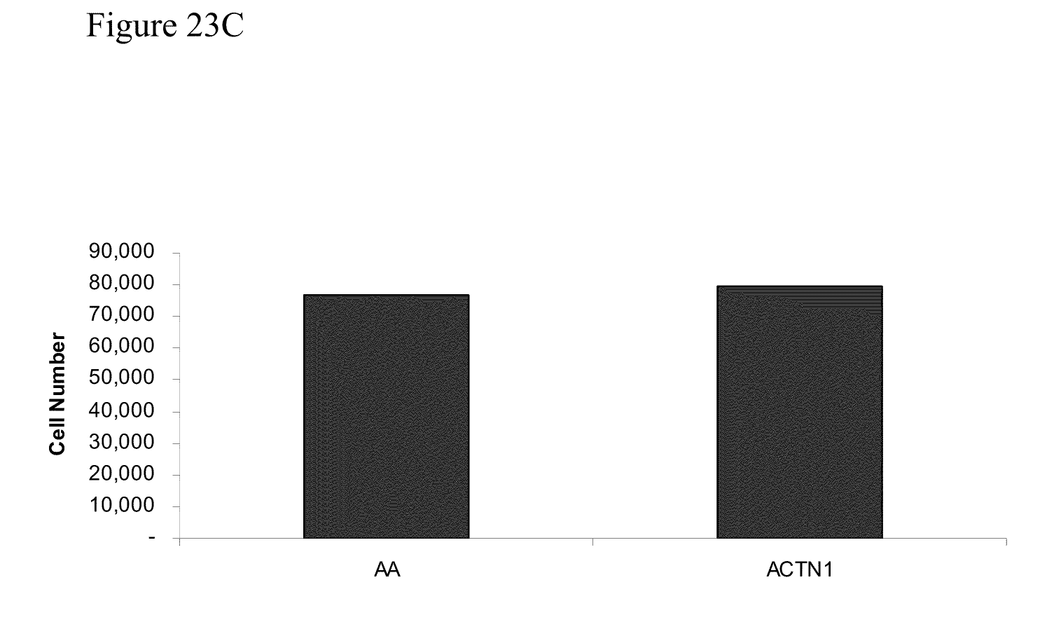

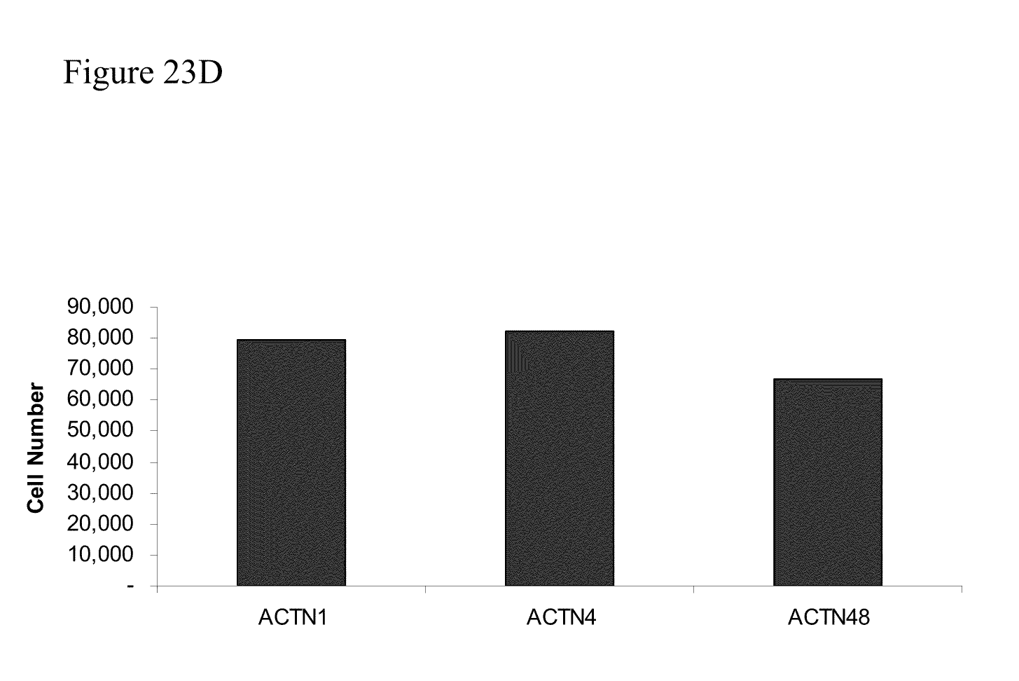

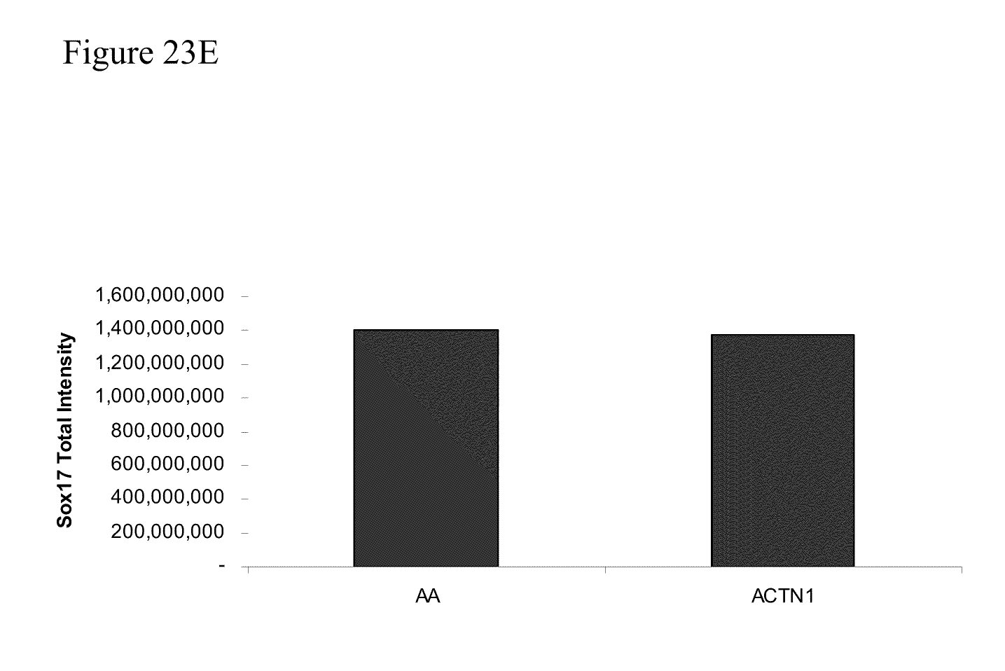

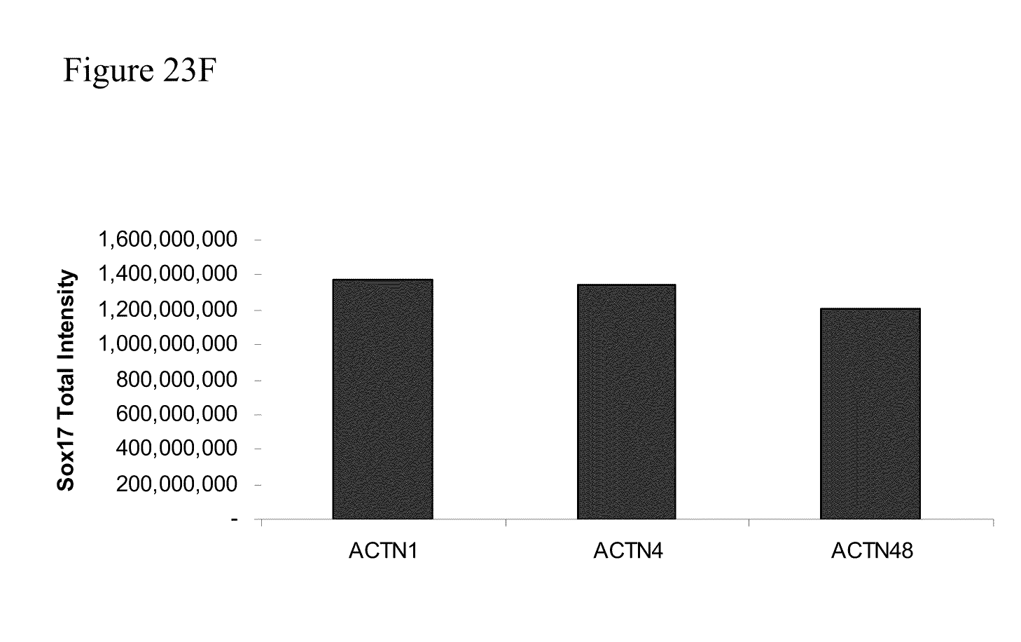

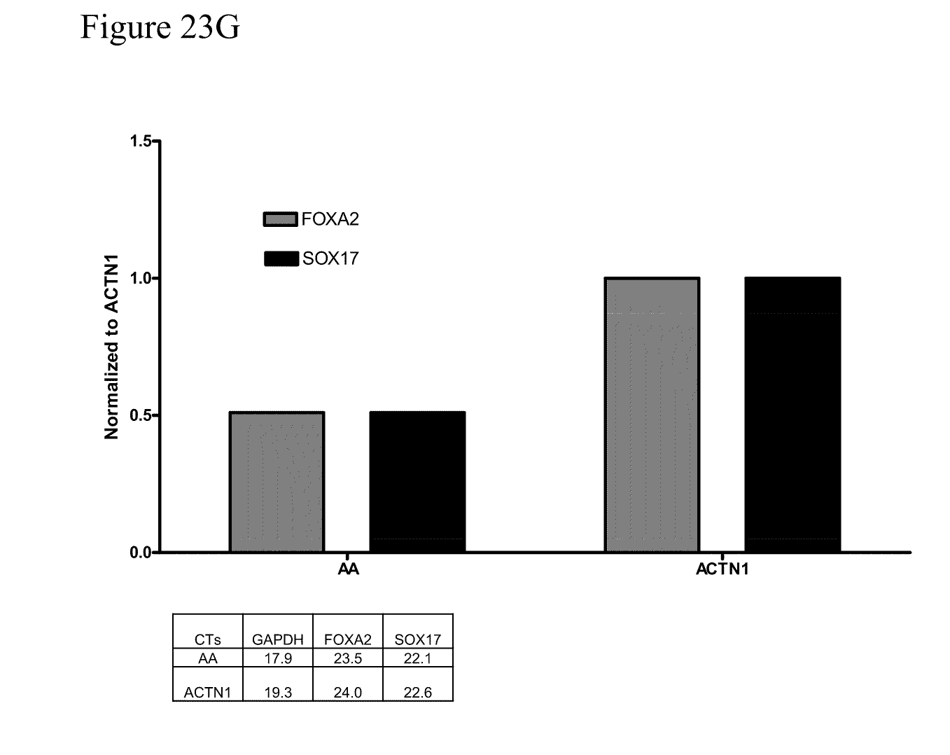

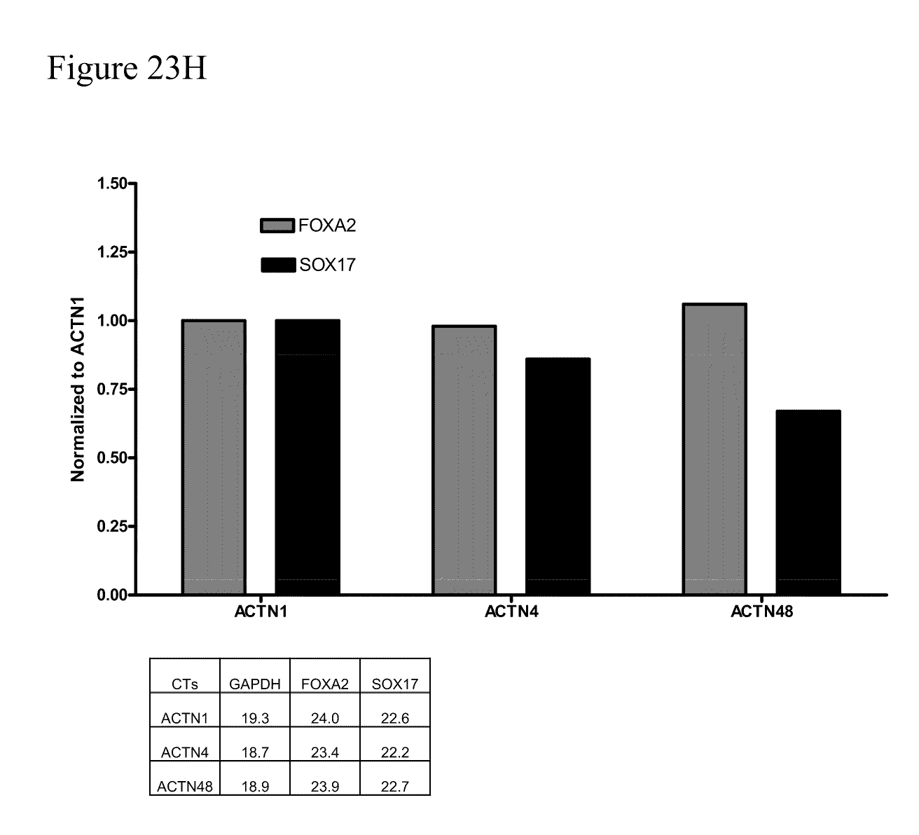

Results: FIG. 23 shows results at the conclusion of the first step of differentiation using flow cytometric, PCR, and high content measure for multiple markers representative of definitive endoderm. FIG. 23A depicts FACS analysis for levels of CXCR4, comparing treatment with a commercial source of activin A versus wild type ACTN1 treatment; results demonstrate equivalent and robust CXCR4 expression for both treatments. FIG. 23B shows CXCR4 expression for two variant peptides (ACTN4 and ACTN48) compared to the wild type ACTN1 peptide; results are equivalent or comparable for all treatments. FIG. 23C through FIG. 23F shows high content analysis for cell number and SOX17 expression at the end of the first step of differentiation, again demonstrating equivalent results for treatment with commercial activin A and the ACNT1 wild type peptide, also showing comparable results with each of the two variant peptides. FIGS. 23G and 23H show RT-PCR results at the conclusion of the first step of differentiation. Relative to the ACTN1 and commercial activin A treatments, samples treated with the ACTN4 and ACTN48 variant peptides have similar expression levels of SOX17 and FOXA2, markers associated with definitive endoderm differentiation.

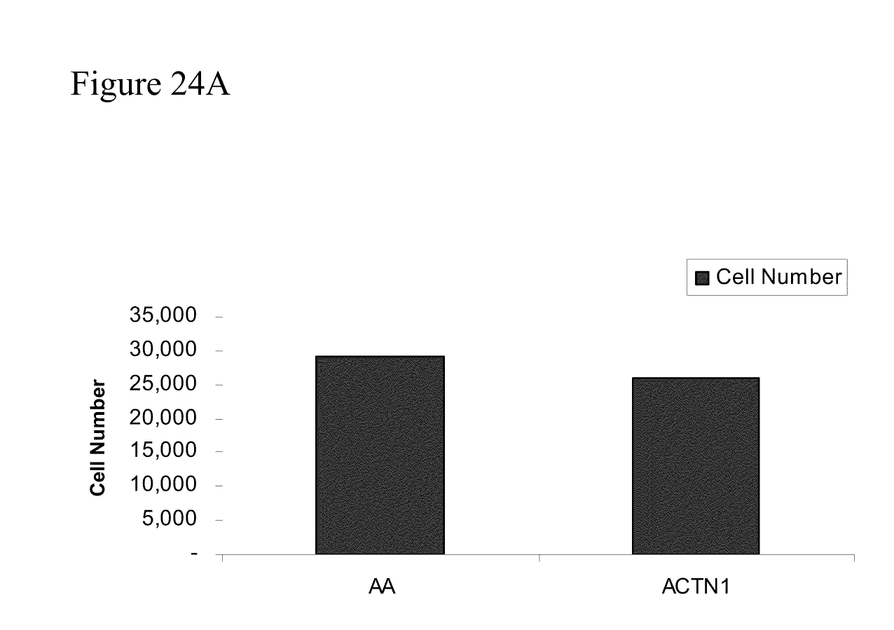

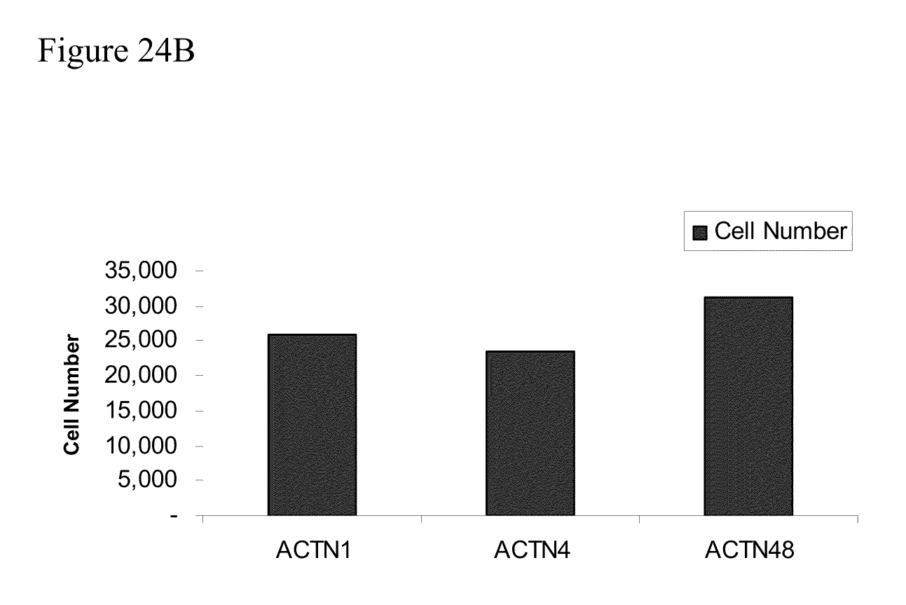

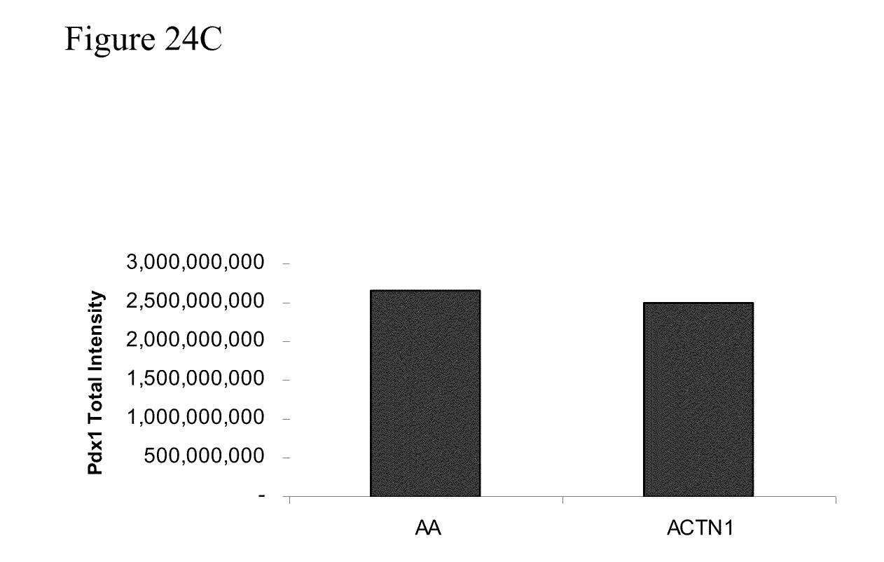

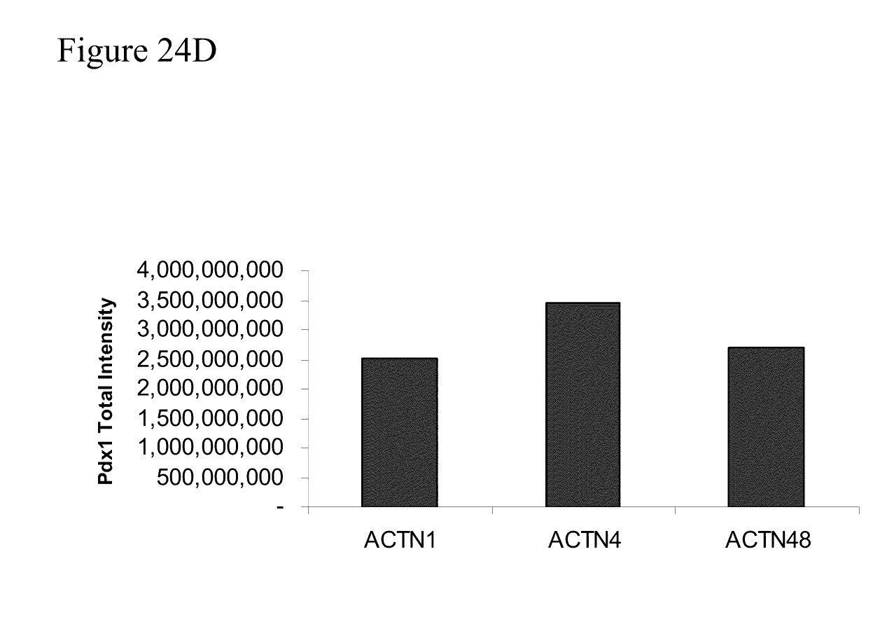

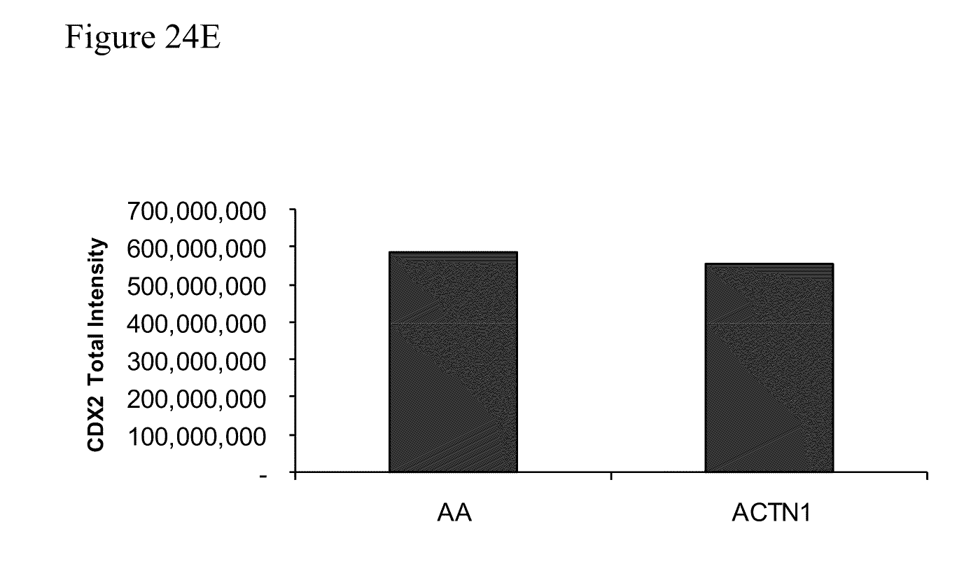

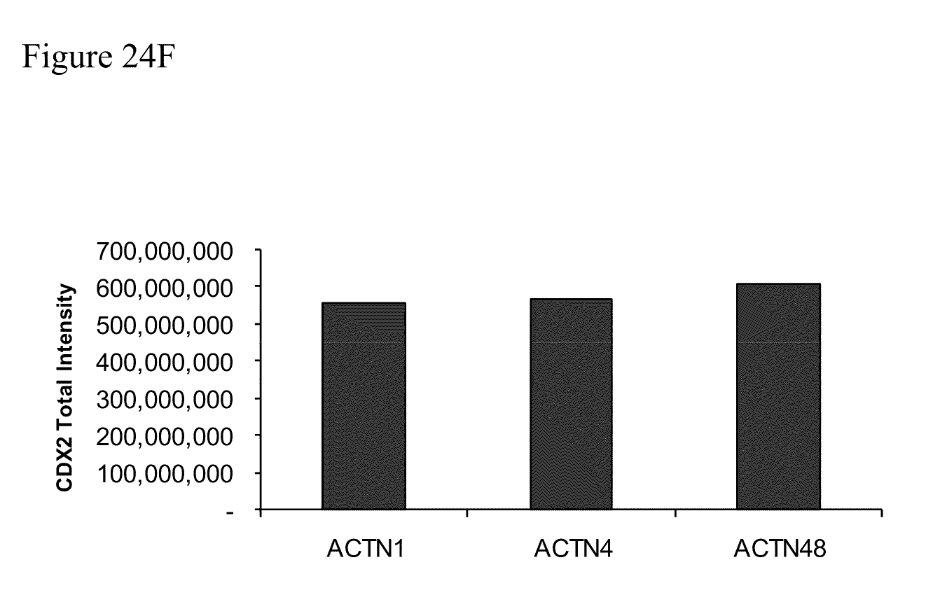

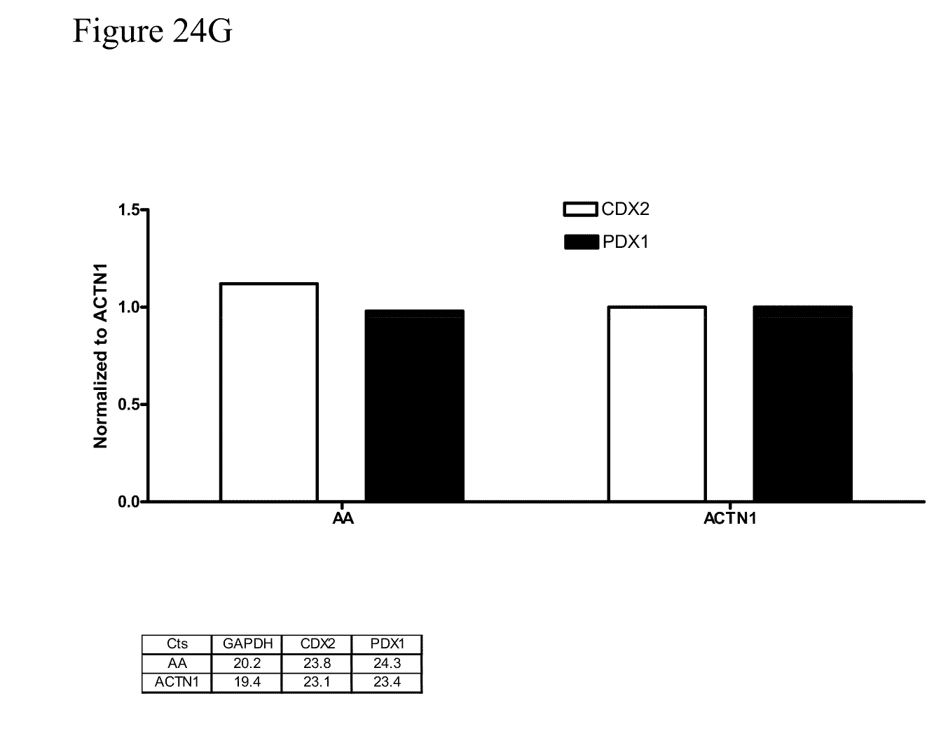

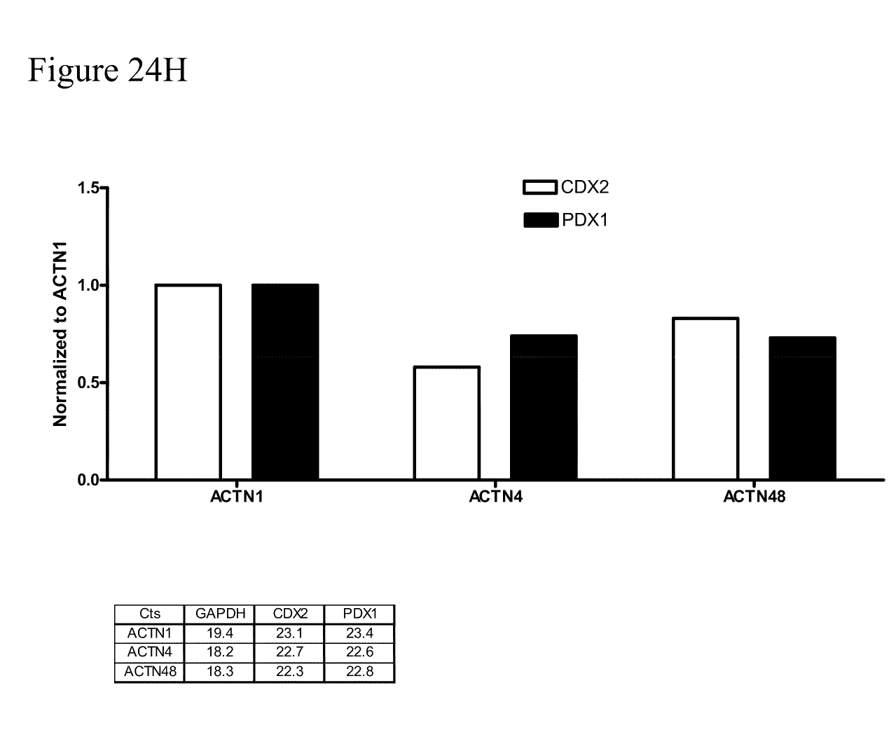

FIG. 24 shows results at the conclusion of the third step of differentiation using PCR and high content analysis measures for multiple markers representative of pancreatic endoderm. Treatment with the ACTN4 and ACTN48 variant peptides yielded equivalent cell numbers and equivalent protein expression of PDX1 and CDX2, comparable to results observed with treatment using commercial activin A or the ACTN1 wild type peptide. RT-PCR results were in agreement.

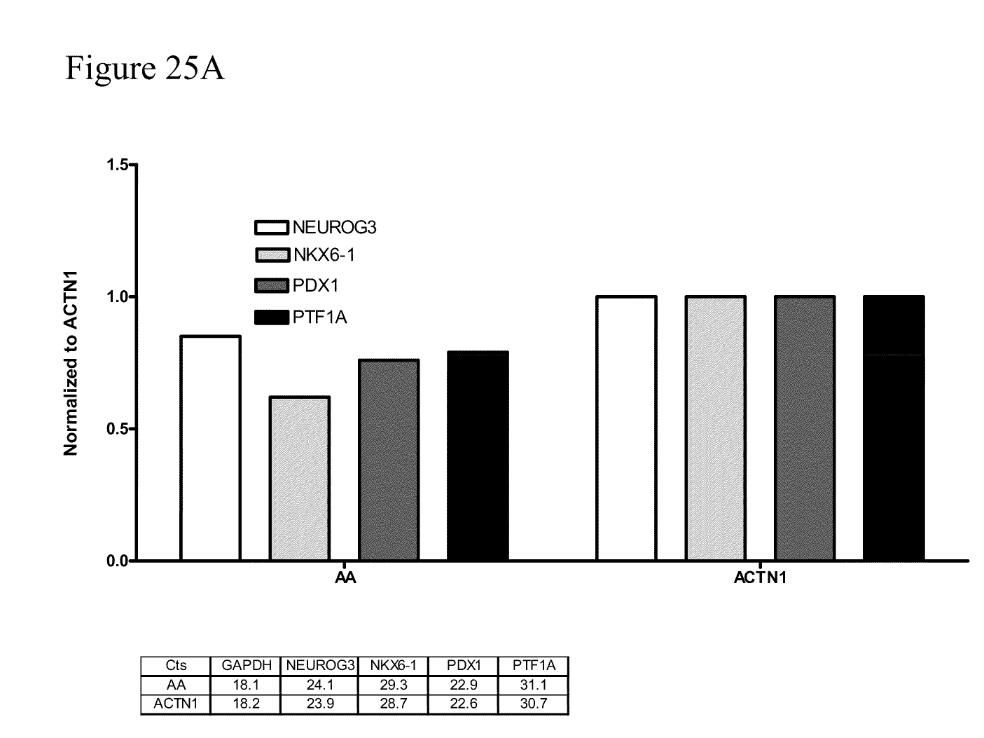

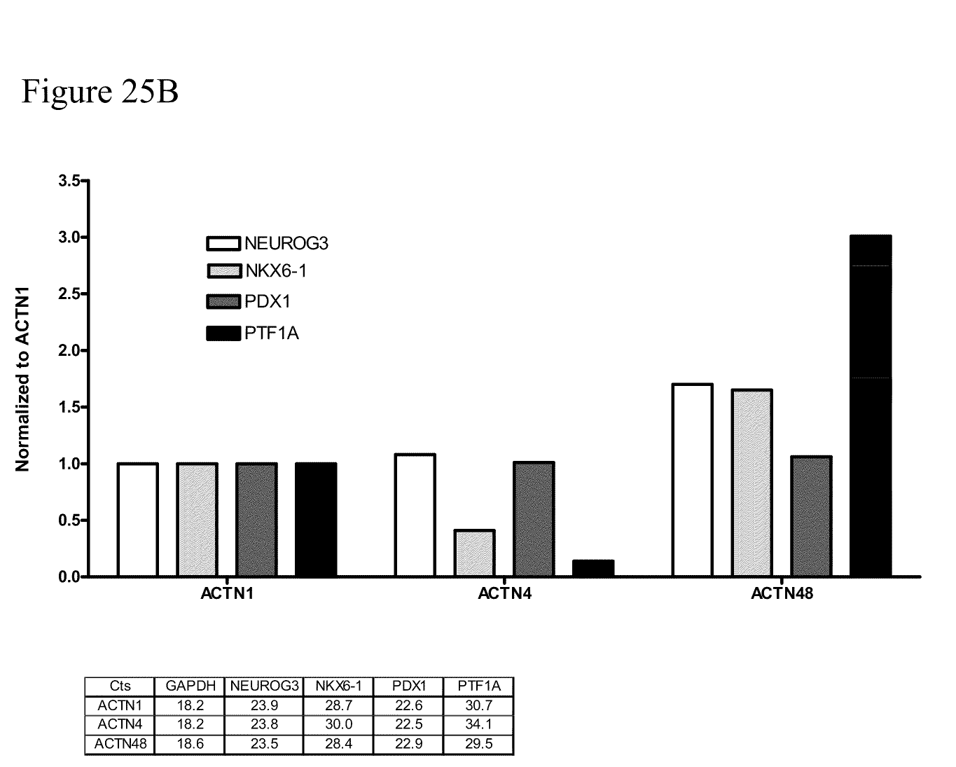

FIG. 25 shows RT-PCR results at the conclusion of step four of differentiation. As before, treatment with the ACTN4 and ACTN48 variant peptides yielded comparable expression of downstream pancreatic differentiation markers relative to treatment with commercial activin A or the ACTN1 wild type peptide.

These collective results demonstrate that the ACTN4 and ACTN48 variant peptides can substitute for Activin A during definitive endoderm differentiation and subsequent pancreatic endoderm and endocrine differentiation.

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

TABLE 1
Amino acid sequences of pro region and mature
protein regions of the peptides of the present
invention

Pro region:
>Wild type Activin A pro region
(SwissProt/UniProt: P08476): SEQ ID 1
MPLLWLRGFLLASCWIIVRSSPTPGSEGHSAAPDCPSCALAALPKDVP
NSQPEMVEAVKKHILNMLHLKKRPDVTQPVPKAALLNAIRKLHVGKVG
ENGYVEIEDDIGRRAEMNELMEQTSEIITFAESGTARKTLHFEISKEG
SDLSVVERAEVWLFLKVPKANRTRTKVTIRLFQQQKHPQGSLDTGEEA
EEVGLKGERSELLLSEKVVDARKSTWHVFPVSSSIQRLLDQGKSSLDV
RIACEQCQESGASLVLLGKKKKKEEEGEGKKKGGGEGGAGADEEKEQS
HRPFLMLQARQSEDHPHRRRRR
Mature protein regions
>ACTN1 (wild type Activin A)
(SwissProt/UniProt: P08476): SEQ ID 2
GLECDGKVNICCKKQFFVSFKDIGWNDWIIAPSGYHANYCEGECPSHI
AGTSGSSLSFHSTVINHYRMRGHSPFANLKSCCVPTKLRPMSMLYYDD
GQNIIKKDIQNMIVEECGCS
>ACTN2: SEQ ID 3
GLECDGKVNLCCKKQNFVSFKDIGWNDWIIAPSGYHANECTGKCPSHI
AGTSGSSLSFHSTVINHYRMRGHSPFSDLGSCCIPTKLRPMSMLYYDD
GQNIIKKDIQNMIVEECGCS
>ACTN3: SEQ ID 4
GLECDGKVNLCCKKQDFVSFKDIGWNDWIIAPSGYHANRCSGKCPSHI
AGTSGSSLSFHSTVINHYRMRGHSPFSNMGSCCIPTKLRPMSMLYYDD
GQNIIKKDIQNMIVEECGCS
>ACTN4: SEQ ID 5
GLECDGKVNLCCKKQLFVSFKDIGWNDWIIAPSGYHANHCSGLCPSHI
AGTSGSSLSFHSTVINHYRMRGHSPFSDMGACCVPTKLRPMSMLYYDD
GQNIIKKDIQNMIVEECGCS
>ACTN5: SEQ ID 6
GLECDGKVNYCCKKQNFVSFKDIGWNDWIIAPSGYHANECSGKCPSHI
AGTSGSSLSFHSTVINHYRMRGHSPFSNLGACCIPTKLRPMSMLYYDD
GQNIIKKDIQNMIVEECGCS
>ACTN6: SEQ ID 7
GLECDGKVNLCCKKQWFVSFKDIGWNDWIIAPSGYHANRCSGKCPSHI
AGTSGSSLSFHSTVINHYRMRGHSPFADMGACCIPTKLRPMSMLYYDD
GQNIIKKDIQNMIVEECGCS
>ACTN7: SEQ ID 8
GLECDGKVNYCCKKQHFVSFKDIGWNDWIIAPSGYHANSCSGLCPSHI
AGTSGSSLSFHSTVINHYRMRGHSPFSQMGSCCIPTKLRPMSMLYYDD
GQNIIKKDIQNMIVEECGCS
>ACTN8: SEQ ID 9
GLECDGKVNYCCKKQDFVSFKDIGWNDWIIAPSGYHANKCSGKCPSHI
AGTSGSSLSFHSTVINHYRMRGHSPFSDMGSCCVPTKLRPMSMLYYDD
GQNIIKKDIQNMIVEECGCS
>ACTN9: SEQ ID 10
GLECDGKVNYCCKKQDFVSFKDIGWNDWIIAPSGYHANKCTGKCPSHI
AGTSGSSLSFHSTVINHYRMRGHSPFADLGACCIPTKLRPMSMLYYDD
GQNIIKKDIQNMIVEECGCS
>ACTN10: SEQ ID 11
GLECDGKVNYCCKKQDFVSFKDIGWNDWIIAPSGYHANRCSGLCPSHI
AGTSGSSLSFHSTVINHYRMRGHSPFAQMGSCCIPTKLRPMSMLYYDD
GQNIIKKDIQNMIVEECGCS
>ACTN11: SEQ ID 12
GLECDGKVNLCCKKQLFVSFKDIGWNDWIIAPSGYHANHCSGLCPSHI
AGTSGSSLSFHSTVINHYRMRGHSPFAQMGACCIPTKLRPMSMLYYDD
GQNIIKKDIQNMIVEECGCS
>ACTN12: SEQ ID 13
GLECDGKVNYCCKKQNFVSFKDIGWNDWIIAPSGYHANECSGLCPSHI
AGTSGSSLSFHSTVINHYRMRGHSPFSQMGSCCIPTKLRPMSMLYYDD
GQNIIKKDIQNMIVEECGCS
>ACTN13: SEQ ID 14
GLECDGKVNLCCKKQDFVSFKDIGWNDWIIAPSGYHANKCSGKCPSHI
AGTSGSSLSFHSTVINHYRMRGHSPFSDMGSCCIPTKLRPMSMLYYDD
GQNIIKKDIQNMIVEECGCS
>ACTN14: SEQ ID 15
GLECDGKVNLCCKKQHFVSFKDIGWNDWIIAPSGYHANRCDGLCPSHI
AGTSGSSLSFHSTVINHYRMRGHSPFAQMGSCCVPTKLRPMSMLYYDD
GQNIIKKDIQNMIVEECGCS
>ACTN15: SEQ ID 16
GLECDGKVNLCCKKQDFVSFKDIGWNDWIIAPSGYHANRCSGLCPSHI
AGTSGSSLSFHSTVINHYRMRGHSPFSNMGSCCIPTKLRPMSMLYYDD
GQNIIKKDIQNMIVEECGCS
>ACTN16: SEQ ID 17
GLECDGKVNLCCKKQLFVSFKDIGWNDWIIAPSGYHANHCSGLCPSHI
AGTSGSSLSFHSTVINHYRMRGHSPFSQMGACCVPTKLRPMSMLYYDD
GQNIIKKDIQNMIVEECGCS
>ACTN17: SEQ ID 18
GLECDGKVNYCCKKQDFVSFKDIGWNDWIIAPSGYHANKCGGKCPSHI
AGTSGSSLSFHSTVINHYRMRGHSPFSDMGACCVPTKLRPMSMLYYDD
GQNIIKKDIQNMIVEECGCS
>ACTN18: SEQ ID 19
GLECDGKVNYCCKKQNFVSFKDIGWNDWIIAPSGYHANKCSGKCPSHI
AGTSGSSLSFHSTVINHYRMRGHSPFSDMGACCIPTKLRPMSMLYYDD
GQNIIKKDIQNMIVEECGCS
>ACTN19: SEQ ID 20
GLECDGKVNLCCKKQDFVSFKDIGWNDWIIAPSGYHANKCGGKCPSHI
AGTSGSSLSFHSTVINHYRMRGHSPFANMGSCCIPTKLRPMSMLYYDD
GQNIIKKDIQNMIVEECGCS
>ACTN20: SEQ ID 21
GLECDGKVNLCCKKQNFVSFKDIGWNDWIIAPSGYHANKCGGLCPSHI
AGTSGSSLSFHSTVINHYRMRGHSPFAQMGACCIPTKLRPMSMLYYDD
GQNIIKKDIQNMIVEECGCS
>ACTN21: SEQ ID 22
GLECDGKVNYCCKKQWFVSFKDIGWNDWIIAPSGYHANKCSGKCPSHI
AGTSGSSLSFHSTVINHYRMRGHSPFANMGSCCIPTKLRPMSMLYYDD
GQNIIKKDIQNMIVEECGCS
>ACTN22: SEQ ID 23
GLECDGKVNLCCKKQDFVSFKDIGWNDWIIAPSGYHANRCDGLCPSHI
AGTSGSSLSFHSTVINHYRMRGHSPFALMGACCIPTKLRPMSMLYYDD
GQNIIKKDIQNMIVEECGCS
>ACTN23: SEQ ID 24
GLECDGKVNYCCKKQDFVSFKDIGWNDWIIAPSGYHANKCDGKCPSHI
AGTSGSSLSFHSTVINHYRMRGHSPFSDMGSCCIPTKLRPMSMLYYDD
GQNIIKKDIQNMIVEECGCS
>ACTN24: SEQ ID 25
GLECDGKVNLCCKKQDFVSFKDIGWNDWIIAPSGYHANRCSGRCPSHI
AGTSGSSLSFHSTVINHYRMRGHSPFSDMGSCCVPTKLRPMSMLYYDD
GQNIIKKDIQNMIVEECGCS
>ACTN25: SEQ ID 26
GLECDGKVNLCCKKQNFVSFKDIGWNDWIIAPSGYHANECSGLCPSHI
AGTSGSSLSFHSTVINHYRMRGHSPFSQMGSCCIPTKLRPMSMLYYDD
GQNIIKKDIQNMIVEECGCS
>ACTN26: SEQ ID 27
GLECDGKVNLCCKKQHFVSFKDIGWNDWIIAPSGYHANRCDGKCPSHI
AGTSGSSLSFHSTVINHYRMRGHSPFANMGACCIPTKLRPMSMLYYDD
GQNIIKKDIQNMIVEECGCS
>ACTN27: SEQ ID 28
GLECDGKVNLCCKKQLFVSFKDIGWNDWIIAPSGYHANHCDGKCPSHI
AGTSGSSLSFHSTVINHYRMRGHSPFANRGACCIPTKLRPMSMLYYDD
GQNIIKKDIQNMIVEECGCS
>ACTN28: SEQ ID 29
GLECDGKVNYCCKKQLFVSFKDIGWNDWIIAPSGYHANHCSGKCPSHI
AGTSGSSLSFHSTVINHYRMRGHSPFANMGSCCIPTKLRPMSMLYYDD
GQNIIKKDIQNMIVEECGCS
>ACTN29: SEQ ID 30
GLECDGKVNLCCKKQLFVSFKDIGWNDWIIAPSGYHANHCSGKCPSHI
AGTSGSSLSFHSTVINHYRMRGHSPFSDMGSCCIPTKLRPMSMLYYDD
GQNIIKKDIQNMIVEECGCS
>ACTN30: SEQ ID 31
GLECDGKVNYCCKKQNFVSFKDIGWNDWIIAPSGYHANKCSGLCPSHI
AGTSGSSLSFHSTVINHYRMRGHSPFSKMGACCVPTKLRPMSMLYYDD
GQNIIKKDIQNMIVEECGCS
>ACTN31: SEQ ID 32
GLECDGKVNTCCKKQLFVSFKDIGWNDWIIAPSGYHANHCSGKCPSHI
AGTSGSSLSFHSTVINHYRMRGHSPFSDMGSCCIPTKLRPMSMLYYDD
GQNIIKKDIQNMIVEECGCS
>ACTN32: SEQ ID 33
GLECDGKVNLCCKKQDFVSFKDIGWNDWIIAPSGYHANRCGGKCPSHI
AGTSGSSLSFHSTVINHYRMRGHSPFSNMGSCCIPTKLRPMSMLYYDD
GQNIIKKDIQNMIVEECGCS
>ACTN33: SEQ ID 34
GLECDGKVNLCCKKQNFVSFKDIGWNDWIIAPSGYHANECMGKCPSHI
AGTSGSSLSFHSTVINHYRMRGHSPFSDMGACCIPTKLRPMSMLYYDD
GQNIIKKDIQNMIVEECGCS
>ACTN34: SEQ ID 35
GLECDGKVNYCCKKQLFVSFKDIGWNDWIIAPSGYHANHCTGKCPSHI
AGTSGSSLSFHSTVINHYRMRGHSPFSDLGSCCVPTKLRPMSMLYYDD
GQNIIKKDIQNMIVEECGCS
>ACTN35: SEQ ID 36
GLECDGKVNLCCKKQDFVSFKDIGWNDWIIAPSGYHANRCSGKCPSHI
AGTSGSSLSFHSTVINHYRMRGHSPFSDMGSCCVPTKLRPMSMLYYDD
GQNIIKKDIQNMIVEECGCS
>ACTN36: SEQ ID 37
GLECDGKVNLCCKKQDFVSFKDIGWNDWIIAPSGYHANRCSGKCPSHI
AGTSGSSLSFHSTVINHYRMRGHSPFANMGACCVPTKLRPMSMLYYDD
GQNIIKKDIQNMIVEECGCS
>ACTN37: SEQ ID 38
GLECDGKVNLCCKKQDFVSFKDIGWNDWIIAPSGYHANRCSGKCPSHI
AGTSGSSLSFHSTVINHYRMRGHSPFSDMGACCIPTKLRPMSMLYYDD
GQNIIKKDIQNMIVEECGCS
>ACTN38: SEQ ID 39
GLECDGKVNLCCKKQDFVSFKDIGWNDWIIAPSGYHANKCGGKCPSHI
AGTSGSSLSFHSTVINHYRMRGHSPFSQLGACCVPTKLRPMSMLYYDD
GQNIIKKDIQNMIVEECGCS
>ACTN39: SEQ ID 40
GLECDGKVNLCCKKQNFVSFKDIGWNDWIIAPSGYHANECSGLCPSHI
AGTSGSSLSFHSTVINHYRMRGHSPFSDMGSCCVPTKLRPMSMLYYDD
GQNIIKKDIQNMIVEECGCS
>ACTN40: SEQ ID 41
GLECDGKVNLCCKKQLFVSFKDIGWNDWIIAPSGYHANHCAGLCPSHI
AGTSGSSLSFHSTVINHYRMRGHSPFSNMGSCCVPTKLRPMSMLYYDD
GQNIIKKDIQNMIVEECGCS
>ACTN41: SEQ ID 42
GLECDGKVNLCCKKQDFVSFKDIGWNDWIIAPSGYHANSCSGLCPSHI
AGTSGSSLSFHSTVINHYRMRGHSPFSDRGACCIPTKLRPMSMLYYDD
GQNIIKKDIQNMIVEECGCS
>ACTN42: SEQ ID 43
GLECDGKVNYCCKKQDFVSFKDIGWNDWIIAPSGYHANKCSGKCPSHI
AGTSGSSLSFHSTVINHYRMRGHSPFSDMGSCCIPTKLRPMSMLYYDD
GQNIIKKDIQNMIVEECGCS
>ACTN43: SEQ ID 44
GLECDGKVNYCCKKQDFVSFKDIGWNDWIIAPSGYHANRCDGKCPSHI
AGTSGSSLSFHSTVINHYRMRGHSPFSDMGACCVPTKLRPMSMLYYDD
GQNIIKKDIQNMIVEECGCS
>ACTN44: SEQ ID 45
GLECDGKVNLCCKKQNFVSFKDIGWNDWIIAPSGYHANECSGKCPSHI
AGTSGSSLSFHSTVINHYRMRGHSPFSDMGACCIPTKLRPMSMLYYDD
GQNIIKKDIQNMIVEECGCS
>ACTN45: SEQ ID 46
GLECDGKVNTCCKKQNFVSFKDIGWNDWIIAPSGYHANECSGKCPSHI
AGTSGSSLSFHSTVINHYRMRGHSPFANMGACCIPTKLRPMSMLYYDD
GQNIIKKDIQNMIVEECGCS
>ACTN46: SEQ ID 47
GLECDGKVNLCCKKQNFVSFKDIGWNDWIIAPSGYHANECGGLCPSHI
AGTSGSSLSFHSTVINHYRMRGHSPHANRGACCIPTKLRPMSMLYYDD
GQNIIKKDIQNMIVEECGCS
>ACTN47: SEQ ID 48
GLECDGKVNYCCKKQNFVSFKDIGWNDWIIAPSGYHANECSGKCPSHI
AGTSGSSLSFHSTVINHYRMRGHSPFSDMGSCCIPTKLRPMSMLYYDD
GQNIIKKDIQNMIVEECGCS
>ACTN48: SEQ ID 49
GLECDGKVNLCCKKQNFVSFKDIGWNDWIIAPSGYHANECSGLCPSHI
AGTSGSSLSFHSTVINHYRMRGHSPFANMGACCIPTKLRPMSMLYYDD
GQNIIKKDIQNMIVEECGCS
>ACTN49: SEQ ID 50
GLECDGKVNLCCKKQDFVSFKDIGWNDWIIAPSGYHANRCDGLCPSHI
AGTSGSSLSFHSTVINHYRMRGHSPFSDMGSCCVPTKLRPMSMLYYDD
GQNIIKKDIQNMIVEECGCS
>ACTN50: SEQ ID 51
GLECDGKVNICCKKQLFGRTKDIGWNDWIIAPSGYHGGGCSGECPSHI
AGTSGSSLSFHSTVINHYRMRGHSPVANLKSCCSPTKLRPMSMLYYDD
GQNIIKKDIQNMKVEECGCT
>ACTN51: SEQ ID 52
GLECDGKVNICCKKQSFGQTKDIGWNDWIIAPSGYHGGGCSGECPSHI
AGTSGSSLSFHSTVINHYRMRGHSPFANLKSCCSPTKLRPMSMLYYDD
GQNIIKKDIQRMVVEECGCT
>ACTN52: SEQ ID 53
GLECDGKVNICCKKQLFGKTKDIGWNDWIIAPSGYHGGSCTGECPSHI
AGTSGSSLSFHSTVINHYRMRGHSPNANLKSCCSPTKLRPMSMLYYDD
GQNIIKKDIQGMKVEECGCT
>ACTN53: SEQ ID 54
GLECDGKVNICCKKQEFGQAKDIGWNDWIIAPSGYHGGGCSGECPSHI
AGTSGSSLSFHSTVINHYRMRGHSPWANLKSCCSPTKLRPMSMLYYDD
GQNIIKKDIQGMKVEECGCT
>ACTN54: SEQ ID 55
GLECDGKVNICCKKQSFAQTKDIGWNDWIIAPSGYHGGGCTGECPSHI
AGTSGSSLSFHSTVINHYRMRGHSPFANLKSCCSPTKLRPMSMLYYDD
GQNIIKKDIQNMVVEECGCT
>ACTN55: SEQ ID 56

GLECDGKVNICCKKQSFGQTKDIGWNDWIIAPSGYHGGGCTGECPSHI
AGTSGSSLSFHSTVINHYRMRGHSPWANLKSCCSPTKLRPMSMLYYDD
GQNIIKKDIQGMVVEECGCT
>ACTN56: SEQ ID 57
GLECDGKVNICCKKQSFGQAKDIGWNDWIIAPSGYHGGSCTGECPSHI
AGTSGSSLSFHSTVINHYRMRGHSPFANLKSCCAPTKLRPMSMLYYDD
GQNIIKKDIQNMVVEECGCV
>ACTN57: SEQ ID 58
GLECDGKVNICCKKQSFGQAKDIGWNDWIIAPSGYHGGGCSGECPSHI
AGTSGSSLSFHSTVINHYRMRGHSPWANLKSCCSPTKLRPMSMLYYDD
GQNIIKKDIQNMKVEECGCT
>ACTN58: SEQ ID 59
GLECDGKVNICCKKQSFGQTKDIGWNDWIIAPSGYHGGGCTGECPSHI
AGTSGSSLSFHSTVINHYRMRGHSPWANLKSCCSPTKLRPMSMLYYDD
GQNIIKKDIQNMKVEECGCT
>ACTN59: SEQ ID 60
GLECDGKVNICCKKQLFGQTKDIGWNDWIIAPSGYHGGGCTGECPSHI
AGTSGSSLSFHSTVINHYRMRGHSPNANLKSCCAPTKLRPMSMLYYDD
GQNIIKKDIQGMKVEECGCV
>ACTN60: SEQ ID 61
GLECDGKVNICCKKQSFGQTKDIGWNDWIIAPSGYHGGGCSGECPSHI
AGTSGSSLSFHSTVINHYRMRGHSPWANLKSCCAPTKLRPMSMLYYDD
GQNIIKKDIQNMVVEECGCV
>ACTN61: SEQ ID 62
GLECDGKVNICCKKQLFGQTKDIGWNDWIIAPSGYHGGSCTGECPSHI
AGTSGSSLS FHSTVINHYRMRGHSPNANLKSCCSPTKLRPMSMLYYD
DGQNIIKKDIQNMKVEECGCT
>ACTN62: SEQ ID 63
GLECDGKVNICCKKQSFGQAKDIGWNDWIIAPSGYHGGGCTGECPSHI
AGTSGSSLSFHSTVINHYRMRGHSPWANLKSCCSPTKLRPMSMLYYDD
GQNIIKKDIQNMVVEECGCT
>ACTN63: SEQ ID 64
GLECDGKVNICCKKQSFSQAKDIGWNDWIIAPSGYHGGGCTGECPSHI
AGTSGSSLSFHSTVINHYRMRGHSPFANLKSCCSPTKLRPMSMLYYDD
GQNIIKKDIQGMVVEECGCT
>ACTN64: SEQ ID 65
GLECDGKVNICCKKQSFGQTKDIGWNDWIIAPSGYHGGGCSGECPSHI
AGTSGSSLSFHSTVINHYRMRGHSPWANLKSCCSPTKLRPMSMLYYDD
GQNIIKKDIQGMVVEECGCT
>ACTN65: SEQ ID 66
GLECDGKVNICCKKQSFGQTKDIGWNDWIIAPSGYHGGSCSGECPSHI
AGTSGSSLSFHSTVINHYRMRGHSPFANLKSCCSPTKLRPMSMLYYDD
GQNIIKKDIQGMKVEECGCT
>ACTN66: SEQ ID 67
GLECDGKVNICCKKQMFGQAKDIGWNDWIIAPSGYHGGGCTGECPSHI
AGTSGSSLSFHSTVINHYRMRGHSPWANLKSCCSPTKLRPMSMLYYDD
GQNIIKKDIQNMVVEECGCT
>ACTN67: SEQ ID 68
GLECDGKVNICCKKQSFGQTKDIGWNDWIIAPSGYHGGGCSGECPSHI
AGTSGSSLSFHSTVINHYRMRGHSPWANLKSCCSPTKLRPMSMLYYDD
GQNIIKKDIQNMKVEECGCT
>ACTN68: SEQ ID 69
GLECDGKVNICCKKQSFGKAKDIGWNDWIIAPSGYHGGGCTGECPSHI
AGTSGSSLSFHSTVINHYRMRGHSPFANLKSCCSPTKLRPMSMLYYDD
GQNIIKKDIQGMKVEECGCT
>ACTN69: SEQ ID 70
GLECDGKVNICCKKQSFGKTKDIGWNDWIIAPSGYHGGGCTGECPSHI
AGTSGSSLSFHSTVINHYRMRGHSPFANLKSCCSPTKLRPMSMLYYDD
GQNIIKKDIQQMVVEECGCT
>ACTN70: SEQ ID 71
GLECDGKVNICCKKQLFGQAKDIGWNDWIIAPSGYHGGGCSGECPSHI
AGTSGSSLSFHSTVINHYRMRGHSPVANLKSCCSPTKLRPMSMLYYDD
GQNIIKKDIQGMVVEECGCT
>ACTN71: SEQ ID 72
GLECDGKVNICCKKQSFGQAKDIGWNDWIIAPSGYHGGGCTGECPSHI
AGTSGSSLSFHSTVINHYRMRGHSPFANLKSCCSPTKLRPMSMLYYDD
GQNIIKKDIQNMKVEECGCT
>ACTN72: SEQ ID 73
GLECDGKVNICCKKQLFGQAKDIGWNDWIIAPSGYHGGSCTGECPSHI
AGTSGSSLSFHSTVINHYRMRGHSPNANLKSCCSPTKLRPMSMLYYDD
GQNIIKKDIQNMVVEECGCT
>ACTN73: SEQ ID 74
GLECDGKVNICCKKQSFGQAKDIGWNDWIIAPSGYHGGGCSGECPSHI
AGTSGSSLSFHSTVINHYRMRGHSPFANLKSCCSPTKLRPMSMLYYDD
GQNIIKKDIQGMVVEECGCT
>ACTN74: SEQ ID 75
GLECDGKVNICCKKQSFGRAKDIGWNDWIIAPSGYHGGGCTGECPSHI
AGTSGSSLSFHSTVINHYRMRGHSPFANLKSCCSPTKLRPMSMLYYDD
GQNIIKKDIQGMVVEECGCT
>ACTN75: SEQ ID 76
GLECDGKVNICCKKQSFGQAKDIGWNDWIIAPSGYHGGGCSGECPSHI
AGTSGSSLSFHSTVINHYRMRGHSPWANLKSCCSPTKLRPMSMLYYDD
GQNIIKKDIQRMKVEECGCT
>ACTN76: SEQ ID 77
GLECDGKVNICCKKQLFGQTKDIGWNDWIIAPSGYHGGGCSGECPSHI
AGTSGSSLSFHSTVINHYRMRGHSPNANLKSCCSPTKLRPMSMLYYDD
GQNIIKKDIQGMKVEECGCT
>ACTN77: SEQ ID 78
GLECDGKVNICCKKQMFGKAKDIGWNDWIIAPSGYHGGGCTGECPSHI
AGTSGSSLSFHSTVINHYRMRGHSPWANLKSCCSPTKLRPMSMLYYDD
GQNIIKKDIQNMVVEECGCT
>ACTN78: SEQ ID 79
GLECDGKVNICCKKQLFGQAKDIGWNDWIIAPSGYHGGGCSGECPSHI
AGTSGSSLSFHSTVINHYRMRGHSPVANLKSCCSPTKLRPMSMLYYDD
GQNIIKKDIQNMVVEECGCT
>ACTN79: SEQ ID 80
GLECDGKVNICCKKQSFGQAKDIGWNDWIIAPSGYFIGGGCTGECPSH
IAGTSGSSLSFHSTVINHYRMRGHSPWANLKSCCSPTKLRPMSMLYYD
DGQNIIKKDIQRMVVEECGCT
>ACTN80: SEQ ID 81
GLECDGKVNICCKKQSFGQTKDIGWNDWIIAPSGYHGGGCSGECPSHI
AGTSGSSLSFHSTVINHYRMRGHSPWANLKSCCAPTKLRPMSMLYYDD
GQNIIKKDIQNMKVEECGCV
>ACTN81: SEQ ID 82
GLECDGKVNICCKKQLFGKTKDIGWNDWIIAPSGYHGGGCTGECPSHI
AGTSGSSLSFHSTVINHYRMRGHSPVANLKSCCSPTKLRPMSMLYYDD
GQNIIKKDIQRMVVEECGCT
>ACTN82: SEQ ID 83
GLECDGKVNICCKKQSFGQTKDIGWNDWIIAPSGYHGGGCTGECPSHI
AGTSGSSLSFHSTVINHYRMRGHSPWANLKSCCSPTKLRPMSMLYYDD
GQNIIKKDIQGMKVEECGCT
>ACTN83: SEQ ID 84
GLECDGKVNICCKKQSFGRAKDIGWNDWIIAPSGYHGGGCTGECPSHI
AGTSGSSLSFHSTVINHYRMRGHSPFANLKSCCSPTKLRPMSMLYYDD
GQNIIKKDIQGMKVEECGCT
>ACTN84: SEQ ID 85
GLECDGKVNICCKKQSFGQAKDIGWNDWIIAPSGYHGGGCSGECPSHI
AGTSGSSLSFHSTVINHYRMRGHSPFANLKSCCSPTKLRPMSMLYYDD
GQNIIKKDIQNMVVEECGCT
>ACTN85: SEQ ID 86
GLECDGKVNICCKKQLFGQAKDIGWNDWIIAPSGYHGGGCTGECPSHI
AGTSGSSLSFHSTVINHYRMRGHSPVANLKSCCSPTKLRPMSMLYYDD
GQNIIKKDIQNMKVEECGCT
>ACTN86: SEQ ID 87
GLECDGKVNICCKKQSFGKTKDIGWNDWIIAPSGYHGGGCSGECPSHI
AGTSGSSLSFHSTVINHYRMRGHSPWANLKSCCSPTKLRPMSMLYYDD
GQNIIKKDIQNMVVEECGCT
>ACTN87: SEQ ID 88
GLECDGKVNICCKKQSFGQAKDIGWNDWIIAPSGYHGGSCSGECPSHI
AGTSGSSLSFHSTVINHYRMRGHSPFANLKSCCSPTKLRPMSMLYYDD
GQNIIKKDIQNMVVEECGCT
>ACTN88: SEQ ID 89
GLECDGKVNICCKKQSFGQTKDIGWNDWIIAPSGYHGGGCSGECPSHI
AGTSGSSLSFHSTVINHYRMRGHSPWANLKSCCSPTKLRPMSMLYYDD
GQNIIKKDIQNMVVEECGCT
>ACTN89: SEQ ID 90
GLECDGKVNICCKKQLFGQTKDIGWNDWIIAPSGYHGGGCSGECPSHI
AGTSGSSLSFHSTVINHYRMRGHSPNANLKSCCSPTKLRPMSMLYYDD
GQNIIKKDIQNMKVEECGCT
>ACTN90: SEQ ID 91
GLECDGKVNICCKKQSFGQTKDIGWNDWIIAPSGYHGGSCSGECPSHI
AGTSGSSLSFHSTVINHYRMRGHSPFANLKSCCSPTKLRPMSMLYYDD
GQNIIKKDIQNMKVEECGCT
>ACTN91: SEQ ID 92
GLECDGKVNICCKKQSFGRTKDIGWNDWIIAPSGYHGGGCSGECPSHI
AGTSGSSLSFHSTVINHYRMRGHSPWANLKSCCSPTKLRPMSMLYYDD
GQNIIKKDIQNMVVEECGCT
>ACTN92: SEQ ID 93
GLECDGKVNICCKKQSFGQAKDIGWNDWIIAPSGYHGGGCSGECPSHI
AGTSGSSLSFHSTVINHYRMRGHSPFANLKSCCSPTKLRPMSMLYYDD
GQNIIKKDIQGMKVEECGCT
>ACTN93: SEQ ID 94
GLECDGKVNICCKKQSFGQTKDIGWNDWIIAPSGYHGGGCTGECPSHI
AGTSGSSLSFHSTVINHYRMRGHSPFANLKSCCSPTKLRPMSMLYYDD
GQNIIKKDIQNMKVEECGCT
>ACTN94: SEQ ID 95
GLECDGKVNICCKKQSFGQAKDIGWNDWIIAPSGYHGGGCTGECPSHI
AGTSGSSLSFHSTVINHYRMRGHSPWANLKSCCSPTKLRPMSMLYYDD
GQNIIKKDIQRMVAEECGCT

TABLE 2
DNA sequences encoding the peptides
of the present invention

Pro region:
>Wild type Activin A pro region: SEQ ID 96
ATGCCCTTGCTTTGGCTGAGAGGATTTCTGTTGGCAAGTTGCTGGATT
ATAGTGAGGAGTTCCCCCACCCCAGGATCCGAGGGGCACAGCGCGGCC
CCCGACTGTCCGTCCTGTGCGCTGGCCGCCCTCCCAAAGGATGTACCC
AACTCTCAGCCAGAGATGGTGGAGGCCGTCAAGAAGCACATTTTAAAC
ATGCTGCACTTGAAGAAGAGACCCGATGTCACCCAGCCGGTACCCAAG
GCGGCGCTTCTGAACGCGATCAGAAAGCTTCATGTGGGCAAAGTCGGG
GAGAACGGGTATGTGGAGATAGAGGATGACATTGGAAGGAGGGCAGAA
ATGAATGAACTTATGGAGCAGACCTCGGAGATCATCACGTTTGCCGAG
TCAGGAACAGCCAGGAAGACGCTGCACTTCGAGATTTCCAAGGAAGGC
AGTGACCTGTCAGTGGTGGAGCGTGCAGAAGTCTGGCTCTTCCTAAAA
GTCCCCAAGGCCAACAGGACCAGGACCAAAGTCACCATCCGCCTCTTC
CAGCAGCAGAAGCACCCGCAGGGCAGCTTGGACACAGGGGAAGAGGCC
GAGGAAGTGGGCTTAAAGGGGGAGAGGAGTGAACTGTTGCTCTCTGAA
AAAGTAGTAGACGCTCGGAAGAGCACCIGGCATGTCTICCCTGTCTCC
AGCAGCATCCAGCGGTTGCTGGACCAGGGCAAGAGCTCCCTGGACGTT
CGGATTGCCTGTGAGCAGTGCCAGGAGAGTGGCGCCAGCTTGGTTCTC
CTGGGCAAGAAGAAGAAGAAAGAAGAGGAGGGGGAAGGGAAAAAGAAG
GGCGGAGGTGAAGGTGGGGCAGGAGCAGATGAGGAAAAGGAGCAGTCG
CACAGACCTTTCCTCATGCTGCAGGCCCGGCAGTCTGAAGACCACCCT
CATCGCCGGCGTCGGCGG
Mature protein regions:
>ACTN1 (wild type Activin A): SEQ ID 97
GGCCTGGAGTGCGACGGCAAGGTGAACATCTGCTGCAAGAAGCAGTTC
TTCGTGAGCTTCAAGGACATCGGCTGGAACGACTGGATCATCGCCCCC
AGCGGCTACCACGCCAACTACTGCGAGGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN2: SEQ ID 98
GGCCTGGAGTGCGACGGCAAGGTGAACCTGTGCTGCAAGAAGCAGAAC
TTCGTGAGCTTCAAGGACATCGGCTGGAACGACTGGATCATCGCCCCC
AGCGGCTACCACGCCAACGAGTGCACCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACCTGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN3: SEQ ID 99
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCAACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN4: SEQ ID 100
GGCCTGGAGTGCGACGGCAAGGTGAACCTGTGCTGCAAGAAGCAGCTG
TTCGTGAGCTTCAAGGACATCGGCTGGAACGACTGGATCATCGCCCCC
AGCGGCTACCACGCCAACCACTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCGCCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN5: SEQ ID 101
GGCCTGGAGTGCGACGGCAAGGTGAACTACTGCTGCAAGAAGCAGAAC
TTCGTGAGCTTCAAGGACATCGGCTGGAACGACTGGATCATCGCCCCC
AGCGGCTACCACGCCAACGAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCAACCTGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN6: SEQ ID 102
GGCCTGGAGTGCGACGGCAAGGTGAACCTGTGCTGCAAGAAGCAGTGG
TTCGTGAGCTTCAAGGACATCGGCTGGAACGACTGGATCATCGCCCCC
AGCGGCTACCACGCCAACAGGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCGACATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN7: SEQ ID 103
GGCCTGGAGTGCGACGGCAAGGTGAACTACTGCTGCAAGAAGCAGCAC
TTCGTGAGCTTCAAGGACATCGGCTGGAACGACTGGATCATCGCCCCC
AGCGGCTACCACGCCAACAGCTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCCAGATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN8: SEQ ID 104
GGCCTGGAGTGCGACGGCAAGGTGAACTACTGCTGCAAGAAGCAGGAC
TTCGTGAGCTTCAAGGACATCGGCTGGAACGACTGGATCATCGCCCCC
AGCGGCTACCACGCCAACAAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN9: SEQ ID 105
GGCCTGGAGTGCGACGGCAAGGTGAACTACTGCTGCAAGAAGCAGGAC
TTCGTGAGCTTCAAGGACATCGGCTGGAACGACTGGATCATCGCCCCC
AGCGGCTACCACGCCAACAAGTGCACCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCGACCTGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTNIO: SEQ ID 106
GGCCTGGAGTGCGACGGCAAGGTGAACTACTGCTGCAAGAAGCAGGAC
TTCGTGAGCTTCAAGGACATCGGCTGGAACGACTGGATCATCGCCCCC
AGCGGCTACCACGCCAACAGGTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCGCCCAGATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN11: SEQ ID 107
GGCCTGGAGTGCGACGGCAAGGTGAACCTGTGCTGCAAGAAGCAGCTG
TTCGTGAGCTTCAAGGACATCGGCTGGAACGACTGGATCATCGCCCCC
AGCGGCTACCACGCCAACCACTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCGCCCAGATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN12: SEQ ID 108
GGCCTGGAGTGCGACGGCAAGGTGAACTACTGCTGCAAGAAGCAGAAC
TTCGTGAGCTTCAAGGACATCGGCTGGAACGACTGGATCATCGCCCCC
AGCGGCTACCACGCCAACGAGTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCCAGATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN13: SEQ ID 109
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN14: SEQ ID 110
GGCCTGGAGTGCGACGGCAAGGTGAACCTGTGCTGCAAGAAGCAGCAC
TTCGTGAGCTTCAAGGACATCGGCTGGAACGACTGGATCATCGCCCCC
AGCGGCTACCACGCCAACAGGTGCGACGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCGCCCAGATGGGCAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN15: SEQ ID 111
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCAACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN16: SEQ ID 112
GGCCTGGAGTGCGACGGCAAGGTGAACCTGTGCTGCAAGAAGCAGCTG
TTCGTGAGCTTCAAGGACATCGGCTGGAACGACTGGATCATCGCCCCC
AGCGGCTACCACGCCAACCACTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCCAGATGGGCGCCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN17: SEQ ID 113
GGCCTGGAGTGCGACGGCAAGGTGAACTACTGCTGCAAGAAGCAGGAC
TTCGTGAGCTTCAAGGACATCGGCTGGAACGACTGGATCATCGCCCCC
AGCGGCTACCACGCCAACAAGTGCGGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCGCCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN18: SEQ ID 114
GGCCTGGAGTGCGACGGCAAGGTGAACTACTGCTGCAAGAAGCAGAAC
TTCGTGAGCTTCAAGGACATCGGCTGGAACGACTGGATCATCGCCCCC
AGCGGCTACCACGCCAACAAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN19: SEQ ID 115
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAAGTGCGGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN20: SEQ ID 116
GGCCTGGAGTGCGACGGCAAGGTGAACCTGTGCTGCAAGAAGCAGAAC
TTCGTGAGCTTCAAGGACATCGGCTGGAACGACTGGATCATCGCCCCC
AGCGGCTACCACGCCAACAAGTGCGGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCGCCCAGATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN21: SEQ ID 117
GGCCTGGAGTGCGACGGCAAGGTGAACTACTGCTGCAAGAAGCAGTGG
TTCGTGAGCTTCAAGGACATCGGCTGGAACGACTGGATCATCGCCCCC
AGCGGCTACCACGCCAACAAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN22: SEQ ID 118
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCGACGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCGCCCTGATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN23: SEQ ID 119
GGCCTGGAGTGCGACGGCAAGGTGAACTACTGCTGCAAGAAGCAGGAC
TTCGTGAGCTTCAAGGACATCGGCTGGAACGACTGGATCATCGCCCCC
AGCGGCTACCACGCCAACAAGTGCGACGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN24: SEQ ID 120
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCAGCGGCAGGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN25: SEQ ID 121
GGCCTGGAGTGCGACGGCAAGGTGAACCTGTGCTGCAAGAAGCAGAAC
TTCGTGAGCTTCAAGGACATCGGCTGGAACGACTGGATCATCGCCCCC
AGCGGCTACCACGCCAACGAGTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCCAGATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN26: SEQ ID 122
GGCCTGGAGTGCGACGGCAAGGTGAACCTGTGCTGCAAGAAGCAGCAC
TTCGTGAGCTTCAAGGACATCGGCTGGAACGACTGGATCATCGCCCCC
AGCGGCTACCACGCCAACAGGTGCGACGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN27: SEQ ID 123
GGCCTGGAGTGCGACGGCAAGGTGAACCTGTGCTGCAAGAAGCAGCTG
TTCGTGAGCTTCAAGGACATCGGCTGGAACGACTGGATCATCGCCCCC
AGCGGCTACCACGCCAACCACTGCGACGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACAGGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN28: SEQ ID 124
GGCCTGGAGTGCGACGGCAAGGTGAACTACTGCTGCAAGAAGCAGCTG
TTCGTGAGCTTCAAGGACATCGGCTGGAACGACTGGATCATCGCCCCC
AGCGGCTACCACGCCAACCACTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN29: SEQ ID 125
GGCCTGGAGTGCGACGGCAAGGTGAACCTGTGCTGCAAGAAGCAGCTG
TTCGTGAGCTTCAAGGACATCGGCTGGAACGACTGGATCATCGCCCCC
AGCGGCTACCACGCCAACCACTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN30: SEQ ID 126
GGCCTGGAGTGCGACGGCAAGGTGAACTACTGCTGCAAGAAGCAGAAC
TTCGTGAGCTTCAAGGACATCGGCTGGAACGACTGGATCATCGCCCCC
AGCGGCTACCACGCCAACAAGTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCAAGATGGGCGCCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN31: SEQ ID 127
GGCCTGGAGTGCGACGGCAAGGTGAACACCTGCTGCAAGAAGCAGCTG
TTCGTGAGCTTCAAGGACATCGGCTGGAACGACTGGATCATCGCCCCC
AGCGGCTACCACGCCAACCACTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN32: SEQ ID 128
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCGGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCAACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN33: SEQ ID 129
GGCCTGGAGTGCGACGGCAAGGTGAACCTGTGCTGCAAGAAGCAGAAC
TTCGTGAGCTTCAAGGACATCGGCTGGAACGACTGGATCATCGCCCCC
AGCGGCTACCACGCCAACGAGTGCATGGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN34: SEQ ID 130
GGCCTGGAGTGCGACGGCAAGGTGAACTACTGCTGCAAGAAGCAGCTG
TTCGTGAGCTTCAAGGACATCGGCTGGAACGACTGGATCATCGCCCCC
AGCGGCTACCACGCCAACCACTGCACCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACCTGGGCAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN35: SEQ ID 131
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN36: SEQ ID 132
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACATGGGCGCCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN37: SEQ ID 133
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN38: SEQ ID 134
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAAGTGCGGCGGCAAGTGCCCCAGCCACATC
CCGGCACCAGCGGCAGCAGCCTGAGCTTCCACAGCACCGTGATCAACC
ACTACAGGATGAGGGGCCACAGCCCCTTCAGCCAGCTGGGCGCCTGCT
GCGTGCCCACCAAGCTGAGGCCCATGAGCATGCTGTACTACGACGACG
GCCAGAACATCATCAAGAAGGACATCCAGAACATGATCGTGGAGGAGT
GCGGCTGCAGCTAA
>ACTN39: SEQ ID 135
GGCCTGGAGTGCGACGGCAAGGTGAACCTGTGCTGCAAGAAGCAGAAC
TTCGTGAGCTTCAAGGACATCGGCTGGAACGACTGGATCATCGCCCCC
AGCGGCTACCACGCCAACGAGTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN40: SEQ ID 136
GGCCTGGAGTGCGACGGCAAGGTGAACCTGTGCTGCAAGAAGCAGCTG
TTCGTGAGCTTCAAGGACATCGGCTGGAACGACTGGATCATCGCCCCC
AGCGGCTACCACGCCAACCACTGCGCCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCAACATGGGCAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN41: SEQ ID 137
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGCTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACAGGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN42: SEQ ID 138
GGCCTGGAGTGCGACGGCAAGGTGAACTACTGCTGCAAGAAGCAGGAC
TTCGTGAGCTTCAAGGACATCGGCTGGAACGACTGGATCATCGCCCCC
AGCGGCTACCACGCCAACAAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN43: SEQ ID 139
GGCCTGGAGTGCGACGGCAAGGTGAACTACTGCTGCAAGAAGCAGGAC
TTCGTGAGCTTCAAGGACATCGGCTGGAACGACTGGATCATCGCCCCC
AGCGGCTACCACGCCAACAGGTGCGACGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCGCCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN44: SEQ ID 140
GGCCTGGAGTGCGACGGCAAGGTGAACCTGTGCTGCAAGAAGCAGAAC
TTCGTGAGCTTCAAGGACATCGGCTGGAACGACTGGATCATCGCCCCC
AGCGGCTACCACGCCAACGAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN45: SEQ ID 141
GGCCTGGAGTGCGACGGCAAGGTGAACACCTGCTGCAAGAAGCAGAAC
TTCGTGAGCTTCAAGGACATCGGCTGGAACGACTGGATCATCGCCCCC
AGCGGCTACCACGCCAACGAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN46: SEQ ID 142
GGCCTGGAGTGCGACGGCAAGGTGAACCTGTGCTGCAAGAAGCAGAAC
TTCGTGAGCTTCAAGGACATCGGCTGGAACGACTGGATCATCGCCCCC
GCGGCTACCACGCCAACGAGTGCGGCGGCCTGTGCCCCAGCCACATCG
CCGGCACCAGCGGCAGCAGCCTGAGCTTCCACAGCACCGTGATCAACC
ACTACAGGATGAGGGGCCACAGCCCCCACGCCAACAGGGGCGCCTGCT
GCATCCCCACCAAGCTGAGGCCCATGAGCATGCTGTACTACGACGACG
GCCAGAACATCATCAAGAAGGACATCCAGAACATGATCGTGGAGGAGT
GCGGCTGCAGCTAA
>ACTN47: SEQ ID 143
GGCCTGGAGTGCGACGGCAAGGTGAACTACTGCTGCAAGAAGCAGAAC
TTCGTGAGCTTCAAGGACATCGGCTGGAACGACTGGATCATCGCCCCC
AGCGGCTACCACGCCAACGAGTGCAGCGGCAAGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN48: SEQ ID 144
GGCCTGGAGTGCGACGGCAAGGTGAACCTGTGCTGCAAGAAGCAGAAC
TTCGTGAGCTTCAAGGACATCGGCTGGAACGACTGGATCATCGCCCCC
AGCGGCTACCACGCCAACGAGTGCAGCGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACATGGGCGCCTGC
TGCATCCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN49: SEQ ID 145
GGCCTGGAGTGCGACGGCAAGGTGAACCTGTGCTGCAAGAAGCAGGAC
TTCGTGAGCTTCAAGGACATCGGCTGGAACGACTGGATCATCGCCCCC
AGCGGCTACCACGCCAACAGGTGCGACGGCCTGTGCCCCAGCCACATC
GCCGGCACCAGCGGCAGCAGCCTGAGCTTCCACAGCACCGTGATCAAC
CACTACAGGATGAGGGGCCACAGCCCCTTCAGCGACATGGGCAGCTGC
TGCGTGCCCACCAAGCTGAGGCCCATGAGCATGCTGTACTACGACGAC
GGCCAGAACATCATCAAGAAGGACATCCAGAACATGATCGTGGAGGAG
TGCGGCTGCAGCTAA
>ACTN50: SEQ ID 146
GGCCTGGAGTGCGACGGCAAGGTGAACATCTGCTGCAAGAAGCAGCTG
TTCGGCAGGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCGTGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN51: SEQ ID 147
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGAGGATGGTGGTGGAGGAG
TGCGGCTGCACCTAA
>ACTN52: SEQ ID 148
GGCCTGGAGTGCGACGGCAAGGTGAACATCTGCTGCAAGAAGCAGCTG
TTCGGCAAGACCAAGGACATCGGCTGGAACGACTGGATCATCGCCCCC
AGCGGCTACCACGGCGGCAGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCAACGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGAAGGTGGAGGAG
TGCGGCTGCACCTAA
>ACTN53: SEQ ID 149
GGCCTGGAGTGCGACGGCAAGGTGAACATCTGCTGCAAGAAGCAGGAG
TTCGGCCAGGCCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGAAGGTGGAGGAG
TGCGGCTGCACCTAA
>ACTN54: SEQ ID 150
GGCCTGGAGTGCGACGGCAAGGTGAACATCTGCTGCAAGAAGCAGAGC
TTCGCCCAGACCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN55: SEQ ID 151
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGGTGGTGGAGGAG
TGCGGCTGCACCTAA
>ACTN56: SEQ ID 152
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCAGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCGCCCCCACCAAGCTGAGGCCCATGAGCATGCTGTACTACGACGAC
GGCCAGAACATCATCAAGAAGGACATCCAGAACATGGTGGTGGAGGAG
TGCGGCTGCGTGTAA
>ACTN57: SEQ ID 153
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN58: SEQ ID 154
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN59: SEQ ID 155
GGCCTGGAGTGCGACGGCAAGGTGAACATCTGCTGCAAGAAGCAGCTG
TTCGGCCAGACCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCAACGCCAACCTGAAGAGCTGC
TGCGCCCCCACCAAGCTGAGGCCCATGAGCATGCTGTACTACGACGAC
GGCCAGAACATCATCAAGAAGGACATCCAGGGCATGAAGGTGGAGGAG
TGCGGCTGCGTGTAA
>ACTN60: SEQ ID 156
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCGCCCCCACCAAGCTGAGGCCCATGAGCATGCTGTACTACGACGAC
GGCCAGAACATCATCAAGAAGGACATCCAGAACATGGTGGTGGAGGAG
TGCGGCTGCGTGTAA
>ACTN61: SEQ ID 157
GGCCTGGAGTGCGACGGCAAGGTGAACATCTGCTGCAAGAAGCAGCTG
TTCGGCCAGACCAAGGACATCGGCTGGAACGACTGGATCATCGCCCCC
AGCGGCTACCACGGCGGCAGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCAACGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN62: SEQ ID 158
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN63: SEQ ID 159
GGCCTGGAGTGCGACGGCAAGGTGAACATCTGCTGCAAGAAGCAGAGC
TTCAGCCAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGGTGGTGGAGGAG
TGCGGCTGCACCTAA
>ACTN64: SEQ ID 160
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGGTGGTGGAGGAG
TGCGGCTGCACCTAA
>ACTN65: SEQ ID 161
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCAGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGAAGGTGGAGGAG
TGCGGCTGCACCTAA
>ACTN66: SEQ ID 162
GGCCTGGAGTGCGACGGCAAGGTGAACATCTGCTGCAAGAAGCAGATG
TTCGGCCAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN67: SEQ ID 163
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN68: SEQ ID 164
GGCCTGGAGTGCGACGGCAAGGTGAACATCTGCTGCAAGAAGCAGAGC
TTCGGCAAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGAAGGTGGAGGAG
GCGGCTGCACCTAA
>ACTN69: SEQ ID 165
GGCCTGGAGTGCGACGGCAAGGTGAACATCTGCTGCAAGAAGCAGAGC
TTCGGCAAGACCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGCAGATGGTGGTGGAGGAG
TGCGGCTGCACCTAA
>ACTN70: SEQ ID 166
GGCCTGGAGTGCGACGGCAAGGTGAACATCTGCTGCAAGAAGCAGCTG
TTCGGCCAGGCCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCGTGGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGGTGGTGGAGGAG
TGCGGCTGCACCTAA
>ACTN71: SEQ ID 167
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN72: SEQ ID 168
GGCCTGGAGTGCGACGGCAAGGTGAACATCTGCTGCAAGAAGCAGCTG
TTCGGCCAGGCCAAGGACATCGGCTGGAACGACTGGATCATCGCCCCC
AGCGGCTACCACGGCGGCAGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCAACGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN73: SEQ ID 169
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGGTGGTGGAGGAG
TGCGGCTGCACCTAA
>ACTN74: SEQ ID 170
GGCCTGGAGTGCGACGGCAAGGTGAACATCTGCTGCAAGAAGCAGAGC
TTCGGCAGGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGGTGGTGGAGGAG
TGCGGCTGCACCTAA
>ACTN75: SEQ ID 171
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAGGATGAAGGTGGAGGAG
TGCGGCTGCACCTAA
>ACTN76: SEQ ID 172
GGCCTGGAGTGCGACGGCAAGGTGAACATCTGCTGCAAGAAGCAGCTG
TTCGGCCAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCAACGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGAAGGTGGAGGAG
TGCGGCTGCACCTAA
>ACTN77: SEQ ID 173
GGCCTGGAGTGCGACGGCAAGGTGAACATCTGCTGCAAGAAGCAGATG
TTCGGCAAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN78: SEQ ID 174
GGCCTGGAGTGCGACGGCAAGGTGAACATCTGCTGCAAGAAGCAGCTG
TTCGGCCAGGCCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCGTGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN79: SEQ ID 175
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAGGATGGTGGTGGAGGAG
TGCGGCTGCACCTAA
>ACTN80: SED ID 176
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCGCCCCCACCAAGCTGAGGCCCATGAGCATGCTGTACTACGACGAC
GGCCAGAACATCATCAAGAAGGACATCCAGAACATGAAGGTGGAGGAG
TGCGGCTGCGTGTAA
>ACTN8I: SEQ ID 177
GGCCTGGAGTGCGACGGCAAGGTGAACATCTGCTGCAAGAAGCAGCTG
TTCGGCAAGACCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCGTGGCCAACCTGAAGAGCTGC
TGCAGCCCCACCAAGCTGAGGCCCATGAGCATGCTGTACTACGACGAC
GGCCAGAACATCATCAAGAAGGACATCCAGAGGATGGTGGTGGAGGAG
TGCGGCTGCACCTAA
>ACTN82: SEQ ID 178
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGAAGGTGGAGGAG
TGCGGCTGCACCTAA
>ACTN83: SEQ ID 179
GGCCTGGAGTGCGACGGCAAGGTGAACATCTGCTGCAAGAAGCAGAGC
TTCGGCAGGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGAAGGTGGAGGAG
TGCGGCTGCACCTAA
>ACTN84: SEQ ID 180
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN85: SEQ ID 181
GGCCTGGAGTGCGACGGCAAGGTGAACATCTGCTGCAAGAAGCAGCTG
TTCGGCCAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCGTGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN86: SEQ ID 182
GGCCTGGAGTGCGACGGCAAGGTGAACATCTGCTGCAAGAAGCAGAGC
TTCGGCAAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN87: SEQ ID 183
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCAGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN88: SEQ ID 184
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN89: SEQ ID 185
GGCCTGGAGTGCGACGGCAAGGTGAACATCTGCTGCAAGAAGCAGCTG
TTCGGCCAGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCAACGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN90: SEQ ID 186
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCAGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN91: SEQ ID 187
GGCCTGGAGTGCGACGGCAAGGTGAACATCTGCTGCAAGAAGCAGAGC
TTCGGCAGGACCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGGTGGTGGAGGAG
TGCGGCTGCACCTAA
>ACTN92: SEQ ID 188
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCAGCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGGGCATGAAGGTGGAGGAG
TGCGGCTGCACCTAA
>ACTN93: SEQ ID 189
GGCCTGGAGTGCGACGGCAAGGTGAACATCTGCTGCAAGAAGCAGAGC
TTCGGCCAGACCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTTCGCCAACCTGAAGAGCTGC
TGCAGCCCCACCAAGCTGAGGCCCATGAGCATGCTGTACTACGACGAC
GGCCAGAACATCATCAAGAAGGACATCCAGAACATGAAGGTGGAGGAG
TGCGGCTGCACCTAA
>ACTN94
GGCCTGGAGTGCGACGGCAAGGTGAACATCTGCTGCAAGAAGCAGAGC
TTCGGCCAGGCCAAGGACATCGGCTGGAACGACTGGATCATCGCCCCC
AGCGGCTACCACGGCGGCGGCTGCACCGGCGAGTGCCCCAGCCACATC
GCCGGCACCAGCGGCAGCAGCCTGAGCTTCCACAGCACCGTGATCAAC
CACTACAGGATGAGGGGCCACAGCCCCTGGGCCAACCTGAAGAGCTGC
TGCAGCCCCACCAAGCTGAGGCCCATGAGCATGCTGTACTACGACGAC
GGCCAGAACATCATCAAGAAGGACATCCAGAGGATGGTGGCCGAGGAG
TGCGGCTGCACCTAA

TABLE 3
Amino acid sequences of the peptides of the
present invention containing histidine substitutes
active Activin A variants and Bis-His variants
(single, double and triple, as of May 15, 2008)

| position | parent | 3 | 5 | 7 | 9 | 10 | 13 | 14 | 15 | 16 | 17 | 18 | 19 | 20 | 37 | 38 | 39 | 41 | 43 | 75 | 76 | 77 | 78 | 79 | 82 | 107 | 109 | 116 | |
|---|---|---|---|---|---|---|---|---|---|---|---|---|---|---|---|---|---|---|---|---|---|---|---|---|---|---|---|---|---|
| ACTN1 | | E | D | K | N | I | K | K | Q | F | F | V | S | F | A | N | Y | E | E | A | N | L | K | S | V | N | I | S | |
| (wt) | | | | | | | | | | | | | | | | | | | | | | | | | | | | | |
| ACTN4 | | — | — | — | — | L | — | — | — | L | — | — | — | — | — | — | H | S | L | S | D | M | G | A | — | — | — | — | — |
| ACTN11 | | — | — | — | — | L | — | — | — | L | — | — | — | — | — | — | H | S | L | — | Q | M | G | A | I | — | — | — | — |
| ACTN12 | | — | — | — | — | Y | — | — | — | N | — | — | — | — | — | — | E | S | L | S | Q | M | G | — | I | — | — | — | — |
| ACTN16 | | — | — | — | — | L | — | — | — | L | — | — | — | — | — | — | H | S | L | S | Q | M | G | — | I | — | — | — | — |
| ACTN27 | | — | — | — | — | L | — | — | — | L | — | — | — | — | — | — | H | D | K | — | — | R | G | A | I | — | — | — | — |
| ACTN28 | | — | — | — | — | Y | — | — | — | L | — | — | — | — | — | — | H | S | K | — | — | M | G | — | I | — | — | — | — |
| ACTN29 | | — | — | — | — | L | — | — | — | L | — | — | — | — | — | — | H | S | K | S | D | M | G | — | I | — | — | — | — |
| ACTN31 | | — | — | — | — | T | — | — | — | L | — | — | — | — | — | — | H | S | K | S | D | M | G | — | I | — | — | — | — |
| ACTN34 | | — | — | — | — | Y | — | — | — | L | — | — | — | — | — | — | H | T | K | S | D | | G | — | — | — | — | — | — |
| ACTN47 | | — | — | — | — | Y | — | — | — | N | — | — | — | — | — | — | E | S | K | S | D | M | G | — | I | — | — | — | — |
| ACTN48 | | — | — | — | — | L | — | — | — | N | — | — | — | — | — | — | E | S | L | — | — | M | G | A | I | — | — | — | — |
| ACTN40 | | — | — | — | — | L | — | — | — | L | — | — | — | — | — | — | H | A | L | S | M | G | — | — | — | — | — | — | — |
| ACTN56 | | — | — | — | — | — | — | — | — | S | — | G | Q | A | G | G | S | T | — | — | — | — | — | — | A | — | V | V | — |
| ACTN65 | | — | — | — | — | — | — | — | — | S | — | G | Q | A | G | G | S | T | — | — | — | — | — | — | S | G | K | T | — |
| ACTN69 | | — | — | — | — | — | — | — | — | S | — | G | K | T | G | G | G | T | — | — | — | — | — | — | S | Q | V | T | — |
| ACTD2 | N1 | H | H | — | — | — | — | — | — | — | — | — | — | — | — | — | — | — | — | — | — | — | — | — | — | — | — | — | — |
| ACTD3 | N1 | — | — | H | H | — | — | — | — | — | — | — | — | — | — | — | — | — | — | — | — | — | — | — | — | — | — | — | — |
| ACTD4 | N1 | — | — | — | — | — | H | | H | — | — | — | — | — | — | — | — | — | — | — | — | — | — | — | — | — | — | — | — |
| ACTD5 | N1 | — | — | — | — | — | — | H | H | — | — | — | — | — | — | — | — | — | — | — | — | — | — | — | — | — | — | — | — |
| ACTD6 | N1 | — | — | — | — | — | — | — | H | — | H | — | — | — | — | — | — | — | — | — | — | — | — | — | — | — | — | — | — |
| ACTD7 | N16 | H | H | — | — | L | — | — | — | L | — | — | — | — | — | — | H | S | L | S | Q | M | G | A | — | — | — | — | — |
| ACTD8 | N16 | — | — | H | H | L | — | — | — | L | — | — | — | — | — | — | H | S | L | S | Q | M | G | A | — | — | — | — | — |
| ACTD9 | N16 | — | — | — | — | L | H | — | H | L | — | — | — | — | — | — | H | S | L | S | Q | M | G | A | — | — | — | — | — |
| ACTD10 | N16 | — | — | — | — | L | — | H | H | L | — | — | — | — | — | — | H | S | L | S | Q | M | G | A | — | — | — | — | — |
| ACTD11 | N16 | — | — | — | — | L | — | — | H | L | H | — | — | — | — | | H | S | L | S | Q | M | G | A | — | — | — | — | — |
| ACTD12 | N34 | H | H | — | — | Y | — | — | — | L | — | — | — | — | — | — | H | T | K | S | D | — | G | — | — | — | — | — | — |
| ACTD13 | N34 | — | — | H | H | Y | — | — | — | L | — | — | — | — | — | — | H | T | K | S | D | — | G | — | — | — | — | — | — |
| ACTD14 | N34 | — | — | — | — | Y | H | — | H | L | — | — | — | — | — | — | H | T | K | S | D | — | G | — | — | — | — | — | — |
| ACTD15 | N34 | — | — | — | — | Y | — | H | H | L | — | — | — | — | — | — | H | T | K | S | D | — | G | — | — | — | — | — | — |
| ACTD16 | N34 | — | — | — | — | Y | — | — | H | L | H | — | — | — | — | H | T | K | S | D | — | G | — | — | — | — | — | — | — |
| ACTD17 | D3, | H | H | H | H | — | — | — | — | — | — | — | — | — | — | — | — | — | — | — | — | — | — | — | — | — | — | — | — |
| | N1 | | | | | | | | | | | | | | | | | | | | | | | | | | | | |
| ACTD18 | D3, | — | — | H | H | — | H | — | H | — | — | — | — | — | — | — | — | — | — | — | — | — | — | — | — | — | — | — | — |
| | N1 | | | | | | | | | | | | | | | | | | | | | | | | | | | | |
| ACTD19 | D8, | H | H | H | H | L | — | — | — | L | — | — | — | — | — | — | H | S | L | S | Q | M | G | A | — | — | — | — | — |
| | N16 | | | | | | | | | | | | | | | | | | | | | | | | | | | | |
| ACTD20 | D8, | — | — | H | H | L | H | — | H | L | — | — | — | — | — | — | H | S | L | S | Q | M | G | A | — | — | — | — | — |
| | N16 | | | | | | | | | | | | | | | | | | | | | | | | | | | | |
| ACTD21 | D13, | H | H | H | H | Y | — | — | — | L | — | — | — | — | — | — | H | T | K | S | D | — | G | — | — | — | — | — | — |
| | N34 | | | | | | | | | | | | | | | | | | | | | | | | | | | | |
| ACTD22 | D13, | — | — | H | H | Y | H | — | H | L | — | — | — | — | — | — | H | T | K | S | D | — | G | — | — | — | — | — | — |
| | N34 | | | | | | | | | | | | | | | | | | | | | | | | | | | | |
| ACTD23 | D21, | H | H | H | H | Y | H | — | H | L | — | — | — | — | — | — | H | T | K | S | D | — | G | — | — | — | — | — | — |
| | N34 | | | | | | | | | | | | | | | | | | | | | | | | | | | | |
| Consensus | | E | D | | N | L | K | K | Q | L | F | V | S | F | A | N | H | S | | S | N | L | G | S | V | N | I | S | |

TABLE 4
Amino acid sequences of the peptides of the present
invention containing histidine substitutes

| | | | | E3H/D5H + |
|---|---|---|---|---|
| Parent | | E3H/D5H + | K7H/N9H + | K7H/N9H + |
| construct | K7H/N9H | K7H/N9H | K13H/Q15H | K13H/Q15H |
| ACTN1 | ACTD3 | ACTD17 | ACTD18 | |
| ACTN16 | ACTD8 | ACTD19 | ACTD20 | |
| ACTN34 | ACTD13 | ACTD21 | ACTD22 | ACTD23 |

TABLE 5
Description of the follistatin variants used in the present invention

| Peptide ID | Description | Construct ID |
|---|---|---|
| ACTA1 | Follistatin FS315 with His tag and GS linker | pDR000001870 |
| | in pUnder | |
| ACTA2 | Follistatin FS288 with His tag and GS linker | pDR000001871 |
| | in pUnder | |
| ACTA3 | Follistatin FS12 with His tag and GS linker | pDR000001872 |
| | in pUnder | |

TABLE 6
Amino acid sequence of the follistatin variants
used in the present invention

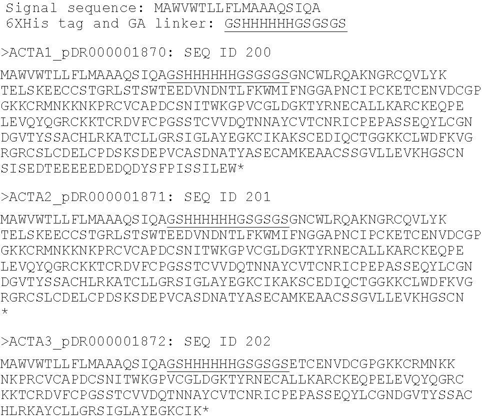

TABLE 7
Nucleic acid sequence of the follistatin variants used in the present invention

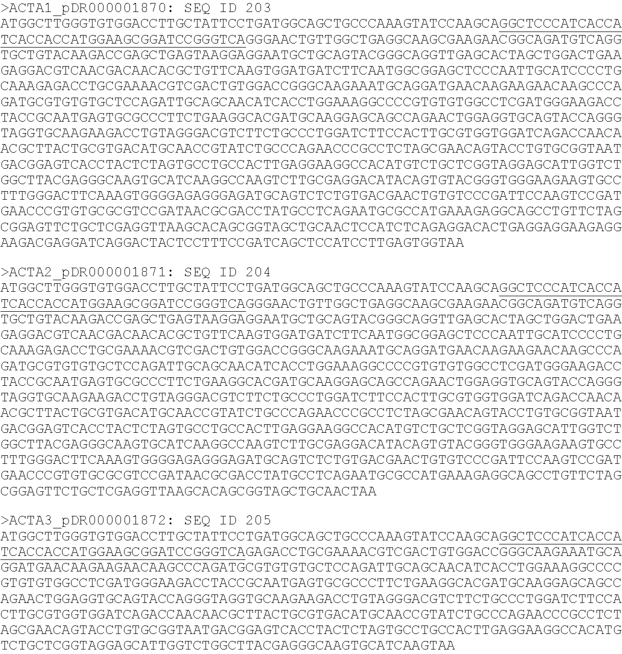

TABLE 8
Primary Screening data: Effect of the peptides of the present
invention on differentiation of pluripotent stem cells
TABLE 8

| | | Supernatant | Assay | Cell Number | | Sox 17 Intensity | | |
|---|---|---|---|---|---|---|---|---|
| Assay Plate | Sample | concentration | Dilution | average | S.D. | average | S.D. | % of control |
| #001 | no Activin A | NA | NA | 548 | 735 | 3.270E+06 | 4.619E+06 | 1.8 |
| #001 | R&D Sys Activin A | 6.25 ng/ml | | 800 | 446 | 5.623E+06 | 3.858E+06 | |
| #001 | R&D Sys Activin A | 12.5 ng/ml | | 1663 | 699 | 1.224E+07 | 5.919E+06 | |
| #001 | R&D Sys Activin A | 25 ng/ml | | 2336 | 450 | 2.168E+07 | 3.686E+06 | |
| #001 | R&D Sys Activin A | 50 ng/ml | | 3685 | 1740 | 4.316E+07 | 8.037E+06 | |
| #001 | R&D Sys Activin A | 100 ng/ml | | 5878 | 2617 | 1.839E+08 | 3.377E+07 | 100 |
| #001 | R&D Sys Activin A | 200 ng/ml | | 8767 | 984 | 3.847E+08 | 4.955E+07 | |
| #001 | R&D Sys Activin A | 400 ng/ml | | 7391 | 1950 | 3.627E+08 | 8.693E+07 | |
| #001 | mock SN | 1x | 1:20 | 390 | 236 | 4.642E+06 | 4.732E+06 | 2.5 |
| #001 | OriGENE WT | 1x | 1:20 | 979 | 133 | 1.819E+07 | 1.010E+07 | 9.9 |
| #001 | OriGENE WT | 10x | 1:20 | 5548 | 1348 | 2.035E+08 | 5.765E+07 | 110.6 |
| #001 | ACTN1 | 1x | 1:20 | 5466 | 1393 | 1.519E+08 | 2.986E+07 | 82.6 |
| #001 | ACTN1 | 10x | 1:20 | 9254 | 3336 | 4.640E+08 | 1.635E+08 | 252.3 |
| #001 | ACTN2 | 1x | 1:20 | 4057 | 3624 | 1.756E+07 | 9.803E+06 | 9.5 |
| #001 | ACTN2 | 10x | 1:20 | 2965 | 484 | 2.299E+07 | 3.753E+06 | 12.5 |
| #001 | ACTN2 | 10x | 1:40 | 2232 | 420 | 1.280E+07 | 7.767E+06 | 7.0 |
| #001 | ACTN4 | 1x | 1:20 | 6380 | 1421 | 9.099E+07 | 1.591E+07 | 49.5 |
| #001 | ACTN4 | 10x | 1:20 | 8916 | 1861 | 2.385E+08 | 1.098E+08 | 129.7 |
| #001 | ACTN4 | 10x | 1:40 | 6548 | 1606 | 2.075E+08 | 4.111E+07 | 112.8 |
| #001 | ACTN5 | 1x | 1:20 | 1261 | 506 | 1.111E+07 | 6.119E+06 | 6.0 |
| #001 | ACTN5 | 10x | 1:20 | 1396 | 875 | 1.031E+07 | 7.316E+06 | 5.6 |
| #001 | ACTN5 | 10x | 1:40 | 1382 | 924 | 1.311E+07 | 5.980E+06 | 7.1 |
| #001 | ACTN6 | 1x | 1:20 | 939 | 605 | 1.234E+07 | 9.445E+06 | 6.7 |
| #001 | ACTN6 | 10x | 1:20 | 2359 | 454 | 2.272E+07 | 3.667E+06 | 12.4 |
| #001 | ACTN6 | 10x | 1:40 | 1790 | 1521 | 1.426E+07 | 4.185E+06 | 7.8 |
| #001 | ACTN7 | 1x | 1:20 | 1133 | 381 | 1.108E+07 | 1.755E+06 | 6.0 |
| #001 | ACTN7 | 10x | 1:20 | 2714 | 1393 | 1.904E+07 | 1.900E+06 | 10.4 |
| #001 | ACTN7 | 10x | 1:40 | 1387 | 1264 | 1.099E+07 | 7.438E+06 | 6.0 |
| #001 | ACTN8 | 1x | 1:20 | 363 | 194 | 5.578E+06 | 3.202E+06 | 3.0 |
| #001 | ACTN8 | 10x | 1:20 | 1419 | 320 | 1.181E+07 | 4.791E+06 | 6.4 |
| #001 | ACTN8 | 10x | 1:40 | 372 | 333 | 3.869E+06 | 3.552E+06 | 2.1 |
| #002 | no Activin A | NA | NA | 5131 | 350 | 6.83E+05 | 5.37E+05 | 2.6 |
| #002 | R&D Sys Activin A | 6.25 ng/ml | | 5358 | 1329 | 7.13E+05 | 4.26E+05 | |
| #002 | R&D Sys Activin A | 12.5 ng/ml | | 4579 | 1767 | 2.23E+06 | 9.56E+05 | |
| #002 | R&D Sys Activin A | 25 ng/ml | | 5265 | 1846 | 4.99E+06 | 1.00E+06 | |
| #002 | R&D Sys Activin A | 50 ng/ml | | 5306 | 785 | 1.02E+07 | 3.99E+06 | |
| #002 | R&D Sys Activin A | 100 ng/ml | | 7828 | 2102 | 2.59E+07 | 4.05E+06 | 100 |
| #002 | R&D Sys Activin A | 200 ng/ml | | 11285 | 3031 | 1.32E+08 | 1.28E+07 | |
| #002 | R&D Sys Activin A | 400 ng/ml | | 10428 | 3534 | 1.56E+08 | 3.08E+07 | |
| #002 | ACTN9 | 1x | 1:20 | 11391 | 2104 | 5.97E+05 | 5.90E+05 | 2.3 |
| #002 | ACTN9 | 10x | 1:20 | 11456 | 4148 | 6.63E+05 | 1.56E+05 | 2.6 |
| #002 | ACTN9 | 10x | 1:40 | 9608 | 1249 | 4.19E+05 | 4.91E+05 | 1.6 |
| #002 | ACTN10 | 1x | 1:20 | 7417 | 1967 | 7.52E+05 | 3.65E+05 | 2.9 |
| #002 | ACTN10 | 10x | 1:20 | 8942 | 522 | 1.08E+06 | 2.35E+05 | 4.2 |
| #002 | ACTN10 | 10x | 1:40 | 7333 | 1509 | 5.59E+05 | 5.48E+05 | 2.2 |
| #002 | ACTN11 | 1x | 1:20 | 5239 | 602 | 3.47E+06 | 7.40E+05 | 13.4 |
| #002 | ACTN11 | 10x | 1:20 | 10321 | 2388 | 4.06E+07 | 8.30E+06 | 156.8 |
| #002 | ACTN11 | 10x | 1:40 | 9493 | 60 | 2.79E+07 | 1.25E+07 | 107.8 |
| #002 | ACTN12 | 1x | 1:20 | 5420 | 2207 | 1.10E+06 | 9.64E+05 | 4.3 |
| #002 | ACTN12 | 10x | 1:20 | 6633 | 666 | 7.65E+06 | 3.54E+06 | 29.6 |
| #002 | ACTN12 | 10x | 1:40 | 6317 | 842 | 2.43E+06 | 1.27E+06 | 9.4 |
| #002 | ACTN14 | 1x | 1:20 | 4968 | 1581 | 1.50E+06 | 1.00E+05 | 5.8 |
| #002 | ACTN14 | 10x | 1:20 | 6278 | 1556 | 3.66E+06 | 2.47E+06 | 14.1 |
| #002 | ACTN14 | 10x | 1:40 | 5584 | 744 | 4.73E+06 | 2.64E+05 | 18.3 |
| #002 | ACTN16 | 1x | 1:20 | 7068 | 1332 | 1.36E+07 | 7.31E+06 | 52.7 |
| #002 | ACTN16 | 10x | 1:20 | 11118 | 1179 | 5.55E+07 | 1.12E+07 | 214.4 |
| #002 | ACTN16 | 10x | 1:40 | 11064 | 1156 | 6.46E+07 | 1.64E+06 | 249.3 |
| #002 | ACTN17 | 1x | 1:20 | 10154 | 2103 | 1.21E+06 | 5.86E+05 | 4.7 |
| #002 | ACTN17 | 10x | 1:20 | 12596 | 2314 | 2.83E+05 | 7.00E+04 | 1.1 |
| #002 | ACTN17 | 10x | 1:40 | 10807 | 2683 | 4.38E+05 | 4.42E+05 | 1.7 |
| #002 | ACTN18 | 1x | 1:20 | 6078 | 2117 | 5.68E+05 | 4.47E+05 | 2.2 |
| #002 | ACTN18 | 10x | 1:20 | 9676 | 1357 | 1.22E+06 | 8.99E+05 | 4.7 |
| #002 | ACTN18 | 10x | 1:40 | 11683 | 3408 | 2.22E+05 | 1.75E+05 | 0.9 |
| #003 | no Activin A | NA | NA | 10933 | 4289 | 1.97E+04 | 1.84E+04 | 0.1 |
| #003 | R&D Sys Activin A | 6.25 ng/ml | | 5816 | 227 | 8.03E+05 | 2.07E+05 | |
| #003 | R&D Sys Activin A | 12.5 ng/ml | | 5927 | 844 | 9.09E+05 | 6.48E+05 | |
| #003 | R&D Sys Activin A | 25 ng/ml | | 7235 | 768 | 4.89E+06 | 1.55E+06 | |
| #003 | R&D Sys Activin A | 50 ng/ml | | 7841 | 821 | 8.35E+06 | 5.28E+05 | |
| #003 | R&D Sys Activin A | 100 ng/ml | | 10034 | 603 | 2.94E+07 | 4.46E+06 | 100 |
| #003 | R&D Sys Activin A | 200 ng/ml | | 12425 | 2392 | 1.50E+08 | 3.44E+07 | |
| #003 | R&D Sys Activin A | 400 ng/ml | | 15451 | 2559 | 2.09E+08 | 2.46E+07 | |
| #003 | ACTN19 | 1x | 1:20 | 11017 | 4164 | 4.09E+04 | 7.08E+04 | 0.1 |
| #003 | ACTN19 | 10x | 1:20 | 11587 | 7847 | 3.96E+04 | 6.86E+04 | 0.1 |
| #003 | ACTN19 | 10x | 1:40 | 12549 | 1654 | 4.86E+03 | 8.41E+03 | 0.0 |
| #003 | ACTN20 | 1x | 1:20 | 9000 | 3265 | 1.26E+04 | 1.09E+04 | 0.0 |
| #003 | ACTN20 | 10x | 1:20 | 10217 | 1604 | 1.52E+05 | 1.43E+05 | 0.5 |
| #003 | ACTN20 | 10x | 1:40 | 12284 | 5364 | 9.03E+03 | 1.56E+04 | 0.0 |
| #003 | ACTN21 | 1x | 1:20 | 8072 | 1928 | 6.33E+03 | 1.10E+04 | 0.0 |
| #003 | ACTN21 | 10x | 1:20 | 11102 | 4407 | 4.89E+05 | 7.86E+05 | 1.7 |
| #003 | ACTN21 | 10x | 1:40 | 10458 | 2550 | 8.77E+04 | 8.56E+04 | 0.3 |
| #003 | ACTN22 | 1x | 1:20 | 9909 | 2201 | 1.32E+05 | 1.88E+05 | 0.4 |
| #003 | ACTN22 | 10x | 1:20 | 8745 | 2985 | 3.69E+05 | 1.94E+05 | 1.3 |
| #003 | ACTN22 | 10x | 1:40 | 9568 | 2146 | 3.76E+05 | 1.62E+05 | 1.3 |
| #003 | ACTN23 | 1x | 1:20 | 6831 | 2235 | 2.89E+04 | 3.37E+04 | 0.1 |
| #003 | ACTN23 | 10x | 1:20 | 10482 | 1338 | 1.63E+05 | 1.83E+03 | 0.6 |
| #003 | ACTN23 | 10x | 1:40 | 8184 | 1000 | 1.47E+05 | 1.77E+05 | 0.5 |
| #003 | ACTN28 | 1x | 1:20 | 7411 | 753 | 3.70E+06 | 1.98E+06 | 12.6 |
| #003 | ACTN28 | 10x | 1:20 | 12587 | 194 | 4.87E+07 | 1.09E+07 | 165.7 |
| #003 | ACTN28 | 10x | 1:40 | 10116 | 613 | 3.57E+07 | 4.81E+06 | 121.5 |
| #003 | ACTN32 | 1x | 1:20 | 16166 | 1771 | 9.11E+04 | 7.58E+04 | 0.3 |
| #003 | ACTN32 | 10x | 1:20 | 14330 | 3723 | 3.97E+04 | 3.40E+04 | 0.1 |
| #003 | ACTN32 | 10x | 1:40 | 11619 | 2679 | 3.43E+05 | 4.41E+05 | 1.2 |
| #003 | ACTN35 | 1x | 1:20 | 8553 | 3509 | 7.94E+04 | 5.11E+04 | 0.3 |
| #003 | ACTN35 | 10x | 1:20 | 6805 | 877 | 1.26E+06 | 6.10E+05 | 4.3 |
| #003 | ACTN35 | 10x | 1:40 | 7926 | 807 | 6.76E+05 | 3.97E+05 | 2.3 |
| #004 | no Activin A | NA | NA | 2542 | 884 | 0.00E+00 | 0.00E+00 | 0.0 |
| #004 | R&D Sys Activin A | 6.25 ng/ml | | 815 | 456 | 5.68E+04 | 4.73E+04 | |
| #004 | R&D Sys Activin A | 12.5 ng/ml | | 553 | 61 | 3.42E+05 | 3.34E+05 | |
| #004 | R&D Sys Activin A | 25 ng/ml | | 823 | 335 | 9.58E+05 | 2.28E+05 | |
| #004 | R&D Sys Activin A | 50 ng/ml | | 795 | 79 | 2.49E+06 | 1.40E+06 | |
| #004 | R&D Sys Activin A | 100 ng/ml | | 1298 | 729 | 9.34E+06 | 4.09E+06 | 100 |
| #004 | R&D Sys Activin A | 200 ng/ml | | 2400 | 407 | 4.81E+07 | 5.92E+06 | |
| #004 | R&D Sys Activin A | 400 ng/ml | | 4702 | 1283 | 7.94E+07 | 2.18E+07 | |
| #004 | ACTN38 | 1x | 1:20 | 2023 | 1181 | 9.26E+04 | 2.77E+04 | 1.0 |
| #004 | ACTN38 | 10x | 1:20 | 3019 | 893 | 3.89E+05 | 2.87E+05 | 4.2 |
| #004 | ACTN38 | 10x | 1:40 | 2520 | 1778 | 3.56E+04 | 4.05E+04 | 0.4 |
| #004 | ACTN39 | 1x | 1:20 | 1596 | 1399 | 5.13E+04 | 2.35E+04 | 0.5 |
| #004 | ACTN39 | 10x | 1:20 | 3362 | 2213 | 1.31E+06 | 1.91E+06 | 14.0 |
| #004 | ACTN39 | 10x | 1:40 | 1331 | 702 | 4.81E+05 | 3.60E+05 | 5.1 |
| #004 | ACTN41 | 1x | 1:20 | 6073 | 1507 | 1.42E+05 | 8.68E+04 | 1.5 |
| #004 | ACTN41 | 10x | 1:20 | 1397 | 75 | 1.46E+05 | 2.20E+05 | 1.6 |
| #004 | ACTN41 | 10x | 1:40 | 2643 | 1070 | 6.30E+04 | 2.44E+04 | 0.7 |
| #004 | ACTN43 | 1x | 1:20 | 657 | 352 | 1.85E+04 | 1.62E+04 | 0.2 |
| #004 | ACTN43 | 10x | 1:20 | 877 | 388 | 2.13E+05 | 1.93E+05 | 2.3 |
| #004 | ACTN43 | 10x | 1:40 | 1251 | 1005 | 5.57E+04 | 5.99E+04 | 0.6 |
| #004 | ACTN44 | 1x | 1:20 | 3657 | 2434 | 5.01E+04 | 4.52E+04 | 0.5 |
| #004 | ACTN44 | 10x | 1:20 | 1508 | 479 | 3.24E+05 | 1.47E+05 | 3.5 |
| #004 | ACTN44 | 10x | 1:40 | 2272 | 242 | 1.58E+05 | 1.34E+05 | 1.7 |
| #004 | ACTN45 | 1x | 1:20 | 4591 | 963 | 1.48E+05 | 7.47E+04 | 1.6 |
| #004 | ACTN45 | 10x | 1:20 | 2058 | 1013 | 1.13E+05 | 7.26E+04 | 1.2 |
| #004 | ACTN45 | 10x | 1:40 | 4482 | 1145 | 2.30E+04 | 2.04E+04 | 0.2 |
| #004 | ACTN47 | 1x | 1:20 | 2624 | 1761 | 7.48E+05 | 8.86E+05 | 8.0 |
| #004 | ACTN47 | 10x | 1:20 | 1399 | 1224 | 4.27E+06 | 3.08E+06 | 45.7 |
| #004 | ACTN47 | 10x | 1:40 | 1610 | 904 | 1.09E+06 | 9.20E+05 | 11.7 |
| #004 | ACTN52 | 1x | 1:20 | 3092 | 1154 | 1.17E+05 | 1.80E+05 | 1.3 |
| #004 | ACTN52 | 10x | 1:20 | 4869 | 783 | 2.64E+04 | 1.80E+04 | 0.3 |
| #004 | ACTN52 | 10x | 1:40 | 3900 | 1956 | 9.50E+04 | 1.10E+05 | 1.0 |
| #005 | no Activin A | NA | NA | 4232 | 1414 | 8.01E+05 | 5.56E+05 | 2.7 |
| #005 | R&D Sys Activin A | 6.25 ng/ml | | 2175 | 647 | 8.53E+05 | 5.63E+05 | |
| #005 | R&D Sys Activin A | 12.5 ng/ml | | 1360 | 504 | 5.86E+05 | 3.36E+05 | |
| #005 | R&D Sys Activin A | 25 ng/ml | | 1303 | 337 | 1.13E+06 | 5.77E+05 | |
| #005 | R&D Sys Activin A | 50 ng/ml | | 2022 | 409 | 7.96E+06 | 4.30E+06 | |
| #005 | R&D Sys Activin A | 100 ng/ml | | 3145 | 834 | 2.95E+07 | 1.22E+07 | 100 |
| #005 | R&D Sys Activin A | 200 ng/ml | | 3706 | 1791 | 6.35E+07 | 2.66E+07 | |
| #005 | R&D Sys Activin A | 400 ng/ml | | 7054 | 3513 | 1.10E+08 | 3.62E+07 | |
| #005 | ACTN53 | 1x | 1:20 | 1610 | 176 | 1.14E+06 | 4.54E+05 | 3.8 |
| #005 | ACTN53 | 10x | 1:20 | 3229 | 1880 | 1.51E+06 | 9.03E+05 | 5.1 |
| #005 | ACTN53 | 10x | 1:40 | 3476 | 3189 | 9.41E+05 | 4.03E+05 | 3.2 |
| #005 | ACTN55 | 1x | 1:20 | 2425 | 347 | 3.41E+05 | 3.26E+05 | 1.2 |
| #005 | ACTN55 | 10x | 1:20 | 3611 | 801 | 2.67E+05 | 2.27E+05 | 0.9 |
| #005 | ACTN55 | 10x | 1:40 | 2761 | 1188 | 5.44E+05 | 5.79E+05 | 1.8 |
| #005 | ACTN57 | 1x | 1:20 | 4485 | 891 | 9.99E+05 | 4.35E+05 | 3.4 |
| #005 | ACTN57 | 10x | 1:20 | 6471 | 379 | 1.74E+06 | 3.08E+05 | 5.9 |
| #005 | ACTN57 | 10x | 1:40 | 4594 | 2303 | 1.30E+06 | 7.33E+05 | 4.4 |
| #005 | ACTN59 | 1x | 1:20 | 2613 | 1680 | 1.09E+06 | 6.75E+05 | 3.7 |
| #005 | ACTN59 | 10x | 1:20 | 3304 | 316 | 2.11E+06 | 3.62E+05 | 7.1 |
| #005 | ACTN59 | 10x | 1:40 | 1776 | 699 | 1.81E+06 | 1.44E+06 | 6.1 |
| #005 | ACTN62 | 1x | 1:20 | 1661 | 757 | 1.05E+06 | 8.02E+05 | 3.6 |
| #005 | ACTN62 | 10x | 1:20 | 5728 | 3055 | 2.45E+05 | 3.32E+05 | 0.8 |
| #005 | ACTN62 | 10x | 1:40 | 3782 | 1515 | 1.33E+06 | 6.03E+05 | 4.5 |
| #005 | ACTN63 | 1x | 1:20 | 3380 | 1583 | 1.07E+06 | 1.24E+06 | 3.6 |
| #005 | ACTN63 | 10x | 1:20 | 1935 | 512 | 2.28E+06 | 7.88E+05 | 7.7 |
| #005 | ACTN63 | 10x | 1:40 | 2718 | 266 | 2.01E+06 | 1.00E+06 | 6.8 |
| #005 | ACTN64 | 1x | 1:20 | 2415 | 404 | 1.30E+06 | 5.48E+05 | 4.4 |
| #005 | ACTN64 | 10x | 1:20 | 2215 | 485 | 1.55E+06 | 5.55E+05 | 5.2 |
| #005 | ACTN64 | 10x | 1:40 | 2688 | 1645 | 1.12E+06 | 4.43E+05 | 3.8 |
| #005 | ACTN71 | 1x | 1:20 | 1621 | 760 | 2.45E+05 | 1.24E+05 | 0.8 |
| #005 | ACTN71 | 10x | 1:20 | 3909 | 1450 | 3.61E+05 | 3.60E+05 | 1.2 |
| #005 | ACTN71 | 10x | 1:40 | 1970 | 894 | 1.01E+06 | 4.85E+05 | 3.4 |
| #006 | no Activin A | NA | NA | 2066 | 824 | 5.62E+05 | 2.27E+05 | 2.5 |
| #006 | R&D Sys Activin A | 6.25 ng/ml | | 1315 | 322 | 3.77E+05 | 1.30E+05 | |
| #006 | R&D Sys Activin A | 12.5 ng/ml | | 984 | 209 | 7.95E+05 | 6.47E+05 | |
| #006 | R&D Sys Activin A | 25 ng/ml | | 1445 | 475 | 2.03E+06 | 6.78E+05 | |
| #006 | R&D Sys Activin A | 50 ng/ml | | 1519 | 682 | 3.67E+06 | 2.71E+06 | |
| #006 | R&D Sys Activin A | 100 ng/ml | | 2068 | 940 | 2.29E+07 | 1.01E+07 | 100 |
| #006 | R&D Sys Activin A | 200 ng/ml | | 3340 | 493 | 5.97E+07 | 5.74E+06 | |
| #006 | R&D Sys Activin A | 400 ng/ml | | 3668 | 1691 | 6.58E+07 | 2.77E+07 | |
| #006 | ACTN1 | 1x | 1:20 | 4284 | 304 | 7.44E+07 | 2.46E+06 | 324.7 |
| #006 | ACTN1 | 10x | 1:20 | 7083 | 2789 | 1.25E+08 | 4.36E+07 | 546.8 |
| #006 | ACTN1 | 10x | 1:40 | 5924 | 1885 | 1.10E+08 | 3.36E+07 | 480.5 |
| #006 | ACTN75 | 1x | 1:20 | 2873 | 1449 | 1.81E+05 | 1.30E+05 | 0.8 |
| #006 | ACTN75 | 10x | 1:20 | 3867 | 2263 | 3.91E+05 | 3.38E+05 | 1.7 |
| #006 | ACTN75 | 10x | 1:40 | 3641 | 2091 | 1.24E+06 | 7.46E+05 | 5.4 |
| #006 | ACTN76 | 1x | 1:20 | 3613 | 1828 | 9.19E+05 | 3.95E+05 | 4.0 |
| #006 | ACTN76 | 10x | 1:20 | 4732 | 2072 | 1.43E+06 | 5.63E+05 | 6.2 |
| #006 | ACTN76 | 10x | 1:40 | 7608 | 2666 | 7.54E+05 | 5.65E+05 | 3.3 |
| #006 | ACTN79 | 1x | 1:20 | 2958 | 885 | 4.07E+05 | 5.13E+05 | 1.8 |
| #006 | ACTN79 | 10x | 1:20 | 7704 | 1033 | 3.83E+05 | 1.24E+05 | 1.7 |
| #006 | ACTN79 | 10x | 1:40 | 2486 | 355 | 6.19E+04 | 8.10E+04 | 0.3 |
| #006 | ACTN84 | 1x | 1:20 | 1976 | 1370 | 3.37E+05 | 2.96E+05 | 1.5 |
| #006 | ACTN84 | 10x | 1:20 | 2272 | 656 | 2.65E+05 | 1.09E+05 | 1.2 |
| #006 | ACTN84 | 10x | 1:40 | 5228 | 1923 | 8.40E+05 | 2.60E+05 | 3.7 |
| #006 | ACTN87 | 1x | 1:20 | 1548 | 919 | 5.85E+05 | 5.88E+04 | 2.6 |
| #006 | ACTN87 | 10x | 1:20 | 3258 | 2198 | 7.89E+05 | 1.06E+06 | 3.4 |
| #006 | ACTN87 | 10x | 1:40 | 3613 | 1941 | 3.99E+05 | 2.80E+05 | 1.7 |
| #006 | ACTN89 | 1x | 1:20 | 5495 | 714 | 3.63E+05 | 2.52E+05 | 1.6 |
| #006 | ACTN89 | 10x | 1:20 | 5558 | 2729 | 5.77E+05 | 4.83E+05 | 2.5 |
| #006 | ACTN89 | 10x | 1:40 | 4474 | 1027 | 7.23E+05 | 2.70E+05 | 3.2 |
| #006 | ACTN90 | 1x | 1:20 | 1727 | 908 | 1.34E+06 | 1.15E+06 | 5.9 |
| #006 | ACTN90 | 10x | 1:20 | 2819 | 603 | 4.13E+05 | 5.74E+05 | 1.8 |
| #006 | ACTN90 | 10x | 1:40 | 3042 | 1374 | 1.33E+06 | 8.60E+05 | 5.8 |
| #007 | no Activin A | NA | NA | 10305 | 653 | 3.07E+05 | 8.60E+04 | 1.4 |
| #007 | R&D Sys Activin A | 6.25 ng/ml | | 3824 | 408 | 8.05E+05 | 2.12E+05 | |
| #007 | R&D Sys Activin A | 12.5 ng/ml | | 3131 | 791 | 1.26E+06 | 5.03E+05 | |
| #007 | R&D Sys Activin A | 25 ng/ml | | 4462 | 414 | 3.68E+06 | 1.44E+06 | |
| #007 | R&D Sys Activin A | 50 ng/ml | | 5146 | 864 | 6.34E+06 | 4.04E+06 | |
| #007 | R&D Sys Activin A | 100 ng/ml | | 8684 | 721 | 2.21E+07 | 3.64E+06 | 100 |
| #007 | R&D Sys Activin A | 200 ng/ml | | 12305 | 476 | 6.47E+07 | 9.28E+06 | |
| #007 | R&D Sys Activin A | 400 ng/ml | | 12756 | 1027 | 8.63E+07 | 1.06E+07 | |
| #007 | mock SN | 10x | 1:20 | 12395 | 1732 | 2.41E+06 | 2.90E+06 | 10.9 |
| #007 | OriGENE WT | 10x | 1:20 | 12727 | 564 | 8.01E+07 | 1.66E+07 | 362.4 |
| #007 | ACTN1 | 10x | 1:20 | 12543 | 2154 | 8.61E+07 | 1.25E+07 | 389.5 |
| #007 | ACTN24 | 10x | 1:20 | 3261 | 1237 | 2.91E+06 | 1.65E+06 | 13.2 |
| #007 | ACTN25 | 10x | 1:20 | 5043 | 112 | 3.55E+06 | 8.54E+05 | 16.1 |
| #007 | ACTN26 | 10x | 1:20 | 4250 | 899 | 5.92E+05 | 7.71E+04 | 2.7 |
| #007 | ACTN29 | 10x | 1:20 | 8943 | 805 | 5.13E+07 | 4.78E+06 | 232.0 |
| #007 | ACTN30 | 10x | 1:20 | 7357 | 1423 | 6.73E+05 | 2.60E+05 | 3.0 |
| #007 | ACTN31 | 10x | 1:20 | 10450 | 2398 | 5.44E+07 | 1.94E+07 | 246.3 |
| #007 | ACTN33 | 10x | 1:20 | 3588 | 1050 | 2.15E+06 | 4.53E+05 | 9.7 |
| #007 | ACTN34 | 10x | 1:20 | 10063 | 2249 | 5.69E+07 | 1.71E+07 | 257.4 |
| #007 | ACTN37 | 10x | 1:20 | 3957 | 336 | 1.35E+06 | 2.37E+05 | 6.1 |
| #007 | ACTN58 | 10x | 1:20 | 11078 | 1555 | 6.70E+05 | 3.42E+05 | 3.0 |
| #007 | ACTN66 | 10x | 1:20 | 13360 | 2677 | 4.84E+05 | 1.36E+05 | 2.2 |
| #007 | ACTN67 | 10x | 1:20 | 12653 | 804 | 4.44E+05 | 1.18E+05 | 2.0 |
| #007 | ACTN68 | 10x | 1:20 | 13395 | 960 | 1.43E+06 | 1.96E+06 | 6.5 |
| #007 | ACTN70 | 10x | 1:20 | 12551 | 709 | 9.67E+05 | 4.90E+05 | 4.4 |
| #007 | ACTN73 | 10x | 1:20 | 10569 | 1074 | 8.27E+05 | 1.69E+05 | 3.7 |
| #007 | ACTN80 | 10x | 1:20 | 9898 | 1537 | 4.08E+05 | 9.53E+03 | 1.8 |
| #007 | ACTN83 | 10x | 1:20 | 12084 | 2300 | 5.39E+05 | 1.59E+05 | 2.4 |
| #007 | ACTN86 | 10x | 1:20 | 11821 | 328 | 7.45E+05 | 2.00E+05 | 3.4 |
| #007 | ACTN88 | 10x | 1:20 | 11583 | 405 | 6.21E+05 | 2.73E+05 | 2.8 |
| #007 | ACTN92 | 10x | 1:20 | 14298 | 558 | 5.60E+05 | 1.41E+05 | 2.5 |
| #007 | ACTN93 | 10x | 1:20 | 13409 | 1062 | 5.87E+05 | 3.82E+05 | 2.7 |
| #008 | no Activin A | NA | NA | 10838 | 654 | 1.78E+06 | 9.81E+04 | 3.2 |
| #008 | R&D Sys Activin A | 6.25 ng/ml | | 2383 | 504 | 2.25E+06 | 4.60E+05 | |
| #008 | R&D Sys Activin A | 12.5 ng/ml | | 3746 | 522 | 7.90E+06 | 1.70E+06 | |
| #008 | R&D Sys Activin A | 25 ng/ml | | 4706 | 2153 | 1.59E+07 | 9.77E+06 | |
| #008 | R&D Sys Activin A | 50 ng/ml | | 5714 | 403 | 2.14E+07 | 2.16E+06 | |
| #008 | R&D Sys Activin A | 100 ng/ml | | 7479 | 942 | 5.53E+07 | 6.25E+06 | 100 |
| #008 | R&D Sys Activin A | 200 ng/ml | | 10212 | 130 | 1.49E+08 | 5.07E+06 | |
| #008 | R&D Sys Activin A | 400 ng/ml | | 13542 | 1687 | 2.56E+08 | 4.52E+07 | |
| #008 | mock SN | 50x | 1:20 | 15783 | 2747 | 4.85E+06 | 2.09E+06 | 8.8 |
| #008 | OriGENE WT | 50x | 1:20 | 12200 | 618 | 2.26E+08 | 1.67E+07 | 409.0 |
| #008 | ACTN1 | 50x | 1:20 | 13673 | 466 | 2.60E+08 | 1.07E+07 | 470.1 |
| #008 | ACTN24 | 50x | 1:20 | 4785 | 1406 | 1.04E+07 | 3.24E+06 | 18.8 |
| #008 | ACTN25 | 50x | 1:20 | 4620 | 699 | 1.52E+07 | 3.13E+05 | 27.6 |
| #008 | ACTN26 | 50x | 1:20 | 5043 | 1644 | 4.78E+06 | 2.70E+06 | 8.6 |
| #008 | ACTN29 | 50x | 1:20 | 8835 | 1388 | 1.03E+08 | 1.21E+07 | 186.2 |
| #008 | ACTN30 | 50x | 1:20 | 5013 | 1835 | 2.87E+06 | 8.58E+05 | 5.2 |
| #008 | ACTN31 | 50x | 1:20 | 11148 | 1327 | 1.49E+08 | 1.78E+07 | 269.7 |
| #008 | ACTN33 | 50x | 1:20 | 3383 | 1050 | 6.50E+06 | 1.77E+06 | 11.8 |
| #008 | ACTN34 | 50x | 1:20 | 12163 | 379 | 1.47E+08 | 2.80E+07 | 266.9 |
| #008 | ACTN37 | 50x | 1:20 | 2902 | 945 | 5.89E+06 | 6.68E+06 | 10.6 |
| #008 | ACTN58 | 50x | 1:20 | 13214 | 2227 | 3.78E+06 | 5.20E+05 | 6.8 |
| #008 | ACTN66 | 50x | 1:20 | 16460 | 1132 | 3.39E+06 | 6.21E+05 | 6.1 |
| #008 | ACTN67 | 50x | 1:20 | 14821 | 1839 | 3.50E+06 | 1.52E+06 | 6.3 |
| #008 | ACTN68 | 50x | 1:20 | 15926 | 1335 | 1.83E+06 | 4.47E+05 | 3.3 |
| #008 | ACTN70 | 50x | 1:20 | 16071 | 2209 | 3.20E+06 | 1.60E+06 | 5.8 |
| #008 | ACTN73 | 50x | 1:20 | 16351 | 910 | 3.13E+06 | 1.41E+06 | 5.7 |
| #008 | ACTN80 | 50x | 1:20 | 14686 | 3087 | 3.52E+06 | 1.23E+06 | 6.4 |
| #008 | ACTN83 | 50x | 1:20 | 15829 | 2323 | 9.01E+06 | 9.79E+06 | 16.3 |
| #008 | ACTN86 | 50x | 1:20 | 17034 | 2001 | 2.34E+06 | 3.56E+05 | 4.2 |
| #008 | ACTN88 | 50x | 1:20 | 15317 | 824 | 2.87E+06 | 7.89E+05 | 5.2 |
| #008 | ACTN92 | 50x | 1:20 | 15009 | 1821 | 3.32E+06 | 1.20E+06 | 6.0 |
| #008 | ACTN93 | 50x | 1:20 | 15900 | 2145 | 2.67E+06 | 1.38E+04 | 4.8 |
| #009 | no Activin A | NA | NA | 13043 | 1790 | 6.93E+05 | 6.89E+05 | 2.8 |
| #009 | R&D Sys Activin A | 6.25 ng/ml | | 9256 | 4615 | 1.71E+06 | 2.46E+06 | |
| #009 | R&D Sys Activin A | 12.5 ng/ml | | 11386 | 458 | 1.30E+06 | 7.22E+05 | |
| #009 | R&D Sys Activin A | 25 ng/ml | | 6396 | 530 | 3.58E+06 | 1.95E+06 | |
| #009 | R&D Sys Activin A | 50 ng/ml | | 5600 | 568 | 4.70E+06 | 8.65E+05 | |
| #009 | R&D Sys Activin A | 100 ng/ml | | 5328 | 1582 | 2.49E+07 | 1.50E+07 | 100 |
| #009 | R&D Sys Activin A | 200 ng/ml | | 9019 | 689 | 8.89E+07 | 1.52E+07 | |
| #009 | R&D Sys Activin A | 400 ng/ml | | 10913 | 1330 | 1.03E+08 | 2.89E+07 | |
| #009 | mock SN | 10x | 1:20 | 13022 | 1802 | 1.49E+06 | 1.40E+06 | 6.0 |
| #009 | OriGENE WT | 10x | 1:20 | 7061 | 1740 | 3.07E+07 | 1.70E+07 | 123.3 |
| #009 | ACTN1 | 10x | 1:20 | 11568 | 1491 | 1.05E+08 | 5.27E+07 | 423.1 |
| #009 | ACTN27 | 10x | 1:20 | 6664 | 843 | 1.39E+07 | 1.32E+07 | 55.7 |
| #009 | ACTN42 | 10x | 1:20 | 9937 | 985 | 3.45E+06 | 2.40E+06 | 13.8 |
| #009 | ACTN48 | 10x | 1:20 | 6030 | 752 | 1.77E+07 | 8.63E+06 | 71.1 |
| #009 | ACTN57 | 10x | 1:20 | 9442 | 1808 | 1.70E+06 | 1.74E+06 | 6.8 |
| #009 | ACTN60 | 10x | 1:20 | 8303 | 4620 | 5.90E+05 | 5.50E+05 | 2.4 |
| #009 | ACTN61 | 10x | 1:20 | 15268 | 1851 | 1.70E+06 | 1.28E+06 | 6.8 |
| #009 | ACTN72 | 10x | 1:20 | 14638 | 3638 | 3.44E+06 | 2.45E+06 | 13.8 |
| #009 | ACTN74 | 10x | 1:20 | 15049 | 579 | 3.30E+06 | 4.01E+06 | 13.2 |
| #009 | ACTN78 | 10x | 1:20 | 12585 | 1110 | 1.11E+06 | 6.28E+05 | 4.4 |
| #009 | mock SN | 50x | 1:20 | 12555 | 1659 | 4.48E+06 | 6.50E+06 | 18.0 |
| #009 | OriGENE WT | 50x | 1:20 | 9025 | 3008 | 7.11E+07 | 3.89E+07 | 285.3 |
| #009 | San Diego WT | 50x | 1:20 | 14552 | 1244 | 1.60E+08 | 1.31E+07 | 643.2 |
| #009 | ACTN27 | 50x | 1:20 | 6857 | 823 | 2.82E+07 | 1.37E+07 | 113.1 |
| #009 | ACTN42 | 50x | 1:20 | 7805 | 1316 | 7.53E+06 | 5.02E+06 | 30.2 |
| #009 | ACTN48 | 50x | 1:20 | 8941 | 1394 | 3.93E+07 | 2.36E+07 | 157.8 |
| #009 | ACTN57 | 50x | 1:20 | 12697 | 2468 | 7.82E+06 | 4.81E+06 | 31.4 |
| #009 | ACTN60 | 50x | 1:20 | 10603 | 2616 | 3.73E+06 | 4.98E+06 | 15.0 |
| #009 | ACTN61 | 50x | 1:20 | 15256 | 1820 | 7.18E+06 | 6.84E+06 | 28.8 |
| #009 | ACTN72 | 50x | 1:20 | 16810 | 1507 | 6.51E+06 | 4.86E+06 | 26.1 |
| #009 | ACTN74 | 50x | 1:20 | 16143 | 1292 | 1.03E+07 | 3.56E+06 | 41.3 |
| #009 | ACTN78 | 50x | 1:20 | 16301 | 1056 | 5.64E+06 | 4.44E+06 | 22.6 |
| #010 | no Activin A | NA | NA | 17442 | 2846 | 1.94E+07 | 2.13E+06 | 13.7 |
| #010 | R&D Sys Activin A | 6.25 ng/ml | | 14185 | 2876 | 3.47E+07 | 7.27E+06 | |
| #010 | R&D Sys Activin A | 12.5 ng/ml | | 10762 | 420 | 6.32E+07 | 3.43E+06 | |
| #010 | R&D Sys Activin A | 25 ng/ml | | 10543 | 1503 | 6.20E+07 | 1.14E+07 | |
| #010 | R&D Sys Activin A | 50 ng/ml | | 9793 | 1151 | 5.63E+07 | 4.97E+06 | |
| #010 | R&D Sys Activin A | 100 ng/ml | | 13013 | 558 | 1.41E+08 | 1.66E+07 | 100 |
| #010 | R&D Sys Activin A | 200 ng/ml | | 14629 | 1632 | 2.77E+08 | 5.08E+07 | |
| #010 | R&D Sys Activin A | 400 ng/ml | | 18418 | 393 | 4.91E+08 | 4.91E+07 | |
| #010 | mock SN | 10x | 1:20 | 20032 | 567 | 1.83E+07 | 2.94E+06 | 13.0 |
| #010 | OriGENE WT | 10x | 1:20 | 11356 | 449 | 1.27E+08 | 5.28E+06 | 89.8 |
| #010 | ACTN1 | 10x | 1:20 | 15112 | 1475 | 2.78E+08 | 9.18E+07 | 197.1 |
| #010 | ACTN13 | 10x | 1:20 | 19861 | 3323 | 2.78E+07 | 1.64E+07 | 19.7 |
| #010 | ACTN15 | 10x | 1:20 | 22700 | 2525 | 2.89E+07 | 1.54E+07 | 20.5 |
| #010 | ACTN36 | 10x | 1:20 | 24664 | 4630 | 2.60E+07 | 7.76E+06 | 18.4 |
| #010 | ACTN40 | 10x | 1:20 | 11321 | 1545 | 1.21E+08 | 3.43E+07 | 85.6 |
| #010 | ACTN46 | 10x | 1:20 | 20757 | 509 | 1.99E+07 | 9.68E+06 | 14.1 |
| #010 | ACTN49 | 10x | 1:20 | 10076 | 425 | 1.60E+07 | 4.38E+06 | 11.3 |
| #010 | ACTN50 | 10x | 1:20 | 22171 | 527 | 1.72E+07 | 2.46E+06 | 12.2 |
| #010 | ACTN51 | 10x | 1:20 | 21780 | 750 | 1.85E+07 | 2.38E+06 | 13.1 |
| #010 | ACTN56 | 10x | 1:20 | 23413 | 753 | 2.79E+07 | 2.45E+06 | 19.8 |
| #010 | ACTN65 | 10x | 1:20 | 22058 | 1985 | 3.23E+07 | 4.64E+06 | 22.9 |
| #010 | ACTN69 | 10x | 1:20 | 20178 | 1849 | 2.73E+07 | 5.43E+06 | 19.4 |
| #010 | ACTN77 | 10x | 1:20 | 17696 | 1981 | 2.82E+07 | 6.02E+06 | 20.0 |
| #010 | ACTN81 | 10x | 1:20 | 19897 | 2154 | 1.60E+07 | 3.00E+06 | 11.4 |
| #010 | ACTN91 | 10x | 1:20 | 17043 | 1619 | 1.12E+07 | 1.71E+06 | 7.9 |
| #010 | ACTN94 | 10x | 1:20 | 20142 | 401 | 1.51E+07 | 3.06E+06 | 10.7 |

TABLE 9
Primary Screening data subset: Effect of the peptides of the present
invention on differentiation of pluripotent stem cells
TABLE 9
Sample

ACTN1 (wildtype)
ACTN2
ACTN4
ACTN6
ACTN7
ACTN11
ACTN12
ACTN14
ACTN16
ACTN24
ACTN25
ACTN27
ACTN28
ACTN29
ACTN31
ACTN34
ACTN39
ACTN40
ACTN42
ACTN47
ACTN48
ACTN56
ACTN57
ACTN61
ACTN65
ACTN69
ACTN72
ACTN74
ACTN77
ACTN83

TABLE 10
Concentration of selected peptides of the present invention, as determined by an activin-A ELISA
Table 10

| | | | | | | | | | | | | | Estimated |
|---|---|---|---|---|---|---|---|---|---|---|---|---|---|
| | ELISA Assay Dilution & Concentration [ng/ml] | | | | | | Calculated Stock Concentration [ng/ml] | | | | | | mid- |
| SAMPLE | 1:40 | 1:80 | 1:160 | 1:320 | 1:600 | 1:1280 | 1:40 | 1:80 | 1:160 | 1:320 | 1:600 | 1:1280 | titration |
| HEK Spent | ND* | ND | ND | ND | ND | ND | ND | ND | ND | ND | ND | ND | |
| Medium (Neg) | | | | | | | | | | | | | |
| ACTN1 (10x) | 11.73 | 11.88 | 12.40 | 12.46 | 7.85 | 4.23 | 469.20 | 950.16 | 1983.20 | 3986.88 | 5025.28 | 5411.84 | 1:1280 |
| ACTN4 (10x) | 12.48 | 6.83 | 3.32 | 2.13 | 1.03 | 0.62 | 499.36 | 546.72 | 531.20 | 680.00 | 657.28 | 798.72 | 1:120 |
| ACTN16 (10x) | ND | ND | ND | ND | ND | ND | ND | ND | ND | ND | ND | ND | |
| ACTN34 (10x) | 11.13 | 6.95 | 3.53 | 2.12 | 1.04 | 0.53 | 445.36 | 555.60 | 565.12 | 677.44 | 663.68 | 675.84 | 1:120 |
| ACTN40 (10x) | 6.44 | 3.40 | 1.77 | 1.04 | 0.48 | 0.19 | 257.64 | 272.08 | 283.36 | 333.44 | 309.76 | 245.76 | 1:60 |

*ND, not detectable

TABLE 11
Concentration of selected peptides of the present
invention, as determined by an activin-A ELISA
Table 11

| | | | | Assay Dilution | | | Calculated Stock | | | | | |
|---|---|---|---|---|---|---|---|---|---|---|---|---|
| | Assay Dilution Series | | | Concentration [ng/ml] | | | Concentration [ng/ml] | | | Mean Results | | |
| | High CCN | | Low CCN | Low | Mid | High | Low | Mid | High | MEAN CCN | | |
| SAMPLE NAME | (1 to X ) | (1 to X) | (1 to X) | CCN | CCN | CCN | CCN | CCN | CCN | [ng/ml] | STDEV | % CV |
| ACTN1 (10x) | 600 | 1200 | 2400 | 1.84 | 4.32 | 8.80 | 4,406 | 5,186 | 5,279 | 4796 | 551.5 | 11.5 |
| ACTN4 (10x) | 60 | 120 | 240 | 3.05 | 5.89 | 9.82 | 731 | 707 | 589 | 719 | 16.8 | 2.3 |
| ACTN16 (10x) | 5 | 10 | 20 | 2.05 | 4.23 | 8.07 | 41 | 42 | 40 | 41 | 1.0 | 2.4 |
| ACTN34 (10x) | 70 | 140 | 280 | 2.97 | 5.92 | 9.49 | 832 | 828 | 665 | 830 | 2.5 | 0.3 |
| ACTN40 (10x) | 35 | 70 | 140 | 4.36 | 7.96 | 10.05 | 610 | 557 | 352 | 584 | 37.5 | 6.4 |

TABLE 12
Differentiation of pluripotent stem cells with peptides of the present invention that have been further modified to contain metal binding sites

| | | | Super- | | | Estimated | | | | | |
|---|---|---|---|---|---|---|---|---|---|---|---|
| | | | natant | ELISA | | Assay | | | Sox 17 Intensity | | |
| | Parent | Bis-His | concen- | concentration | Assay | Concentration | Cell Number | | | | % of |
| Sample | Peptide | Mutation | tration | (ng/ml) | Dilution | (ng/ml) | average | S.D. | average | S.D. | control |
| no Activin A | NA | NA | NA | NA | NA | NA | 10846 | 565 | 5.58E+06 | 5.04E+06 | 13 |
| R&D Sys | NA | NA | NA | NA | NA | 6.25 | 5540 | 612 | 5.49E+06 | 4.93E+06 | 13 |
| Activin A | | | | | | | | | | | |
| R&D Sys | NA | NA | NA | NA | NA | 12.5 | 5784 | 1446 | 8.10E+06 | 2.44E+06 | 19 |
| Activin A | | | | | | | | | | | |
| R&D Sys | NA | NA | NA | NA | NA | 25 | 6212 | 96 | 1.69E+07 | 5.56E+06 | 39 |
| Activin A | | | | | | | | | | | |
| R&D Sys | NA | NA | NA | NA | NA | 50 | 6218 | 1047 | 1.45E+07 | 3.53E+06 | 33 |
| Activin A | | | | | | | | | | | |
| R&D Sys | NA | NA | NA | NA | NA | 100 | 8680 | 309 | 4.37E+07 | 1.38E+07 | 100 |
| Activin A | | | | | | | | | | | |
| R&D Sys | NA | NA | NA | NA | NA | 200 | 12032 | 841 | 1.25E+08 | 2.07E+07 | 285 |
| Activin A | | | | | | | | | | | |
| R&D Sys | NA | NA | NA | NA | NA | 400 | 13961 | 1174 | 1.73E+08 | 2.64E+07 | 396 |
| Activin A | | | | | | | | | | | |
| ACTN1 | ACTN1 | none | 10x | 984 | 1:20 | 49 | 12233 | 1152 | 1.15E+08 | 2.69E+07 | 263 |
| | wildtype | | | | | | | | | | |
| ACTD2 | ACTN1 | E3H/D5H | 10x | 762 | 1:20 | 38 | 13329 | 1426 | 1.32E+08 | 3.36E+07 | 303 |
| | wildtype | | | | | | | | | | |
| ACTD3 | ACTN1 | K7H/N9H | 4x | nd* | 1:20 | nd | 12637 | 470 | 1.34E+08 | 8.04E+06 | 306 |
| | wildtype | | | | | | | | | | |
| ACTD4 | ACTN1 | K13H/Q15H | 10x | 3206 | 1:20 | 160 | 12036 | 1102 | 1.13E+08 | 1.28E+07 | 259 |
| | wildtype | | | | | | | | | | |
| ACTD5 | ACTN1 | K14H/Q15H | 10x | 2759 | 1:20 | 138 | 11575 | 994 | 1.07E+08 | 2.14E+07 | 244 |
| | wildtype | | | | | | | | | | |
| ACTD6 | ACTN1 | Q15H/F17H | 10x | low | 1:20 | low | 11533 | 538 | 8.78E+07 | 2.54E+07 | 201 |
| | wildtype | | | | | | | | | | |
| ACTN16 | ACTN16 | none | 10x | 968 | 1:20 | 48 | 8775 | 681 | 3.89E+07 | 9.46E+06 | 89 |
| ACTD7 | ACTN16 | E3H/D5H | 10x | 340 | 1:20 | 17 | 7230 | 715 | 1.48E+07 | 2.48E+06 | 34 |
| ACTD8 | ACTN16 | K7H/N9H | 10x | 360 | 1:20 | 18 | 8127 | 456 | 2.40E+07 | 1.04E+07 | 55 |
| ACTD9 | ACTN16 | K13H/Q15H | 10x | 81 | 1:20 | 4 | 7430 | 1393 | 1.52E+07 | 2.76E+06 | 35 |
| ACTD10 | ACTN16 | K14H/Q15H | 10x | 356 | 1:20 | 18 | 9608 | 1229 | 4.55E+07 | 1.50E+07 | 104 |
| ACTD11 | ACTN16 | Q15H/F17H | 10x | 13 | 1:20 | 1 | 8734 | 1830 | 1.51E+07 | 7.49E+06 | 35 |
| ACTN34 | ACTN34 | none | 10x | 1161 | 1:20 | 58 | 11794 | 825 | 8.08E+07 | 1.17E+07 | 185 |
| ACTD12 | ACTN34 | E3H/D5H | 10x | 151 | 1:20 | 8 | 7944 | 1042 | 2.90E+07 | 8.66E+06 | 66 |
| ACTD13 | ACTN34 | K7H/N9H | 10x | 662 | 1:20 | 33 | 10503 | 1769 | 6.12E+07 | 1.21E+07 | 140 |
| ACTD14 | ACTN34 | K13H/Q15H | 10x | 45 | 1:20 | 2 | 8955 | 2176 | 2.37E+07 | 5.18E+06 | 54 |
| ACTD15 | ACTN34 | K14H/Q15H | 10x | 900 | 1:20 | 45 | 8371 | 995 | 3.90E+07 | 1.25E+07 | 89 |
| ACTD16 | ACTN34 | Q15H/F17H | 10x | 52 | 1:20 | 3 | 7320 | 203 | 2.45E+07 | 6.52E+06 | 56 |

*nd, not detectable

TABLE 13
Concentration of a selection of the peptides of the present invention

| Variant | ELISA concentration (ng/ml) |
|---|---|
| ACTN1 | 10,500 |
| ACTN4 | 1,874 |
| ACTN11 | 386 |
| ACTN12 | 310 |
| ACTN16 | 2,113 |
| ACTN27 | 115 |
| ACTN28 | 810 |
| ACTN29 | 1,392 |
| ACTN31 | 3,243 |
| ACTN34 | 1,154 |
| ACTN40 | 2,041 |
| ACTN47 | 179 |
| ACTN48 | 7,587 |
| ACTN56 | not detectable |
| ACTN65 | not detectable |
| ACTN69 | not detectable |

What is claimed is:

1. A method to differentiate pluripotent stem cells into cells expressing markers characteristic of the definitive endoderm lineage, comprising treating the pluripotent stem cells with a medium containing a peptide comprising the amino acid sequence of activin A containing at least one point mutation, for a period of time sufficient for the pluripotent stem cells to differentiate into cells expressing markers characteristic of the definitive endoderm lineage.

2. The method of claim 1, wherein the pluripotent stem cells are embryonic stem cells.

3. The method of claim 1, wherein the at least one point mutation is at least one of the amino acid residues in the amino acid sequence of activin A selected from the group consisting of: 10I, 16F, 39Y, 41E, 43E, 74F, 75A, 76N, 77L, 78K, 79S, and 82V.

4. The method of claim 3, wherein the at least one point mutation is selected from the group consisting of: a deletion, an insertion and a substitution.

5. The method of claim 1, wherein the at least one point mutation at least one of the amino acid residues in the amino acid sequence of activin A selected from the group consisting of: 16F, 18V, 19S, 20F, 37A, 38N, 39Y, 41E, 74F, 82V, 107N, 109I, 110V, and 116S.

6. The method of claim 5, wherein the at least one point mutation is selected from the group consisting of: a deletion, an insertion and a substitution.

7. The method of claim 1, wherein the peptide comprising the amino acid sequence of activin A containing at least one point mutation is further modified to contain at least one region that is capable of specifically binding to a ligand on a solid substrate in an affinity purification column.

8. The method of claim 7, wherein the at least one region that is capable of specifically binding to a ligand on a solid substrate in an affinity purification column is a metal binding site.

9. The method of claim 8, wherein the metal binding site comprises a pair of histidine residues.


SEQUENCE LISTING (196 sequences)

SEQ ID NO: 1 - protein, 310 aa, Artificial Sequence
  Synthetic Construct
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRR

SEQ ID NO: 2 - protein, 116 aa, Artificial Sequence [sequence omitted]

SEQ ID NO: 3 - protein, 116 aa, Artificial Sequence
  Synthetic Construct
GLECDGKVNLCCKKQNFVSFKDIGWNDWIIAPSGYHANECTGKCPSHIAGTSGSSLSFHSTVINHYRMRGHSPFSDLGSCCIPTKLRPMSMLYYDDGQNIIKKDIQNMIVEECGCS

SEQ ID NO: 4 - protein, 116 aa, Artificial Sequence
  Synthetic Construct
GLECDGKVNLCCKKQDFVSFKDIGWNDWIIAPSGYHANRCSGKCPSHIAGTSGSSLSFHSTVINHYRMRGHSPFSNMGSCCIPTKLRPMSMLYYDDGQNIIKKDIQNMIVEECGCS

SEQ ID NO: 5 - protein, 116 aa, Artificial Sequence
  Synthetic Construct
GLECDGKVNLCCKKQLFVSFKDIGWNDWIIAPSGYHANHCSGLCPSHIAGTSGSSLSFHSTVINHYRMRGHSPFSDMGACCVPTKLRPMSMLYYDDGQNIIKKDIQNMIVEECGCS

SEQ ID NO: 6 - protein, 116 aa, Artificial Sequence
  Synthetic Construct
GLECDGKVNYCCKKQNFVSFKDIGWNDWIIAPSGYHANECSGKCPSHIAGTSGSSLSFHSTVINHYRMRGHSPFSNLGACCIPTKLRPMSMLYYDDGQNIIKKDIQNMIVEECGCS

SEQ ID NO: 7 - protein, 116 aa, Artificial Sequence
  Sytnthetic Construct
GLECDGKVNLCCKKQWFVSFKDIGWNDWIIAPSGYHANRCSGKCPSHIAGTSGSSLSFHSTVINHYRMRGHSPFADMGACCIPTKLRPMSMLYYDDGQNIIKKDIQNMIVEECGCS

SEQ ID NO: 8 - protein, 116 aa, Artificial Sequence
  Synthetic Construct
GLECDGKVNYCCKKQHFVSFKDIGWNDWIIAPSGYHANSCSGLCPSHIAGTSGSSLSFHSTVINHYRMRGHSPFSQMGSCCIPTKLRPMSMLYYDDGQNIIKKDIQNMIVEECGCS

SEQ ID NO: 9 - protein, 116 aa, Artificial Sequence
  Synthetic Construct
GLECDGKVNYCCKKQDFVSFKDIGWNDWIIAPSGYHANKCSGKCPSHIAGTSGSSLSFHSTVINHYRMRGHSPFSDMGSCCVPTKLRPMSMLYYDDGQNIIKKDIQNMIVEECGCS

SEQ ID NO: 10 - protein, 116 aa, Artificial Sequence
  Synthetic Construct
GLECDGKVNYCCKKQDFVSFKDIGWNDWIIAPSGYHANKCTGKCPSHIAGTSGSSLSFHSTVINHYRMRGHSPFADLGACCIPTKLRPMSMLYYDDGQNIIKKDIQNMIVEECGCS

SEQ ID NO: 11 - protein, 116 aa, Artificial Sequence
  Synthetic Construct
GLECDGKVNYCCKKQDFVSFKDIGWNDWIIAPSGYHANRCSGLCPSHIAGTSGSSLSFHSTVINHYRMRGHSPFAQMGSCCIPTKLRPMSMLYYDDGQNIIKKDIQNMIVEECGCS

SEQ ID NO: 12 - protein, 116 aa, Artificial Sequence
  Synthetic Construct
GLECDGKVNLCCKKQLFVSFKDIGWNDWIIAPSGYHANHCSGLCPSHIAGTSGSSLSFHSTVINHYRMRGHSPFAQMGACCIPTKLRPMSMLYYDDGQNIIKKDIQNMIVEECGCS

SEQ ID NO: 13 - protein, 116 aa, Artificial Sequence
  Synthetic Construct
GLECDGKVNYCCKKQNFVSFKDIGWNDWIIAPSGYHANECSGLCPSHIAGTSGSSLSFHSTVINHYRMRGHSPFSQMGSCCIPTKLRPMSMLYYDDGQNIIKKDIQNMIVEECGCS

SEQ ID NO: 14 - protein, 116 aa, Artificial Sequence
  Synthetic Construct
GLECDGKVNLCCKKQDFVSFKDIGWNDWIIAPSGYHANKCSGKCPSHIAGTSGSSLSFHSTVINHYRMRGHSPFSDMGSCCIPTKLRPMSMLYYDDGQNIIKKDIQNMIVEECGCS

SEQ ID NO: 15 - protein, 116 aa, Artificial Sequence
  Synthetic Construct
GLECDGKVNLCCKKQHFVSFKDIGWNDWIIAPSGYHANRCDGLCPSHIAGTSGSSLSFHSTVINHYRMRGHSPFAQMGSCCVPTKLRPMSMLYYDDGQNIIKKDIQNMIVEECGCS

SEQ ID NO: 16 - protein, 116 aa, Artificial Sequence
  Synthetic Construct
GLECDGKVNLCCKKQDFVSFKDIGWNDWIIAPSGYHANRCSGLCPSHIAGTSGSSLSFHSTVINHYRMRGHSPFSNMGSCCIPTKLRPMSMLYYDDGQNIIKKDIQNMIVEECGCS

SEQ ID NO: 17 - protein, 116 aa, Artificial Sequence
  Synthetic Construct
GLECDGKVNLCCKKQLFVSFKDIGWNDWIIAPSGYHANHCSGLCPSHIAGTSGSSLSFHSTVINHYRMRGHSPFSQMGACCVPTKLRPMSMLYYDDGQNIIKKDIQNMIVEECGCS

SEQ ID NO: 18 - protein, 116 aa, Artificial Sequence
  Synthetic Construct
GLECDGKVNYCCKKQDFVSFKDIGWNDWIIAPSGYHANKCGGKCPSHIAGTSGSSLSFHSTVINHYRMRGHSPFSDMGACCVPTKLRPMSMLYYDDGQNIIKKDIQNMIVEECGCS

SEQ ID NO: 19 - protein, 116 aa, Artificial Sequence
  Synthetic Construct
GLECDGKVNYCCKKQNFVSFKDIGWNDWIIAPSGYHANKCSGKCPSHIAGTSGSSLSFHSTVINHYRMRGHSPFSDMGACCIPTKLRPMSMLYYDDGQNIIKKDIQNMIVEECGCS

SEQ ID NO: 20 - protein, 116 aa, Artificial Sequence
  Synthetic Construct
GLECDGKVNLCCKKQDFVSFKDIGWNDWIIAPSGYHANKCGGKCPSHIAGTSGSSLSFHSTVINHYRMRGHSPFANMGSCCIPTKLRPMSMLYYDDGQNIIKKDIQNMIVEECGCS

SEQ ID NO: 21 - protein, 116 aa, Artificial Sequence
  Synthetic Construct
GLECDGKVNLCCKKQNFVSFKDIGWNDWIIAPSGYHANKCGGLCPSHIAGTSGSSLSFHSTVINHYRMRGHSPFAQMGACCIPTKLRPMSMLYYDDGQNIIKKDIQNMIVEECGCS

SEQ ID NO: 22 - protein, 116 aa, Artificial Sequence
  Synthetic Construct
GLECDGKVNYCCKKQWFVSFKDIGWNDWIIAPSGYHANKCSGKCPSHIAGTSGSSLSFHSTVINHYRMRGHSPFANMGSCCIPTKLRPMSMLYYDDGQNIIKKDIQNMIVEECGCS

SEQ ID NO: 23 - protein, 116 aa, Artificial Sequence
  Synthetic Construct
GLECDGKVNLCCKKQDFVSFKDIGWNDWIIAPSGYHANRCDGLCPSHIAGTSGSSLSFHSTVINHYRMRGHSPFALMGACCIPTKLRPMSMLYYDDGQNIIKKDIQNMIVEECGCS

SEQ ID NO: 24 - protein, 116 aa, Artificial Sequence
  Synthetic Construct
GLECDGKVNYCCKKQDFVSFKDIGWNDWIIAPSGYHANKCDGKCPSHIAGTSGSSLSFHSTVINHYRMRGHSPFSDMGSCCIPTKLRPMSMLYYDDGQNIIKKDIQNMIVEECGCS

SEQ ID NO: 25 - protein, 116 aa, Artificial Sequence
  Synthetic Construct
GLECDGKVNLCCKKQDFVSFKDIGWNDWIIAPSGYHANRCSGRCPSHIAGTSGSSLSFHSTVINHYRMRGHSPFSDMGSCCVPTKLRPMSMLYYDDGQNIIKKDIQNMIVEECGCS

SEQ ID NO: 26 - protein, 116 aa, Artificial Sequence
  Synthetic Construct
GLECDGKVNLCCKKQNFVSFKDIGWNDWIIAPSGYHANECSGLCPSHIAGTSGSSLSFHSTVINHYRMRGHSPFSQMGSCCIPTKLRPMSMLYYDDGQNIIKKDIQNMIVEECGCS

SEQ ID NO: 27 - protein, 116 aa, Artificial Sequence
  Synthetic Construct
GLECDGKVNLCCKKQHFVSFKDIGWNDWIIAPSGYHANRCDGKCPSHIAGTSGSSLSFHSTVINHYRMRGHSPFANMGACCIPTKLRPMSMLYYDDGQNIIKKDIQNMIVEECGCS

SEQ ID NO: 28 - protein, 116 aa, Artificial Sequence
  Synthetic Construct
GLECDGKVNLCCKKQLFVSFKDIGWNDWIIAPSGYHANHCDGKCPSHIAGTSGSSLSFHSTVINHYRMRGHSPFANRGACCIPTKLRPMSMLYYDDGQNIIKKDIQNMIVEECGCS

SEQ ID NO: 29 - protein, 116 aa, Artificial Sequence
  Synthetic Construct
GLECDGKVNYCCKKQLFVSFKDIGWNDWIIAPSGYHANHCSGKCPSHIAGTSGSSLSFHSTVINHYRMRGHSPFANMGSCCIPTKLRPMSMLYYDDGQNIIKKDIQNMIVEECGCS

SEQ ID NO: 30 - protein, 116 aa, Artificial Sequence
  Synthetic Construct
GLECDGKVNLCCKKQLFVSFKDIGWNDWIIAPSGYHANHCSGKCPSHIAGTSGSSLSFHSTVINHYRMRGHSPFSDMGSCCIPTKLRPMSMLYYDDGQNIIKKDIQNMIVEECGCS

SEQ ID NO: 31 - protein, 116 aa, Artificial Sequence
  Synthetic Construct
GLECDGKVNYCCKKQNFVSFKDIGWNDWIIAPSGYHANKCSGLCPSHIAGTSGSSLSFHSTVINHYRMRGHSPFSKMGACCVPTKLRPMSMLYYDDGQNIIKKDIQNMIVEECGCS

SEQ ID NO: 32 - protein, 116 aa, Artificial Sequence
  Synthetic Construct
GLECDGKVNTCCKKQLFVSFKDIGWNDWIIAPSGYHANHCSGKCPSHIAGTSGSSLSFHSTVINHYRMRGHSPFSDMGSCCIPTKLRPMSMLYYDDGQNIIKKDIQNMIVEECGCS

SEQ ID NO: 33 - protein, 116 aa, Artificial Sequence
  Synthetic Construct
GLECDGKVNLCCKKQDFVSFKDIGWNDWIIAPSGYHANRCGGKCPSHIAGTSGSSLSFHSTVINHYRMRGHSPFSNMGSCCIPTKLRPMSMLYYDDGQNIIKKDIQNMIVEECGCS

SEQ ID NO: 34 - protein, 116 aa, Artificial Sequence
  Synthetic Construct
GLECDGKVNLCCKKQNFVSFKDIGWNDWIIAPSGYHANECMGKCPSHIAGTSGSSLSFHSTVINHYRMRGHSPFSDMGACCIPTKLRPMSMLYYDDGQNIIKKDIQNMIVEECGCS

SEQ ID NO: 35 - protein, 116 aa, Artificial Sequence
  Synthetic Construct
GLECDGKVNYCCKKQLFVSFKDIGWNDWIIAPSGYHANHCTGKCPSHIAGTSGSSLSFHSTVINHYRMRGHSPFSDLGSCCVPTKLRPMSMLYYDDGQNIIKKDIQNMIVEECGCS

SEQ ID NO: 36 - protein, 116 aa, Artificial Sequence
  Synthetic Construct
GLECDGKVNLCCKKQDFVSFKDIGWNDWIIAPSGYHANRCSGKCPSHIAGTSGSSLSFHSTVINHYRMRGHSPFSDMGSCCVPTKLRPMSMLYYDDGQNIIKKDIQNMIVEECGCS

SEQ ID NO: 37 - protein, 116 aa, Artificial Sequence
  Synthetic Construct
GLECDGKVNLCCKKQDFVSFKDIGWNDWIIAPSGYHANRCSGKCPSHIAGTSGSSLSFHSTVINHYRMRGHSPFANMGACCVPTKLRPMSMLYYDDGQNIIKKDIQNMIVEECGCS

SEQ ID NO: 38 - protein, 116 aa, Artificial Sequence
  Synthetic Construct
GLECDGKVNLCCKKQDFVSFKDIGWNDWIIAPSGYHANRCSGKCPSHIAGTSGSSLSFHSTVINHYRMRGHSPFSDMGACCIPTKLRPMSMLYYDDGQNIIKKDIQNMIVEECGCS

SEQ ID NO: 39 - protein, 116 aa, Artificial Sequence
  Synthetic Construct
GLECDGKVNLCCKKQDFVSFKDIGWNDWIIAPSGYHANKCGGKCPSHIAGTSGSSLSFHSTVINHYRMRGHSPFSQLGACCVPTKLRPMSMLYYDDGQNIIKKDIQNMIVEECGCS

SEQ ID NO: 40 - protein, 116 aa, Artificial Sequence
  Synthetic Construct
GLECDGKVNLCCKKQNFVSFKDIGWNDWIIAPSGYHANECSGLCPSHIAGTSGSSLSFHSTVINHYRMRGHSPFSDMGSCCVPTKLRPMSMLYYDDGQNIIKKDIQNMIVEECGCS

SEQ ID NO: 41 - protein, 116 aa, Artificial Sequence
  Synthetic Construct
GLECDGKVNLCCKKQLFVSFKDIGWNDWIIAPSGYHANHCAGLCPSHIAGTSGSSLSFHSTVINHYRMRGHSPFSNMGSCCVPTKLRPMSMLYYDDGQNIIKKDIQNMIVEECGCS

SEQ ID NO: 42 - protein, 116 aa, Artificial Sequence
  Synthetic Construct
GLECDGKVNLCCKKQDFVSFKDIGWNDWIIAPSGYHANSCSGLCPSHIAGTSGSSLSFHSTVINHYRMRGHSPFSDRGACCIPTKLRPMSMLYYDDGQNIIKKDIQNMIVEECGCS

SEQ ID NO: 43 - protein, 116 aa, Artificial Sequence
  Synthetic Construct
GLECDGKVNYCCKKQDFVSFKDIGWNDWIIAPSGYHANKCSGKCPSHIAGTSGSSLSFHSTVINHYRMRGHSPFSDMGSCCIPTKLRPMSMLYYDDGQNIIKKDIQNMIVEECGCS

SEQ ID NO: 44 - protein, 116 aa, Artificial Sequence
  Synthetic Construct
GLECDGKVNYCCKKQDFVSFKDIGWNDWIIAPSGYHANRCDGKCPSHIAGTSGSSLSFHSTVINHYRMRGHSPFSDMGACCVPTKLRPMSMLYYDDGQNIIKKDIQNMIVEECGCS

SEQ ID NO: 45 - protein, 116 aa, Artificial Sequence
  Synthetic Construct
GLECDGKVNLCCKKQNFVSFKDIGWNDWIIAPSGYHANECSGKCPSHIAGTSGSSLSFHSTVINHYRMRGHSPFSDMGACCIPTKLRPMSMLYYDDGQNIIKKDIQNMIVEECGCS

SEQ ID NO: 46 - protein, 116 aa, Artificial Sequence
  Synthetic Construct
GLECDGKVNTCCKKQNFVSFKDIGWNDWIIAPSGYHANECSGKCPSHIAGTSGSSLSFHSTVINHYRMRGHSPFANMGACCIPTKLRPMSMLYYDDGQNIIKKDIQNMIVEECGCS

SEQ ID NO: 47 - protein, 116 aa, Artificial Sequence
  Synthetic Construct
GLECDGKVNLCCKKQNFVSFKDIGWNDWIIAPSGYHANECGGLCPSHIAGTSGSSLSFHSTVINHYRMRGHSPHANRGACCIPTKLRPMSMLYYDDGQNIIKKDIQNMIVEECGCS

SEQ ID NO: 48 - protein, 116 aa, Artificial Sequence
  Synthetic Construct
GLECDGKVNYCCKKQNFVSFKDIGWNDWIIAPSGYHANECSGKCPSHIAGTSGSSLSFHSTVINHYRMRGHSPFSDMGSCCIPTKLRPMSMLYYDDGQNIIKKDIQNMIVEECGCS

SEQ ID NO: 49 - protein, 116 aa, Artificial Sequence
  Synthetic Construct
GLECDGKVNLCCKKQNFVSFKDIGWNDWIIAPSGYHANECSGLCPSHIAGTSGSSLSFHSTVINHYRMRGHSPFANMGACCIPTKLRPMSMLYYDDGQNIIKKDIQNMIVEECGCS

SEQ ID NO: 50 - protein, 116 aa, Artificial Sequence
  Synthetic Construct
GLECDGKVNLCCKKQDFVSFKDIGWNDWIIAPSGYHANRCDGLCPSHIAGTSGSSLSFHSTVINHYRMRGHSPFSDMGSCCVPTKLRPMSMLYYDDGQNIIKKDIQNMIVEECGCS

SEQ ID NO: 51 - protein, 116 aa, Artificial Sequence
  Synthetic Construct
GLECDGKVNICCKKQLFGRTKDIGWNDWIIAPSGYHGGGCSGECPSHIAGTSGSSLSFHSTVINHYRMRGHSPVANLKSCCSPTKLRPMSMLYYDDGQNIIKKDIQNMKVEECGCT

SEQ ID NO: 52 - protein, 116 aa, Artificial Sequence
  Synthetic Construct
GLECDGKVNICCKKQSFGQTKDIGWNDWIIAPSGYHGGGCSGECPSHIAGTSGSSLSFHSTVINHYRMRGHSPFANLKSCCSPTKLRPMSMLYYDDGQNIIKKDIQRMVVEECGCT

SEQ ID NO: 53 - protein, 116 aa, Artificial Sequence
  Synthetic Construct
GLECDGKVNICCKKQLFGKTKDIGWNDWIIAPSGYHGGSCTGECPSHIAGTSGSSLSFHSTVINHYRMRGHSPNANLKSCCSPTKLRPMSMLYYDDGQNIIKKDIQGMKVEECGCT

SEQ ID NO: 54 - protein, 116 aa, Artificial Sequence
  Synthetic Construct
GLECDGKVNICCKKQEFGQAKDIGWNDWIIAPSGYHGGGCSGECPSHIAGTSGSSLSFHSTVINHYRMRGHSPWANLKSCCSPTKLRPMSMLYYDDGQNIIKKDIQGMKVEECGCT

SEQ ID NO: 55 - protein, 116 aa, Artificial Sequence
  Synthetic Construct
GLECDGKVNICCKKQSFAQTKDIGWNDWIIAPSGYHGGGCTGECPSHIAGTSGSSLSFHSTVINHYRMRGHSPFANLKSCCSPTKLRPMSMLYYDDGQNIIKKDIQNMVVEECGCT

SEQ ID NO: 56 - protein, 116 aa, Artificial Sequence
  Synthetic Construct
GLECDGKVNICCKKQSFGQTKDIGWNDWIIAPSGYHGGGCTGECPSHIAGTSGSSLSFHSTVINHYRMRGHSPWANLKSCCSPTKLRPMSMLYYDDGQNIIKKDIQGMVVEECGCT

SEQ ID NO: 57 - protein, 116 aa, Artificial Sequence
  Synthetic Construct
GLECDGKVNICCKKQSFGQAKDIGWNDWIIAPSGYHGGSCTGECPSHIAGTSGSSLSFHSTVINHYRMRGHSPFANLKSCCAPTKLRPMSMLYYDDGQNIIKKDIQNMVVEECGCV

SEQ ID NO: 58 - protein, 116 aa, Artificial Sequence
  Synthetic Construct
GLECDGKVNICCKKQSFGQAKDIGWNDWIIAPSGYHGGGCSGECPSHIAGTSGSSLSFHSTVINHYRMRGHSPWANLKSCCSPTKLRPMSMLYYDDGQNIIKKDIQNMKVEECGCT

SEQ ID NO: 59 - protein, 116 aa, Artificial Sequence
  Synthetic Construct
GLECDGKVNICCKKQSFGQTKDIGWNDWIIAPSGYHGGGCTGECPSHIAGTSGSSLSFHSTVINHYRMRGHSPWANLKSCCSPTKLRPMSMLYYDDGQNIIKKDIQNMKVEECGCT

SEQ ID NO: 60 - protein, 116 aa, Artificial Sequence
  Synthetic Construct
GLECDGKVNICCKKQLFGQTKDIGWNDWIIAPSGYHGGGCTGECPSHIAGTSGSSLSFHSTVINHYRMRGHSPNANLKSCCAPTKLRPMSMLYYDDGQNIIKKDIQGMKVEECGCV

SEQ ID NO: 61 - protein, 116 aa, Artificial Sequence
  Synthetic Construct
GLECDGKVNICCKKQSFGQTKDIGWNDWIIAPSGYHGGGCSGECPSHIAGTSGSSLSFHSTVINHYRMRGHSPWANLKSCCAPTKLRPMSMLYYDDGQNIIKKDIQNMVVEECGCV

SEQ ID NO: 62 - protein, 116 aa, Artificial Sequence
  Synthetic Construct
GLECDGKVNICCKKQLFGQTKDIGWNDWIIAPSGYHGGSCTGECPSHIAGTSGSSLSFHSTVINHYRMRGHSPNANLKSCCSPTKLRPMSMLYYDDGQNIIKKDIQNMKVEECGCT

SEQ ID NO: 63 - protein, 116 aa, Artificial Sequence
  Synthetic Construct
GLECDGKVNICCKKQSFGQAKDIGWNDWIIAPSGYHGGGCTGECPSHIAGTSGSSLSFHSTVINHYRMRGHSPWANLKSCCSPTKLRPMSMLYYDDGQNIIKKDIQNMVVEECGCT

SEQ ID NO: 64 - protein, 116 aa, Artificial Sequence
  Synthetic Construct
GLECDGKVNICCKKQSFSQAKDIGWNDWIIAPSGYHGGGCTGECPSHIAGTSGSSLSFHSTVINHYRMRGHSPFANLKSCCSPTKLRPMSMLYYDDGQNIIKKDIQGMVVEECGCT

SEQ ID NO: 65 - protein, 116 aa, Artificial Sequence
  Synthetic Construct
GLECDGKVNICCKKQSFGQTKDIGWNDWIIAPSGYHGGGCSGECPSHIAGTSGSSLSFHSTVINHYRMRGHSPWANLKSCCSPTKLRPMSMLYYDDGQNIIKKDIQGMVVEECGCT

SEQ ID NO: 66 - protein, 116 aa, Artificial Sequence
  Synthetic Construct
GLECDGKVNICCKKQSFGQTKDIGWNDWIIAPSGYHGGSCSGECPSHIAGTSGSSLSFHSTVINHYRMRGHSPFANLKSCCSPTKLRPMSMLYYDDGQNIIKKDIQGMKVEECGCT

SEQ ID NO: 67 - protein, 116 aa, Artificial Sequence
  Synthetic Construct
GLECDGKVNICCKKQMFGQAKDIGWNDWIIAPSGYHGGGCTGECPSHIAGTSGSSLSFHSTVINHYRMRGHSPWANLKSCCSPTKLRPMSMLYYDDGQNIIKKDIQNMVVEECGCT

SEQ ID NO: 68 - protein, 116 aa, Artificial Sequence
  Synthetic Construct
GLECDGKVNICCKKQSFGQTKDIGWNDWIIAPSGYHGGGCSGECPSHIAGTSGSSLSFHSTVINHYRMRGHSPWANLKSCCSPTKLRPMSMLYYDDGQNIIKKDIQNMKVEECGCT

SEQ ID NO: 69 - protein, 116 aa, Artificial Sequence
  Synthetic Construct
GLECDGKVNICCKKQSFGKAKDIGWNDWIIAPSGYHGGGCTGECPSHIAGTSGSSLSFHSTVINHYRMRGHSPFANLKSCCSPTKLRPMSMLYYDDGQNIIKKDIQGMKVEECGCT

SEQ ID NO: 70 - protein, 116 aa, Artificial Sequence
  Synthetic Construct
GLECDGKVNICCKKQSFGKTKDIGWNDWIIAPSGYHGGGCTGECPSHIAGTSGSSLSFHSTVINHYRMRGHSPFANLKSCCSPTKLRPMSMLYYDDGQNIIKKDIQQMVVEECGCT

SEQ ID NO: 71 - protein, 116 aa, Artificial Sequence
  Synthetic Construct
GLECDGKVNICCKKQLFGQAKDIGWNDWIIAPSGYHGGGCSGECPSHIAGTSGSSLSFHSTVINHYRMRGHSPVANLKSCCSPTKLRPMSMLYYDDGQNIIKKDIQGMVVEECGCT

SEQ ID NO: 72 - protein, 116 aa, Artificial Sequence
  Synthetic Construct
GLECDGKVNICCKKQSFGQAKDIGWNDWIIAPSGYHGGGCTGECPSHIAGTSGSSLSFHSTVINHYRMRGHSPFANLKSCCSPTKLRPMSMLYYDDGQNIIKKDIQNMKVEECGCT

SEQ ID NO: 73 - protein, 116 aa, Artificial Sequence
  Synthetic Construct
GLECDGKVNICCKKQLFGQAKDIGWNDWIIAPSGYHGGSCTGECPSHIAGTSGSSLSFHSTVINHYRMRGHSPNANLKSCCSPTKLRPMSMLYYDDGQNIIKKDIQNMVVEECGCT

SEQ ID NO: 74 - protein, 116 aa, Artificial Sequence
  Synthetic Construct
GLECDGKVNICCKKQSFGQAKDIGWNDWIIAPSGYHGGGCSGECPSHIAGTSGSSLSFHSTVINHYRMRGHSPFANLKSCCSPTKLRPMSMLYYDDGQNIIKKDIQGMVVEECGCT

SEQ ID NO: 75 - protein, 116 aa, Artificial Sequence
  Synthetic Construct
GLECDGKVNICCKKQSFGRAKDIGWNDWIIAPSGYHGGGCTGECPSHIAGTSGSSLSFHSTVINHYRMRGHSPFANLKSCCSPTKLRPMSMLYYDDGQNIIKKDIQGMVVEECGCT

SEQ ID NO: 76 - protein, 116 aa, Artificial Sequence
  Synthetic Construct
GLECDGKVNICCKKQSFGQAKDIGWNDWIIAPSGYHGGGCSGECPSHIAGTSGSSLSFHSTVINHYRMRGHSPWANLKSCCSPTKLRPMSMLYYDDGQNIIKKDIQRMKVEECGCT

SEQ ID NO: 77 - protein, 116 aa, Artificial Sequence
  Synthetic Construct
GLECDGKVNICCKKQLFGQTKDIGWNDWIIAPSGYHGGGCSGECPSHIAGTSGSSLSFHSTVINHYRMRGHSPNANLKSCCSPTKLRPMSMLYYDDGQNIIKKDIQGMKVEECGCT

SEQ ID NO: 78 - protein, 116 aa, Artificial Sequence
  Synthetic Construct
GLECDGKVNICCKKQMFGKAKDIGWNDWIIAPSGYHGGGCTGECPSHIAGTSGSSLSFHSTVINHYRMRGHSPWANLKSCCSPTKLRPMSMLYYDDGQNIIKKDIQNMVVEECGCT

SEQ ID NO: 79 - protein, 116 aa, Artificial Sequence
  Synthetic Construct
GLECDGKVNICCKKQLFGQAKDIGWNDWIIAPSGYHGGGCSGECPSHIAGTSGSSLSFHSTVINHYRMRGHSPVANLKSCCSPTKLRPMSMLYYDDGQNIIKKDIQNMVVEECGCT

SEQ ID NO: 80 - protein, 116 aa, Artificial Sequence
  Synthetic Construct
GLECDGKVNICCKKQSFGQAKDIGWNDWIIAPSGYHGGGCTGECPSHIAGTSGSSLSFHSTVINHYRMRGHSPWANLKSCCSPTKLRPMSMLYYDDGQNIIKKDIQRMVVEECGCT

SEQ ID NO: 81 - protein, 116 aa, Artificial Sequence
  Synthetic Construct
GLECDGKVNICCKKQSFGQTKDIGWNDWIIAPSGYHGGGCSGECPSHIAGTSGSSLSFHSTVINHYRMRGHSPWANLKSCCAPTKLRPMSMLYYDDGQNIIKKDIQNMKVEECGCV

SEQ ID NO: 82 - protein, 116 aa, Artificial Sequence
  Synthetic Construct
GLECDGKVNICCKKQLFGKTKDIGWNDWIIAPSGYHGGGCTGECPSHIAGTSGSSLSFHSTVINHYRMRGHSPVANLKSCCSPTKLRPMSMLYYDDGQNIIKKDIQRMVVEECGCT

SEQ ID NO: 83 - protein, 116 aa, Artificial Sequence
  Synthetic Construct
GLECDGKVNICCKKQSFGQTKDIGWNDWIIAPSGYHGGGCTGECPSHIAGTSGSSLSFHSTVINHYRMRGHSPWANLKSCCSPTKLRPMSMLYYDDGQNIIKKDIQGMKVEECGCT

SEQ ID NO: 84 - protein, 116 aa, Artificial Sequence
  Synthetic Construct
GLECDGKVNICCKKQSFGRAKDIGWNDWIIAPSGYHGGGCTGECPSHIAGTSGSSLSFHSTVINHYRMRGHSPFANLKSCCSPTKLRPMSMLYYDDGQNIIKKDIQGMKVEECGCT

SEQ ID NO: 85 - protein, 116 aa, Artificial Sequence
  Synthetic Construct
GLECDGKVNICCKKQSFGQAKDIGWNDWIIAPSGYHGGGCSGECPSHIAGTSGSSLSFHSTVINHYRMRGHSPFANLKSCCSPTKLRPMSMLYYDDGQNIIKKDIQNMVVEECGCT

SEQ ID NO: 86 - protein, 116 aa, Artificial Sequence
  Synthetic Construct
GLECDGKVNICCKKQLFGQAKDIGWNDWIIAPSGYHGGGCTGECPSHIAGTSGSSLSFHSTVINHYRMRGHSPVANLKSCCSPTKLRPMSMLYYDDGQNIIKKDIQNMKVEECGCT

SEQ ID NO: 87 - protein, 116 aa, Artificial Sequence
  Synthetic Construct
GLECDGKVNICCKKQSFGKTKDIGWNDWIIAPSGYHGGGCSGECPSHIAGTSGSSLSFHSTVINHYRMRGHSPWANLKSCCSPTKLRPMSMLYYDDGQNIIKKDIQNMVVEECGCT

SEQ ID NO: 88 - protein, 116 aa, Artificial Sequence
  Synthetic Construct
GLECDGKVNICCKKQSFGQAKDIGWNDWIIAPSGYHGGSCSGECPSHIAGTSGSSLSFHSTVINHYRMRGHSPFANLKSCCSPTKLRPMSMLYYDDGQNIIKKDIQNMVVEECGCT

SEQ ID NO: 89 - protein, 116 aa, Artificial Sequence
  Synthetic Construct
GLECDGKVNICCKKQSFGQTKDIGWNDWIIAPSGYHGGGCSGECPSHIAGTSGSSLSFHSTVINHYRMRGHSPWANLKSCCSPTKLRPMSMLYYDDGQNIIKKDIQNMVVEECGCT

SEQ ID NO: 90 - protein, 116 aa, Artificial Sequence
  Synthetic Construct
GLECDGKVNICCKKQLFGQTKDIGWNDWIIAPSGYHGGGCSGECPSHIAGTSGSSLSFHSTVINHYRMRGHSPNANLKSCCSPTKLRPMSMLYYDDGQNIIKKDIQNMKVEECGCT

SEQ ID NO: 91 - protein, 116 aa, Artificial Sequence
  Synthetic Construct
GLECDGKVNICCKKQSFGQTKDIGWNDWIIAPSGYHGGSCSGECPSHIAGTSGSSLSFHSTVINHYRMRGHSPFANLKSCCSPTKLRPMSMLYYDDGQNIIKKDIQNMKVEECGCT

SEQ ID NO: 92 - protein, 116 aa, Artificial Sequence
  Synthetic Construct
GLECDGKVNICCKKQSFGRTKDIGWNDWIIAPSGYHGGGCSGECPSHIAGTSGSSLSFHSTVINHYRMRGHSPWANLKSCCSPTKLRPMSMLYYDDGQNIIKKDIQNMVVEECGCT

SEQ ID NO: 93 - protein, 116 aa, Artificial Sequence
  Synthetic Construct
GLECDGKVNICCKKQSFGQAKDIGWNDWIIAPSGYHGGGCSGECPSHIAGTSGSSLSFHSTVINHYRMRGHSPFANLKSCCSPTKLRPMSMLYYDDGQNIIKKDIQGMKVEECGCT

SEQ ID NO: 94 - protein, 116 aa, Artificial Sequence
  Synthetic Construct
GLECDGKVNICCKKQSFGQTKDIGWNDWIIAPSGYHGGGCTGECPSHIAGTSGSSLSFHSTVINHYRMRGHSPFANLKSCCSPTKLRPMSMLYYDDGQNIIKKDIQNMKVEECGCT

SEQ ID NO: 95 - protein, 116 aa, Artificial Sequence
  Synthetic Construct
GLECDGKVNICCKKQSFGQAKDIGWNDWIIAPSGYHGGGCTGECPSHIAGTSGSSLSFHSTVINHYRMRGHSPWANLKSCCSPTKLRPMSMLYYDDGQNIIKKDIQRMVAEECGCT

SEQ ID NO: 96 - DNA, 930 bp, Artificial Sequence
  SYNTHETIC CONSTRUCT
atgcccttgctttggctgagaggatttctgttggcaagttgctggattatagtgaggagttcccccaccccaggatccgaggggcacagcgcggcccccgactgtccgtcctgtgcgctggccgccctcccaaaggatgtacccaactctcagccagagatggtggaggccgtcaagaagcacattttaaacatgctgcacttgaagaagagacccgatgtcacccagccggtacccaaggcggcgcttctgaacgcgatcagaaagcttcatgtgggcaaagtcggggagaacgggtatgtggagatagaggatgacattggaaggagggcagaaatgaatgaacttatggagcagacctcggagatcatcacgtttgccgagtcaggaacagccaggaagacgctgcacttcgagatttccaaggaaggcagtgacctgtcagtggtggagcgtgcagaagtctggctcttcctaaaagtccccaaggccaacaggaccaggaccaaagtcaccatccgcctcttccagcagcagaagcacccgcagggcagcttggacacaggggaagaggccgaggaagtgggcttaaagggggagaggagtgaactgttgctctctgaaaaagtagtagacgctcggaagagcacctggcatgtcttccctgtctccagcagcatccagcggttgctggaccagggcaagagctccctggacgttcggattgcctgtgagcagtgccaggagagtggcgccagcttggttctcctgggcaagaagaagaagaaagaagaggagggggaagggaaaaagaagggcggaggtgaaggtggggcaggagcagatgaggaaaaggagcagtcgcacagacctttcctcatgctgcaggcccggcagtctgaagaccaccctcatcgccggcgtcggcgg

SEQ ID NO: 97 - DNA, 351 bp, Artificial Sequence
  SYNTHETIC CONSTRUCT
ggcctggagtgcgacggcaaggtgaacatctgctgcaagaagcagttcttcgtgagcttcaaggacatcggctggaacgactggatcatcgcccccagcggctaccacgccaactactgcgagggcgagtgccccagccacatcgccggcaccagcggcagcagcctgagcttccacagcaccgtgatcaaccactacaggatgaggggccacagccccttcgccaacctgaagagctgctgcgtgcccaccaagctgaggcccatgagcatgctgtactacgacgacggccagaacatcatcaagaaggacatccagaacatgatcgtggaggagtgcggctgcagctaa

SEQ ID NO: 98 - DNA, 351 bp, Artificial Sequence
  SYNTHETIC CONSTRUCT
ggcctggagtgcgacggcaaggtgaacctgtgctgcaagaagcagaacttcgtgagcttcaaggacatcggctggaacgactggatcatcgcccccagcggctaccacgccaacgagtgcaccggcaagtgccccagccacatcgccggcaccagcggcagcagcctgagcttccacagcaccgtgatcaaccactacaggatgaggggccacagccccttcagcgacctgggcagctgctgcatccccaccaagctgaggcccatgagcatgctgtactacgacgacggccagaacatcatcaagaaggacatccagaacatgatcgtggaggagtgcggctgcagctaa

SEQ ID NO: 99 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcagcggcaagtgccccagccacatcgccggcaccagcggcagcagcctgagcttccacagcaccgtgatcaaccactacaggatgaggggccacagccccttcagcaacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 100 - DNA, 351 bp, Artificial Sequence
  SYNTHETIC CONSTRUCT
ggcctggagtgcgacggcaaggtgaacctgtgctgcaagaagcagctgttcgtgagcttcaaggacatcggctggaacgactggatcatcgcccccagcggctaccacgccaaccactgcagcggcctgtgccccagccacatcgccggcaccagcggcagcagcctgagcttccacagcaccgtgatcaaccactacaggatgaggggccacagccccttcagcgacatgggcgcctgctgcgtgcccaccaagctgaggcccatgagcatgctgtactacgacgacggccagaacatcatcaagaaggacatccagaacatgatcgtggaggagtgcggctgcagctaa

SEQ ID NO: 101 - DNA, 351 bp, Artificial Sequence
  SYNTHETIC CONSTRUCT
ggcctggagtgcgacggcaaggtgaactactgctgcaagaagcagaacttcgtgagcttcaaggacatcggctggaacgactggatcatcgcccccagcggctaccacgccaacgagtgcagcggcaagtgccccagccacatcgccggcaccagcggcagcagcctgagcttccacagcaccgtgatcaaccactacaggatgaggggccacagccccttcagcaacctgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 102 - DNA, 351 bp, Artificial Sequence
  SYNTHETIC CONSTRUCT
ggcctggagtgcgacggcaaggtgaacctgtgctgcaagaagcagtggttcgtgagcttcaaggacatcggctggaacgactggatcatcgcccccagcggctaccacgccaacaggtgcagcggcaagtgccccagccacatcgccggcaccagcggcagcagcctgagcttccacagcaccgtgatcaaccactacaggatgaggggccacagccccttcgccgacatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 103 - DNA, 351 bp, Artificial Sequence
  SYNTHETIC CONSTRUCT
ggcctggagtgcgacggcaaggtgaactactgctgcaagaagcagcacttcgtgagcttcaaggacatcggctggaacgactggatcatcgcccccagcggctaccacgccaacagctgcagcggcctgtgccccagccacatcgccggcaccagcggcagcagcctgagcttccacagcaccgtgatcaaccactacaggatgaggggccacagccccttcagccagatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 104 - DNA, 351 bp, Artificial Sequence
  SYNTHETIC CONSTRUCT
ggcctggagtgcgacggcaaggtgaactactgctgcaagaagcaggacttcgtgagcttcaaggacatcggctggaacgactggatcatcgcccccagcggctaccacgccaacaagtgcagcggcaagtgccccagccacatcgccggcaccagcggcagcagcctgagcttccacagcaccgtgatcaaccactacaggatgaggggccacagccccttcagcgacatgggcagctgctgcgtgcccaccaagctgaggcccatgagcatgctgtactacgacgacggccagaacatcatcaagaaggacatccagaacatgatcgtggaggagtgcggctgcagctaa

SEQ ID NO: 105 - DNA, 351 bp, Artificial Sequence
  SYNTHETIC CONSTRUCT
ggcctggagtgcgacggcaaggtgaactactgctgcaagaagcaggacttcgtgagcttcaaggacatcggctggaacgactggatcatcgcccccagcggctaccacgccaacaagtgcaccggcaagtgccccagccacatcgccggcaccagcggcagcagcctgagcttccacagcaccgtgatcaaccactacaggatgaggggccacagccccttcgccgacctgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 106 - DNA, 351 bp, Artificial Sequence
  SYNTHETIC CONSTRUCT
ggcctggagtgcgacggcaaggtgaactactgctgcaagaagcaggacttcgtgagcttcaaggacatcggctggaacgactggatcatcgcccccagcggctaccacgccaacaggtgcagcggcctgtgccccagccacatcgccggcaccagcggcagcagcctgagcttccacagcaccgtgatcaaccactacaggatgaggggccacagccccttcgcccagatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 107 - DNA, 351 bp, Artificial Sequence
  SYNTHETIC CONSTRUCT
ggcctggagtgcgacggcaaggtgaacctgtgctgcaagaagcagctgttcgtgagcttcaaggacatcggctggaacgactggatcatcgcccccagcggctaccacgccaaccactgcagcggcctgtgccccagccacatcgccggcaccagcggcagcagcctgagcttccacagcaccgtgatcaaccactacaggatgaggggccacagccccttcgcccagatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 108 - DNA, 351 bp, Artificial Sequence
  SYNTHETIC CONSTRUCT
ggcctggagtgcgacggcaaggtgaactactgctgcaagaagcagaacttcgtgagcttcaaggacatcggctggaacgactggatcatcgcccccagcggctaccacgccaacgagtgcagcggcctgtgccccagccacatcgccggcaccagcggcagcagcctgagcttccacagcaccgtgatcaaccactacaggatgaggggccacagccccttcagccagatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 109 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaagtgcagcggcaagtgccccagccacatcgccggcaccagcggcagcagcctgagcttccacagcaccgtgatcaaccactacaggatgaggggccacagccccttcagcgacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 110 - DNA, 351 bp, Artificial Sequence
  SYNTHETIC CONSTRUCT
ggcctggagtgcgacggcaaggtgaacctgtgctgcaagaagcagcacttcgtgagcttcaaggacatcggctggaacgactggatcatcgcccccagcggctaccacgccaacaggtgcgacggcctgtgccccagccacatcgccggcaccagcggcagcagcctgagcttccacagcaccgtgatcaaccactacaggatgaggggccacagccccttcgcccagatgggcagctgctgcgtgcccaccaagctgaggcccatgagcatgctgtactacgacgacggccagaacatcatcaagaaggacatccagaacatgatcgtggaggagtgcggctgcagctaa

SEQ ID NO: 111 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcagcggcctgtgccccagccacatcgccggcaccagcggcagcagcctgagcttccacagcaccgtgatcaaccactacaggatgaggggccacagccccttcagcaacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 112 - DNA, 351 bp, Artificial Sequence
  SYNTHETIC CONSTRUCT
ggcctggagtgcgacggcaaggtgaacctgtgctgcaagaagcagctgttcgtgagcttcaaggacatcggctggaacgactggatcatcgcccccagcggctaccacgccaaccactgcagcggcctgtgccccagccacatcgccggcaccagcggcagcagcctgagcttccacagcaccgtgatcaaccactacaggatgaggggccacagccccttcagccagatgggcgcctgctgcgtgcccaccaagctgaggcccatgagcatgctgtactacgacgacggccagaacatcatcaagaaggacatccagaacatgatcgtggaggagtgcggctgcagctaa

SEQ ID NO: 113 - DNA, 351 bp, Artificial Sequence
  SYNTHETIC CONSTRUCT
ggcctggagtgcgacggcaaggtgaactactgctgcaagaagcaggacttcgtgagcttcaaggacatcggctggaacgactggatcatcgcccccagcggctaccacgccaacaagtgcggcggcaagtgccccagccacatcgccggcaccagcggcagcagcctgagcttccacagcaccgtgatcaaccactacaggatgaggggccacagccccttcagcgacatgggcgcctgctgcgtgcccaccaagctgaggcccatgagcatgctgtactacgacgacggccagaacatcatcaagaaggacatccagaacatgatcgtggaggagtgcggctgcagctaa

SEQ ID NO: 114 - DNA, 351 bp, Artificial Sequence
  SYNTHETIC CONSTRUCT
ggcctggagtgcgacggcaaggtgaactactgctgcaagaagcagaacttcgtgagcttcaaggacatcggctggaacgactggatcatcgcccccagcggctaccacgccaacaagtgcagcggcaagtgccccagccacatcgccggcaccagcggcagcagcctgagcttccacagcaccgtgatcaaccactacaggatgaggggccacagccccttcagcgacatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 115 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaagtgcggcggcaagtgccccagccacatcgccggcaccagcggcagcagcctgagcttccacagcaccgtgatcaaccactacaggatgaggggccacagccccttcgccaacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 116 - DNA, 351 bp, Artificial Sequence
  SYNTHETIC CONSTRUCT
ggcctggagtgcgacggcaaggtgaacctgtgctgcaagaagcagaacttcgtgagcttcaaggacatcggctggaacgactggatcatcgcccccagcggctaccacgccaacaagtgcggcggcctgtgccccagccacatcgccggcaccagcggcagcagcctgagcttccacagcaccgtgatcaaccactacaggatgaggggccacagccccttcgcccagatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 117 - DNA, 351 bp, Artificial Sequence
  SYNTHETIC CONSTRUCT
ggcctggagtgcgacggcaaggtgaactactgctgcaagaagcagtggttcgtgagcttcaaggacatcggctggaacgactggatcatcgcccccagcggctaccacgccaacaagtgcagcggcaagtgccccagccacatcgccggcaccagcggcagcagcctgagcttccacagcaccgtgatcaaccactacaggatgaggggccacagccccttcgccaacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 118 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcgacggcctgtgccccagccacatcgccggcaccagcggcagcagcctgagcttccacagcaccgtgatcaaccactacaggatgaggggccacagccccttcgccctgatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 119 - DNA, 351 bp, Artificial Sequence
  SYNTHETIC CONSTRUCT
ggcctggagtgcgacggcaaggtgaactactgctgcaagaagcaggacttcgtgagcttcaaggacatcggctggaacgactggatcatcgcccccagcggctaccacgccaacaagtgcgacggcaagtgccccagccacatcgccggcaccagcggcagcagcctgagcttccacagcaccgtgatcaaccactacaggatgaggggccacagccccttcagcgacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 120 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcagcggcaggtgccccagccacatcgccggcaccagcggcagcagcctgagcttccacagcaccgtgatcaaccactacaggatgaggggccacagccccttcagcgacatgggcagctgctgcgtgcccaccaagctgaggcccatgagcatgctgtactacgacgacggccagaacatcatcaagaaggacatccagaacatgatcgtggaggagtgcggctgcagctaa

SEQ ID NO: 121 - DNA, 351 bp, Artificial Sequence
  SYNTHETIC CONSTRUCT
ggcctggagtgcgacggcaaggtgaacctgtgctgcaagaagcagaacttcgtgagcttcaaggacatcggctggaacgactggatcatcgcccccagcggctaccacgccaacgagtgcagcggcctgtgccccagccacatcgccggcaccagcggcagcagcctgagcttccacagcaccgtgatcaaccactacaggatgaggggccacagccccttcagccagatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 122 - DNA, 351 bp, Artificial Sequence
  SYNTHETIC CONSTRUCT
ggcctggagtgcgacggcaaggtgaacctgtgctgcaagaagcagcacttcgtgagcttcaaggacatcggctggaacgactggatcatcgcccccagcggctaccacgccaacaggtgcgacggcaagtgccccagccacatcgccggcaccagcggcagcagcctgagcttccacagcaccgtgatcaaccactacaggatgaggggccacagccccttcgccaacatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 123 - DNA, 351 bp, Artificial Sequence
  SYNTHETIC CONSTRUCT
ggcctggagtgcgacggcaaggtgaacctgtgctgcaagaagcagctgttcgtgagcttcaaggacatcggctggaacgactggatcatcgcccccagcggctaccacgccaaccactgcgacggcaagtgccccagccacatcgccggcaccagcggcagcagcctgagcttccacagcaccgtgatcaaccactacaggatgaggggccacagccccttcgccaacaggggcgcctgctgcatccccaccaagctgaggcccatgagcatgctgtactacgacgacggccagaacatcatcaagaaggacatccagaacatgatcgtggaggagtgcggctgcagctaa

SEQ ID NO: 124 - DNA, 351 bp, Artificial Sequence
  SYNTHETIC CONSTRUCT
ggcctggagtgcgacggcaaggtgaactactgctgcaagaagcagctgttcgtgagcttcaaggacatcggctggaacgactggatcatcgcccccagcggctaccacgccaaccactgcagcggcaagtgccccagccacatcgccggcaccagcggcagcagcctgagcttccacagcaccgtgatcaaccactacaggatgaggggccacagccccttcgccaacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 125 - DNA, 351 bp, Artificial Sequence
  SYNTHETIC CONSTRUCT
ggcctggagtgcgacggcaaggtgaacctgtgctgcaagaagcagctgttcgtgagcttcaaggacatcggctggaacgactggatcatcgcccccagcggctaccacgccaaccactgcagcggcaagtgccccagccacatcgccggcaccagcggcagcagcctgagcttccacagcaccgtgatcaaccactacaggatgaggggccacagccccttcagcgacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 126 - DNA, 351 bp, Artificial Sequence
  SYNTHETIC CONSTRUCT
ggcctggagtgcgacggcaaggtgaactactgctgcaagaagcagaacttcgtgagcttcaaggacatcggctggaacgactggatcatcgcccccagcggctaccacgccaacaagtgcagcggcctgtgccccagccacatcgccggcaccagcggcagcagcctgagcttccacagcaccgtgatcaaccactacaggatgaggggccacagccccttcagcaagatgggcgcctgctgcgtgcccaccaagctgaggcccatgagcatgctgtactacgacgacggccagaacatcatcaagaaggacatccagaacatgatcgtggaggagtgcggctgcagctaa

SEQ ID NO: 127 - DNA, 351 bp, Artificial Sequence
  SYNTHETIC CONSTRUCT
ggcctggagtgcgacggcaaggtgaacacctgctgcaagaagcagctgttcgtgagcttcaaggacatcggctggaacgactggatcatcgcccccagcggctaccacgccaaccactgcagcggcaagtgccccagccacatcgccggcaccagcggcagcagcctgagcttccacagcaccgtgatcaaccactacaggatgaggggccacagccccttcagcgacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 128 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcggcggcaagtgccccagccacatcgccggcaccagcggcagcagcctgagcttccacagcaccgtgatcaaccactacaggatgaggggccacagccccttcagcaacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 129 - DNA, 351 bp, Artificial Sequence
  SYNTHETIC CONSTRUCT
ggcctggagtgcgacggcaaggtgaacctgtgctgcaagaagcagaacttcgtgagcttcaaggacatcggctggaacgactggatcatcgcccccagcggctaccacgccaacgagtgcatgggcaagtgccccagccacatcgccggcaccagcggcagcagcctgagcttccacagcaccgtgatcaaccactacaggatgaggggccacagccccttcagcgacatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 130 - DNA, 351 bp, Artificial Sequence
  SYNTHETIC CONSTRUCT
ggcctggagtgcgacggcaaggtgaactactgctgcaagaagcagctgttcgtgagcttcaaggacatcggctggaacgactggatcatcgcccccagcggctaccacgccaaccactgcaccggcaagtgccccagccacatcgccggcaccagcggcagcagcctgagcttccacagcaccgtgatcaaccactacaggatgaggggccacagccccttcagcgacctgggcagctgctgcgtgcccaccaagctgaggcccatgagcatgctgtactacgacgacggccagaacatcatcaagaaggacatccagaacatgatcgtggaggagtgcggctgcagctaa

SEQ ID NO: 131 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcagcggcaagtgccccagccacatcgccggcaccagcggcagcagcctgagcttccacagcaccgtgatcaaccactacaggatgaggggccacagccccttcagcgacatgggcagctgctgcgtgcccaccaagctgaggcccatgagcatgctgtactacgacgacggccagaacatcatcaagaaggacatccagaacatgatcgtggaggagtgcggctgcagctaa

SEQ ID NO: 132 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcagcggcaagtgccccagccacatcgccggcaccagcggcagcagcctgagcttccacagcaccgtgatcaaccactacaggatgaggggccacagccccttcgccaacatgggcgcctgctgcgtgcccaccaagctgaggcccatgagcatgctgtactacgacgacggccagaacatcatcaagaaggacatccagaacatgatcgtggaggagtgcggctgcagctaa

SEQ ID NO: 133 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcagcggcaagtgccccagccacatcgccggcaccagcggcagcagcctgagcttccacagcaccgtgatcaaccactacaggatgaggggccacagccccttcagcgacatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 134 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaagtgcggcggcaagtgccccagccacatcgccggcaccagcggcagcagcctgagcttccacagcaccgtgatcaaccactacaggatgaggggccacagccccttcagccagctgggcgcctgctgcgtgcccaccaagctgaggcccatgagcatgctgtactacgacgacggccagaacatcatcaagaaggacatccagaacatgatcgtggaggagtgcggctgcagctaa

SEQ ID NO: 135 - DNA, 351 bp, Artificial Sequence
  SYNTHETIC CONSTRUCT
ggcctggagtgcgacggcaaggtgaacctgtgctgcaagaagcagaacttcgtgagcttcaaggacatcggctggaacgactggatcatcgcccccagcggctaccacgccaacgagtgcagcggcctgtgccccagccacatcgccggcaccagcggcagcagcctgagcttccacagcaccgtgatcaaccactacaggatgaggggccacagccccttcagcgacatgggcagctgctgcgtgcccaccaagctgaggcccatgagcatgctgtactacgacgacggccagaacatcatcaagaaggacatccagaacatgatcgtggaggagtgcggctgcagctaa

SEQ ID NO: 136 - DNA, 351 bp, Artificial Sequence
  SYNTHETIC CONSTRUCT
ggcctggagtgcgacggcaaggtgaacctgtgctgcaagaagcagctgttcgtgagcttcaaggacatcggctggaacgactggatcatcgcccccagcggctaccacgccaaccactgcgccggcctgtgccccagccacatcgccggcaccagcggcagcagcctgagcttccacagcaccgtgatcaaccactacaggatgaggggccacagccccttcagcaacatgggcagctgctgcgtgcccaccaagctgaggcccatgagcatgctgtactacgacgacggccagaacatcatcaagaaggacatccagaacatgatcgtggaggagtgcggctgcagctaa

SEQ ID NO: 137 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacagctgcagcggcctgtgccccagccacatcgccggcaccagcggcagcagcctgagcttccacagcaccgtgatcaaccactacaggatgaggggccacagccccttcagcgacaggggcgcctgctgcatccccaccaagctgaggcccatgagcatgctgtactacgacgacggccagaacatcatcaagaaggacatccagaacatgatcgtggaggagtgcggctgcagctaa

SEQ ID NO: 138 - DNA, 351 bp, Artificial Sequence
  SYNTHETIC CONSTRUCT
ggcctggagtgcgacggcaaggtgaactactgctgcaagaagcaggacttcgtgagcttcaaggacatcggctggaacgactggatcatcgcccccagcggctaccacgccaacaagtgcagcggcaagtgccccagccacatcgccggcaccagcggcagcagcctgagcttccacagcaccgtgatcaaccactacaggatgaggggccacagccccttcagcgacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 139 - DNA, 351 bp, Artificial Sequence
  SYNTHETIC CONSTRUCT
ggcctggagtgcgacggcaaggtgaactactgctgcaagaagcaggacttcgtgagcttcaaggacatcggctggaacgactggatcatcgcccccagcggctaccacgccaacaggtgcgacggcaagtgccccagccacatcgccggcaccagcggcagcagcctgagcttccacagcaccgtgatcaaccactacaggatgaggggccacagccccttcagcgacatgggcgcctgctgcgtgcccaccaagctgaggcccatgagcatgctgtactacgacgacggccagaacatcatcaagaaggacatccagaacatgatcgtggaggagtgcggctgcagctaa

SEQ ID NO: 140 - DNA, 351 bp, Artificial Sequence
  SYNTHETIC CONSTRUCT
ggcctggagtgcgacggcaaggtgaacctgtgctgcaagaagcagaacttcgtgagcttcaaggacatcggctggaacgactggatcatcgcccccagcggctaccacgccaacgagtgcagcggcaagtgccccagccacatcgccggcaccagcggcagcagcctgagcttccacagcaccgtgatcaaccactacaggatgaggggccacagccccttcagcgacatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 141 - DNA, 351 bp, Artificial Sequence
  SYNTHETIC CONSTRUCT
ggcctggagtgcgacggcaaggtgaacacctgctgcaagaagcagaacttcgtgagcttcaaggacatcggctggaacgactggatcatcgcccccagcggctaccacgccaacgagtgcagcggcaagtgccccagccacatcgccggcaccagcggcagcagcctgagcttccacagcaccgtgatcaaccactacaggatgaggggccacagccccttcgccaacatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 142 - DNA, 351 bp, Artificial Sequence
  SYNTHETIC CONSTRUCT
ggcctggagtgcgacggcaaggtgaacctgtgctgcaagaagcagaacttcgtgagcttcaaggacatcggctggaacgactggatcatcgcccccagcggctaccacgccaacgagtgcggcggcctgtgccccagccacatcgccggcaccagcggcagcagcctgagcttccacagcaccgtgatcaaccactacaggatgaggggccacagcccccacgccaacaggggcgcctgctgcatccccaccaagctgaggcccatgagcatgctgtactacgacgacggccagaacatcatcaagaaggacatccagaacatgatcgtggaggagtgcggctgcagctaa

SEQ ID NO: 143 - DNA, 351 bp, Artificial Sequence
  SYNTHETIC CONSTRUCT
ggcctggagtgcgacggcaaggtgaactactgctgcaagaagcagaacttcgtgagcttcaaggacatcggctggaacgactggatcatcgcccccagcggctaccacgccaacgagtgcagcggcaagtgccccagccacatcgccggcaccagcggcagcagcctgagcttccacagcaccgtgatcaaccactacaggatgaggggccacagccccttcagcgacatgggcagctgctgcatccccaccaagctgaggcccatgagcatgctgtactacgacgacggccagaacatcatcaagaaggacatccagaacatgatcgtggaggagtgcggctgcagctaa

SEQ ID NO: 144 - DNA, 351 bp, Artificial Sequence
  SYNTHETIC CONSTRUCT
ggcctggagtgcgacggcaaggtgaacctgtgctgcaagaagcagaacttcgtgagcttcaaggacatcggctggaacgactggatcatcgcccccagcggctaccacgccaacgagtgcagcggcctgtgccccagccacatcgccggcaccagcggcagcagcctgagcttccacagcaccgtgatcaaccactacaggatgaggggccacagccccttcgccaacatgggcgcctgctgcatccccaccaagctgaggcccatgagcatgctgtactacgacgacggccagaacatcatcaagaaggacatccagaacatgatcgtggaggagtgcggctgcagctaa

SEQ ID NO: 145 - DNA, 351 bp, Artificial Sequence
  SYNTHETIC CONSTRUCT
ggcctggagtgcgacggcaaggtgaacctgtgctgcaagaagcaggacttcgtgagcttcaaggacatcggctggaacgactggatcatcgcccccagcggctaccacgccaacaggtgcgacggcctgtgccccagccacatcgccggcaccagcggcagcagcctgagcttccacagcaccgtgatcaaccactacaggatgaggggccacagccccttcagcgacatgggcagctgctgcgtgcccaccaagctgaggcccatgagcatgctgtactacgacgacggccagaacatcatcaagaaggacatccagaacatgatcgtggaggagtgcggctgcagctaa

SEQ ID NO: 146 - DNA, 351 bp, Artificial Sequence
  SYNTHETIC CONSTRUCT
ggcctggagtgcgacggcaaggtgaacatctgctgcaagaagcagctgttcggcaggaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccgtggccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 147 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagaggatggtggtggaggagtgcggctgcacctaa

SEQ ID NO: 148 - DNA, 351 bp, Artificial Sequence
  SYNTHETIC CONSTRUCT
ggcctggagtgcgacggcaaggtgaacatctgctgcaagaagcagctgttcggcaagaccaaggacatcggctggaacgactggatcatcgcccccagcggctaccacggcggcagctgcaccggcgagtgccccagccacatcgccggcaccagcggcagcagcctgagcttccacagcaccgtgatcaaccactacaggatgaggggccacagccccaacgccaacctgaagagctgctgcagccccaccaagctgaggcccatgagcatgctgtactacgacgacggccagaacatcatcaagaaggacatccagggcatgaaggtggaggagtgcggctgcacctaa

SEQ ID NO: 149 - DNA, 351 bp, Artificial Sequence
  SYNTHETIC CONSTRUCT
ggcctggagtgcgacggcaaggtgaacatctgctgcaagaagcaggagttcggccaggccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagggcatgaaggtggaggagtgcggctgcacctaa

SEQ ID NO: 150 - DNA, 351 bp, Artificial Sequence
  SYNTHETIC CONSTRUCT
ggcctggagtgcgacggcaaggtgaacatctgctgcaagaagcagagcttcgcccagaccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 151 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagggcatggtggtggaggagtgcggctgcacctaa

SEQ ID NO: 152 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcagctgcaccggcgagtgccccagccacatcgccggcaccagcggcagcagcctgagcttccacagcaccgtgatcaaccactacaggatgaggggccacagccccttcgccaacctgaagagctgctgcgcccccaccaagctgaggcccatgagcatgctgtactacgacgacggccagaacatcatcaagaaggacatccagaacatggtggtggaggagtgcggctgcgtgtaa

SEQ ID NO: 153 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 154 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 155 - DNA, 351 bp, Artificial Sequence
  SYNTHETIC CONSTRUCT
ggcctggagtgcgacggcaaggtgaacatctgctgcaagaagcagctgttcggccagaccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccaacgccaacctgaagagctgctgcgcccccaccaagctgaggcccatgagcatgctgtactacgacgacggccagaacatcatcaagaaggacatccagggcatgaaggtggaggagtgcggctgcgtgtaa

SEQ ID NO: 156 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcgcccccaccaagctgaggcccatgagcatgctgtactacgacgacggccagaacatcatcaagaaggacatccagaacatggtggtggaggagtgcggctgcgtgtaa

SEQ ID NO: 157 - DNA, 351 bp, Artificial Sequence
  SYNTHETIC CONSTRUCT
ggcctggagtgcgacggcaaggtgaacatctgctgcaagaagcagctgttcggccagaccaaggacatcggctggaacgactggatcatcgcccccagcggctaccacggcggcagctgcaccggcgagtgccccagccacatcgccggcaccagcggcagcagcctgagcttccacagcaccgtgatcaaccactacaggatgaggggccacagccccaacgccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 158 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 159 - DNA, 351 bp, Artificial Sequence
  SYNTHETIC CONSTRUCT
ggcctggagtgcgacggcaaggtgaacatctgctgcaagaagcagagcttcagccaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagggcatggtggtggaggagtgcggctgcacctaa

SEQ ID NO: 160 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagggcatggtggtggaggagtgcggctgcacctaa

SEQ ID NO: 161 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcagctgcagcggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagggcatgaaggtggaggagtgcggctgcacctaa

SEQ ID NO: 162 - DNA, 351 bp, Artificial Sequence
  SYNTHETIC CONSTRUCT
ggcctggagtgcgacggcaaggtgaacatctgctgcaagaagcagatgttcggccaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 163 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 164 - DNA, 351 bp, Artificial Sequence
  SYNTHETIC CONSTRUCT
ggcctggagtgcgacggcaaggtgaacatctgctgcaagaagcagagcttcggcaaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagggcatgaaggtggaggagtgcggctgcacctaa

SEQ ID NO: 165 - DNA, 351 bp, Artificial Sequence
  SYNTHETIC CONSTRUCT
ggcctggagtgcgacggcaaggtgaacatctgctgcaagaagcagagcttcggcaagaccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagcagatggtggtggaggagtgcggctgcacctaa

SEQ ID NO: 166 - DNA, 351 bp, Artificial Sequence
  SYNTHETIC CONSTRUCT
ggcctggagtgcgacggcaaggtgaacatctgctgcaagaagcagctgttcggccaggccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccgtggccaacctgaagagctgctgcagccccaccaagctgaggcccatgagcatgctgtactacgacgacggccagaacatcatcaagaaggacatccagggcatggtggtggaggagtgcggctgcacctaa

SEQ ID NO: 167 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 168 - DNA, 351 bp, Artificial Sequence
  SYNTHETIC CONSTRUCT
ggcctggagtgcgacggcaaggtgaacatctgctgcaagaagcagctgttcggccaggccaaggacatcggctggaacgactggatcatcgcccccagcggctaccacggcggcagctgcaccggcgagtgccccagccacatcgccggcaccagcggcagcagcctgagcttccacagcaccgtgatcaaccactacaggatgaggggccacagccccaacgccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 169 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagggcatggtggtggaggagtgcggctgcacctaa

SEQ ID NO: 170 - DNA, 351 bp, Artificial Sequence
  SYNTHETIC CONSTRUCT
ggcctggagtgcgacggcaaggtgaacatctgctgcaagaagcagagcttcggcagggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagggcatggtggtggaggagtgcggctgcacctaa

SEQ ID NO: 171 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaggatgaaggtggaggagtgcggctgcacctaa

SEQ ID NO: 172 - DNA, 351 bp, Artificial Sequence
  SYNTHETIC CONSTRUCT
ggcctggagtgcgacggcaaggtgaacatctgctgcaagaagcagctgttcggccagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccaacgccaacctgaagagctgctgcagccccaccaagctgaggcccatgagcatgctgtactacgacgacggccagaacatcatcaagaaggacatccagggcatgaaggtggaggagtgcggctgcacctaa

SEQ ID NO: 173 - DNA, 351 bp, Artificial Sequence
  SYNTHETIC CONSTRUCT
ggcctggagtgcgacggcaaggtgaacatctgctgcaagaagcagatgttcggcaaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 174 - DNA, 351 bp, Artificial Sequence
  SYNTHETIC CONSTRUCT
ggcctggagtgcgacggcaaggtgaacatctgctgcaagaagcagctgttcggccaggccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccgtggccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 175 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaggatggtggtggaggagtgcggctgcacctaa

SEQ ID NO: 176 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcgcccccaccaagctgaggcccatgagcatgctgtactacgacgacggccagaacatcatcaagaaggacatccagaacatgaaggtggaggagtgcggctgcgtgtaa

SEQ ID NO: 177 - DNA, 351 bp, Artificial Sequence
  SYNTHETIC CONSTRUCT
ggcctggagtgcgacggcaaggtgaacatctgctgcaagaagcagctgttcggcaagaccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccgtggccaacctgaagagctgctgcagccccaccaagctgaggcccatgagcatgctgtactacgacgacggccagaacatcatcaagaaggacatccagaggatggtggtggaggagtgcggctgcacctaa

SEQ ID NO: 178 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagggcatgaaggtggaggagtgcggctgcacctaa

SEQ ID NO: 179 - DNA, 351 bp, Artificial Sequence
  SYNTHETIC CONSTRUCT
ggcctggagtgcgacggcaaggtgaacatctgctgcaagaagcagagcttcggcagggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagggcatgaaggtggaggagtgcggctgcacctaa

SEQ ID NO: 180 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 181 - DNA, 351 bp, Artificial Sequence
  SYNTHETIC CONSTRUCT
ggcctggagtgcgacggcaaggtgaacatctgctgcaagaagcagctgttcggccaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccgtggccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 182 - DNA, 351 bp, Artificial Sequence
  SYNTHETIC CONSTRUCT
ggcctggagtgcgacggcaaggtgaacatctgctgcaagaagcagagcttcggcaagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 183 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcagctgcagcggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 184 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 185 - DNA, 351 bp, Artificial Sequence
  SYNTHETIC CONSTRUCT
ggcctggagtgcgacggcaaggtgaacatctgctgcaagaagcagctgttcggccagaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccaacgccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 186 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcagctgcagcggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 187 - DNA, 351 bp, Artificial Sequence
  SYNTHETIC CONSTRUCT
ggcctggagtgcgacggcaaggtgaacatctgctgcaagaagcagagcttcggcaggaccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaacatggtggtggaggagtgcggctgcacctaa

SEQ ID NO: 188 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcagcggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagggcatgaaggtggaggagtgcggctgcacctaa

SEQ ID NO: 189 - DNA, 351 bp, Artificial Sequence
  SYNTHETIC CONSTRUCT
ggcctggagtgcgacggcaaggtgaacatctgctgcaagaagcagagcttcggccagaccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagccccttcgccaacctgaagagctgctgcagccccaccaagctgaggcccatgagcatgctgtactacgacgacggccagaacatcatcaagaaggacatccagaacatgaaggtggaggagtgcggctgcacctaa

SEQ ID NO: 190 - DNA, 351 bp, Artificial Sequence
  SYNTHETIC CONSTRUCT
ggcctggagtgcgacggcaaggtgaacatctgctgcaagaagcagagcttcggccaggccaaggacatcggctggaacgactggatcatcgcccccagcggctaccacggcggcggctgcaccggcgagtgccccagccacatcgccggcaccagcggcagcagcctgagcttccacagcaccgtgatcaaccactacaggatgaggggccacagcccctgggccaacctgaagagctgctgcagccccaccaagctgaggcccatgagcatgctgtactacgacgacggccagaacatcatcaagaaggacatccagaggatggtggccgaggagtgcggctgcacctaa

SEQ ID NO: 191 - protein, 348 aa, Artificial Sequence
  SYNTHETIC CONSTRUCT
MAWVWTLLFLMAAAQSIQAGSHHHHHHGSGSGSGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 192 - protein, 321 aa, Artificial Sequence
  SYNTHETIC CONSTRUCT
MAWVWTLLFLMAAAQSIQAGSHHHHHHGSGSGSGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCN

SEQ ID NO: 193 - protein, 182 aa, Artificial Sequence
  SYNTHETIC CONSTRUCT
MAWVWTLLFLMAAAQSIQAGSHHHHHHGSGSGSETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIK

SEQ ID NO: 194 - DNA, 1047 bp, Artificial Sequence
  SYNTHETIC CONSTRUCT
atggcttgggtgtggaccttgctattcctgatggcagctgcccaaagtatccaagcaggctcccatcaccatcaccaccatggaagcggatccgggtcagggaactgttggctgaggcaagcgaagaacggcagatgtcaggtgctgtacaagaccgagctgagtaaggaggaatgctgcagtacgggcaggttgagcactagctggactgaagaggacgtcaacgacaacacgctgttcaagtggatgatcttcaatggcggagctcccaattgcatcccctgcaaagagacctgcgaaaacgtcgactgtggaccgggcaagaaatgcaggatgaacaagaagaacaagcccagatgcgtgtgtgctccagattgcagcaacatcacctggaaaggccccgtgtgtggcctcgatgggaagacctaccgcaatgagtgcgcccttctgaaggcacgatgcaaggagcagccagaactggaggtgcagtaccagggtaggtgcaagaagacctgtagggacgtcttctgccctggatcttccacttgcgtggtggatcagaccaacaacgcttactgcgtgacatgcaaccgtatctgcccagaacccgcctctagcgaacagtacctgtgcggtaatgacggagtcacctactctagtgcctgccacttgaggaaggccacatgtctgctcggtaggagcattggtctggcttacgagggcaagtgcatcaaggccaagtcttgcgaggacatacagtgtacgggtgggaagaagtgcctttgggacttcaaagtggggagagggagatgcagtctctgtgacgaactgtgtcccgattccaagtccgatgaacccgtgtgcgcgtccgataacgcgacctatgcctcagaatgcgccatgaaagaggcagcctgttctagcggagttctgctcgaggttaagcacagcggtagctgcaactccatctcagaggacactgaggaggaagaggaagacgaggatcaggactactcctttccgatcagctccatccttgagtggtaa

SEQ ID NO: 195 - DNA, 966 bp, Artificial Sequence
  SYNTHETIC CONSTRUCT
atggcttgggtgtggaccttgctattcctgatggcagctgcccaaagtatccaagcaggctcccatcaccatcaccaccatggaagcggatccgggtcagggaactgttggctgaggcaagcgaagaacggcagatgtcaggtgctgtacaagaccgagctgagtaaggaggaatgctgcagtacgggcaggttgagcactagctggactgaagaggacgtcaacgacaacacgctgttcaagtggatgatcttcaatggcggagctcccaattgcatcccctgcaaagagacctgcgaaaacgtcgactgtggaccgggcaagaaatgcaggatgaacaagaagaacaagcccagatgcgtgtgtgctccagattgcagcaacatcacctggaaaggccccgtgtgtggcctcgatgggaagacctaccgcaatgagtgcgcccttctgaaggcacgatgcaaggagcagccagaactggaggtgcagtaccagggtaggtgcaagaagacctgtagggacgtcttctgccctggatcttccacttgcgtggtggatcagaccaacaacgcttactgcgtgacatgcaaccgtatctgcccagaacccgcctctagcgaacagtacctgtgcggtaatgacggagtcacctactctagtgcctgccacttgaggaaggccacatgtctgctcggtaggagcattggtctggcttacgagggcaagtgcatcaaggccaagtcttgcgaggacatacagtgtacgggtgggaagaagtgcctttgggacttcaaagtggggagagggagatgcagtctctgtgacgaactgtgtcccgattccaagtccgatgaacccgtgtgcgcgtccgataacgcgacctatgcctcagaatgcgccatgaaagaggcagcctgttctagcggagttctgctcgaggttaagcacagcggtagctgcaactaa

SEQ ID NO: 196 - DNA, 549 bp, Artificial Sequence
  SYNTHETIC CONSTRUCT
atggcttgggtgtggaccttgctattcctgatggcagctgcccaaagtatccaagcaggctcccatcaccatcaccaccatggaagcggatccgggtcagagacctgcgaaaacgtcgactgtggaccgggcaagaaatgcaggatgaacaagaagaacaagcccagatgcgtgtgtgctccagattgcagcaacatcacctggaaaggccccgtgtgtggcctcgatgggaagacctaccgcaatgagtgcgcccttctgaaggcacgatgcaaggagcagccagaactggaggtgcagtaccagggtaggtgcaagaagacctgtagggacgtcttctgccctggatcttccacttgcgtggtggatcagaccaacaacgcttactgcgtgacatgcaaccgtatctgcccagaacccgcctctagcgaacagtacctgtgcggtaatgacggagtcacctactctagtgcctgccacttgaggaaggccacatgtctgctcggtaggagcattggtctggcttacgagggcaagtgcatcaagtaa